(19) United States
(12) Patent Application Publication
(10) Pub. No.: US 2006/0024806 A1
(43) Pub. Date: Feb. 2, 2006

(54) Mycolactone locus: an assembly line for producing novel polyketides, therapeutic and prophylactic uses

(76) Inventors: Timothy P. Stinear, Benvich (AU); Stewart T. Cole, Clamart (FR); Peter F. Leadlay, Cambridge (GB); Pamela L.C. Small, Knoxville, TN (US); Paul D.R. Johnson, Clifton Hill (AU); Grant Adam Jenkin, Kew (AU); John K. Davies, Ferntree Gully (AU); Stephen F. Haydock, Cottenham (GB)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/175,689

(22) Filed: Jul. 7, 2005

Related U.S. Application Data
(63) Continuation of application No. 10/987,592, filed on Nov. 15, 2004.
(60) Provisional application No. 60/519,864, filed on Nov. 14, 2003.

(51) Int. Cl. C07H 21/04; C12N 9/02

(52) U.S. Cl. 435/189; 536/23.2

(57) ABSTRACT

The present invention relates to Mycobacterium ulcerans virulence plasmid, pMUM001 and particularly to a cluster of genes carried by this plasmid that encode polyketide synthases (PKSs) and polyketide-modifying enzymes necessary and sufficient for mycolactone biosynthesis. More particularly this invention is directed to novel purified or isolated polypeptides, the polynucleotides encoding such polypeptides, processes for production of such polypeptides, antibodies generated against these polypeptides, the use of such polynucleotides and polypeptides in Diagnostic methods, kits, vaccines, therapy and for the production of mycolactone derivatives or novel polyketides by combinatorial synthesis.

### CROSS-REFERENCE TO RELATED APPLICATIONS

This application is based on and claims the benefit of U.S. Provisional Application No. 60/519,864, filed Nov. 14, 2003, (Attorney Docket No. 3495.6096) The entire disclosure of this application is relied upon and incorporated by reference herein.

### FIELD OF THE INVENTION

The present invention relates to Mycobacterium ulcerans virulence plasmid, pMUM001 and particularly to a cluster of genes carried by this plasmid that encode polyketide synthases (PKSs) and polyketide-modifying enzymes necessary and sufficient for mycolactone biosynthesis. More particularly this invention is directed to novel purified or isolated polypeptides, the polynucleotides encoding such polypeptides, processes for production of such polypeptides, antibodies generated against these polypeptides, the use of such polynucleotides and polypeptides in diagnostic methods, kits, vaccines, therapy and for the production of mycolactone derivatives or novel polyketides by combinatorial synthesis.

### BACKGROUND OF THE INVENTION

Biosynthesis of complex polyketides in bacteria is accomplished on so-called modular polyketide synthases (PKSs), giant multienzymes which constitute molecular assembly lines in which each set or module of fatty acid synthase-related activities governs a single specific cycle of polyketide chain extension (Rawlings B J: Biosynthesis of polyketides (other than actinomycete macrolides). Nat. Prod. Rep. (1999) 16:425-84. Rawlings B J: Type I polyketide biosynthesis in bacteria (Part A—erythromycin biosynthesis). Nat. Prod. Rep. (2001) 18:190-227; Rawlings B J: Type I polyketide biosynthesis in bacteria (Part B). Nat. Prod. Rep. (2001) 18:231-281; Staunton J, Weissman K J: Polyketide biosynthesis: a millennium review. Nat. Prod. Rep. (2001) 18:380-416).

For classical modular PKSs, the paradigm is the erythromycin PKS, or DEBS, which synthesises 6-deoxyerythronolide B (DEB) the aglycone core of the antibiotic erythromycin A in Saccharopolyspora erythraea. (Cortés J. et al.: An unusually large multifunctional polypeptide in the erythromycin-producing polyketide synthase of Saccharopolyspora erythraea. Nature (1990) 348:176-178; Donadio S. et al.: Modular organization of genes required for complex polyketide biosynthesis. Science (1991) 252:675-679.

The paradigm was extended in 1995 with the disclosure of the rapamycin PKS from Streptomyces hygroscopicus, which utilises a starter unit derived from shikimate, catalyses 14 cycles of polyketide chain extension, and then inserts an amino acid unit utilising an extension module from a non-ribosomal peptide synthetase (NRPS) (Schwecke T, et al.: The biosynthetic cluster for the polyketide immunosuppressant rapamycin. Proc. Natl. Acad. Sci. USA 1995, 92:7839-7843.). The molecular logic of polyketide and peptide assembly thus allows the biosynthesis of mixed polyketide-peptides, and other examples of this have since been disclosed, including bleomycin, epothilone, myxalamid and leinamycin (Du L, Shen, B: Biosynthesis of hybrid peptide-polyketide natural products. Curr. Opin. Drug Discov. Devel. (2001) 4:215-28; Staunton J, Wilkinson B: Combinatorial biosynthesis of polyketides and nonribosomal peptides. Curr. Opin. Chem. Biol. 2001 5:159-164).

Non-classical modular PKSs are exemplified by the so-called PksX from Bacillus subtilis, identified from genome sequencing and whose polyketide product is unknown (Albertini A M, et al.: Sequence around the 159 degrees region of the Bacillus subtilis genome: the pksX locus spans 33.6 kb. Microbiology 1995, 141:299-309); by TA antibiotic from Myxococcus xanthus (Paitan Y, et al.: The first gene in the biosynthesis of the polyketide antibiotic TA of Myxococcus xanthus codes for a unique PKS module coupled to a peptide synthetase. J. Mol. Biol. 1999, 286:465-474); by pederin from a bacterial symbiont of Paederus beetles (Piel J: A polyketide synthase-peptide synthetase gene cluster from an uncultured bacterial symbiont of Paederus beetles. Proc. Natl. Acad. Sci. USA 2002, 99:14002-14007); by the antibiotic mupirocin from Pseudomonas sp. (El-Sayed AKet al.: Characterization of the mupirocin biosynthesis gene cluster from Pseudomonas fluorescens NCIMB 10586. Chem. Biol. 2003, 10:419-430.); and by leinamycin from a Streptomyces sp. (Cheng Y G, et al.: Type I polyketide synthase requiring a discrete acyltransferase for polyketide biosynthesis. Proc. Natl. Acad. Sci USA 2003, 100:3149-3154.). In these PKS gene clusters the encoded module constitution is not so regular or as well understood as in the classical modular PKS multienzymes; and in particular none of the modules contains an AT domain. Rather, the AT activity is supplied in trans by a discrete AT enzyme, which has malonyl-CoA:ACP transferase activity; and the variation in sidechains of the polyketide is achieved not through selection of methylmalonyl-CoA as an extender unit in specific extension modules rather than malonyl-CoA but rather by the inclusion of an S-adenosylmethionine-dependent methyltransferase domain in specific extension modules.

Other non-classical modular PKSs are known in which the number of modules is fewer than the observed number of extension cycles achieved, and there is evidence that the synthesis is achieved by one module “stuttering”, that is, carrying out either two or three cycles rather than the conventional single cycle of chain extension, before passing the elongated chain to the next extension module in the PKS. In the case of the lankacidin PKS, it appears that more than one copy of certain modules may be utilised within the multienzyme assembly (Mochizuki Set al.: The large linear plasmid pSLA2-L of Streptomyces rochei has an unusually condensed gene organization for secondary metabolism. Mol. Microbiol. 2003, 48:1501-1510).

For all of these enzyme systems, the characteristic use, in a substantial part of the polyketide assembly, of different sets of enzymes for initiation and for each cycle of chain extension, means that they are capable of genetic manipulation to produce altered products, by the methods already established for the engineering of classic modular PKSs.

The engineering of modular PKSs to create hybrids was disclosed in 1996 (WO9801546; WO9801571; US5876991; and in subsequent publications Oliynyk, Met al.: A hybrid modular polyketide synthase obtained by domain swapping. Chem. Biol. (1996) 3: 833-839). The essence of this approach is to splice one or more contiguous domains, or one or more contiguous modules from a natural PKS into a second natural PKS, in such a way that the splice sites or junctions are made in the linker regions between domains, or in the conserved amino acid sequence at the margins of domains. This approach has been widely exemplified in the last few years (WO9849315), subsequently, these same technologies have been used to create a collection of hybrid PKSs based on the erythromycin PKS and which produce different altered 14-membered macrolides in recombinant cells (see e.g. WO0024907). This collection of recombinants constitutes a small library of modular PKSs. The productivity of these recombinant strains was determined to vary from reasonable to essentially zero (McDaniel R, et al: Multiple genetic modifications of the erythromycin polyketide synthase to produce a library of novel ‘unnatural’ natural products. Proc. Nat. Acad. Sci. USA (1999) 96:1846-1851.). A number of other improvements have been published or disclosed but in general the hybrid multienzymes so generated are less active than the parent PKSs in polyketide biosynthesis (Yoon, Y J et al. Generation of multiple bioactive macrolides by hybrid modular polyketide synthases in Streptomyces venezuelae Chem Biol. (2002) 9:203-14).

The reasons for the diminished productivity of such hybrid PKSs have been widely examined and discussed. There are several chief factors considered to play a role. One factor relates to the level of enzyme present: the expression of the hybrid PKS in the chosen recombinant cell may be suboptimal, and/or the protein may fold incorrectly or fail to dimerise to form the active enzyme. This aspect of construction of hybrid PKSs has been addressed by a number of conventional approaches and it is not considered further here. Similarly, there may be suboptimal levels of required chemical precursor molecules present in the recombinant cell, and obvious routes to optimise these are well-established in the art (Roberts G A, et al: Heterologous expression in Escherichia coli of an intact multienzyme component of the erythromycin-producing polyketide synthase. Eur. J. Biochem. (1993) 214: 305-311; Kao C M, et al.: Engineered biosynthesis of a complete macrolactone in a heterologous host. Science (1994) 265: 509-512. Pfeifer B A, et al.: Biosynthesis of complex polyketides in a metabolically engineered strain of E. coli. Science (2001) 291:1790-1792).

A second factor is that because of local unfavourable protein: protein interactions which inevitably arise between the heterologous domains which have been brought into apposition by the engineering, the structure is distorted from the conformation which is required for activity, and in particular for the essential passing on of the growing substrate chain from one active site to the next which is the essential feature of these multienzyme synthases. Thus the rapamycin PKS catalyses in total some 80 reactions at separate active sites before the product is released, and if any one of these individual reactions fails the overall process will fail. In the absence of detailed structural information for any modular PKS, the contribution of this factor is hard to quantify, but the person skilled in the art would be well aware that it constitutes a real barrier to success.

A third factor is that the key enzyme in each extension module, the ketosynthase (KS) which catalyses the C—C bond forming reaction between the growing polyketide chain and the incoming extension unit, is believed to have evolved to exhibit a definite substrate specificity and stereospecificity for both reaction partners. Thus, the KS of extension module N of a modular PKS is believed to catalyse the transfer to itself of the polyketide chain residing on the ACP domain of the upstream extension module N-1, only when the polyketide acyl chain bome by the ACP has achieved the correct level of reduction. Premature transfer would be expected to lead to a mixture of products which is not generally seen. Likewise, if the stereochemistry of the polyketide acyl chain is incorrect, or its pattern of substitution is incorrect, it is believed that the KS will discriminate against loading of that acyl group. A second stage of discrimination will operate for the condensation reaction itself, and if the structure of either the extension unit or of the polyketide acyl unit is different from that naturally processed by the KS domain of module N then this will decrease the rate of reaction. Published studies on purified modular PKS domains in vitro have provided evidence that such substrate specificity and stereospecificity is indeed an important feature of those PKSs which have so far been studied, which include the DEBS and the pikromycin PKS (Chen S, et al.: Mechanisms of molecular recognition in the pikromycin polyketide synthase. Chem. Biol. 2000, 7:907-918; Beck, B J et al.: Substrate recognition and channeling of monomodules from the pikromycin polyketide synthase. J Am Chem Soc. (2003) 125:12551-7).

Similar considerations are likely to apply to the other enzymes in the module: the ketoreductase (KR), dehydrase (DH) and enoylreductase (ER) enzymes are all believed to exercise a specificity and selectivity towards their substrates. However, the KS-ACP interaction is believed to be the key determinant in efficient intermodule transfer and processing of intermediates (Ranganathan A, et al.: Knowledge-based design of bimodular and trimodular polyketide synthases based on domain and module swaps: a route to simple statin analogues. Chem. Biol. (1999) 6:731-741; Wu N, et al.: Quantitative analysis of the relative contributions of donor acyl carrier proteins, acceptor ketosynthases, and linker regions to intermodular transfer of intermediates in hybrid polyketide synthases. Biochemistry 2002, 41:5056-5066).

The person skilled in the art would be aware that there are available several methods of improvement of enzyme activity by forced or directed evolution via gene shuffling and allied technologies. Such methods rely absolutely on the existence of an assay or screen enabling “successful” variant enzymes to be identified and isolated for further rounds of improvement. However, such methods without undue experimentation are unlikely to lead to a combinatorial library of hybrid modular PKSs which have high catalytic activity, because of the difficulty of simultaneously optimising up to 20 critical KS domains for the broadest possible specificity while also optimising inter-modular protein:protein contacts between up to 20 modules which may be heterologous to each other.

The person skilled in the art would also be aware that methods have been introduced for the site-specific mutagenesis of individual active sites in a modular PKS, with the aim of reducing the impact of unfavourable protein:protein interactions which are caused when entire domains are swapped to create hybrid PKSs. Thus, it has been disclosed (WO0214482 (2002; WO0314312 (2003).) that the active site of the AT domains of DEBS can be altered by site-specific mutagenesis so as to alter the specificity for the extension unit or for the starter unit. Analogously the KR domains of modular PKS are known to belong to the same enzyme family of short-chain dehydrogenases as the tropinone reductases and it has been shown that the stereospecificity of reduction of tropinone can be switched by site-directed mutagenesis (Nakajima, K et al.: Site-directed mutagenesis of putative substrate-binding residues reveals a mechanism controlling the different stereospecificities of two tropinone reductases. J Biol. Chem. (1999) Jun 4; 274:16563-8.) so it would now be obvious to the person skilled in the art that such methods could be employed for modular PKSs. However, such approaches are unlikely without undue experimentation to lead to the desired combinatorial library of hybrid modular PKSs, and are more appropriate for improvement of an individual hybrid PKS synthesising a desired product.

In summary, although it has been appreciated in the prior art that there are serious problems with currently available methods of constructing functional combinatorial libraries of modular PKSs, no one has had any idea how to discover or develop such PKSs. Neither was it anticipated that any natural modular PKS would be discovered that inherently possessed such properties.

There remains an urgent need to develop efficient ways of generating such combinatorial libraries of functional modular PKSs which in turn in appropriate settings (either in vivo or in vitro) efficiently produce polyketide compounds which are themselves biologically active or which can be transformed by well-known processes of post-PKS enzymatic modification into valuable bioactive substances (references to publications on glycosylation engineering and other post-PKS steps). By modular PKSs is meant here not only classical modular PKSs but also non-classical modular PKSs and mixed PKS-NRPS modular systems.

The present invention discloses the existence and detailed structural organisation of the entire biosynthetic gene cluster governing the biosynthesis of mycolactone, a polyketide toxin from Mycobacterium ulcerans (MU). Mycobacterium ulcerans, an emerging human pathogen harboured by aquatic insects, is the causative agent of Buruli ulcer, a devastating skin disease rife throughout Central and West Africa. A single Buruli ulcer, which can cover more than 15% of a person's skin surface, contains huge numbers of extracellular bacteria. Despite their abundance and extensive tissue damage there is a remarkable absence of an acute inflammatory response to the bacteria and the lesions are often painless (1). This unique pathology is attributed to mycolactone, a macrolide toxin consisting of a polyketide side chain attached to a 12-membered core that appears to have cytotoxic, analgesic and immunosuppressive activities. Its mode of action is unclear but in a guinea pig model of the disease, purified mycolactone injected subcutaneously reproduces the natural pathology and mycolactone negative variants are avirulent implying a key role for the toxin in pathogenesis (2).

### SUMMARY OF INVENTION

The present invention concerns the characterization of the genes cluster governing the biosynthesis of mycolactone and carried by the Mycobacterium ulcerans plasmid pMUM001.

More precisely, this invention encompasses a purified or isolated polynucleotide comprising the DNA sequence of SEQ ID NO:1-6 and a purified or isolated polynucleotide encoding the polypeptide of amino acid sequence SEQ ID NO:7-12. The invention also encompasses polynucleotides complementary to these sequences, double-stranded polynucleotides comprising the DNA sequence of SEQ ID NO:1-6 and of polynucleotides encoding the polypeptides of amino acid sequence SEQ ID NO:7-12. Both single-stranded and double-stranded RNA and DNA polynucleotides are encompassed by the invention. These molecules can be used as probes to detect both single-stranded and double-stranded RNA and DNA variants for encoding polypeptides of amino acid sequence SEQ ID NO:7-12. A double-stranded DNA probe allows the detection of polynucleotides equivalent to either strand of the DNA probe.

Purified or isolated polynucleotides that hybridize to a denatured, double-stranded DNA comprising the DNA sequence of SEQ ID NO:1-6 or a purified or isolated polynucleotide encoding the polypeptide of amino acid sequence SEQ ID NO:7-12 under conditions of high stringency are encompassed by the invention.

The invention further encompasses purified or isolated polynucleotides derived by in vitro mutagenesis from polynucleotides of sequence SEQ ID NO:1-6. In vitro mutagenesis includes numerous techniques known in the art including, but not limited to, site-directed mutagenesis, random mutagenesis, and in vitro nucleic acid synthesis.

The invention also encompasses purified or isolated polynucleotides of sequence degenerate from SEQ ID NO:1-6 as a result of the genetic code, purified or isolated polynucleotides, which are allelic variants of polynucleotides of sequence SEQ ID NO:1-6 or a species-homolog thereof.

The purified or isolated polynucleotides of the invention, which include DNA and RNA, are referred to herein as “MLS polynucleotide”.

The invention also encompasses recombinant vectors that direct the expression of these MLS polynucleotides and host cells transformed or transfected with these vectors.

An object of the present invention is to provide an isolated or purified polypeptide comprising an amino acid sequence encoded by the MLS polynucleotides as described above and/or biologically active fragments thereof.

A further object of the invention is to provide an isolated or purified polypeptide having at least 80% sequence identity with amino acid sequence of SEQ ID NO:7-12.

The purified or isolated polypeptides of the invention are referred to herein as “MLS polypeptides.”

This invention also provides labeled MLS polypeptides. Preferably, the labeled polypeptides are in purified form. It is also preferred that the unlabeled or labeled polypeptide is capable of being immunologically recognized by human body fluid containing antibodies to MU. The polypeptides can be labeled, for example, with an immunoassay label selected from the group consisting of radioactive, enzymatic, fluorescent, chemiluminescent labels, and chromophores.

The invention further encompasses methods for the production of MLS polypeptides, including culturing a host cell under conditions promoting expression, and recovering the polypeptide from the culture medium. Especially, the expression of MLS polypeptides in bacteria, yeast, plant, and animal cells is encompassed by the invention.

Purified polyclonal or monoclonal antibodies that bind to MLS polypeptides are encompassed by the invention.

Immunological complexes between the MLS polypeptides of the invention and antibodies recognizing the polypeptides are also provided. The immunological complexes can be labeled with an immunoassay label selected from the group consisting of radioactive, enzymatic, fluorescent, chemiluminescent labels, and chromophores.

Furthermore, this invention provides a method for detecting infection by MU. The method comprises providing a composition comprising a biological material suspected of being infected with MU, and assaying for the presence of MLS polypeptide of MU. The polypeptides are typically assayed by electrophoresis or by immunoassay with antibodies that are immunologically reactive with MLS polypeptides of the invention.

This invention also provides an in vitro diagnostic method for the detection of the presence or absence of antibodies, which bind to an antigen comprising a MLS polypeptide or mixtures of the MLS polypeptides. The method comprises contacting the antigen with a biological fluid for a time and under conditions sufficient for the antigen and antibodies in the biological fluid to form an antigen-antibody complex, and then detecting the formation of the immunological complex. The detecting step can further comprising measuring the formation of the antigen-antibody complex. The formation of the antigen-antibody complex is preferably measured by immunoassay based on Western blot technique, ELISA (enzyme linked immunosorbent assay), indirect immunofluorescent assay, or immunoprecipitation assay.

A diagnostic kit for the detection of the presence or absence of antibodies, which bind to a MLS polypeptide or mixtures of the MLS polypeptides, contains antigen comprising a MLS polypeptide, or mixtures of the MLS polypeptides, and means for detecting the formation of immune complex between the antigen and antibodies. The antigens and the means are present in an amount sufficient to perform the detection.

This invention also provides an immunogenic composition comprising a MLS polypeptide or a mixture thereof in an amount sufficient to induce an immunogenic or protective response in vivo, in association with a pharmaceutically acceptable carrier therefor. A vaccine composition of the invention comprises a protective amount of a MLS polypeptide or a mixture thereof and a pharmaceutically acceptable carrier therefor.

The polypeptides of this invention are thus useful as a portion of a diagnostic composition for detecting the presence of antibodies to antigenic proteins associated with MU.

In addition, the MLS polypeptides can be used to raise antibodies for detecting the presence of antigenic proteins associated with MU.

The polypeptides of the invention can be also employed to raise neutralizing antibodies that either inactivate MU, reduce the viability of MU in vivo, or inhibit or prevent bacterial replication. The ability to elicit MU-neutralizing antibodies is especially important when the polypeptides of the invention are used in immunizing or vaccinating compositions to activate the B-cell arm of the immune response or induce a cytotoxic T lymphocyte response (CTL) in the recipient host.

This invention provides a method for detecting the presence or absence of MU comprising:
(1) contacting a sample suspected of containing bacterial genetic material of MU with at least one nucleotide probe, and
(2) detecting hybridization between the nucleotide probe and the bacterial genetic material in the sample, wherein said nucleotide probe has a sequence complementary to the sequence of the purified or isolated polynucleotides of the invention or a part thereof.

In addition, this invention provides a process to produce variants of mycolactone comprising the following steps.
a) mutagenesis of the isolated or purified polynucleotide of any one of SEQ ID NOS:1-6,
b) expression of the said mutated polynucleotide in a Mycobacterium strain,
c) selection of Mycobacterium mutants altered in the production of mycolactone by DNA sequencing of and mass spectrometry,
d) culture of the selected transfected Mycobacterium, and
e) extraction of mycolactone variants from the culture of said culture. In a preferred embodiment, the isolated or purified polynucleotide has a nucleic acid sequence being at least 80% identical to the sequence SEQ ID NO:4 or fragments thereof.

Further, this invention provides a process to produce mycolactone in a fast-growing mycobacterium comprising the following steps:
a) cloning at least the three isolated polynucleotides comprising the DNA sequences of SEQ ID NO:1, 2 and 3 or three isolated polynucleotides that hybridize to either strand of denatured, double-stranded DNAs comprising the nucleotide sequences SEQ ID NO:1, 2 and 3 in a fast-growing mycobacterium,
b) expressing the isolated polynucleotides by growing the recombinant mycobacterium in appropiate culture conditions, and
c) purifying the produced mycolactone. In a preferred embodiment, the isolated polynucleotides comprise the DNA sequences of SEQ ID NO:1 to 6 or isolated polynucleotides that hybridize to either strand of denatured, double-stranded DNAs comprising the nucleotide sequences SEQ ID NO:1 to 6.

Sequences of polynucleotides and polypeptides of the invention are included in the drawings. The SEQ ID NO and corresponding Figure containing the sequence of the SEQ ID NO follows:

| FIG. | SEQ ID NO: |
|---|---|
| 6A-6Q | 1 |
| 7A-7C | 2 |
| 8A-8N | 3 |
| 9 | 4 |
| 10 | 5 |
| 11 | 6 |
| 12A-12E | 7 |
| 13 | 8 |
| 14A-14D | 9 |
| 15 | 10 |
| 16 | 11 |
| 17 | 12 |

### BRIEF SUMMARY OF THE DRAWINGS

This invention will be described with reference to the drawings in which

FIG. 1. Demonstration of the mycolactone plasmid. (A) Pulsed field gel electrophoresis and (B) Southern hybridization analyses of MU Agy99 (lanes 1 and 2) and MU 1615 (lanes 3 and 4), showing the presence of the linearised form of the plasmid in non-digested genomic DNA (lanes 1 and 3) and after digestion with XbaI (lanes 2 and 4), hybridized to a combination probe derived from mlsA, mlsB, mup038 and mup045. Lane M is the Lambda low-range DNA size ladder (NEB).

FIG. 2. Circular representation of pMUM001. The scale is shown in kilobases by the outer black circle. Moving in from the outside, the next two circles show forward and reverse strand CDS, respectively, with colours representing the functional classification (red, replication; light blue, regulation; light green; hypothetical protein; dark green, cell wall and cell processes; orange, conserved hypothetical protein; cyan, IS elements; yellow, intermediate metabolism; grey, lipid metabolism). This is followed by the GC skew (G-C)/(G+C) and finally the G+C content using a 1 kb window. The arrangement of the mycolactone biosynthetic cluster (mup053, mup045, mlsA1, mlsA2, mup038 and mlsB) has been highlighted and the location of all XbaI sites indicated. Hind III restriction sites are shown by H1: 1289, H2: 5209, H3: 71532, H4: 71846, H5: 73953, H6: 136357, H7: 136671, H8: 138778, H9: 152732, H10: 168846 and H11: 173190.

FIG. 3. Domain and module organisation of the mycolactone PKS genes. Within each of the three genes (mlsA1, mlsA2 and mlsB) different domains are represented by a numbered block. The domain designation is described in the key. White blocks represent inter-domain regions of 100% identity. Module arrangements are depicted below each gene and the modules are number coded to indicate identity both in function and sequence (>98%). For example module 5 of MLSA1 is identical to modules 1 and 2 of MLSB. The crosses through four of the DH domains indicate they are predicted to be inactive based on a point mutation in the active site sequence. The structure of mycolactone has also been number coded to match the module responsible for a particular chain extension.

FIG. 4. Mycolactone transposon mutants. Mycolactone negative mutants were identified as non-pigmented colonies (insert). 1×10⁷ bacteria and 50 μl culture filtrate were added to a semi-confluent monolayer of L929 fibroblasts for detection of cytotoxicity. Treated cells shown at 24 h. (A) MU1615::Tn104 containing an insertion in mlsB, (B) WT MU 1615, (C) Untreated control cells, (D) MU 1615::Tn141 containing an insertion in mlsA (20×).

FIG. 5. Mass spectroscopic analyses of the mycolactone transposon mutants. (A) MU1615::Tn104 containing an insertion in mlsB, showing the absence of the mycolactone ion m/z 765 and the presence of the lactone core ion at m/z 447, (B) WT MU 1615 showing the presence of the mycolactone ion m/z 765, (C) Control mutant MU1615::Tn99 containing a non-MLS insertion, showing the presence of the mycolactone ion m/z 765, (D) MU 1615::Tn141 containing an insertion in mlsA, showing the absence of both the mycolactone ion m/z 765 and the lactone core ion at m/z 447.

FIG. 6: nucleic acid sequence of the coding sequence of mlsA1 gene.

FIG. 7: nucleic acid sequence of the coding sequence of mlsA2 gene.

FIG. 8: nucleic acid sequence of the coding sequence of mlsB gene.

FIG. 9: nucleic acid sequence of the coding sequence of mup045 gene.

FIG. 10: nucleic acid sequence of the coding sequence of mup053 gene.

FIG. 11: nucleic acid sequence of the coding sequence of mup038 gene.

FIG. 12: amino acid sequence of the protein encoded by mlsA1 gene.

FIG. 13: amino acid sequence of the protein encoded by mlsA2 gene.

FIG. 14: amino acid sequence of the protein encoded by mlsB gene.

FIG. 15: amino acid sequence of the protein encoded by mup045 gene.

FIG. 16: amino acid sequence of the protein encoded by mup053 gene.

FIG. 17: amino acid sequence of the protein encoded by mup038 gene.

FIG. 18: complete sequence of Mycobacterium ulcerans plasmid pMUM001 (38 pages).

FIG. 19 is a linear map of pMUM001. The position of the 81 predicted protein-coding DNA sequences (CDS) is indicated as different coloured blocks, labelled sequentially as MUP001 (repA) through to MUP081. Forward and reverse strand CDS are shown above and below the black line respectively and the colours represent different functional classifications (red, replication; light blue, regulation; light green, hypothetical protein; dark green, cell wall and cell processes; orange, conserved hypothetical protein; cyan, insertion sequence elements; yellow, intermediate metabolism; grey, lipid metabolism). The black arrows indicate the region cloned into pcDNA2.1 to produce the shuttle vector pMUDNA2.1. The regions covered by the light grey, shaded boxes indicate 8 kb of identical nucleotide sequence, encompassing the start of the mycolactone PKS genes, mlsA1 and mlsB. The scale is given in bp and each minor division represents 1000 bp

FIG. 20 shows the replication origin of pMUM001. The beginning of the repA and MUP081 genes are marked in blue uppercase text and the direction of transcription is shown by the arrows. The sequence underlined (lower case and upper case) indicates a region of high nucleotide sequence conservation between pMUM001 and the M. fortuitum plasmid pJAZ38. The 70 bp sequence in shaded in green within this region is conserved among several mycobacterial plasmids (Picardeau et al., 2000). The 16 bp iteron sequences are shown in red and the partial inverted repeat of the iteron is shown in yellow.

FIG. 21 is a schematic representation of the mycobacterial/E. coli shuttle vector pMUDNA2.1, constructed as described in the methods section. The dotted line delineates the junction between the 6 kb fragment overlapping the putative ori of pMUM001 and pcDNA2.1. Unique restriction enzymes sites are marked. The grey inner segments represent the regions removed from the two deletion constructs pMUDNA2.1-1 and pMUDNA2.1-3.

FIG. 22 depicts the results of agarose gel electrophoresis (A) and Southern hybridization analysis (B) of SpeI-digested DNA from M. marinum M strain (lane 1) and M. marinum M strain transformed with pMUDNA2.1 (lane 2). Purified, SpeI-digested pMUDNA2.1 was included as a positive control (lane 3). The probe was derived from a 413 bp internal region the repA gene of pMUM001.

FIG. 23 depicts the stability of pMUDNA2.1 in M. marinum M strain grown in the absence of apramycin. The percentage of CFUs containing recombinant plasmid over successive time points are indicated by the persistence of cells resistant to apramycin; expressed as a percentage of the total number of CFUs in the absence of apramycin. For the total CFU counts, each time point is the mean±standard error for three biological repeats.

FIG. 24 is an analysis of the flanking sequences of ten copies of IS2404 in M. ulcerans strain Agy99. The ends of the 41 bp perfect inverted repeats are boxed and the intervening IS2404 sequence is inferred by a series of three dots within the boxed area. The different target site duplications are marked in underlined bold type-face.

FIG. 25 depicts the structures of mycolactone A (Z-4′,5′) and B ( ) ([M+Na]+ at m/z 765).

FIG. 26 is a dotter analysis of the pMUM001 DNA sequence, highlighting regions of repetitive DNA sequence. Direct repeat sequences are shown as lines running parallel to the main diagonal, while inverted repeats run perpendicular. The sites of homologous recombination surrounding the start of mlsA1 and mlsB that led to the creation of plasmid deletion derivatives are highlighted by the shaded circles.

FIG. 27 depicts mapping of the deletion variants of pMUM001.
A) Scaled, circular maps of pMUM001 and the two types of deletion derivative, with a proposed model for recombination-mediated deletion. The positions of all HindIII sites are marked. On the outer circles, the black arrows show the location of several key genes. The sites of recombination are encircled and indicated by the crossed, dotted lines. The inner grey circles show the sequences spanned by BAC clones. For the deletion derivatives, the HindIII sites where the vector pBeloBAC11 was cloned are also shown.
B) Expanded view of the regions of recombination within pMUM001 surrounding the loading modules at the start of mlsA1 and mlsB that gave rise to the deletion variants. All HindIII and PstI sites are marked. The grey shaded block between the dotted lines indicates the zone of 100% nucleotide indentity that was subject to recombination. The 200 bp sequence hybridizing to probe 74 is also shown.
C) Gel electrophoresis with the results of PstI RE digestion of 21 MUAgy99 BAC clones, showing the presence of two sub-families that span the mlsB and the mlsA genes, respectively.
D) Southern hybridization analysis of (C), confirming the presence of two copies of the mls loading module sequences in pMUM001 and single copies in the deletion variants. The 30 different sizes of the hybridizing bands are due to the sites of cloning into pBeloBAC11, which contains three PstI sites.

FIG. 28 shows the results of mapping of pMUM in seven MU strains.
A) PFGE and Southern hybridization with five, selected PCR-derived probes from pMUM001 against non-digested and XbaI-digested DNA, extracted from MU and M. marinum. Lane identification is as follows: Lane 1: MUAgy99; lane 2: MUKob; lane 3: MU1615; lane 4: MUChant; lane 5: MU105425; lane 6: MU5114; lane 7: MU941331; lane 8: M. marinum M strain.
B) Physical maps of pMUM for the seven MU strains, deduced from the Southern hybridization experiments shown in (A), showing plasmid size, the position of all XbaI sites and the toxin status of each strain as determined by LC-MS/MS. Question marks indicate that the exact region deleted from the mls locus could not be determined.

FIG. 29 depicts the results of LC-MS analysis of the lipid extract from the Australian isolate MUChant showing the absence of mycolactone ([M+Na]+: 765.5) and the presence of the non-hydroxylated mycolactone ([M+Na]+: 749.5). A) Ion trace for m/z=765.5; B) Ion trace for m/z=749.5.

FIG. 30 is a phylogenetic analysis of ten MU strains using selected plasmid markers.
(A) Alignment of 1266 bp sequences derived from the four concatenated pMUM protein-coding loci present in all ten MU strains. Only variable nucleotides are shown. A period indicates identity with the strain MU94133.
(B) Alignment of 2208 bp sequences derived from the seven concatenated pMUM protein-coding loci present in six MU strains.
(C) Neighbour-joining tree of the phylogenetic relationship among the ten MU strains, inferred from comparisons of the 1266 bp sequences.
(D) Neighbour-joining tree of the phylogenetic relationship among the six MU strains, inferred from comparisons of the 2208 bp sequences.
(E) Neighbour-joining tree of the phylogenetic relationship among six MU and five M. marinum genotypes as revealed by previous sequence analysis of seven chromosomally encoded protein-coding loci among 18 MU isolates and 22 M. marinum isolates (28).
(F) Clustal W alignment of the predicted aa sequences of a 348 bp region of MUP053 among the five MU strains positive for this gene.

FIG. 31 shows the structures of mycolactone A (Z-Δ^{4′,5′}) and B (E-Δ^{4′,5′}) from the African strain MUAgy99 (1) and from the Chinese strain MU98912 (2).

FIG. 32 depicts the MS/MS spectra of mycolactone precursor ions at m/z 765 (from MUAgy99) and at m/z 779, 777 and 761 (from MU98912).

FIG. 33 shows the proposed structures of fragment ions C, D and E from the MUAgy99 and of the corresponding fragment ions from the MU98912.

FIG. 34 is a schematic representation of the domain structure of extension modules 6 and 7 in MlsB from MUAgy99 and module 7 from MU98912, showing the position of the oligonucleotides used for PCR and the altered AT7 domain substrate specificity identified by DNA sequencing of the PCR product from strain MU98912 compared with strain MUAgy99.

FIG. 35 is an amino acid sequence comparison between the AT6 and AT7 domains of MUAgy99 with the AT7 domain of MU98912. The region of dark grey shading indicates the AT domain. Boxed sequences are residues known to be critical for AT substrate specificity. The light grey shading indicates the start of the DH domain.

FIG. 36 depicts the construction of novel MLS modules.

FIG. 37 depicts the arrangement of modified cosmid vector to support the expression of combinational polyketide libraries in E. coli.

### DETAILED DESCRIPTION OF THE INVENTION

1. Polynucleotides and Polypeptides

In a first embodiment, the present invention concerned isolated or purified polynucleotides encoding M. ulcerans enzymes involved in the biosynthesis of mycolactone, namely polyketide synthases and polyketide-modifying enzymes. The term “MLS polynucleotides”, as used herein, refers generally to the isolated or purified polynucleotides of the invention.

Therefore, the isolated or purified polynucleotide of the invention comprises at least one nucleic acid sequence which is selected among the sequences having at least 80% identity to part or all of SEQ ID NO:1-6 or among the nucleic acid sequences encoding the polypeptides of amino acid sequence SEQ ID NO:7-12.

As used herein, the terms “isolated or purified” means altered “by the hand of man” from its natural state, i.e., if it occurs in nature, it has been changed or removed from its original environment, or both. For example, a polynucleotide or a protein/peptide naturally present in a living organism is neither “isolated” nor purified, the same polynucleotide separated from the coexisting materials of its natural state, obtained by cloning, amplification and/or chemical synthesis is “isolated” as the term is employed herein. Moreover, a polynucleotide or a protein/peptide that is introduced into an organism by transformation, genetic manipulation or by any other recombinant method is “isolated” even if it is still present in said organism. The term “purified” as used herein, means that the polypeptides of the invention are essentially free of association with other proteins or polypeptides, for example, as a purification product of recombinant host cell culture or as a purified product from a non-recombinant source. The term “substantially purified” as used herein, refers to a mixture that contains MLS polypeptides and is essentially free of association with other proteins or polypeptides, but for the presence of known proteins that can be removed using a specific antibody, and which substantially purified MLS polypeptides can be used as antigens.

Amino acid or nucleic acid sequence “identity” and “similarity” are determined from an optimal global alignment between the two sequences being compared. An optimal global alignment is achieved using, for example, the Needleman-Wunsch algorithm (Needleman and Wunsch, 1970, J. Mol. Biol. 48:443-453). “Identity” means that an amino acid or nucleic acid at a particular position in a first polypeptide or polynucleotide is identical to a corresponding amino acid or nucleic acid in a second polypeptide or polynucleotide that is in an optimal global alignment with the first polypeptide or polynucleotide. In contrast to identity, “similarity” encompasses amino acids that are conservative substitutions. A “conservative” substitution is any substitution that has a positive score in the blosum62 substitution matrix (Hentikoff and Hentikoff, 1992, Proc. Natl. Acad. Sci. USA 89: 10915-10919). By the statement “sequence A is n % similar to sequence B” is meant that n % of the positions of an optimal global alignment between sequences A and B consists of identical residues or nucleotides and conservative substitutions. By the statement “sequence A is n % identical to sequence B” is meant that n % of the positions of an optimal global alignment between sequences A and B consists of identical residues or nucleotides.

As used herein, the term “polynucleotide(s)” generally refers to any polyribonucleotide or poly-deoxyribonucleotide, which may be unmodified RNA or DNA or modified RNA or DNA. This definition includes, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. “Polynucleotide(s)” embraces short polynucleotides or fragments often referred to as oligonucleotide(s). The term “polynucleotide(s)” as it is employed herein thus embraces such chemically, enzymatically or metabolically modified forms of polynucleotides, as well as the chemical forms of DNA and RNA characteristic of viruses and cells, including, for example, simple and complex cells which exhibits the same biological function as the polypeptides encoded by SEQ ID NO.1-6. The term “polynucleotide(s)” also embraces short nucleotides or fragments, often referred to as “oligonucleotides”, that due to mutagenesis are not 100% identical but nevertheless code for the same amino acid sequence.

Therefore, isolated or purified single strand polynucleotides comprising a sequence selected among SEQ ID NO:1-6 and the complementary sequences of SEQ ID NO:1-6, and isolated or purified multiple strands polynucleotides whose one strand comprises a sequence selected among SEQ ID NO:1-6 also form part of the invention.

Polynucleotides within the scope of the invention include isolated or purified polynucleotides that hybridize to the MLS polynucleotides disclosed above under conditions of moderate or severe stringency, and which encode MLS polypeptides. As used herein, conditions of moderate stringency, as known to those having ordinary skill in the art, and as defined by Sambrook et al. Molecular Cloning: A Laboratory Manual, 2 ed. Vol. 1, pp. 1.101-104, Cold Spring Harbor Laboratory Press, (1989), include use of a prehybridization solution for the nitrocellulose filters 5×SSC, 0.5% SDS, 1.0 mM EDTA (pH 8.0), hybridization conditions of 50% formamide, 6×SSC at 42° C. (or other similar hybridization solution, such as Stark's solution, in 50% formamide at 42° C.), and washing conditions of about 60° C., 0.5×SSC, 0.1% SDS. Conditions of high stringency are defined as hybridization conditions as above, and with washing at 68° C., 0.2×SSC, 0.1% SDS. The skilled artisan will recognize that the temperature and wash solution salt concentration can be adjusted as necessary according to factors such as the length of the probe.

The invention provides equivalent isolated or purified polynucleotides encoding MLS polypeptides that is degenerate as a result of the genetic code to the nucleic acid sequences SEQ ID NO:1-6. Equivalent polynucleotides can result from silent mutations (e.g., occurring during PCR amplification), or can be the product of deliberate mutagenesis of a sequence SEQ ID NO: 1-6. All these equivalent polynucleotides still encode a MLS polypeptide having the amino acid sequence of SEQ D NO:7-12 and then are included in the present invention.

The present invention further embraces isolated or purified fragments and oligonucleotides derived from the MLS polynucleotides as described above. These fragments and oligonucleotides can be used, for example, as probes or primers for the diagnostic of an infection by MU.

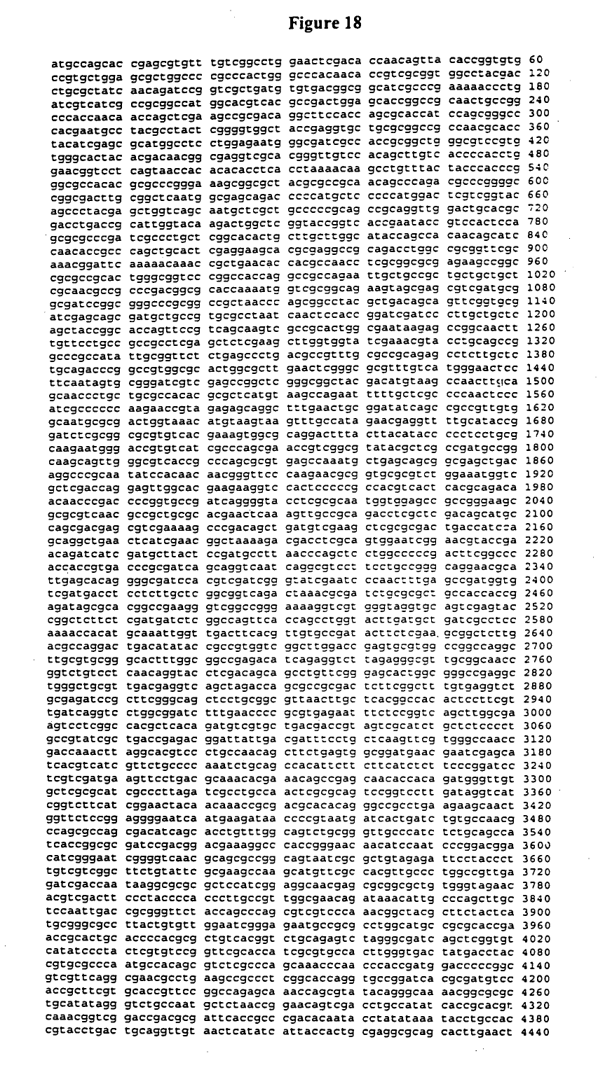

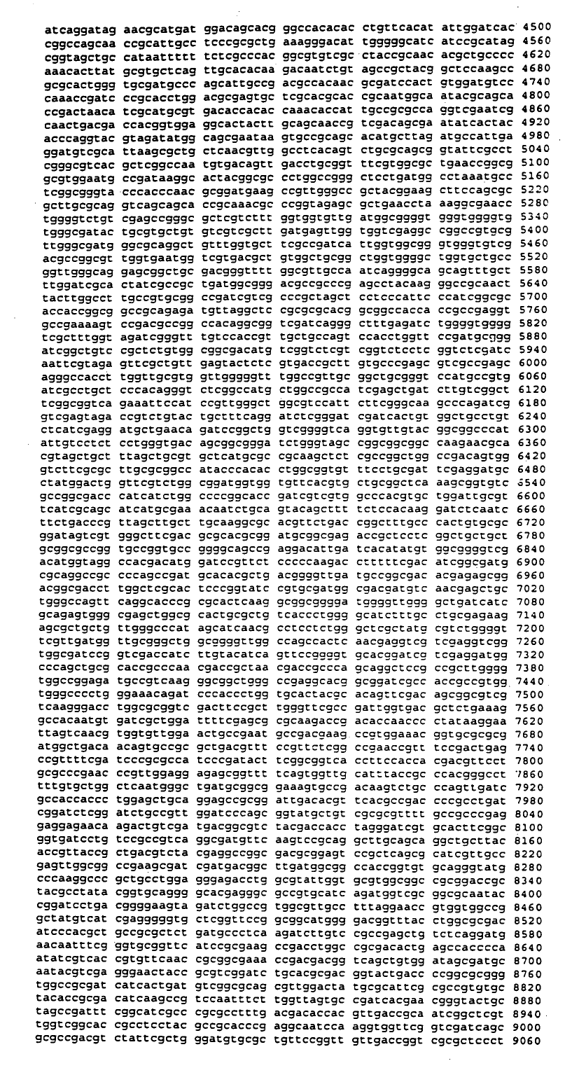

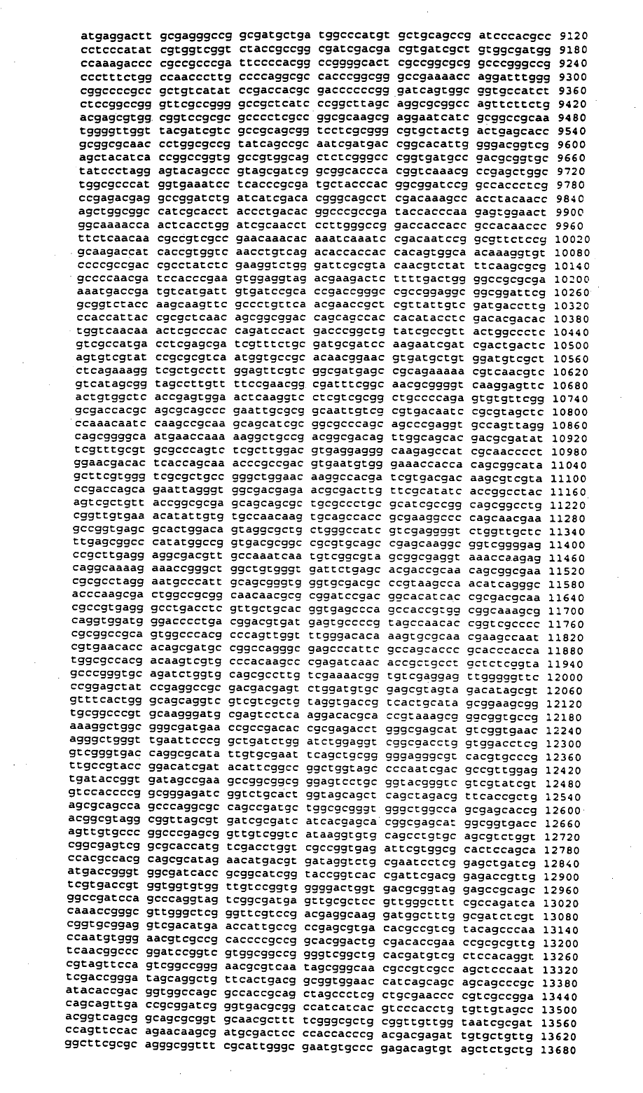

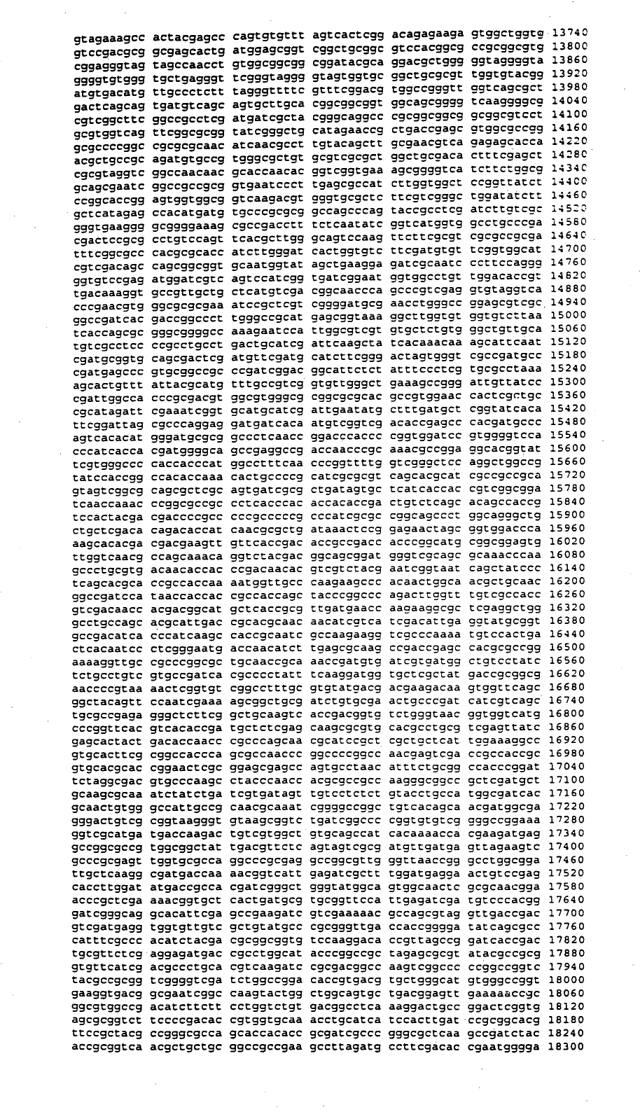

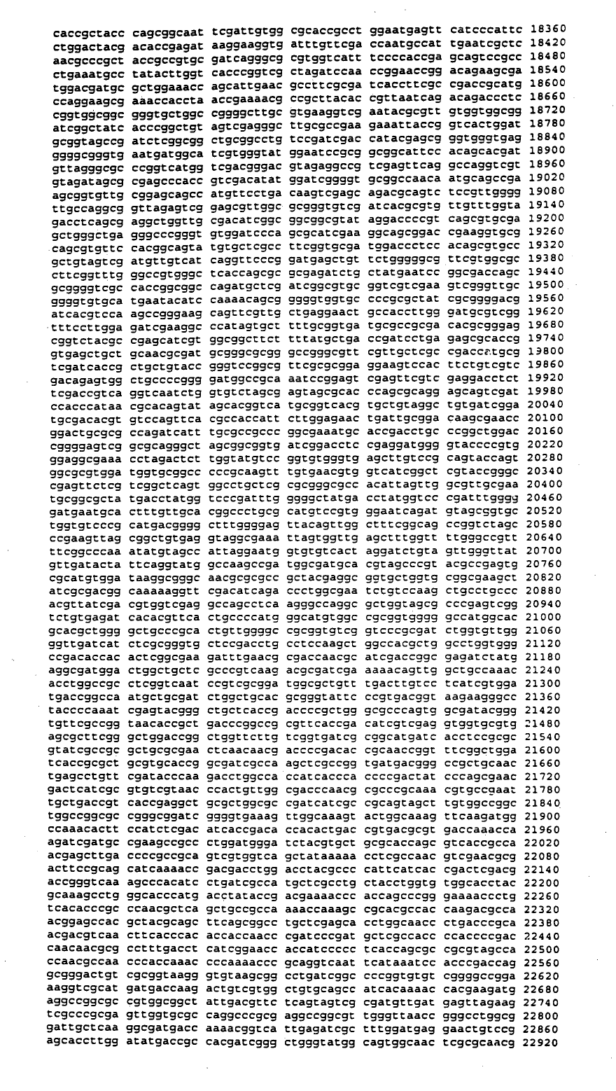

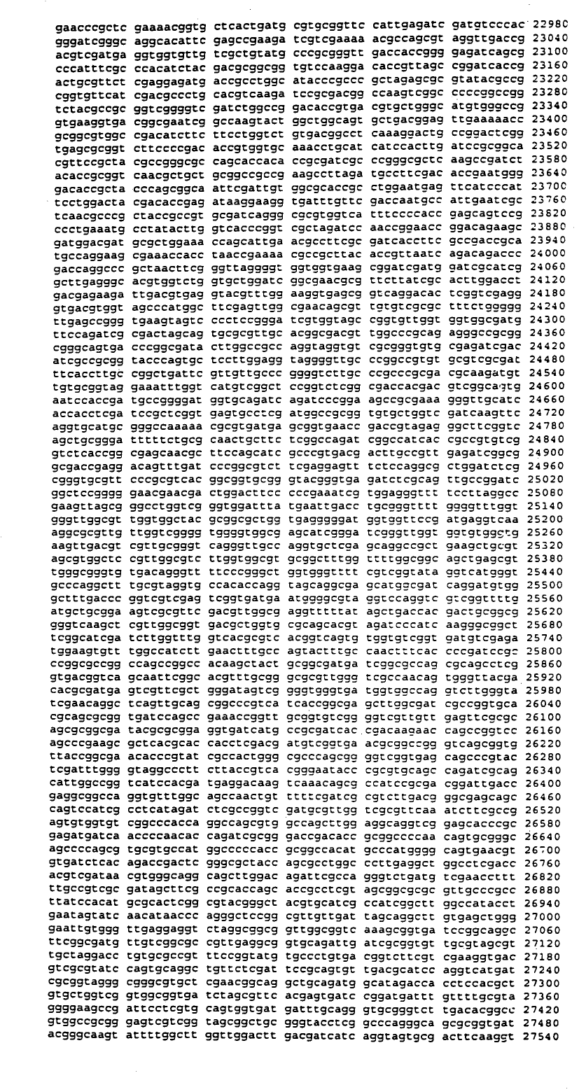

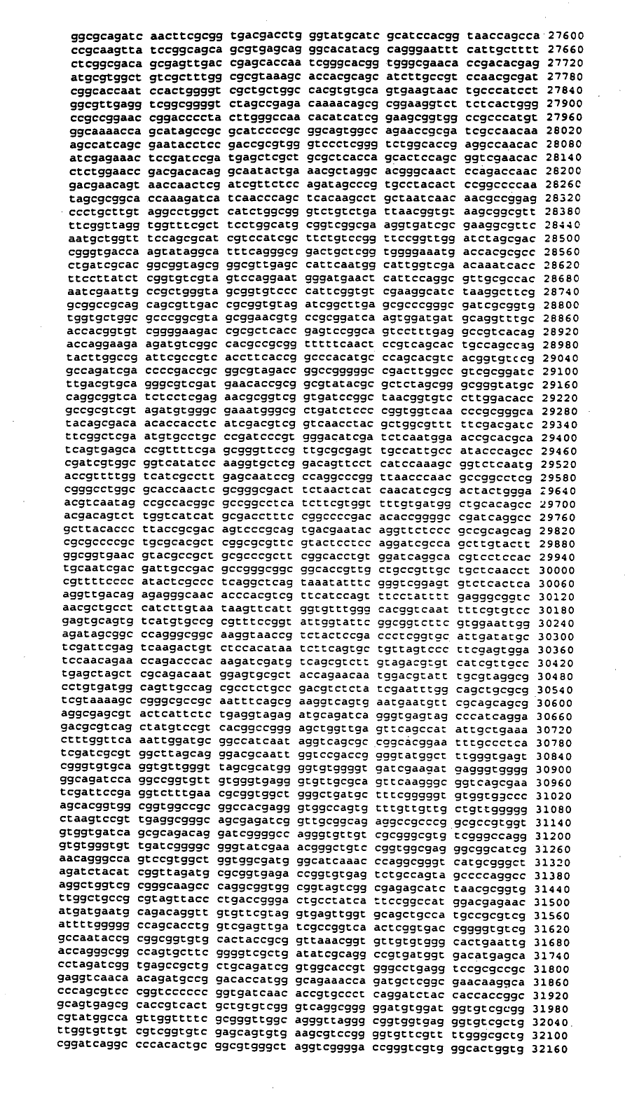

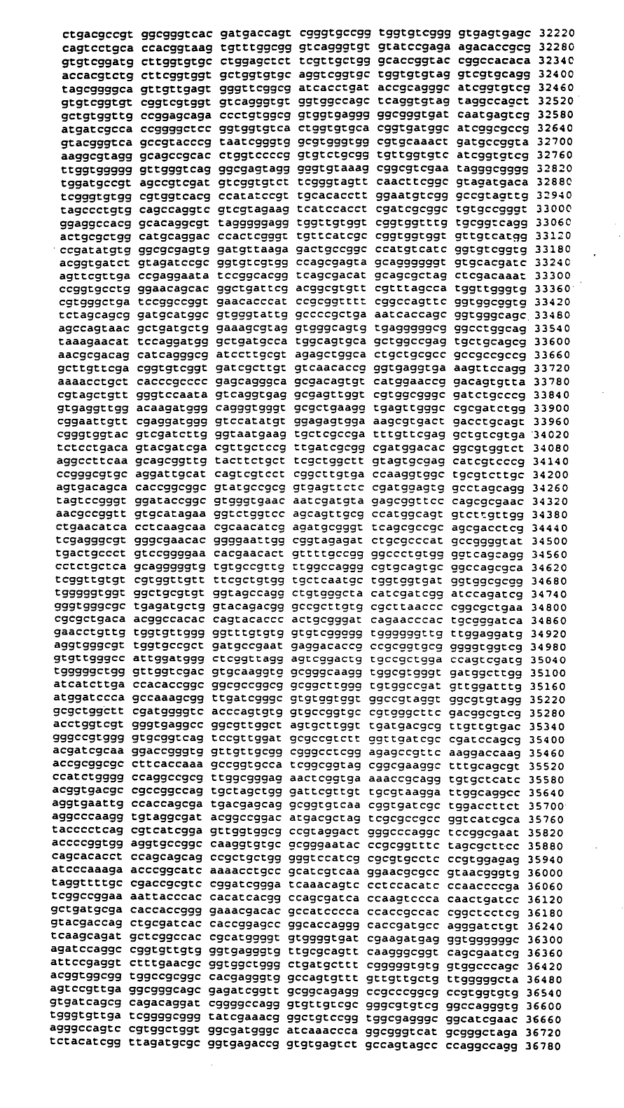

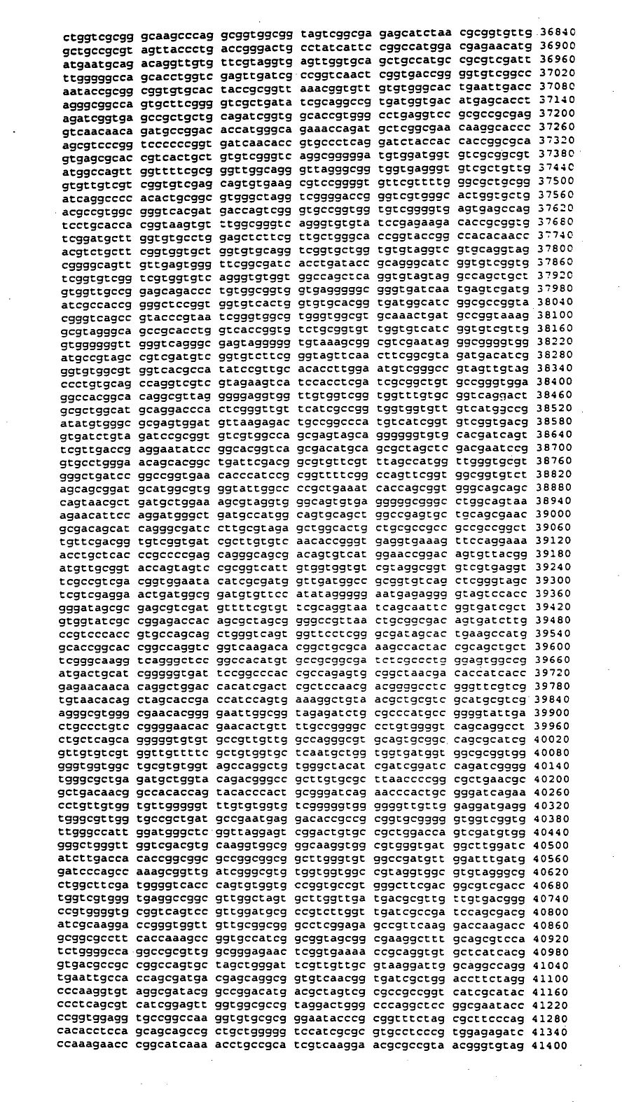

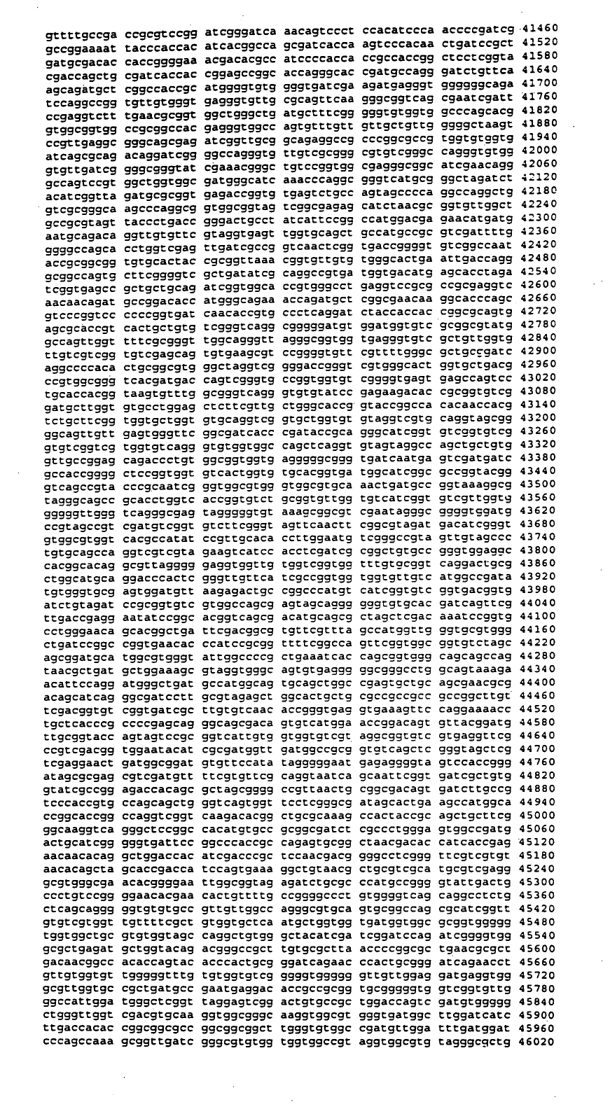

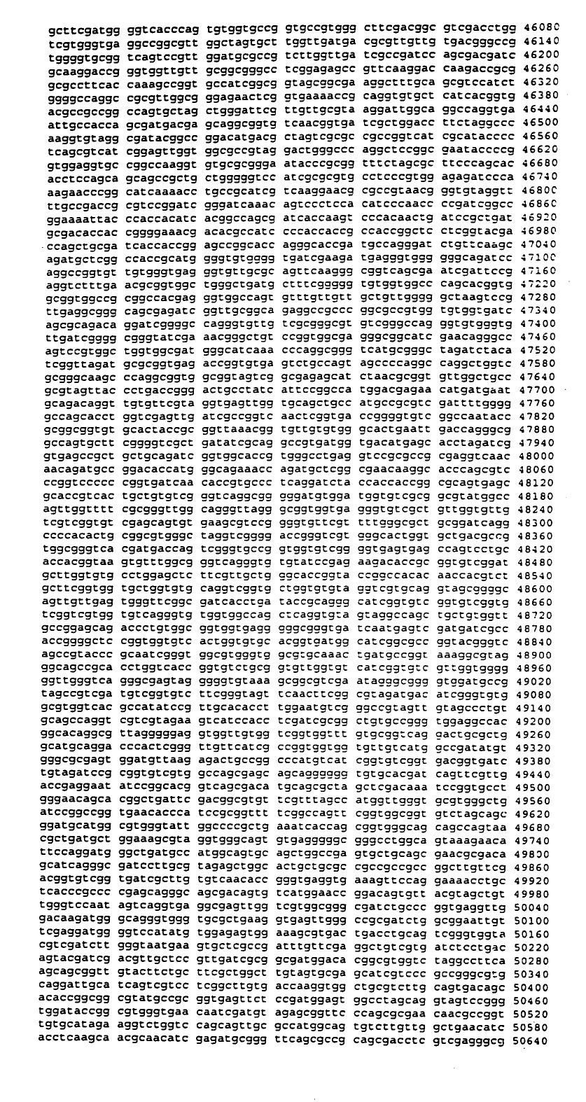

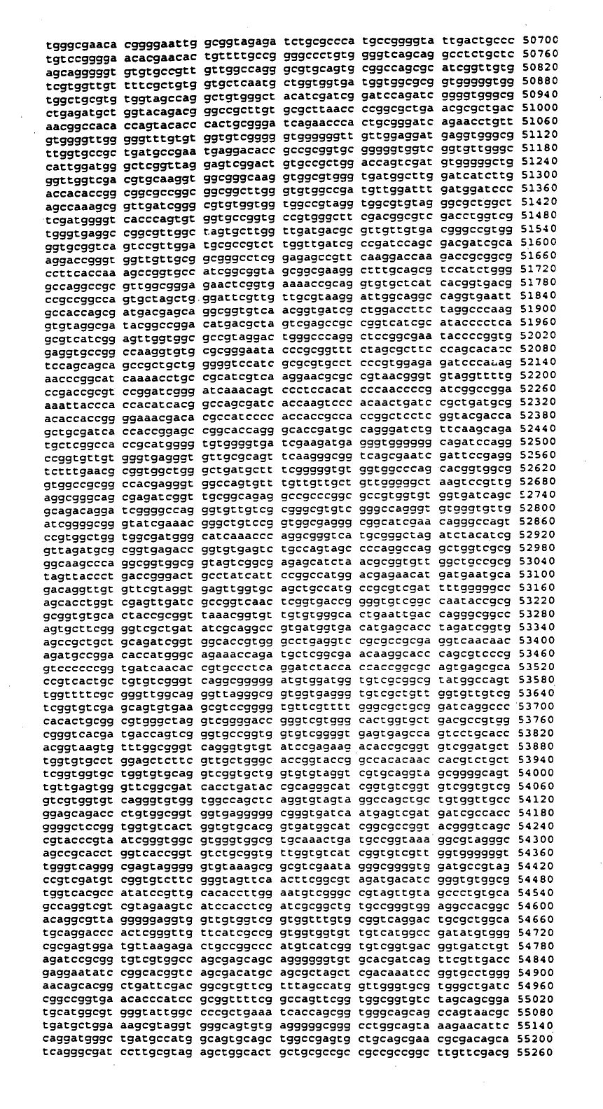

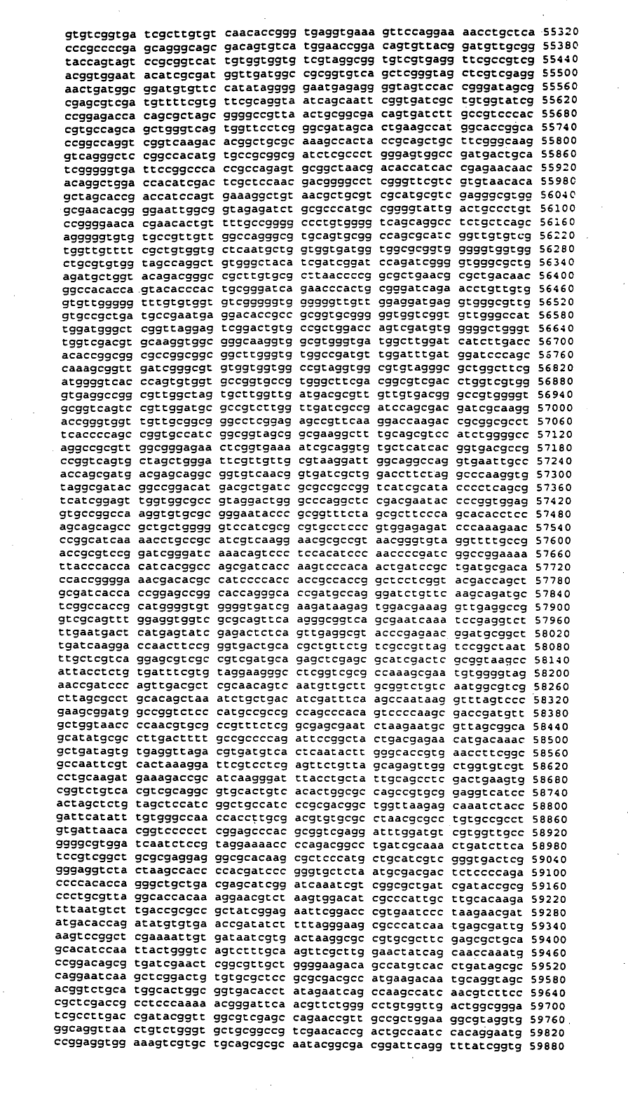

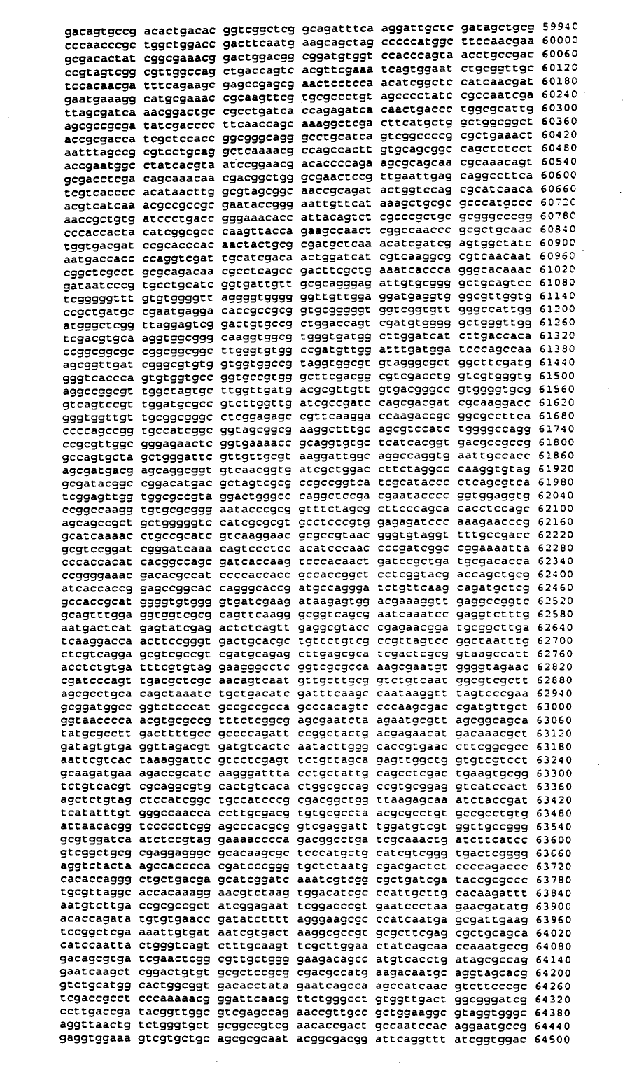

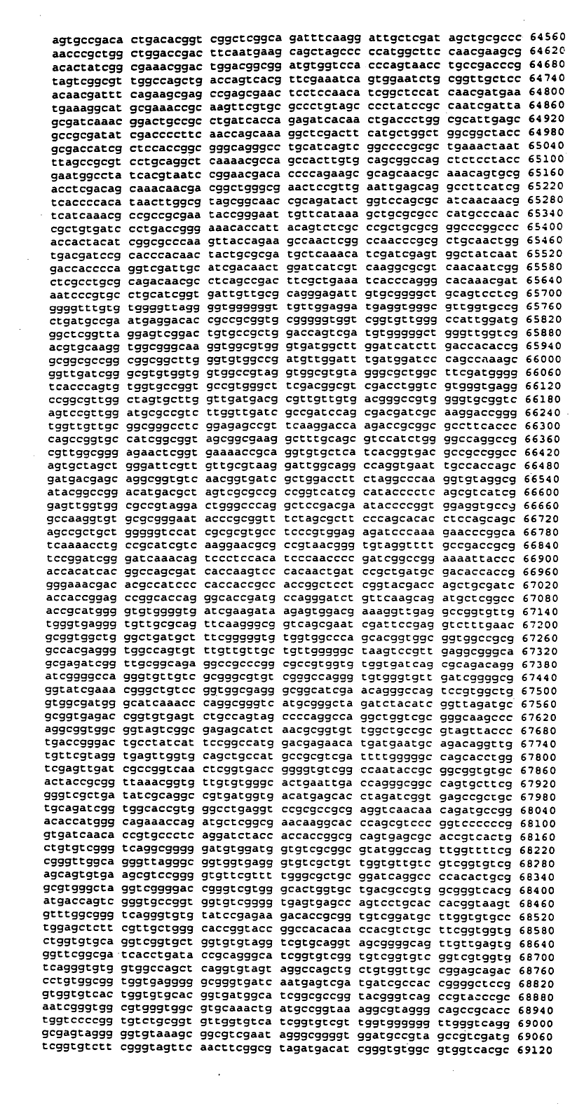

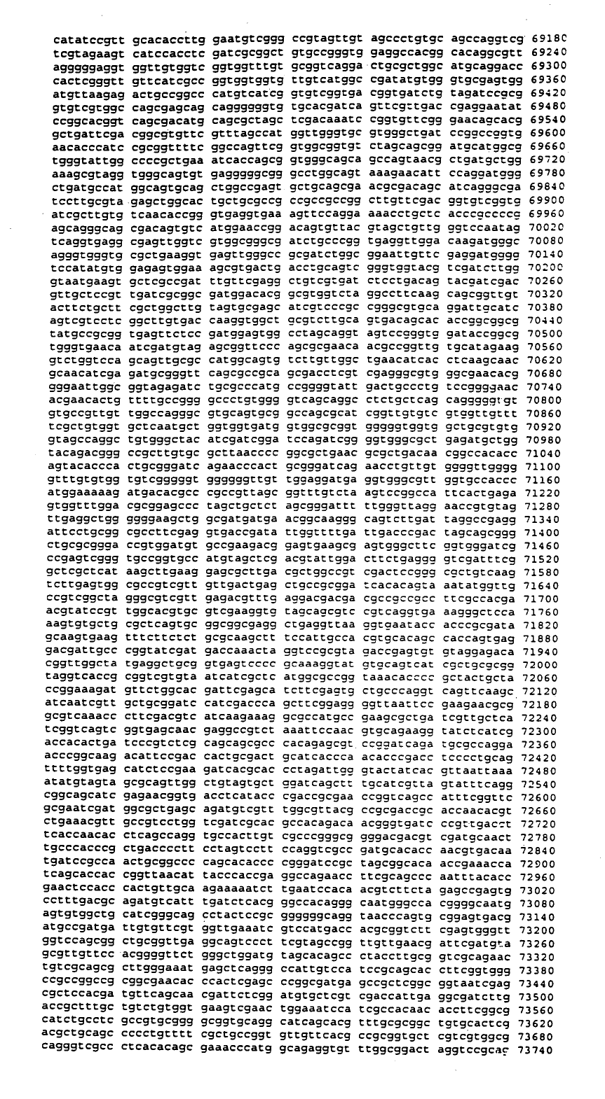

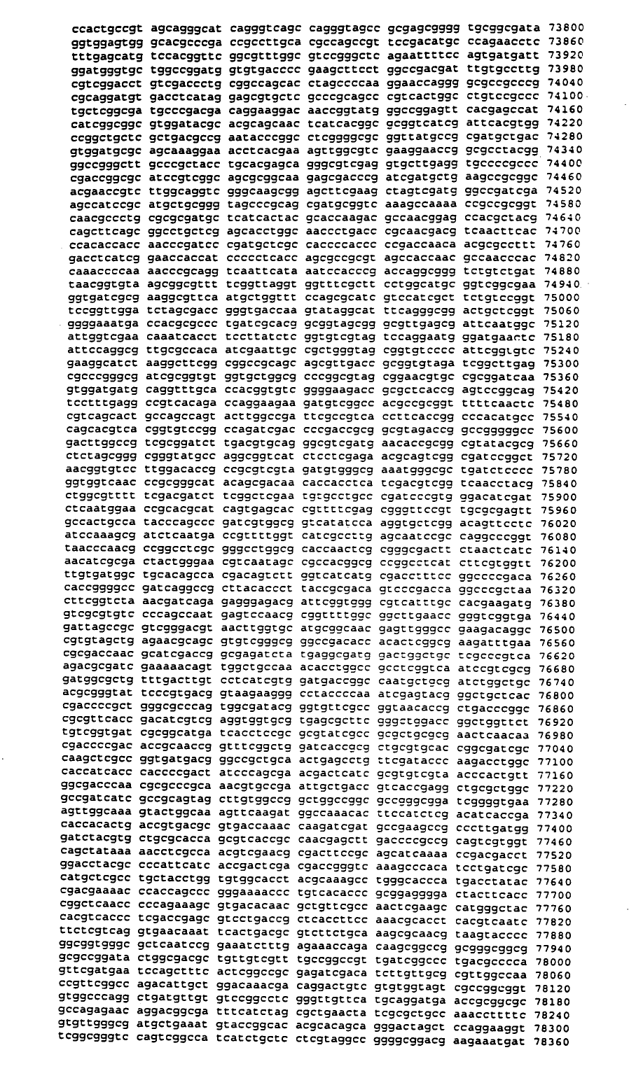

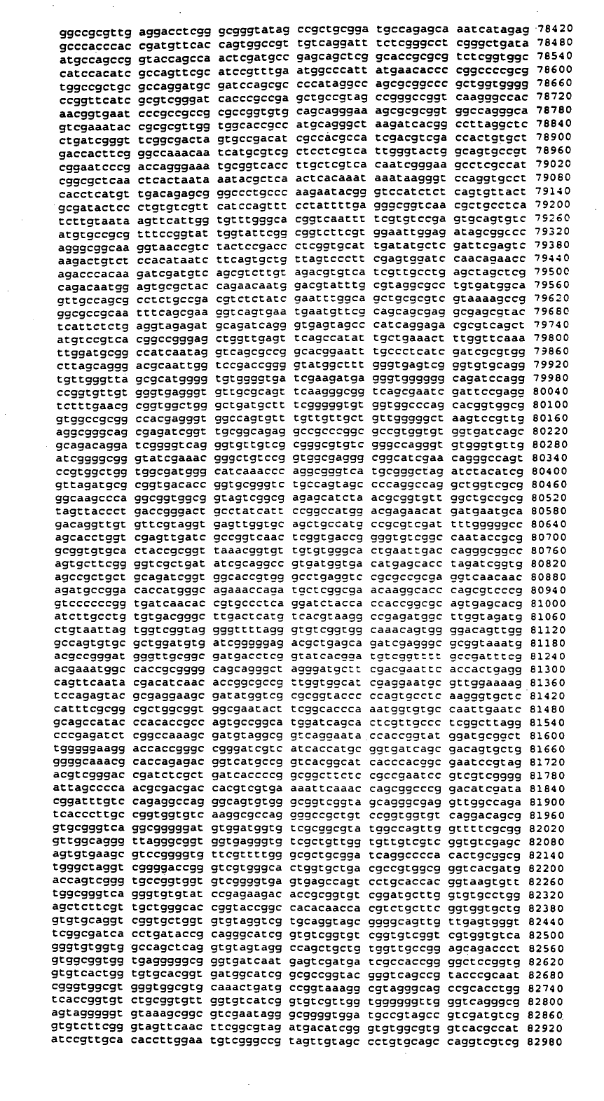

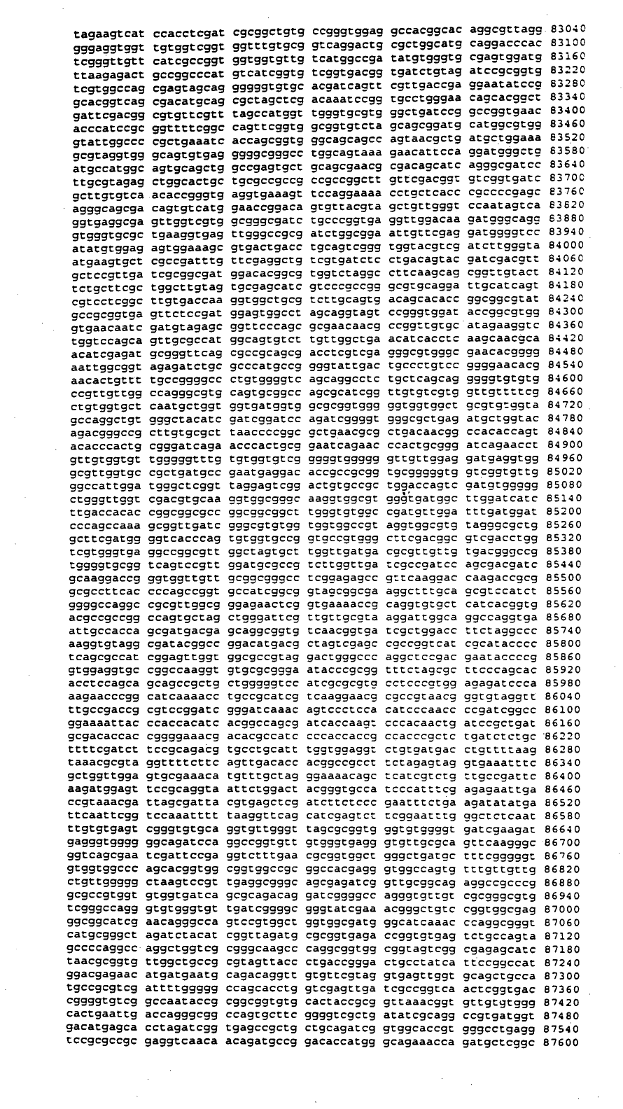

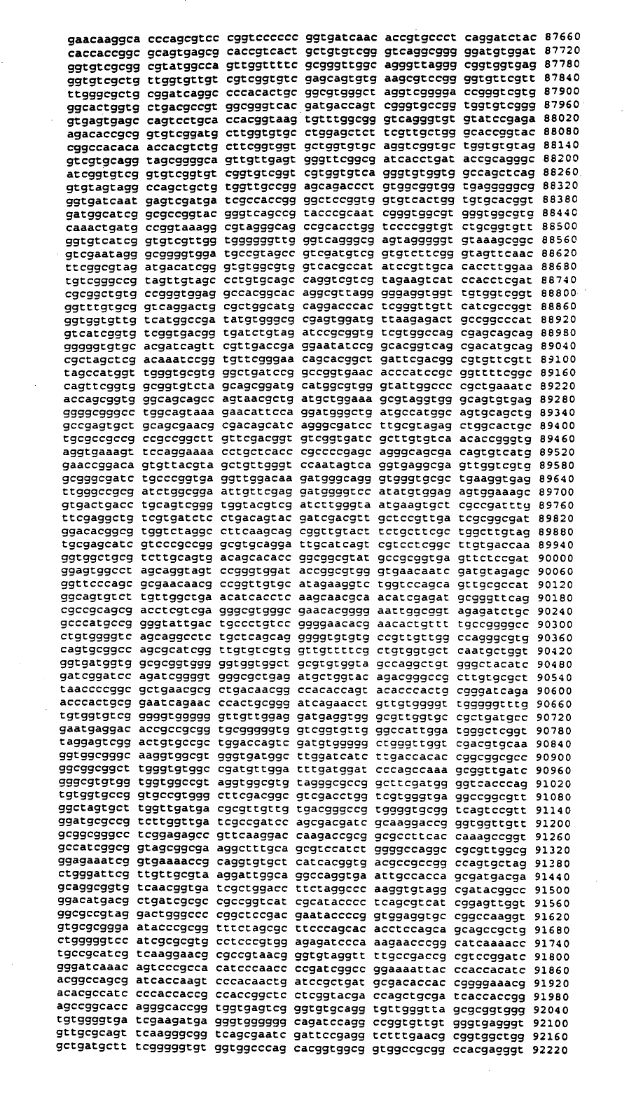

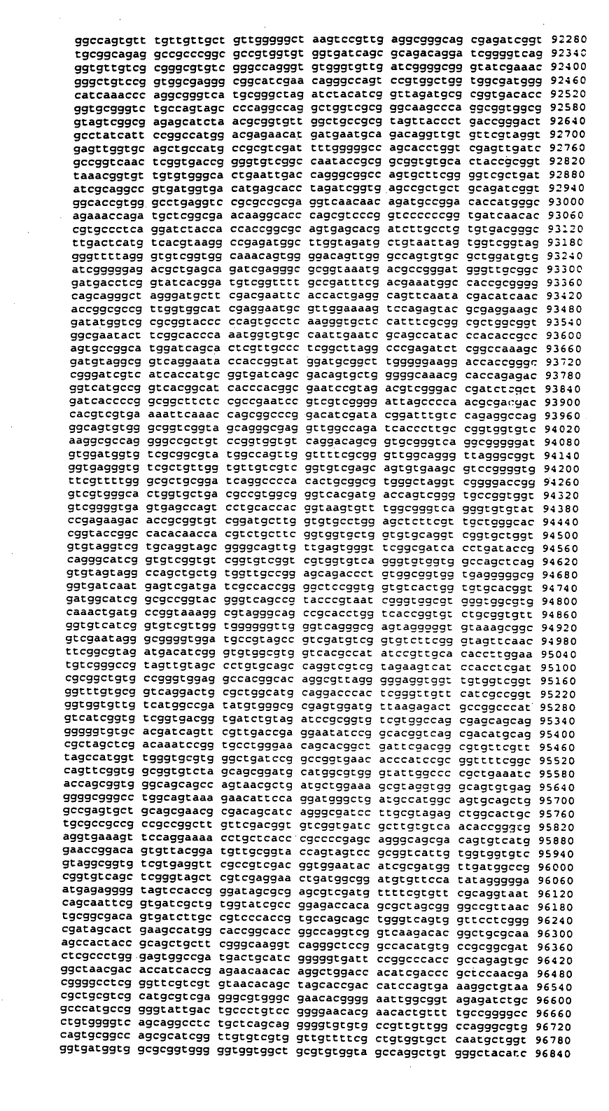

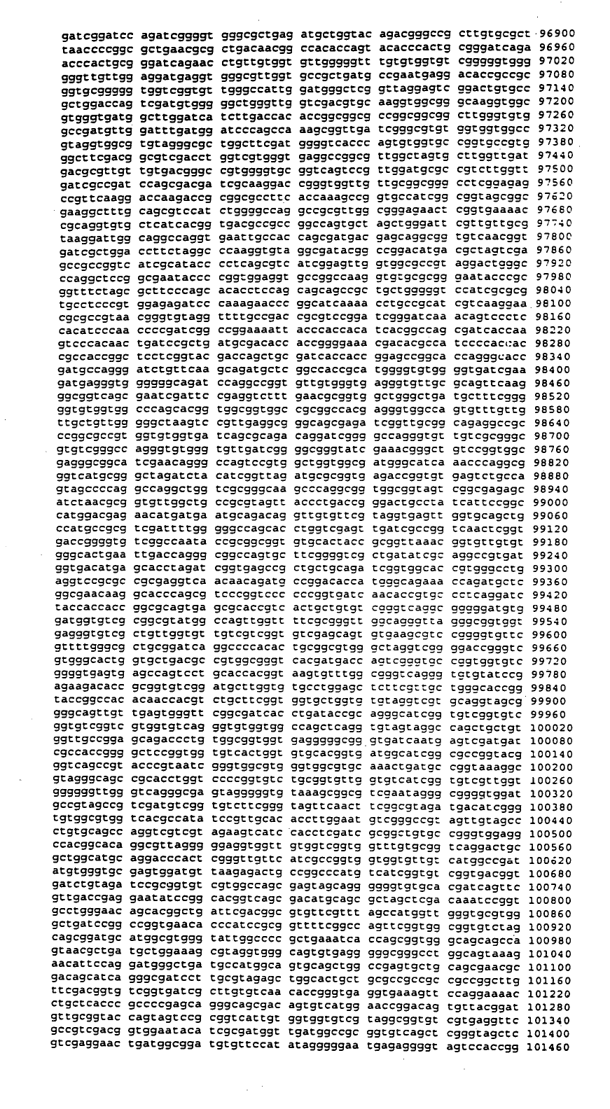

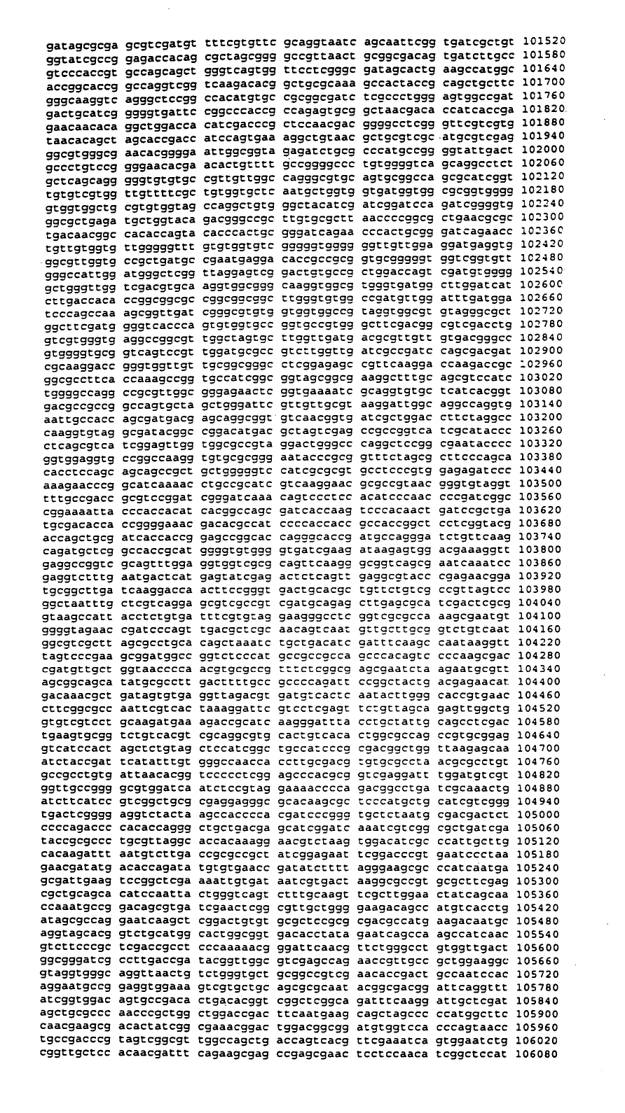

In a preferred embodiment, the polynucleotide of the invention is the isolated or purified pMUM001 plasmid of MU under circular or linear form. The sequence of pMUM001 is described in FIG. 18. The plasmid pMUM001 comprises the following ORFs referenced hereunder:

| | localization of the CDS (numbers as | | length of the encoded protein |
|---|---|---|---|
| CDS (coding sequence) | referred in sequence of FIG. 18) | encoded protein | (aa) |
| mup001 | 1 . . . 1107 | replication protein Rep | 368 |
| MUP002c | complement(1117 . . . 1431) | Hypothetical protein | 104 |
| MUP003 | 1694 . . . 2290 | Hypothetical protein | 198 |
| MUP004c | complement(2310 . . . 2924) | Hypothetical protein | 204 |
| MUP005c | complement(2921 . . . 3901) | Possible chromosome | 326 |
| | | partitioning protein ParA | |
| MUP006c | complement(5640 . . . 6386) | Hypothetical protein | 248 |
| MUP007c | complement(6383 . . . 6604) | Conserved hypothetical protein | 73 |
| MUP008c | complement(6612 . . . 7160) | Possible nucleic acid binding | 182 |
| | | protein | |
| MUP009 | 7188 . . . 7616 | Hypothetical protein | 142 |
| MUP010 | 7630 . . . 8421 | Hypothetical protein | 263 |
| MUP011 | 8430 . . . 10412 | Probable transmembrane | 660 |
| | | serine/threonine-protein | |
| MUP012c | complement(10429 . . . 10692) | Hypothetical protein | 87 |
| MUP013c | complement(10689 . . . 11147) | Possible conserved membrane | 152 |
| | | protein | |
| MUP014c | complement(11149 . . . 11922) | Putative integral membrane | 257 |
| | | protein | |
| MUP015c | complement(11916 . . . 12692) | Possible secreted protein | 258 |
| MUP016c | complement(12689 . . . 13480) | Hypothetical protein | 263 |
| MUP017c | complement(13477 . . . 13929) | Possible conserved | 150 |
| | | transmembrane protein | |
| MUP018c | complement(13973 . . . 15061) | Probable forkhead-associated | 362 |
| | | protein | |
| MUP019 | 15406 . . . 16440 | Probable conserved membrane | 344 |
| | | protein | |
| MUP020 | 16430 . . . 16612 | Conserved hypothetical protein | 60 |
| MUP021 | 16609 . . . 16872 | Possible transcriptional | 87 |
| | | regulatory protein | |
| MUP022 | 17287 . . . 18621 | Probable transposase for the | 444 |
| | | insertion element IS2606 | |
| MUP023c | complement(18772 . . . 19404) | Hypothetical protein | 210 |
| MUP024c | complement(19401 . . . 19988) | Hypothetical protein | 195 |
| MUP025 | 20718 . . . 22457 | Putative transposase | 579 |
| MUP026 | 22629 . . . 23963 | Probable transposase for IS2606 | 444 |
| MUP027c | complement(24162 . . . 24980) | Putative transposase | 272 |
| MUP028c | complement(25197 . . . 26936) | Putative transposase | 579 |
| MUP029c | complement(26980 . . . 27321) | Probable transposase for the | 113 |
| | | insertion element IS2404 | |
| | | (fragment) | |
| MUP030c | complement(27322 . . . 28026) | Probable transposase for the | 234 |
| | | insertion element IS2404 | |
| | | (fragment) | |
| MUP031c | complement(28386 . . . 29720) | Probable transposase for the | 444 |
| | | insertion element IS2606 | |
| MUP032c, mlsB | complement(30054 . . . 72446) | Type I modular polyketide | 14130 |
| | | synthase | |
| MUP033c | complement(72536 . . . 72910) | Putative transposase | 124 |
| MUP034c | complement(73008 . . . 73547) | Putative transposase | 179 |
| MUP035 | 74138 . . . 74851 | Putative transposase | 237 |
| MUP036c | complement(74905 . . . 76239) | Probable transposase for the | 444 |
| | | insertion element IS2606 | |
| MUP037 | 76556 . . . 77911 | Putative transposase | 451 |
| MUP038c | complement(78019 . . . 78924) | Possible thioesterase | 301 |
| MUP039c, mlsA2 | complement(79080 . . . 86312) | Type I modular | 2410 |
| | | FT polyketide synthase | |
| MUP040c, mlsA1 | complement(86299 . . . 137271) | Type 1 modular polyketide | 16990 |
| | | synthase | |
| MUP041c | complement(137361 . . . 137735) | Putative transposase | 124 |
| MUP042c | complement(137833 . . . 138372) | Putative transposase | 179 |
| MUP043 | 138963 . . . 140018 | Putative transposase | 351 |
| MUP044c | complement(140008 . . . 140148) | Putative truncated ransposase | 46 |
| MUP045 | 140606 . . . 141592 | Probable beta-ketoacyl | 328 |
| | | synthase-like protein | |
| MUP046 | 142322 . . . 142615 | Possible membrane protein | 97 |
| MUP047 | 143012 . . . 143716 | Probable transposase for the | 234 |
| | | insertion element IS2404 | |
| MUP048 | 143717 . . . 144058 | Probable transposase for the | 113 |
| | | insertion element IS2404 | |
| MUP049c | complement(144304 . . . 144693) | Putative transposase | 129 |
| MUP050 | 144660 . . . 145994 | Probable transposase for the | 444 |
| | | insertion element IS2606 | |
| MUP051 | 146252 . . . 146533 | Putative transposase | 93 |
| MUP052 | 146563 . . . 147396 | Putative transposase | 277 |
| MUP053c, cyp150 | complement(147546 . . . 148859) | Probable cytochrome p450 150 | 437 |
| | | cyp150 | |
| MUP054c | complement(148856 . . . 149359) | Possible integrase ragment | 167 |
| MUP055 | 149323 . . . 150657 | Probable transposase for the | 444 |
| | | insertion element IS2606 | |
| MUP056c | complement(150862 . . . 151242) | Hypothetical protein | 126 |
| MUP057c | complement(151341 . . . 152117) | Possible lipoprotein | 258 |
| MUP058c | complement(152314 . . . 153351) | Possible site-specific | 345 |
| | | recombinase | |
| MUP059c | complement(153595 . . . 154641) | Probable transposase for the | 348 |
| | | insertion element IS2404 | |
| MUP060 | 155147 . . . 155668 | Probable transposase for the | 173 |
| | | insertion element IS2606 | |
| MUP061 | 155574 . . . 156482 | Probable transposase for the | 302 |
| | | insertion element IS2606 | |
| MUP062 | 156842 . . . 157546 | Probable transposase for the | 234 |
| | | insertion element IS2404 | |
| MUP063 | 157547 . . . 157888 | Probable transposase for the | 113 |
| | | insertion element IS2404 | |
| MUP064c | complement(157889 . . . 158251) | Possible conserved membrane | 120 |
| | | protein | |
| MUP065c | complement(158471 . . . 159352) | Conserved hypothetical protein | 293 |
| MUP066c | complement(159824 . . . 160330) | Conserved hypothetical protein | 168 |
| MUP067c | complement(160417 . . . 161049) | Conserved hypothetical protein | 210 |
| MUP068c | complement(161085 . . . 162215) | Conserved membrane rotein | 376 |
| MUP069c | complement(162445 . . . 163779) | Probable transposase for the | 444 |
| | | insertion element IS2606 | |
| MUP070c | complement(163727 . . . 164824) | Conserved hypothetical protein | 365 |
| MUP071c | complement(164673 . . . 165089) | Conserved hypothetical protein | 138 |
| MUP072c | complement(165161 . . . 166357) | Conserved hypothetical protein | 398 |
| MUP073c | complement(166354 . . . 167547) | Conserved hypothetical protein | 397 |
| MUP074c | complement(167568 . . . 168152) | Possible membrane protein | 194 |
| MUP075c | complement(168149 . . . 168487) | Hypothetical protein | 112 |
| MUP076c | complement(168487 . . . 169158) | Possible membrane protein | 223 |
| MUP077c | complement(169192 . . . 169584) | Conserved hypothetical protein | 130 |
| MUP078c | complement(169759 . . . 171342) | Conserved hypothetical protein | 527 |
| MUP079c | complement(171361 . . . 171660) | Conserved hypothetical protein | 99 |
| MUP080c | complement(171667 . . . 171939) | Conserved hypothetical protein | 90 |
| MUP081c | complement(172002 . . . 173546) | Conserved hypothetical protein | 514 |

The term “complement”means that the CDS is on the complementary strand to the strand shown in FIG. 18.

In a second embodiment, the present invention concerns an isolated or purified polypeptide having an amino acid sequence encoded by a polynucleotide as defined previously. The polypeptide of the present invention preferably comprises an amino acid sequence having at least 80% homology, or even preferably 85% homology to part or all of SEQ ID NO:7-12. Yet, more preferably, the polypeptide comprises an amino acid sequence substantially the same or having 100% identity with at least one amino acid sequence selected among the sequences SEQ ID NO:7-12 and biologically active fragments thereof.

As used herein, the expression “biological active” refers to a polypeptide or fragment(s) thereof that substantially retain the enzymatic capacity of the polypeptide from which it is derived.

According to another preferred embodiment, the polypeptide of the present invention comprises an amino acid sequence encoded by a polynucleotide which hybridizes under stringent conditions to the complement of SEQ ID NO:1-6 or fragments thereof. Such a polypeptide substantially retains the enzymatic capacity of the polypeptide from which it is derived in the mycolactone biosynthesis. As used herein, to hybridize under conditions of a specified stringency describes the stability of hybrids formed between two single-stranded DNA fragments and refers to the conditions of ionic strength and temperature at which such hybrids are washed, following annealing under conditions of stringency less than or equal to that of the washing step. Typically high, medium and low stringency encompass the following conditions or equivalent conditions thereto:
1) high stringency: 0.1×SSPE or SSC, 0.1% SDS, 65° C.
2) medium stringency: 0.2×SSPE or SSC, 0.1% SDS, 50° C.
3) low stringency: 1.0×SSPE or SSC, 0.1% SDS, 50° C.

As used herein, the term “polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. A peptide according to the invention preferably comprises from 2 to 20 amino acids, more preferably from 2 to 10 amino acids, and most preferably from 2 to 5 amino acids. Polypeptides may contain amino acids other than the 20 gene-encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation, selenoylation, sulfation and transfer-RNA mediated addition of amino acids to proteins, such as arginylation, and ubiquitination. See, for instance: PROTEINS—STRUCTURE AND MOLECULAR PROPERTIES, 2nd Ed., T. E. Creighton, W.H. Freeman and Company, New York (1993); Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990); and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

The homology percentage of polypeptides can be determined, for example by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

Homologous polypeptides can comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gin and Asn. Other such conservative substitutions, for example, substitutions of entire regions having similar hydrophobicity characteristics, are well known. Naturally occurring homologous MLS polypeptides are also encompassed by the invention. Examples of such homologous polypeptides are polypeptides that result from alternate mRNA splicing events or from proteolytic cleavage of the MLS polypeptides. Variations attributable to proteolysis include, for example, differences in the termini upon expression in different types of host cells, due to proteolytic removal of one or more terminal amino acids from the MLS polypeptides. Variations attributable to frameshifling include, for example, differences in the termini upon expression in different types of host cells due to different amino acids. Homologous MLS polypeptides can also be obtained by mutations of nucleotide sequences coding for polypeptides of sequence SEQ ID NO:7-12. Alterations of the amino acid sequence can be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an homologous polypeptide having the desired amino acid insertion, substitution, or deletion. Alternatively, oligonucleotide-directed site-specific mutagenesis procedures can be employed to provide an altered polynucleotide wherein predetermined codons can be altered by substitution, deletion, or insertion. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462, all of which are incorporated by reference.

The invention also encompasses polypeptides encoded by the fragments and oligonucleotides derived from the nucleotide sequences of SEQ ID NO:1-6.

It will also be understood that the invention encompasses equivalent proteins having substantially the same biological and immunogenic properties. Thus, this invention is intended to cover serotypic variants of the proteins of the invention.

Depending on the use to be made of the MLS polypeptides of the invention, it may be desirable to label them. Examples of suitable labels are radioactive labels, enzymatic labels, fluorescent labels, chemiluminescent labels, and chromophores. The methods for labeling polypeptides of the invention do not differ in essence from those widely used for labeling immunoglobulin. The need to label may be avoided by using labeled antibody directed against the polypeptide of the invention or anti-immunoglobulin to the antibodies to the polypeptide as an indirect marker.

2. Vectors and Cells

In a third embodiment, the invention is further directed to cloning or expression vector comprising a polynucleotide as defined above, and more particularly directed to a cloning or expression vector which is capable of directing expression of the polypeptide encoded by the polynucleotide sequence in a vector-containing cell.

As used herein, the term “vector” refers to a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector.

A number of vectors suitable for stable transfection of cells and bacteria are available to the public (e.g. plasmids, adenoviruses, baculoviruses, yeast baculoviruses, plant viruses, adeno-associated viruses, retroviruses, Herpes Simplex Viruses, Alphaviruses, Lentiviruses), as are methods for constructing such cell lines. It will be understood that the present invention encompasses any type of vector comprising any of the polynucleotide molecule of the invention.

Recombinant expression vectors containing a polynucleotide encoding MLS polypeptides can be prepared using well known methods. The expression vectors include a MLS polynucleotide operably linked to suitable transcriptional or translational regulatory sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation, and termination. The term “operably linked” means that the regulatory sequence functionally relates to the MLS DNA. Thus, a promoter is operably linked to a MLS polynucleotide if the promoter controls the transcription of the MLS polynucleotide. The ability to replicate in the desired host cells, usually conferred by an origin of replication, and a selection gene by which transformants are identified can additionally be incorporated into the expression vector.

In addition, nucleic acids encoding appropriate signal peptides that are not naturally associated with MLS polynucleotide can be incorporated into expression vectors. For example, a nucleic acid coding for a signal peptide (secretory leader) can be fused in-frame to the MLS polynucleotide so that the MLS polypeptide is initially translated as a fusion protein comprising the signal peptide. A signal peptide that is functional in the intended host cells enhances extracellular secretion of the MLS polypeptide. The signal peptide can be cleaved from the MLS polypeptide upon secretion of MLS polypeptide from the cell.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids. Commercially available vectors include those that are specifically designed for the expression of proteins. These include pMAL-p2 and pMAL-c2 vectors, which are used for the expression of proteins fused to maltose binding protein (New England Biolabs, Beverly, Mass., USA).

Promoter commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776), and tac promoter (Maniatis, Molecular Cloning: A Laboratory Manual, Cold Spring Harbor Laboratory, p. 412, 1982).

In a fourth embodiment, the invention is also directed to a host, such as a genetically modified cell, comprising any of the polynucleotide or vector according to the invention and more preferably, a host capable of expressing the polypeptide encoded by this polynucleotide.

The host cell may be any type of cell (a transiently-transfected mammalian cell line, an isolated primary cell, or insect cell, yeast (Saccharomyces cerevisiae, Ktuyveromyces lactis, Pichia pastoris), plant cell, microorganism, or a bacterium (such as E. coli). More preferably the host is Escherichia coli bacterium. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems can also be employed to produce MLS polypeptides using RNAs derived from MSL polynucleotide disclosed herein.

The following biological deposits named MU0022B04 and MU022D03 relating to Escherichia coli comprising respectively the BAC vector pMU0022B04 and pMU022D03 were registered at the Collection Nationale de Cultures de Microorganismes (C.N.C.M.), of Institut Pasteur, 28, rue du Docteur Roux, F-75724 Paris, Cedex 15, France, on Nov. 3, 2003, under the following Accession Numbers:

| RECOMBINANT | ACCESSION |
|---|---|
| ESCHERICHIA COLI | NO. |
| MU0022B04 | I-3121 |
| MU022D03 | I-3122 |

Copies of Deposit Receipts (11 pages) are attached.

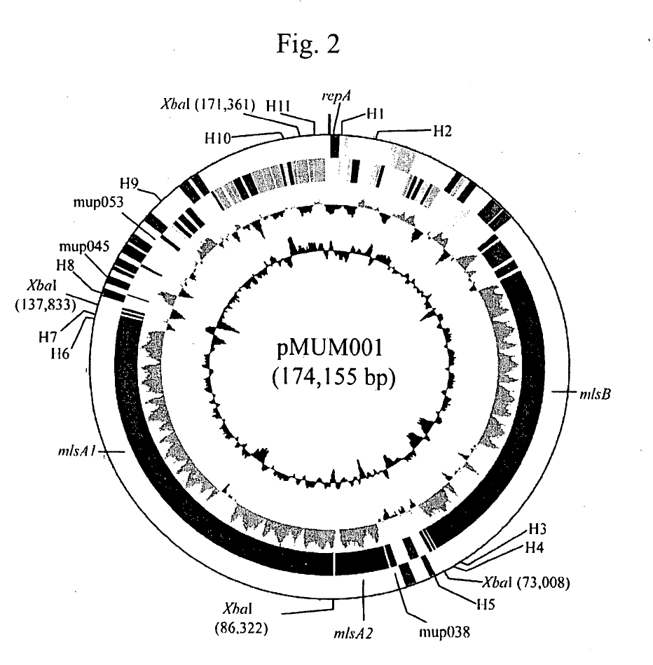

The BAC vector pMU0022B04 comprises a 80 kbp fragment of the plasmid pMUM001 of MU cloned from the Hind III site at position 71,846 (referred H4 in FIG. 2) to the HindIII site at position 152,732 (referred as H9 in FIG. 2) and containing mup038, mlsA2, mlsA1, mup045 and mup053 genes.

The BAC vector pMU022D03 comprises a 109 kbp fragment of the plasmid pMUM001 of MU cloned at the HindIII site at position 173,190 (site H11 as referred in FIG. 2), this fragment corresponds to the entire sequence of plasmid pMUM001 but with the 65 kpb region between the Hind III site at position 73,953 (referred as H5 in FIG. 2) to the HindIII site at position 138,778 (referred as H8 in FIG. 2) deleted. Then the 109 kpb fragment contains the mup045, mup053 and mlsB genes.

3. Antibodies

In a fifth embodiment, the invention features purified antibodies that specifically bind to isolated or purified polypeptides as defined above or fragments thereof, and more particularly to polypeptides of amino acid sequence SEQ ID NO;7-12. The antibodies of the invention may be prepared by a variety of methods using the MLS polypeptides described above. For example, MLS polypeptide, or antigenic fragments thereof, may be administered to an animal (for example, horses, cows, goats, sheep, dogs, chickens, rabbits, mice, or rats) in order to induce the production of polyclonal antibodies. Techniques to immunize an animal host are well-known in the art. Such techniques usually involve inoculation, but they may involve other modes of administration. A sufficient amount of the polypeptide is administered to create an immunogenic response in the animal host. Any host that produces antibodies to the antigen of the invention can be used. Once the animal has been immunized and sufficient time has passed for it to begin producing antibodies to the antigen, polyclonal antibodies can be recovered. The general method comprises removing blood from the animal and separating the serum from the blood. The serum, which contains antibodies to the antigen, can be used as an antiserum to the antigen. Alternatively, the antibodies can be recovered from the serum. Affinity purification is a preferred technique for recovering purified polyclonal antibodies to the antigen, from the serum.

Alternatively, antibodies used as described herein may be monoclonal antibodies, which are prepared using hybridoma technology (see, e.g., Hammerling et al., In Monoclonal Antibodies and T-Cell Hybridomas, Elsevier, N.Y., 1981).

As mentioned above, the present invention is preferably directed to antibodies that specifically bind MLS polypeptides, or fragments thereof. In particular, the invention features “neutralizing” antibodies. By “neutralizing” antibodies is meant antibodies that interfere with any of the biological activities of any of the MLS polypeptides, particularly the ability of MU to synthetize mycolactone and induce cutaneous infection. Any standard assay known to one skilled in the art may be used to assess potentially neutralizing antibodies. Once produced, monoclonal and polyclonal antibodies are preferably tested for specific MLS polypeptides recognition by Western blot, immunoprecipitation analysis or any other suitable method.

Antibodies that recognize MLS polypeptides expressing cells and antibodies that specifically recognize MLS polypeptides, such as those described herein, are considered useful to the invention. Such an antibody may be used in any standard immunodetection method for the detection, quantification, and purification of native MLS polypeptides. The antibody may be a monoclonal or a polyclonal antibody and may be modified for diagnostic purposes. The antibodies of the invention may, for example, be used in an immunoassay to monitor MLS polypeptides expression levels, to determine the amount of MLS polypeptides or fragment thereof in a biological sample and evaluate the presence or not of Mycobacterium ulcerans. In addition, the antibodies may be coupled to compounds for diagnostic and/or therapeutic uses such as gold particles, alkaline phosphatase, peroxidase for imaging and therapy. The antibodies may also be labeled (e.g. immunofluorescence) for easier detection.

With respect to antibodies of the invention, the term “specifically binds to” refers to antibodies that bind with a relatively high affinity to one or more epitopes of a protein of interest, but which do not substantially recognize and bind molecules other than the one(s) of interest. As used herein, the term “relatively high affinity” means a binding affinity between the antibody and the protein of interest of at least 10⁶ M⁻¹, and preferably of at least about 10⁷ M⁻¹ and even more preferably 10⁸ M⁻¹ to 10¹⁰ M⁻¹. Determination of such affinity is preferably conducted under standard competitive binding immunoassay conditions which is common knowledge to one skilled in the art (for example, Scatchard et al., Ann. N. Y Acad. Sci., 51:660 (1949)).

As used herein, “antibody” and “antibodies” include all of the possibilities mentioned hereinafter: antibodies or fragments thereof obtained by purification, proteolytic treatment or by genetic engineering, artificial constructs comprising antibodies or fragments thereof and artificial constructs designed to mimic the binding of antibodies or fragments thereof. Such antibodies are discussed in Colcher et al. (Q J Nucl Med 1998; 42: 225-241). They include complete antibodies, F(ab′)₂ fragments, Fab fragments, Fv fragments, scFv fragments, other fragments, CDR peptides and mimetics. These can easily be obtained and prepared by those skilled in the art. For example, enzyme digestion can be used to obtain F(ab′)₂ and Fab fragments by subjecting an IgG molecule to pepsin or papain cleavage respectively. Recombinant antibodies are also covered by the present invention.

Alternatively, the antibody of the invention may be an antibody derivative. Such an antibody may comprise an antigen-binding region linked or not to a non-immunoglobulin region. The antigen binding region is an antibody light chain variable domain or heavy chain variable domain. Typically, the antibody comprises both light and heavy chain variable domains, that can be inserted in constructs such as single chain Fv (scFv) fragments, disulfide-stabilized Fv (dsFv) fragments, multimeric scFv fragments, diabodies, minibodies or other related forms (Colcher et al. Q J Nucl Med 1998; 42: 225-241). Such a derivatized antibody may sometimes be preferable since it is devoid of the Fc portion of the natural antibody that can bind to several effectors of the immune system and elicit an immune response when administered to a human or an animal. Indeed, derivatized antibody normally do not lead to immuno-complex disease and complement activation (type III hypersensitivity reaction).

Alternatively, a non-immunoglobulin region is fused to the antigen-binding region of the antibody of the invention. The non-immunoglobulin region is typically a non-immunoglobulin moiety and may be an enzyme, a region derived from a protein having known binding specificity, a region derived from a protein toxin or indeed from any protein expressed by a gene, or a chemical entity showing inhibitory or blocking activity(ies) against the MU mycolactone biosynthesis-associated polypeptides. The two regions of that modified antibody may be connected via a cleavable or a permanent linker sequence. Preferably, the antibody of the invention is a human or animal immunoglobulin such as IgG1, IgG2, IgG3, IgG4, IgM, IgA, IgE or IgD carrying rat or mouse variable regions (chimeric) or CDRs (humanized or “animalized”). Furthermore, the antibody of the invention may also be conjugated to any suitable carrier known to one skilled in the art in order to provide, for instance, a specific delivery and prolonged retention of the antibody, either in a targeted local area or for a systemic application.

The term “humanized antibody” refers to an antibody derived from a non-human antibody, typically murine, that retains or substantially retains the antigen-binding properties of the parent antibody but which is less immunogenic in humans. This may be achieved by various methods including (a) grafting only the non-human CDRs onto human framework and constant regions with or without retention of critical framework residues, or (b) transplanting the entire non-human variable domains, but “cloaking” them with a human-like section by replacement of surface residues. Such methods are well known to one skilled in the art.

As mentioned above, the antibody of the invention is immunologically specific to the polypeptide of the present invention and immunological derivatives thereof. As used herein, the term “immunological derivative” refers to a polypeptide that possesses an immunological activity that is substantially similar to the immunological activity of the whole polypeptide, and such immunological activity refers to the capacity of stimulating the production of antibodies immunologically specific to the MU mycolactone biosynthesis-associated polypeptides or derivative thereof. The term “immunological derivative” therefore encompass “fragments”, “segments”, “variants”, or “analogs” of a polypeptide.

The term “antigen” refers to a molecule that provokes an immune response such as, for example, a T lymphocyte response or a B lymphocyte response or which can be recognized by the immune system. In this regard, an antigen includes any agent that when introduced into an immunocompetent animal stimulates the production of a cellular-mediated response or the production of a specific antibody or antibodies that can combine with the antigen.

4. Compositions and Vaccines

The polypeptides of the present invention, the polynucleotides coding the same, and polyclonal or monoclonal antibodies produced according to the invention, may be used in many ways for the diagnosis, the treatment or the prevention of Mycobacterium ulcerans related diseases and in particular Buruli ulcer.

In a sixth embodiment, the present invention relates to a composition for eliciting an immune response or a protective immunity against Mycobacterium ulcerans. According to a related aspect, the present invention relates to a vaccine for preventing and/or treating a Mycobacterium ulcerans associated disease. As used herein, the term “treating” refers to a process by which the symptoms of Buruli ulcer are alleviated or completely eliminated. As used herein, the term “preventing” refers to a process by which a Mycobacterium ulcerans associated disease is obstructed or delayed. The composition or the vaccine of the invention comprises a polynucleotide, a polypeptide and/or an antibody as defined above and an acceptable carrier.

As used herein, the expression “an acceptable carrier” means a vehicle for containing the polynucleotide, a polypeptide and/or an antibody that can be injected into a mammalian host without adverse effects. Suitable carriers known in the art include, but are not limited to, gold particles, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

Further agents can be added to the composition and vaccine of the invention. For instance, the composition of the invention may also comprise agents such as drugs, immunostimulants (such as α-interferon, β-interferon, γ-interferon, granulocyte macrophage colony stimulator factor (GM-CSF), macrophage colony stimulator factor (M-CSF), interleukin 2 (IL2), interleukin 12 (IL12), CpG oligonucleotides, aluminum phosphate and aluminum hydroxide gel, or any other adjuvant described in McCluskie et Weeratna, Current Drug Targets-Infectious Disorders, 2001, 1, 263-271), antioxidants, surfactants, flavoring agents, volatile oils, buffering agents, dispersants, propellants, and preservatives. To potentiate the immune response in the host, the MLS polypeptides can be bound to lipid membranes or incorporated in lipid membranes to form liposomes. The use of nonpyrogenic lipids free of nucleic acids and other extraneous matter can be employed for this purpose. For preparing such compositions, methods well known in the art may be used.

The amount of polynucleotide, a polypeptide and/or an antibody present in the compositions or in the vaccines of the present invention is preferably a therapeutically effective amount. A therapeutically effective amount of polynucleotide, a polypeptide and/or an antibody is that amount necessary to allow the same to perform their immunological role without causing, overly negative effects in the host to which the composition is administered. The exact amount of polynucleotide, a polypeptide and/or an antibody to be used and the composition/vaccine to be administered will vary according to factors such as the type of condition being treated, the mode of administration, as well as the other ingredients in the composition.

5. Methods of Use

Methods for Treating and/or Preventing M. ulcerans Related Diseases

In a seventh embodiment, the present invention relates to methods for treating and/or preventing MU related diseases, such as Buruli ulcer in a mammal are provided.

These methods have the major purpose to provoke or potentiate the immune response in an MU-infected mammal in order to inactivate the free MU and eliminate MU infected cells that have the potential to release pathogens. The B-cell arm of the immune response has the major responsibility for inactivating free MU. The principal manner in which this is achieved is by neutralization of infectivity. Another major mechanism for destruction of the MU-infected cells is provided by cytotoxic T lymphocytes (CTL) that recognize MLS antigens expressed in combination with class I histocompatibility antigens at the cell surface. The CTLs recognize MLS polypeptides processed within cells from a MLS protein that is produced, for example, by the infected cell or that is internalized by a phagocytic cell. Thus, this invention can be employed to stimulate a B-cell response to MLS polypeptides, as well as immunity mediated by a CTL response following MU infection. The CTL response can play an important role in mediating recovery from primary MU infection and in accelerating recovery during subsequent infections.

These methods comprise the step of administering to the mammal an effective amount of an isolated or purified MLS polynucleotide, an isolated or purified MLS polypeptide, the composition as defined above and/or the vaccine as defined above.

The vaccine, antibody and composition of the invention may be given to a an individual through various routes of administration. In embodiments, the individual is an animal, and is preferably a mammal. More preferably, the mammal is a human. For instance, the composition may be administered in the form of sterile injectable preparations, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection, by infusion or per os. The vaccine and the composition of the invention may also be formulated as creams, ointments, lotions, gels, drops, suppositories, sprays, liquids or powders for topical administration. They may also be administered into the airways of a subject by way of a pressurized aerosol dispenser, a nasal sprayer, a nebulizer, a metered dose inhaler, a dry powder inhaler, or a capsule.

Suitable dosages will vary, depending upon factors such as the amount of each of the components in the composition, the desired effect (short or long term), the route of administration, the age and the weight of the mammal to be treated. In any event, the amount administered should be at least sufficient to protect the host against substantial immunosuppression, even though MU infection may not be entirely prevented. An immunogenic response can be obtained by administering the polypeptides of the invention to the host in an amount of about 0.1 to about 5000 micrograms antigen per kilogram of body weight, preferably about 0.1 to about 1000 micrograms antigen per kilogram of body weight, and more preferably about 0.1 to about 100 micrograms antigen per kilogram of body weight. As an example of common schedule, a single does of the vaccine of the invention can be administered to the host or a primary course of immunization can be followed in which several doses at intervals of time are administered. Subsequent doses used as boosters can be administered as need following the primary course. Any other methods well known in the art may be used for administering the vaccine, antibody and the composition of the invention.

Regarding the methods of treating by administering immunogenic compositions comprising MLS polynucleotides, those of skill in the art are cognizant of the concept, application, and effectiveness of nucleic acid vaccines (e.g., DNA vaccines) and nucleic acid vaccine technology. The nucleic acid based technology allows the administration of MLS polynucleotides, naked or encapsulated, directly to tissues and cells without the need for production of encoded proteins prior to administration. The technology is based on the ability of these nucleic acids to be taken up by cells of the recipient organism and expressed to produce an immunogenic determinant to which the recipient's immune system responds. Typically, the expressed antigens are displayed on the surface of cells that have taken up and expressed the nucleic acids, but expression and export of the encoded antigens into the circulatory system of the recipient individual is also within the scope of the present invention. Such nucleic acid vaccine technology includes, but is not limited to, delivery of naked DNA and RNA and delivery of expression vectors encoding MLS polypeptides. Although the technology is termed “vaccine”, it is equally applicable to immunogenic compositions that do not result in a protective response. Such non-protection inducing compositions and methods are encompassed within the present invention.

Although it is within the present invention to deliver MLS nucleic acids and carrier molecules as naked nucleic acid, the present invention also encompasses delivery of nucleic acids as part of larger or more complex compositions. Included among these delivery systems are viruses, virus-like particles, or bacteria containing the MLS nucleic acid. Also, complexes of the invention's nucleic acids and carrier molecules with cell permeabilizing compounds, such as liposomes, are included within the scope of the invention. Other compounds, such as molecular vectors (EP 696,191, Samain et al.) and delivery systems for nucleic acid vaccines are known to the skilled artisan and exemplified in, for example, WO 93 06223 and WO 90 11092, U.S. Pat. No. 5,580,859, and U.S. Pat. No. 5,589,466 (Vical's patents), which are incorporated by reference herein, and can be made and used without undue or excessive experimentation.

In Vitro Diagnostic Method

The MLS polypeptides can be used as antigens to identify antibodies to MU in a biological material and to determine the concentration of the antibodies in this biological material. Thus, the MLS polypeptides can be used for qualitative or quantitative determination of MU in a biological material. Such biological material of course includes human tissue and human cells, as well as biological fluids, such as human body fluids, including human sera.

More particularly, the present invention is directed to an in vitro diagnostic method for the detection of the presence or absence of antibodies to MU, which bind with a MLS polypeptide as defined above to form an immune complex. Such method comprises the steps of:
a) contacting the polypeptide of the present invention with a biological material for a time and under conditions sufficient to form an immune complex;
b) detecting the presence or absence of the immune complex formed in a); and optionally
c) measuring the immune complex formed.

More particularly, the MLS polypeptides can be employed for the detection of MU by means of immunoassays that are well known for use in detecting or quantifying humoral components in fluids. Thus, antigen-antibody interactions can be directly observed or determined by secondary reactions, such as precipitation or agglutination. In addition, immunoelectrophoresis techniques can also be employed. For example, the classic combination of electrophoresis in agar followed by reaction with anti-serum can be utilized, as well as two-dimensional electrophoresis, rocket electrophoresis, and immunolabeling of polyacrylamide gel patterns (Western Blot or immunoblot). Other immunoassays in which the MLS polypeptides can be employed include, but are not limited to, radioimmunoassay, competitive immunoprecipitation assay, enzyme immunoassay, and immunofluorescence assay. It will be understood that turbidimetric, colorimetric, and nephelometric techniques can be employed. An immunoassay based on Western Blot technique is preferred.

Immunoassays can be carried out by immobilizing one of the immunoreagents, either an antigen of the invention or an antibody of the invention to the antigen, on a carrier surface while retaining immunoreactivity of the reagent. The reciprocal immunoreagent can be unlabeled or labeled in such a manner that immunoreactivity is also retained. These techniques are especially suitable for use in enzyme immunoassays, such as enzyme linked immunosorbent assay (ELISA) and competitive inhibition enzyme immunoassay (CIEIA).

When either the MLS polypeptides or the antibody to the MLS polypeptides is attached to a solid support, the support is usually a glass or plastic material. Plastic materials molded in the form of plates, tubes, beads, or disks are preferred. Examples of suitable plastic materials are polystyrene and polyvinyl chloride. If the immunoreagent does not readily bind to the solid support, a carrier material can be interposed between the reagent and the support. Examples of suitable carrier materials are proteins, such as bovine serum albumin, or chemical reagents, such as gluteraldehyde or urea. Coating of the solid phase can be carried out using conventional techniques.

In a further embodiment, a diagnostic kit for the detection of the presence or absence of antibodies indicative of MU is provided. Accordingly, the kit comprises:
a polypeptide as defined above;
a reagent to detect polypeptide-antibody immune complex;
a biological reference sample lacking antibodies that immunologically bind with the polypeptide; and
a comparison sample comprising antibodies which can specifically bind to the polypeptide;
   wherein the polypeptide, reagent, biological reference sample, and comparison sample are present in an amount sufficient to perform the detection.

The present invention also proposes an in vitro diagnostic method for the detection of the presence or absence of polypeptides indicative of MU, which bind with the antibody of the present invention to form an immune complex, comprising the steps of:
a) contacting the antibody of the invention with a biological sample for a time and under conditions sufficient to form an immune complex;
b) detecting the presence or absence of the immune complex formed in a); and optionally
c) measuring the immune complex formed.

In a further embodiment, a diagnostic kit for the detection of the presence or absence of polypeptides indicative of MU is provided. Accordingly, the kit comprises:
an antibody as defined above;
a reagent to detect polypeptide-antibody immune complex;
a biological reference sample lacking polypeptides that immunologically bind with the antibody; and
a comparison sample comprising polypeptides which can specifically bind to the antibody;
   wherein said antibody, reagent, biological reference sample, and comparison sample are present in an amount sufficient to perform the detection.

To further achieve the objects and in accordance with the purposes of the present invention, an in vitro diagnostic method for the detection of the presence or absence of a polynucleotide indicative of MU is provided. Accordingly, the method comprises the steps of:
a) contacting at least one probe as defined above with a biological material for a time and under conditions sufficient for said probe to hybridize to said polynucleotide; and
b) detecting the presence or absence of an hybridization between the probe and the polynucleotide.

Different diagnostic techniques can be used which include, but are not limited to: (1) Southern blot procedures to identify cellular DNA which may or may not be digested with restriction enzymes; (2) Northern blot techniques to identify RNA extracted from cells; (3) dot blot techniques, i.e., direct filtration of the sample through an ad hoc membrane, such as nitrocellulose or nylon, without previous separation on agarose gel and (4) PCR techniques to amplify nucleic acids with.

Yet, according to a further embodiment, a diagnostic kit for the detection of the presence or absence of polynucleotide indicative of MU is provided accordingly, the kit comprises:
a probe as defined above;
a reagent to detect polynucleotide-probe hybridization complex;
a biological reference sample lacking polynucleotides that hybridise with the probe; and
a comparison sample comprising polynucleotides which can specifically hybridise to the probe;
   wherein said probe, reagent, biological reference sample, and comparison sample are present in an amount sufficient to perform the detection.

The present invention will be more readily understood by referring to the following examples. These examples are illustrative of the wide range of applicability of the present invention and is not intended to limit its scope. Modifications and variations can be made therein without departing from the spirit and scope of the invention. Although any methods and materials similar or equivalent to those described herein can be used in the practice for testing of the present invention, the preferred methods and materials are described.

### EXAMPLE 1

### Identification of the Plasmid pMUM001

MU and Mycobacterium marinum (MM) share over 98% DNA sequence identity, they occupy aquatic environments and both cause cutaneous infections (3). However, MM produces a granulomatous intracellular lesion, typical for pathogenic mycobacteria and totally distinct from Buruli ulcer in which MU are mainly found extracellularly. The fact that MM does not produce mycolactone suggested that it might be possible to identify genes for mycolactone synthesis by performing genomic subtraction experiments between MU and MM. Fragments of MU-specific PKS genes were identified from these experiments (4). The subsequent investigation of these sequences led to the discovery of the MU virulence plasmid, pMUM001, and the extraordinary PKS locus it encodes.

Material and Methods

Bacterial Strains and Growth Conditions

MU strain Agy99 is a recent clinical isolate from the West African epidemic. MU1615 (ATCC 35840), originally isolated from a Malaysian patient, was obtained from the Trudeau Collection. Strains were cultivated using Middlebrook 7H9 broth (Difco) and Middlebrook 7H10 (Difco) at 32° C.

Plasmid Sequence Determination

A bacterial artificial chromosome (BAC) library was made of M. ulcerans strain Agy99, using the vector pBeloBAC11 and nucleotide end-sequences were determined as previously described (5). This library was then screened by PCR for MU-specific PKS sequences that had been identified in subtractive hybridization experiments between MU and MM (4). The complete sequences of selected BAC clones were obtained by shotgun sub-cloning and sequencing as previously described (6). To overcome the difficulties associated with the highly repetitive PKS sequences two additional BAC subclone libraries were made from (i) total PstI digests and (ii) partial Sau3AI sub-clones with insert sizes of 6-10 kb. Sau3AI subclones that represented a single module (i.e. a single non-repetitive unit) were then subjected to primer-walking. Sequences were assembled using Gap4 (6, 7). The ARTEMIS tool (www.sanger.ac.uk/Software) was used for the plasmid annotation, with comparisons to public and in-house databases performed by using the BLAST suite and FASTA. The conditions for PFGE and Southern hybridization were as previously described (3, 5).

Results

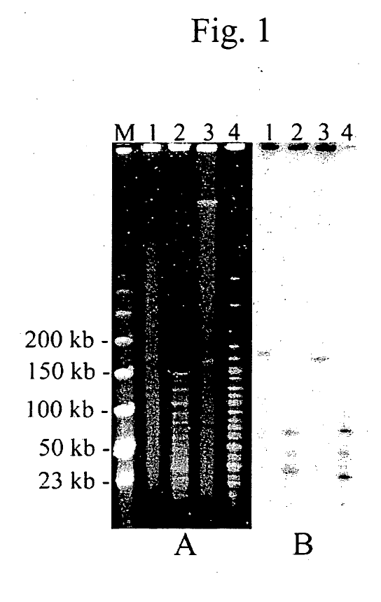

Genomic subtraction experiments led to the identification of several fragments of MU-specific polyketide synthase (PKS) genes (4). In the present work, when undigested MU genomic DNA was analysed by pulsed field gel electrophoresis a band of ˜170 kb was detected (FIG. 1A), that hybridized with the MU-specific PKS probes, suggesting that the PKS genes were plasmid-encoded (FIG. 1B). Several positively hybridizing clones were isolated from a bacterial artificial chromosome (BAC) library of the epidemic MU strain Agy99 and characterized by BAC end-sequencing, insert sizing and restriction fragment profiling. Three BACs were subsequently shotgun-sequenced with the resultant composite sequence confirming the existence in MU of a circular plasmid, designated pMUM001, comprising 174,155 bp, with a GC content of 62.8% and carrying 81 CDS (FIG. 2). Among these three BACs, one BAC named pM0022B04 has an insert of pMUM001 DNA of 80 kpb in length and one BAC named pM0022D03 has an insert of pMUM001 DNA of 110 kpb in length. The DNA inserts of the two BAC, pM0022B04 and pM0022D03, are partially overlapping and complementary to reconstruct the entire sequence of the plasmid pMUM001 as shown in FIG. 2.

In one sense the plasmid appears very simple with no identifiable transfer or maintenance genes. Replication appears to be initiated by the predicted product of repA, which shares 68.3% aa identity with RepA from the cryptic Mycobacterium fortuitum plasmid, pJAZ38 (10). Two different direct repeat regions were identified 500 bp to 1000 bp upstream of repA, suggesting possible replication origins (ori). GC-skew plots [(G−C/(G+C)], which highlight compositional biases between leading and lagging DNA strands, displayed a random pattern and did not help pinpoint a possible ori (FIG. 2). Approximately 2 kb downstream of repA is parA, a gene encoding a chromosome partioning protein, required for plasmid segregation upon cell division. In this region there is also a potential regulatory gene cluster composed of a serine/threonine protein kinase (mup008), a gene encoding a protein of unknown function (mup018) but containing a phosphopeptide recognition domain, a domain found in many regulatory proteins (11), and a WhiB-like transcriptional regulator (mup021). This arrangement shares synteny with a region near oriC of the Mycobacterium tuberculosis (MTB) H37Rv genome. Further upstream of repA is a 5 kb region encoding conserved proteins of unknown function and again there is synteny with the oriC region of MTB. There are 6 genes with products of unknown function but predicted to have membrane-associated domains. None of these displayed similarity to proteins involved in lipid export such as the MMPLs (12) or to any other export systems. The plasmid is rich in insertion sequences (IS), with 26 examples, including four copies of IS2404 and eight copies of IS2606 (13). However the primary function of pMUM001 appears to be toxin production. This is the first report of a plasmid mediating mycobacterial virulence.

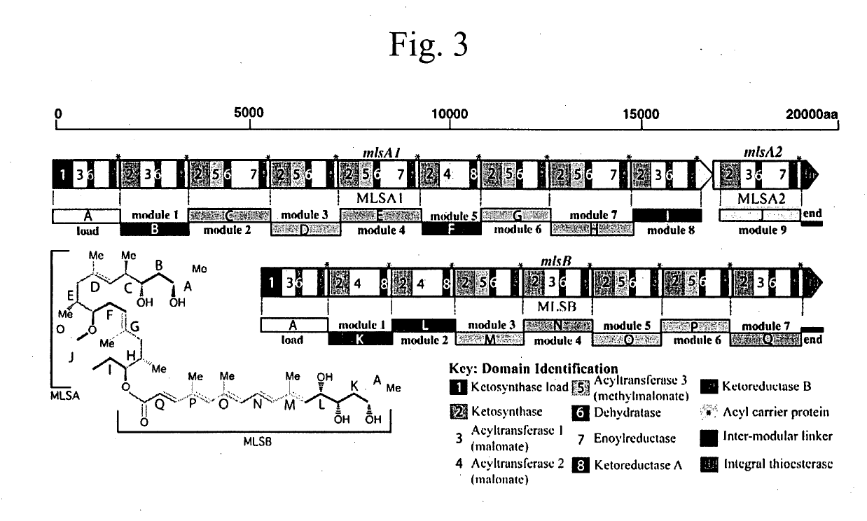

Most of pMUM001 (˜105 kb) consists of six genes coding for proteins involved in mycolactone synthesis (FIG. 2). Mycolactone core-producing PKS are encoded by mlsA1 (50,973 bp) and mlsA2 (7,233 bp) and the side chain enzyme by mlsB (42,393 bp). All three PKS genes are highly related, with stretches of up to 27 kb of near identical nucleotide sequence (99.7%). The entire 105 kb mycolactone locus essentially contains only 9.5 kb of unique, non-repetitive DNA sequence. The repetitive, recombinant and recent nature of the MLS locus is highlighted in the GC-skew plot (FIG. 2), as it traces the start and end of each of the two loading and 16 extension modules that these genes encode (see FIG. 3 and the following section). Ancestral genes of mlsA and mlsB apparently underwent duplication, followed by in-frame deletions and limited divergence. There are also three genes coding for potential polyketide-modifying enzymes including a P450 monooxygenase (mup053), probably responsible for hydroxylation at carbon 12 of the side chain; and an enzyme resembling FabH-like type III ketosynthases (KS) (mup045). The latter has mutations in each of three amino acids critical for KS activity. Similar changes have been detected in KS-like enzymes that catalyse C—O bond formation (14). The product of mup045 may likewise catalyse ester bond formation between the mycolactone core and side chain. Alternatively, attachment of the sidechain may be mediated directly by the C-terminal thioesterase (TE) on MLSB. It is intriguing that the mup045 gene has a GC content of 52.8%, significantly lower than the rest of the plasmid, suggesting that it has been acquired by recent horizontal transfer. Immediately 3′ of mlsA2 is mup037, a gene encoding a type II thioesterase which may be required for removal of short acyl chains from the PKS loading modules, arising by aberrant decarboxylation (15).

### EXAMPLE 2

### Analysis of the Mycolactone PKS Cluster

The modular arrangement of the mycolactone PKS closely follows the established paradigm for “assembly-line” multienzymes (16, 17). The core of mycolactone is produced by MLSA1 and MLSA2. MLSA1 contains a decarboxylating loading module (18) and eight extension modules, while MLSA2 bears the ninth and final extension module and the integral C-terminal thioesterase/cyclase (TE) domain which serves to release the product by forming a 12-membered lactone ring (FIG. 3). The pattern of malonate and methylmalonate incorporation predicted by sequence analysis of the acyltransferase (AT) domains in each module exactly matches that found in mycolactone (19). Similarly, the oxidation state produced at each stage of chain extension almost wholly corresponds to that predicted on the basis of the mycolactone structure (16, 17). The exception is extension module 2, where dehydratase (DH) and enoylreductase (ER) domains appear from sequence comparisons to be active, although the structure of the product does not require these steps. However, there is a precedent from previously-characterised PKS gene clusters for such non-utilisation of reductive domains (19). Likewise, the side-chain of mycolactone is produced by MLSB which contains a decarboxylating loading module, and seven extension modules, plus an integral TE domain, and here the pattern of extender unit incorporation, the oxidation state and the stereochemistry of ketoreductase (KR) reduction (20) are exactly as predicted.

On closer inspection, however, the mycolactone PKS presents some highly unusual features that have an important bearing on our view of the structural basis of the specificity of polyketide chain growth on such multienzymes. First, the PKS proteins are of unprecedented size, with MLSA comprising one multienzyme of eight consecutive extension modules (MLSA1) and predicted molecular mass (1.8 MDa); and a second (MLSA2, 0.26 MDa) harbouring the last extension module and the TE. The recognition process between MLSA1 and MLSA2 is mediated in part by specific “docking domains” as in other modular PKSs (21). Meanwhile, MLSB contains all of its seven consecutive extension modules in a single multienzyme (1.2 MDa). These are among the largest proteins predicted to be found in any living cell. The most startling feature of the mycolactone PKS is the extreme mutual sequence similarity between comparable domains in all 16 extension modules (FIG. 3). While modular PKSs routinely show 40-70% sequence identity when domains from the same PKS are compared, and lower identity when domains from different PKS are compared (19), the identity scores for the DH, ER, A-type and B-type KR domains in the mycolactone locus ranged between 98.7 and 100%.

There were three distinct sequence types for the AT domains; two with predicted malonate specificity and the third, methylmalonate. Within each of the three AT domain types identity scores were 100% (FIG. 3) while between the sequence types the identity was 34%. Interestingly, one of the malonate AT domain types was always linked to the A-type KR domain. This divergent domain combination was found in module 5 of MLSA1 and modules 1 and 2 of MLSB (FIG. 3) and were 100% identical for both their aa and DNA sequences. The most likely explanation is recent acquistion by horizontal transfer followed by duplication. This is supported by the significantly lower GC content of this block compared to the surrounding sequences (58% versus 63%, FIG. 2).

For the KS domains, which catalyse the critical C—C bond-forming steps, the mutual sequence identity within all of the MLS modules is over 97%. Only 11 residues out of 420 show variation and none of this variation appears systematic. Other modular PKSs demonstrate sequence identity between KS domains in the range of 32-67% (Table 1).

TABLE 1
Shared percentage amino acid identity amongst the KS domains of four PKS

| | MLSA, B | RAPS1, 2, 3 | DEBSI1, 2,3 | PikAI, II, III, IV |
|---|---|---|---|---|
| | (mycolactone¹⁶*) | (rapamycin¹⁴) | (erythromycin⁶) | (pikromycin⁶) |
| MLSA, B | 97 | | | |
| (mycolactone¹⁶) | | | | |
| RAPS1, 2, 3 | 66 | 67 | | |
| (rapamycin¹⁴) | | | | |
| DEBS1, 2, 3 | 38 | 32 | 38 | |
| (erythromycin⁶) | | | | |
| PikAI, II, III, IV | 47 | 39 | 32 | 51 |
| (pikromycin⁶) | | | | |

*indicates number of extension modules

The synthetic operations catalysed by various KS domains of the mycolactone PKS involve significant structural variation in both the growing polyketide chain and the incoming extender unit. Mass-spectrometry (LC-MS) experiments on mycolactone-containing extracts of MU have, however, confirmed that MLSA apparently produces only one product, while MLSB only shows minor variation in two or three out of seven modules (22).

These data lead to the unexpected conclusion that the KS domains in this PKS play no significant role in determining the specificity of polyketide chain growth.

A practical outcome of this finding is that the mycolactone PKS modules might furnish the basis of a set of “universal” extension units in engineered hybrid modular PKSs, with potentially far-reaching implications for combinatorial biosynthesis (see Example 6). In conclusion, the singularly high level of DNA sequence homology suggests that the mycolactone system has evolved very recently, arising from multiple recombination and duplication events. It also suggests a high level of genetic instability. Indeed, heterogeneity has been reported both in structure and cytotoxicity of mycolactones produced by MU isolates from different regions (9). High mutability may explain the sudden appearance of Buruli ulcer epidemics as some strains produce mycolactones that confer a fitness advantage for an environmental niche such as the salivary glands of particular aquatic insects (23). This might be accompanied by an increase in virulence or transmissibility to humans. Loss or gain of pMUM001 may also contribute to these events (24). In any event, the deciphering of the mycolactone biosynthetic pathway permits new approaches to be used to prevent and combat M. ulcerans infection.

### EXAMPLE 3

### Construction and Analysis of Mycolactone Negative Mutants

Material and Methods

Phage MycoMarT7 was propagated in M. smegmatis mc²155. It consists of a temperature sensitive mutant of phageTM4 containing the mariner transposon C9 Himar1 and a kanamycin cassette (8). An MU 1615 cell suspension, containing approximately 10⁹ bacteria, was infected with 10¹⁰ phages for 4 h at 37° C. and then plated directly onto solid media containing kanamycin and cultured at 32° C. Non-pigmented colonies were purified and individual mutants subcultured in broth and grown for 5 weeks. Bacteria, culture filtrate and lipid extracts were assayed for cytotoxicity using L929 murine fibroblasts as previously described (9). Lipids were further analyzed by mass spectroscopy for the presence or absence of ions characteristic of mycolactone: the molecular ion [M+Na]+(m/z 765.5), and the core ion [M+Na]+m/z 447 (9).

Results

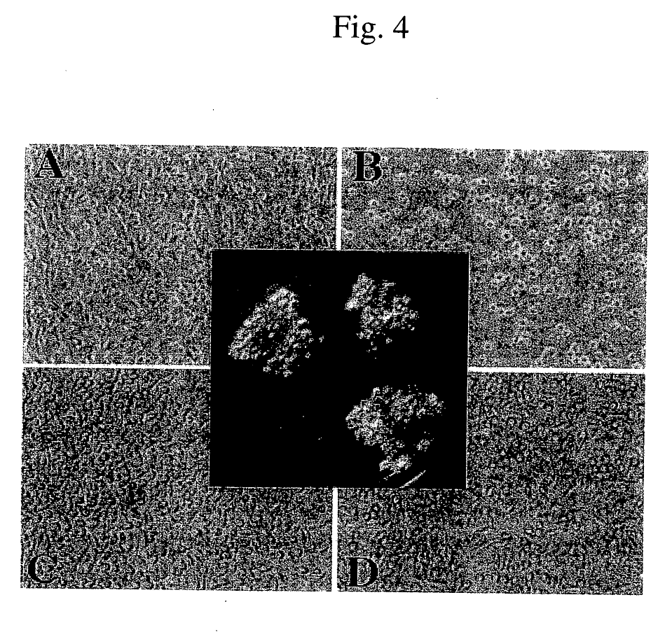

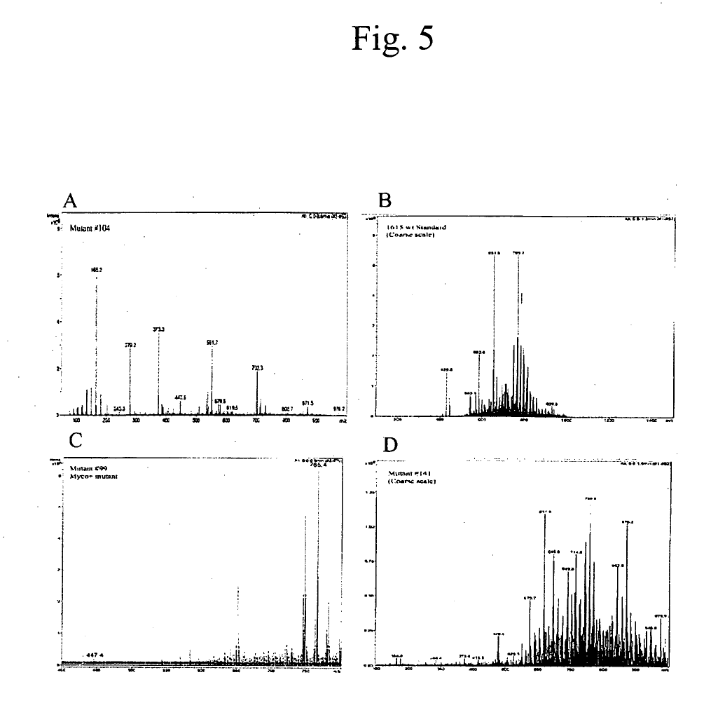

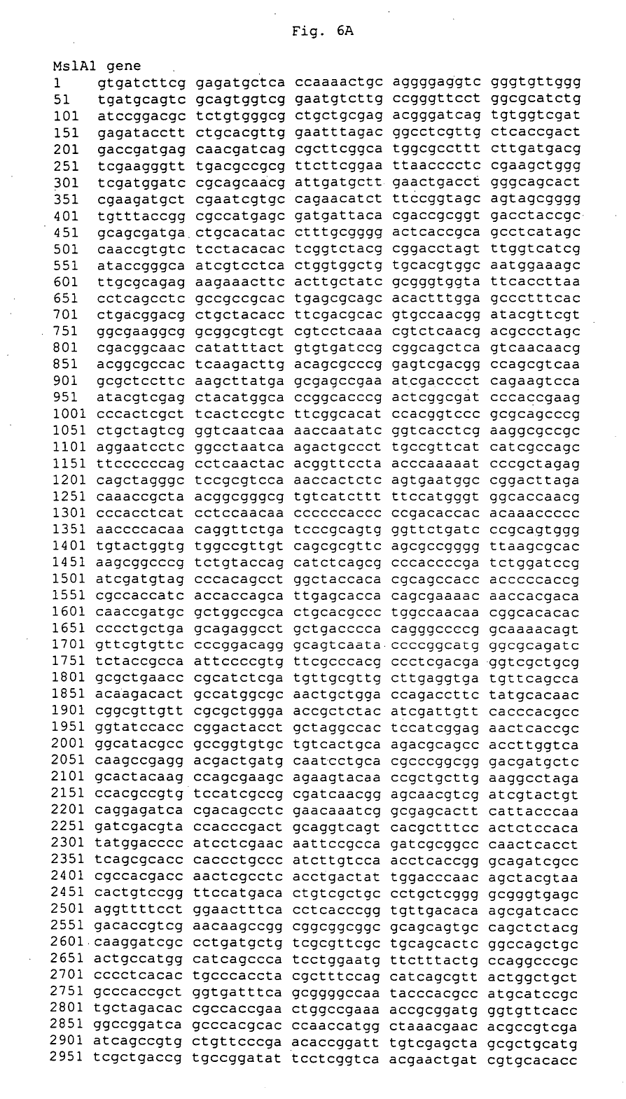

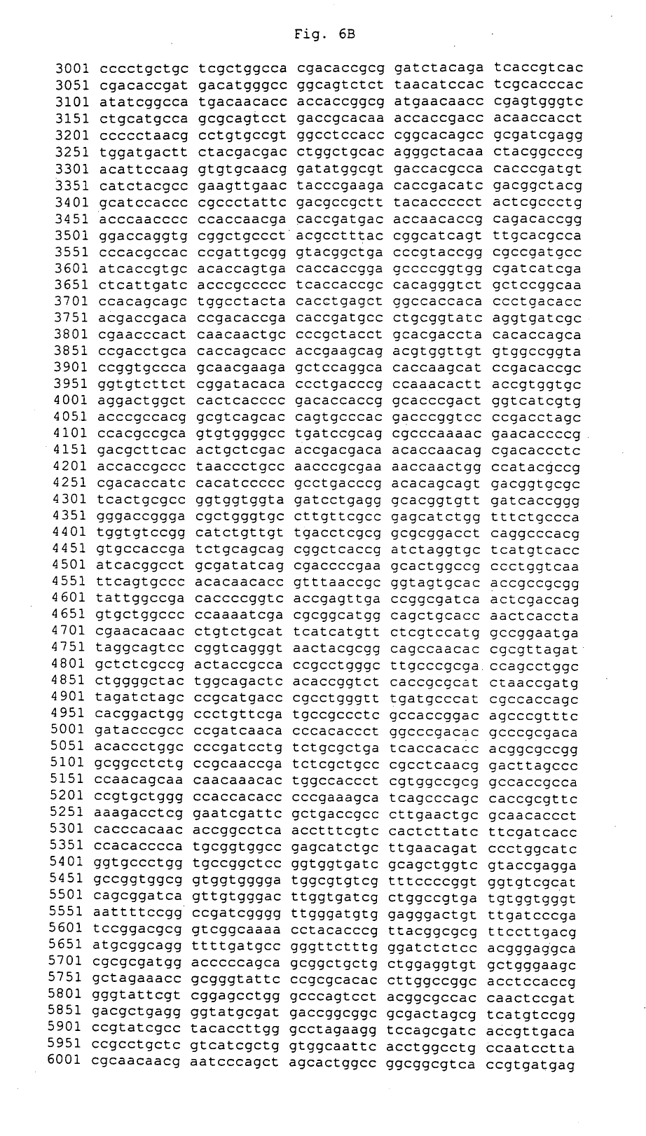

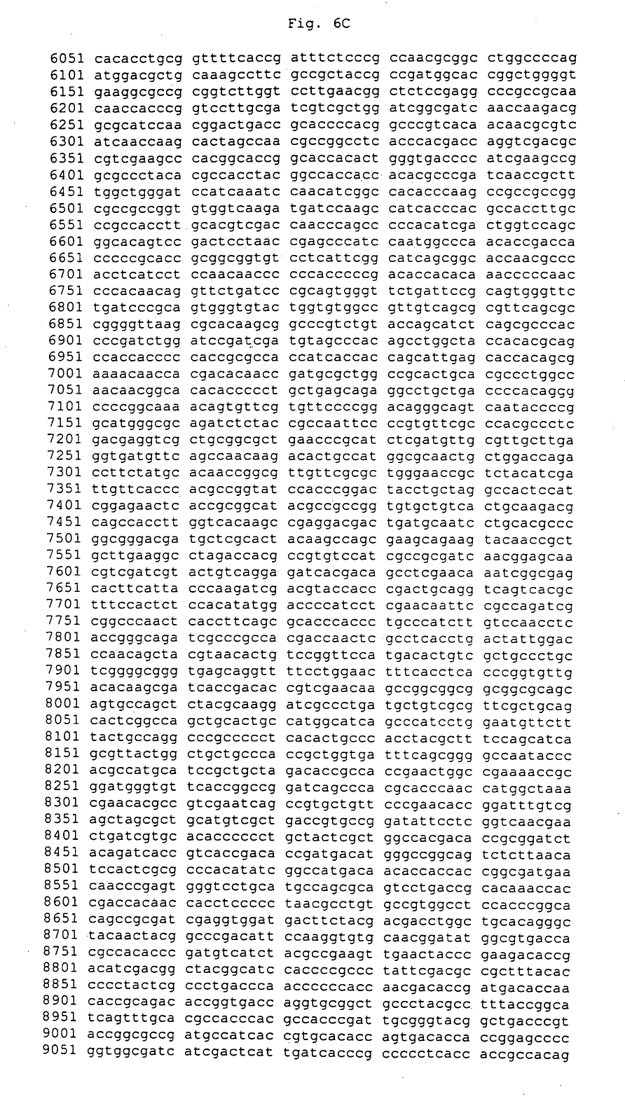

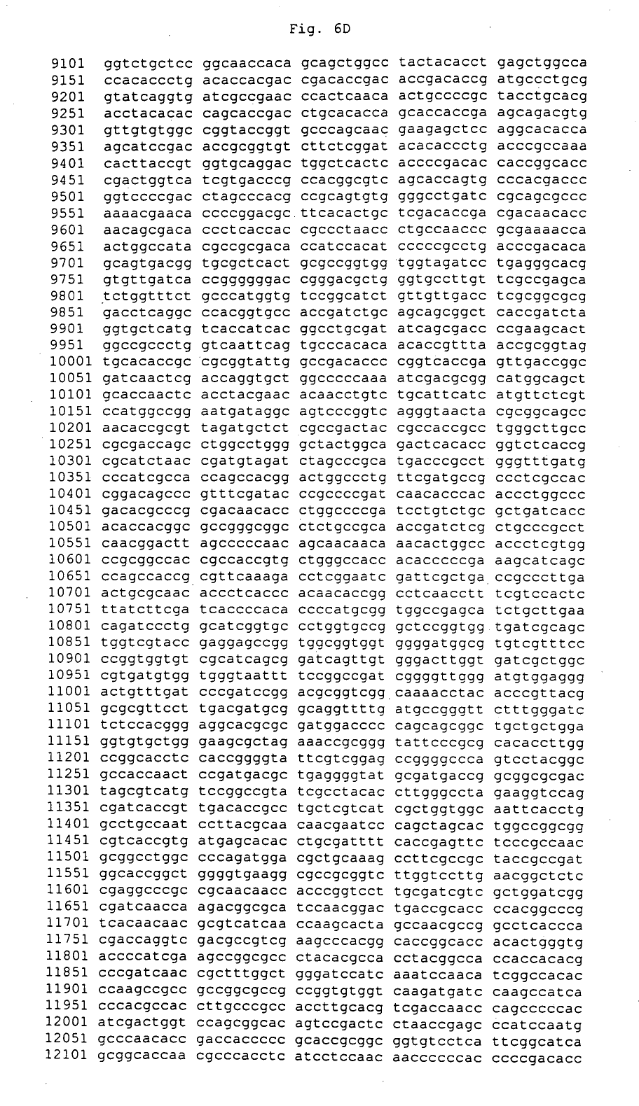

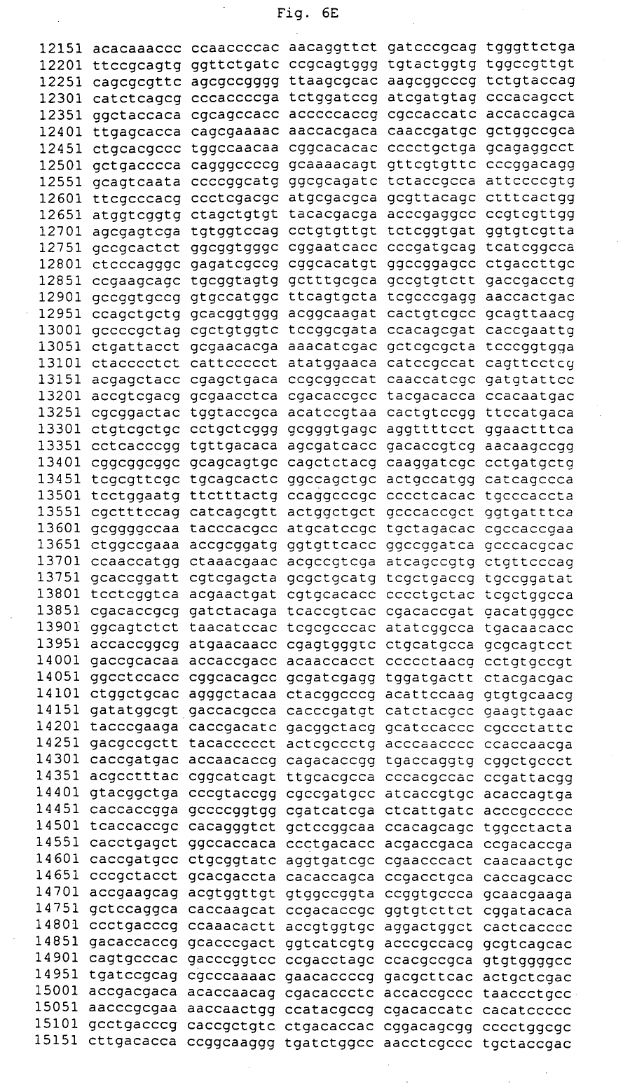

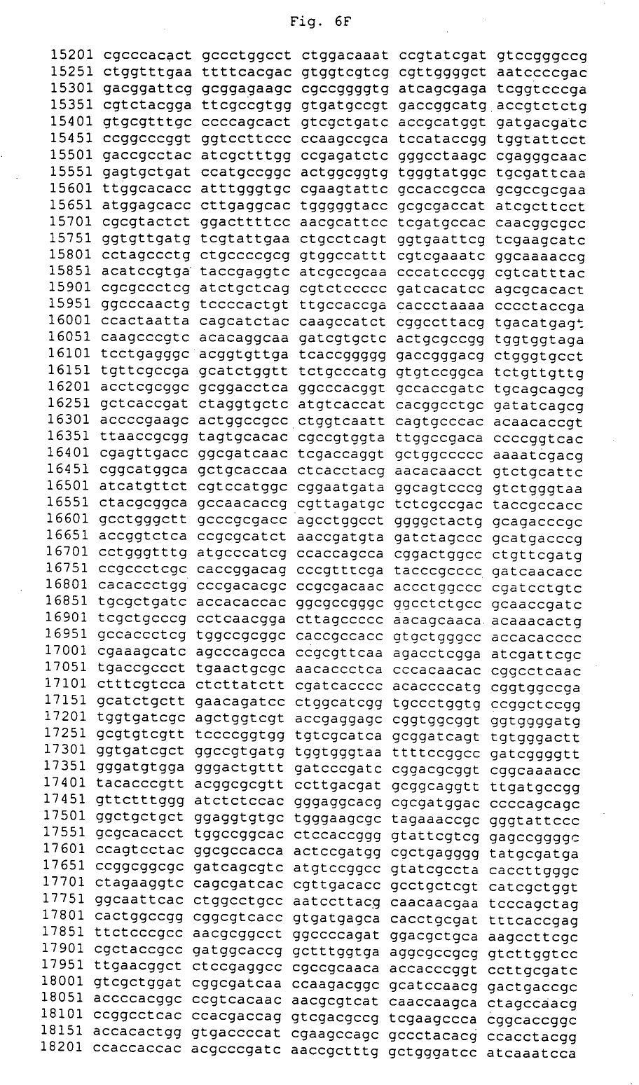

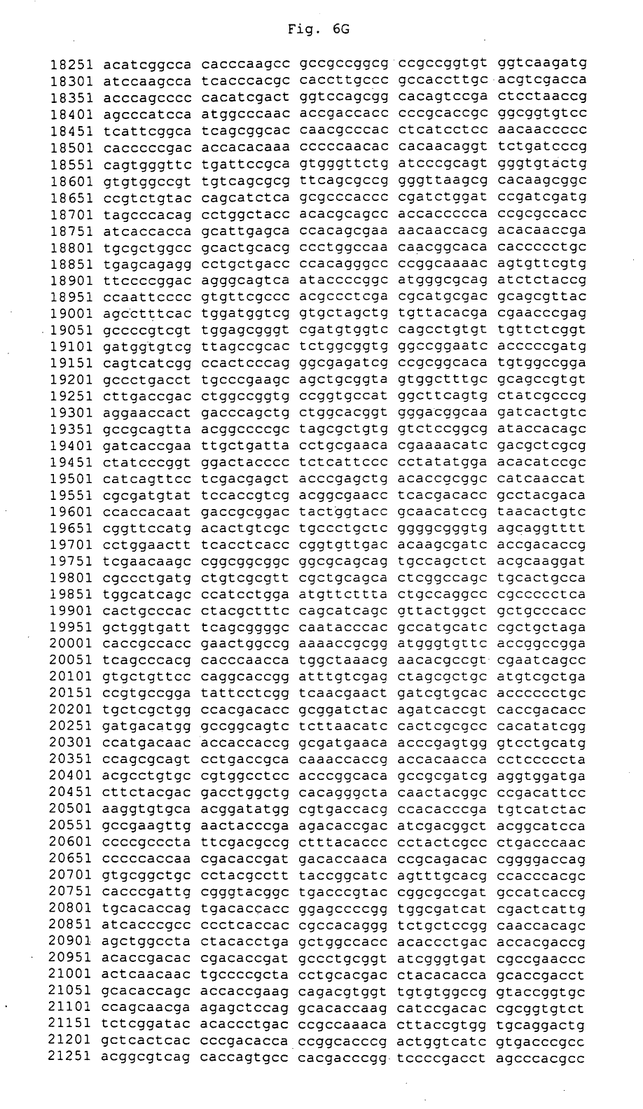

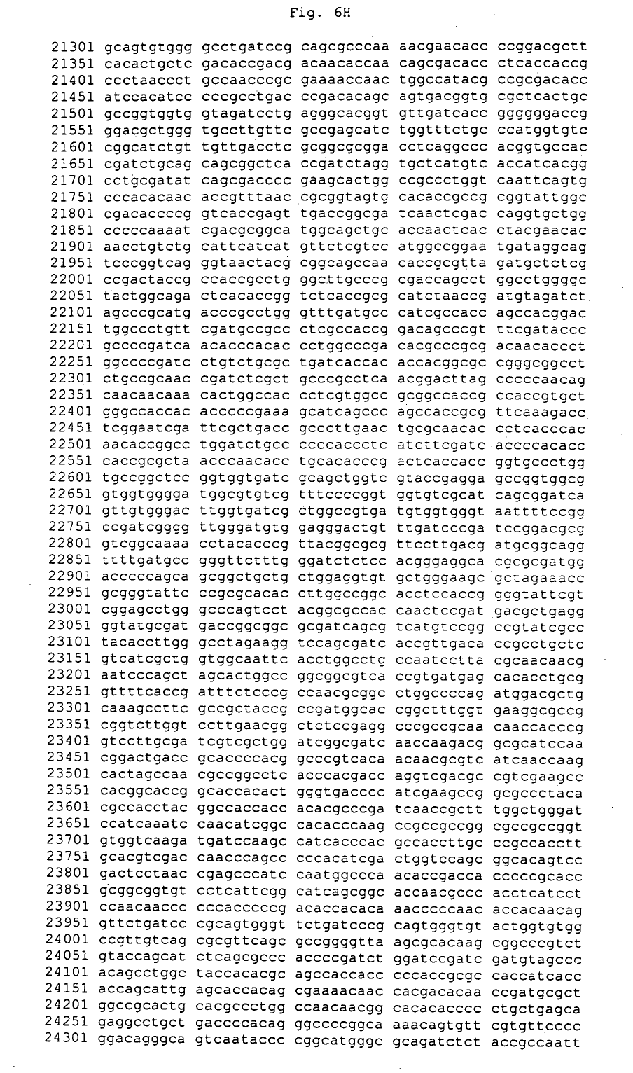

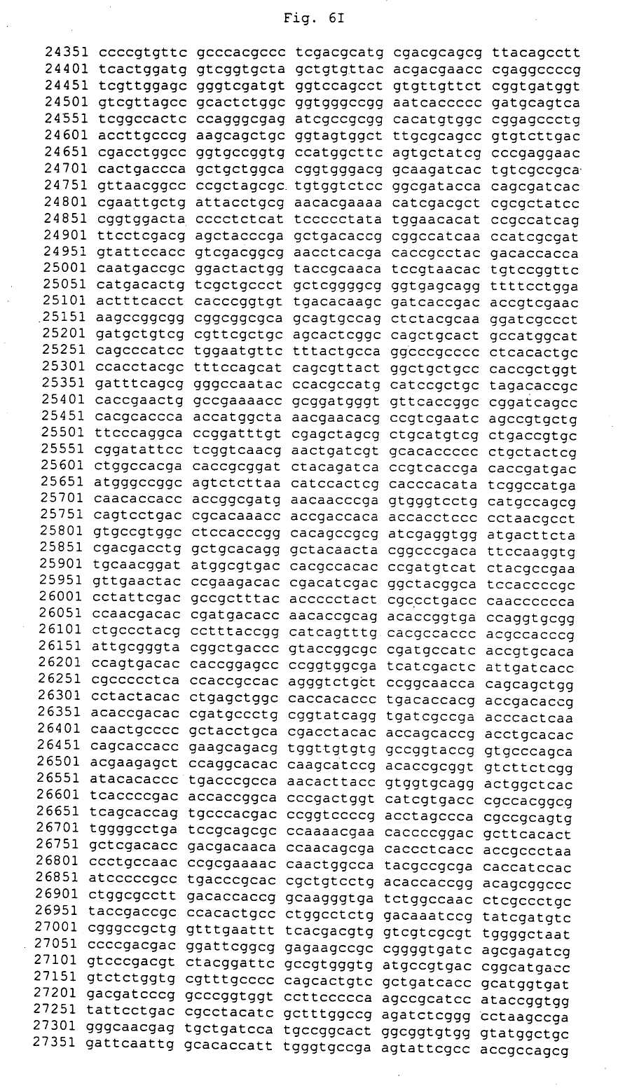

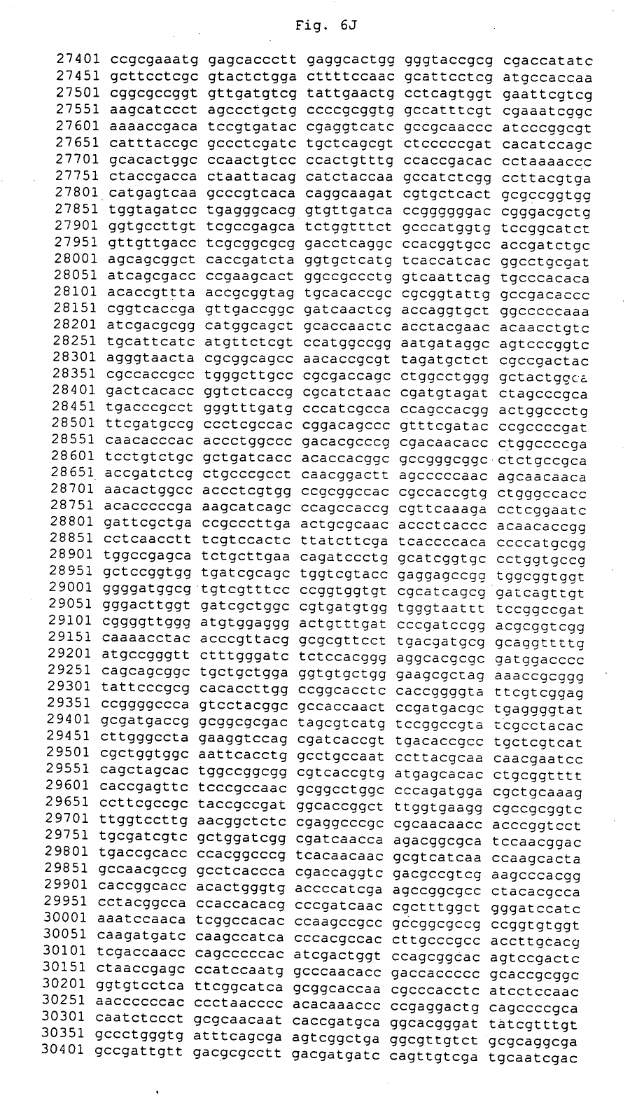

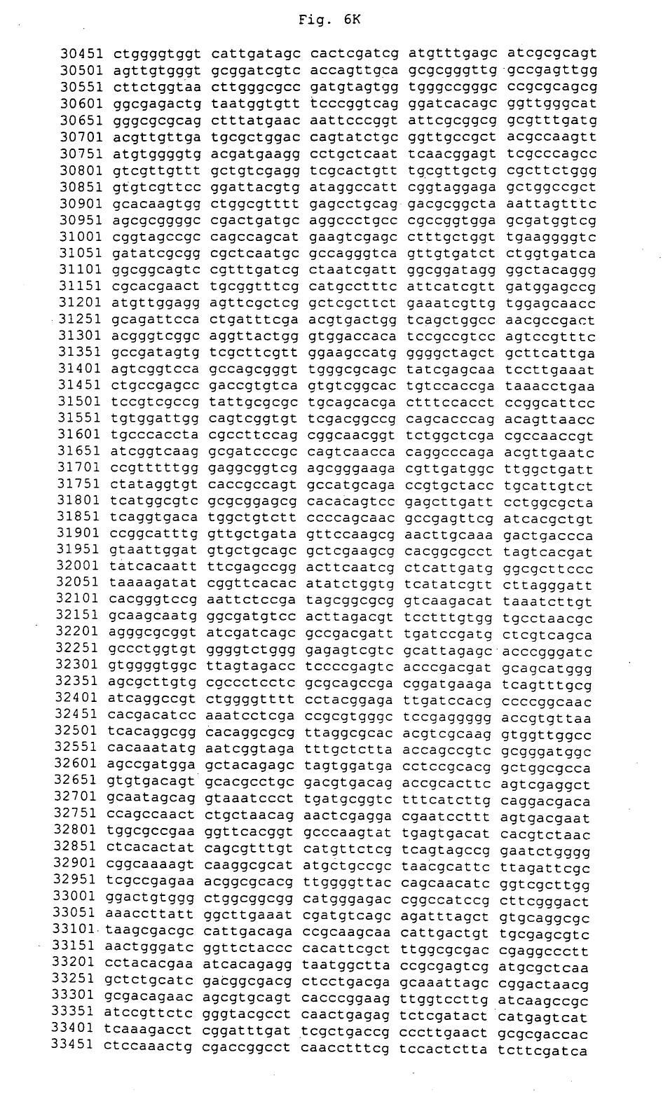

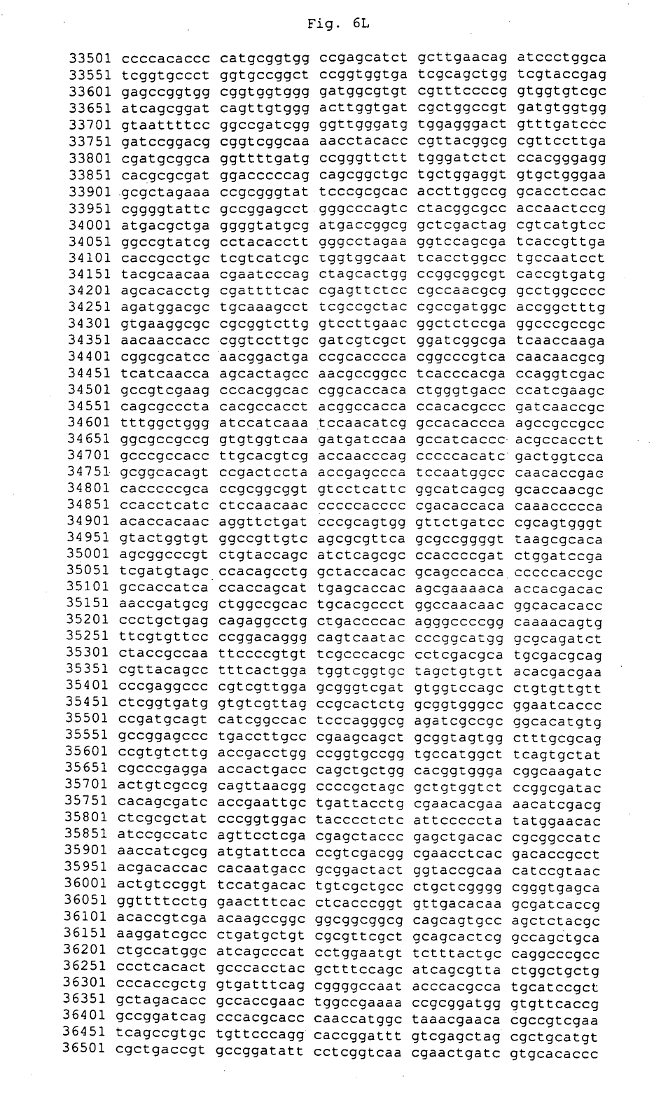

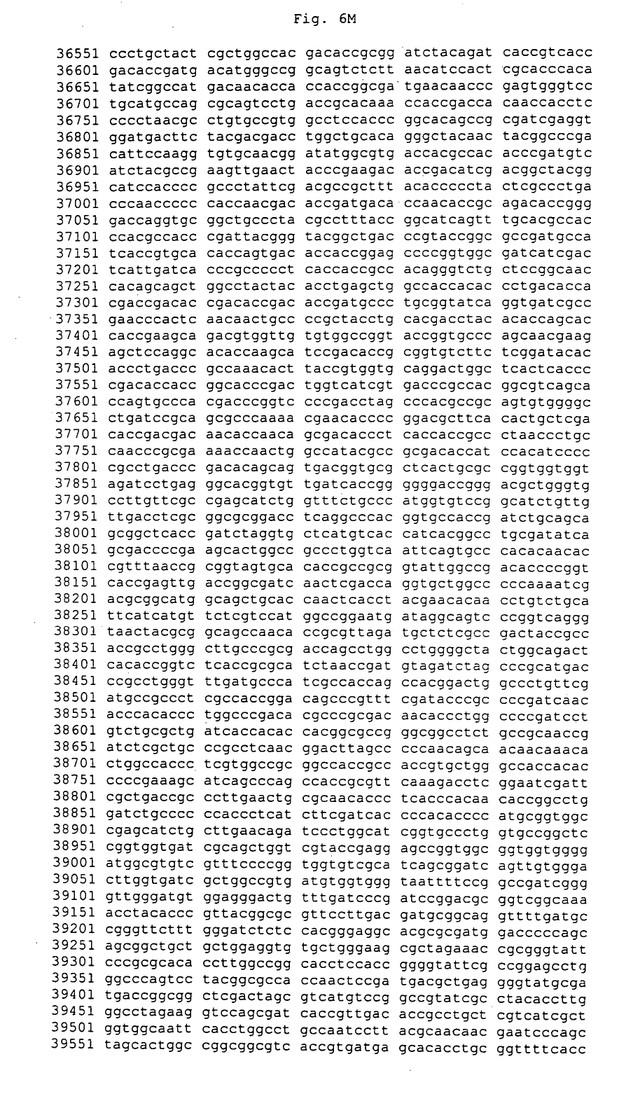

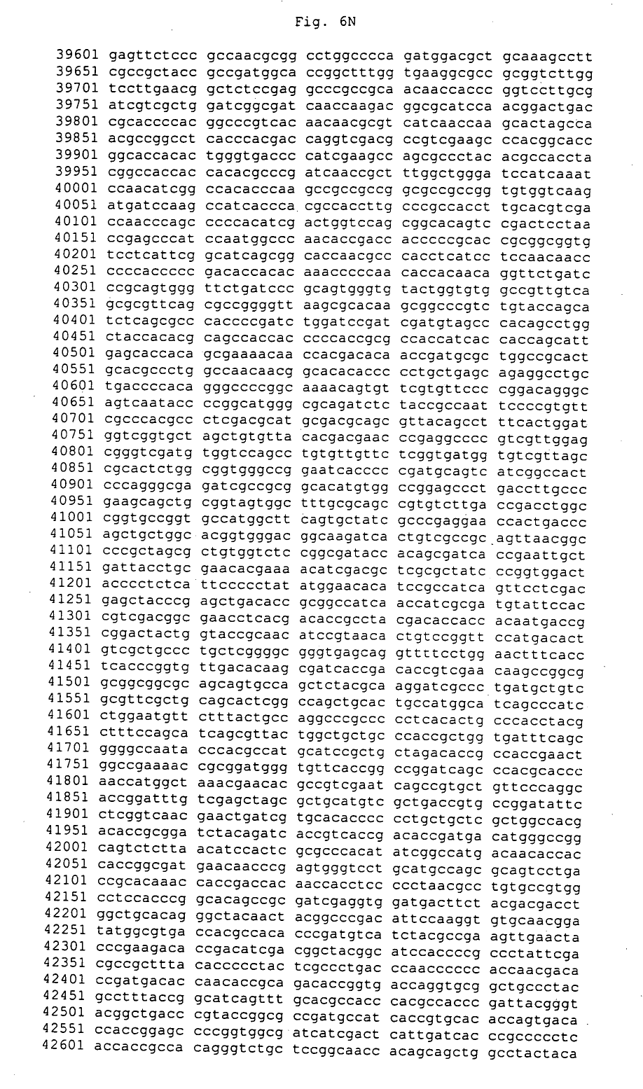

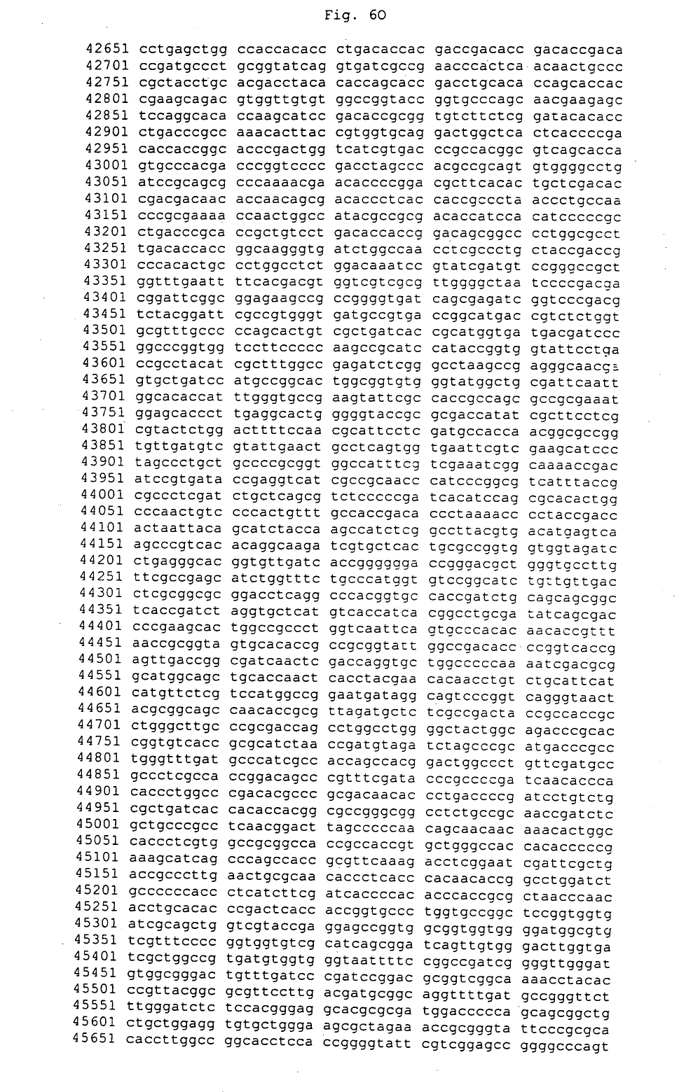

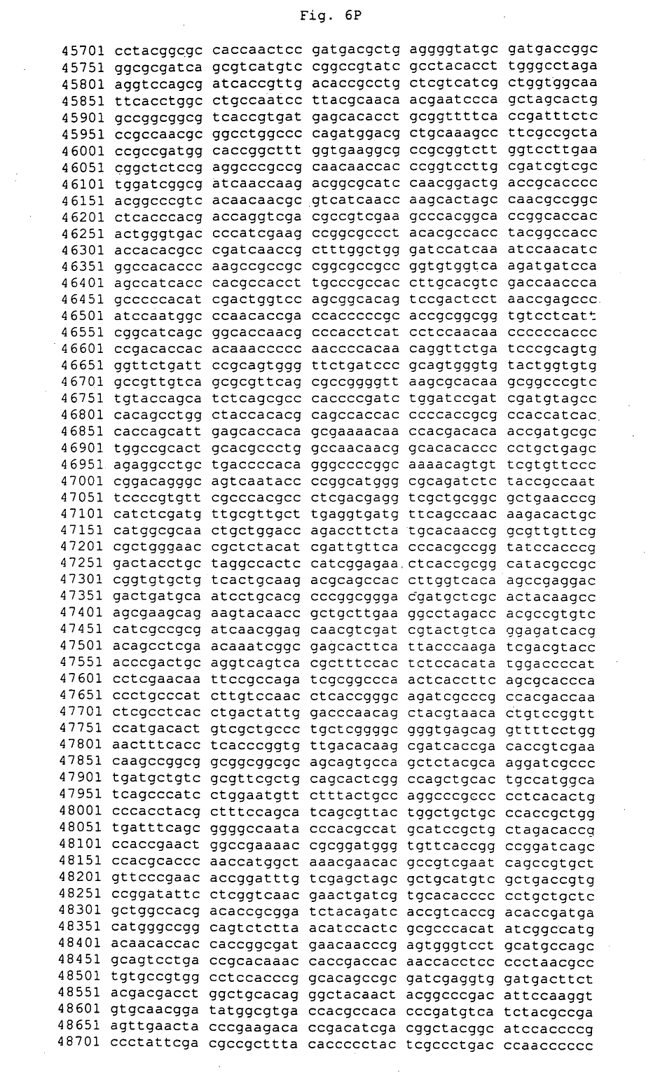

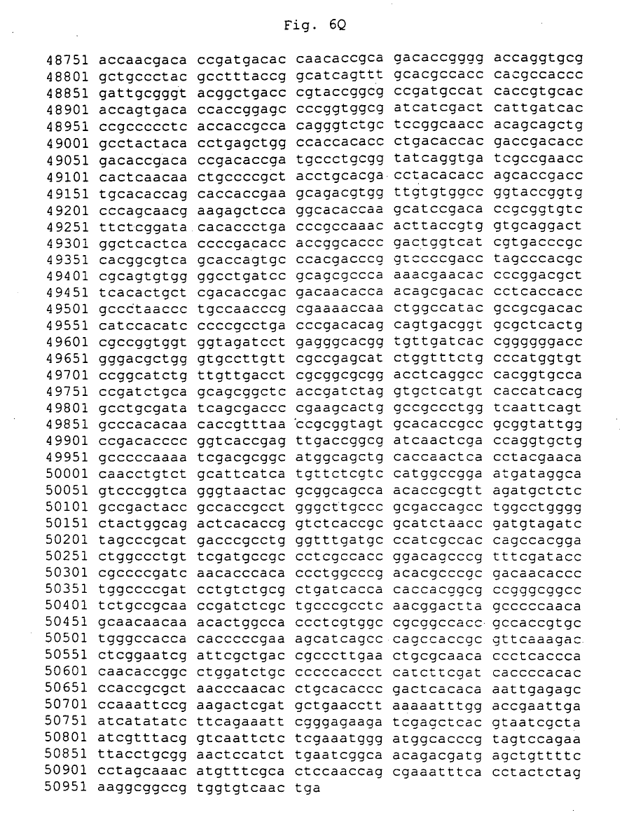

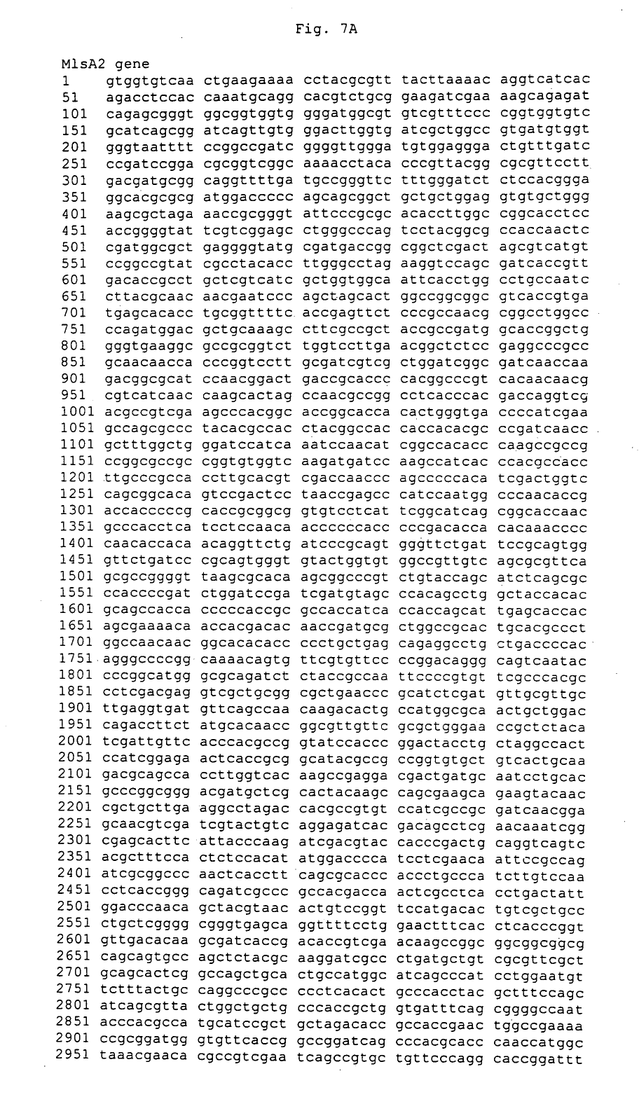

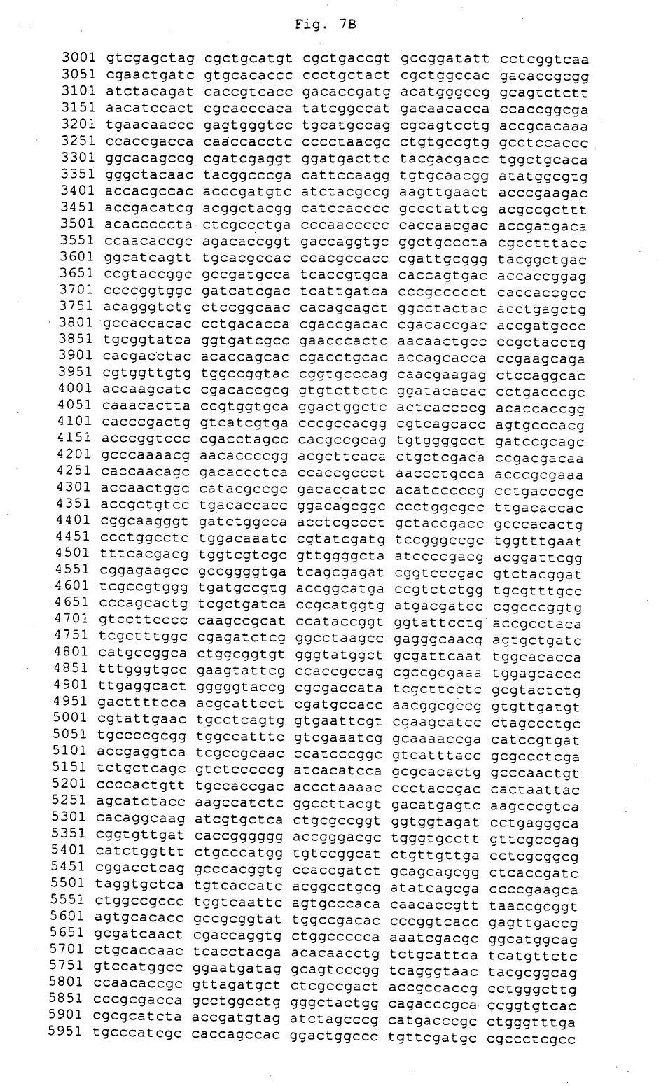

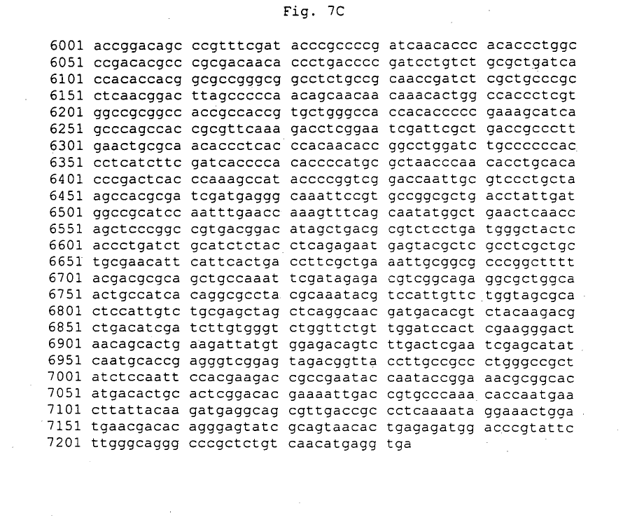

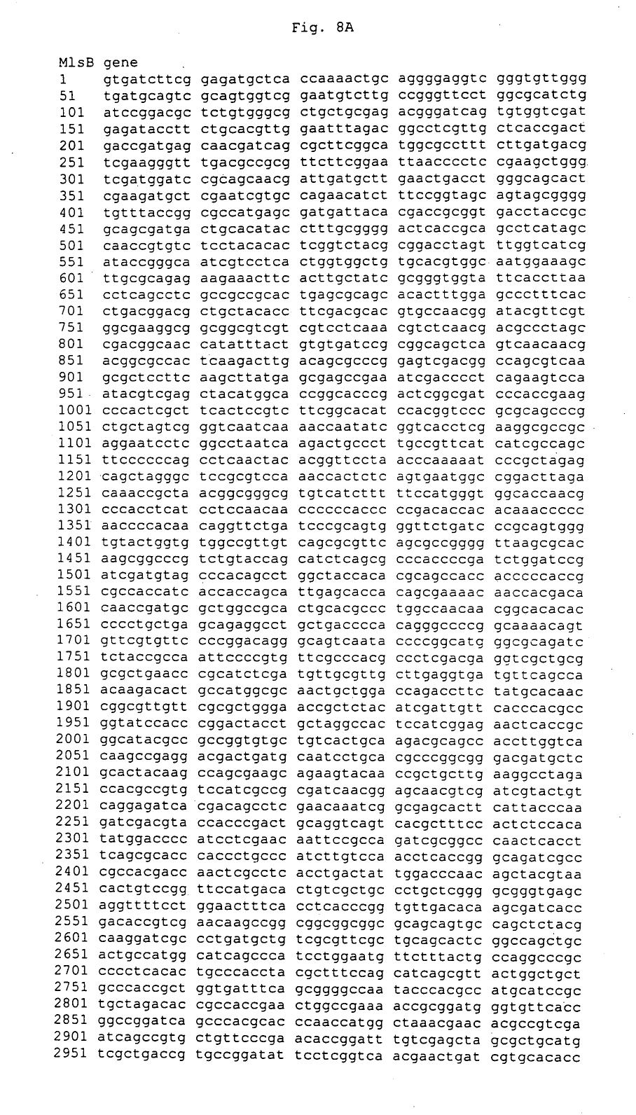

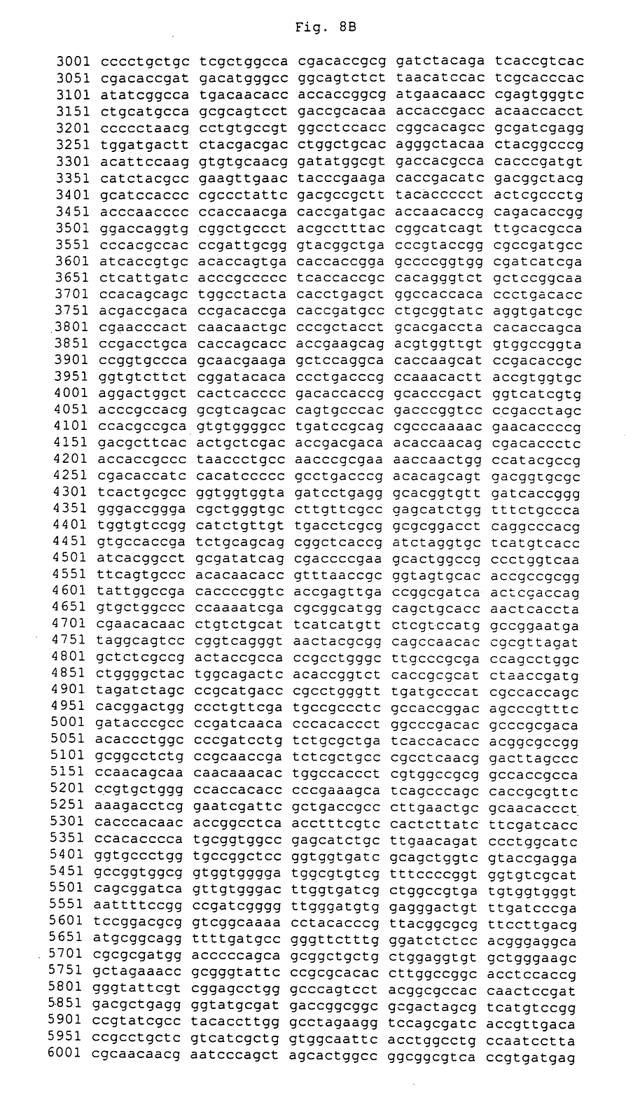

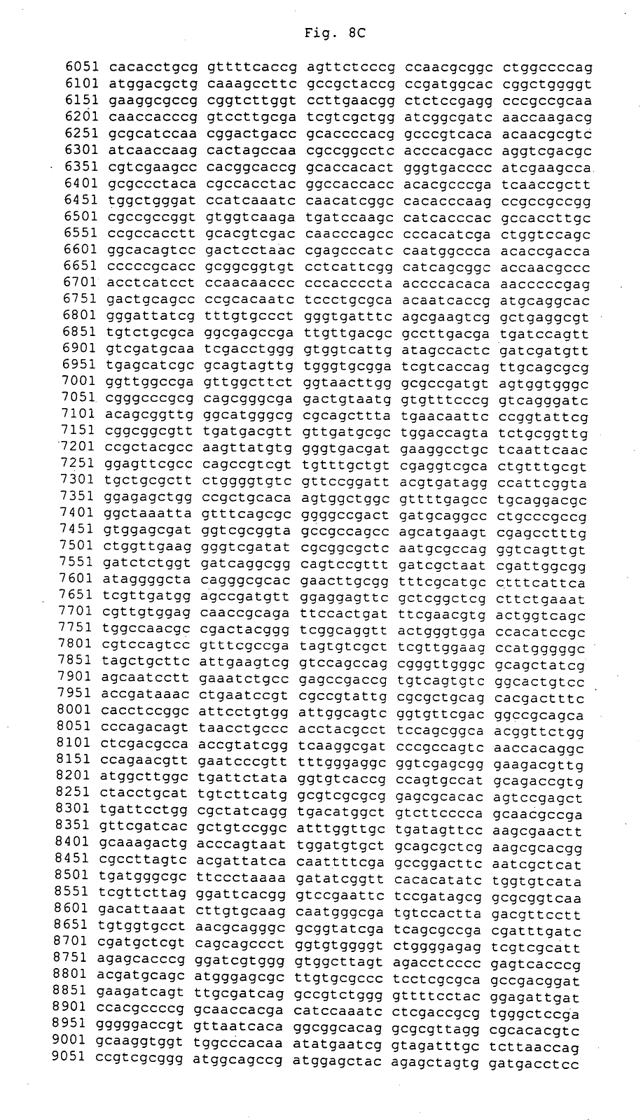

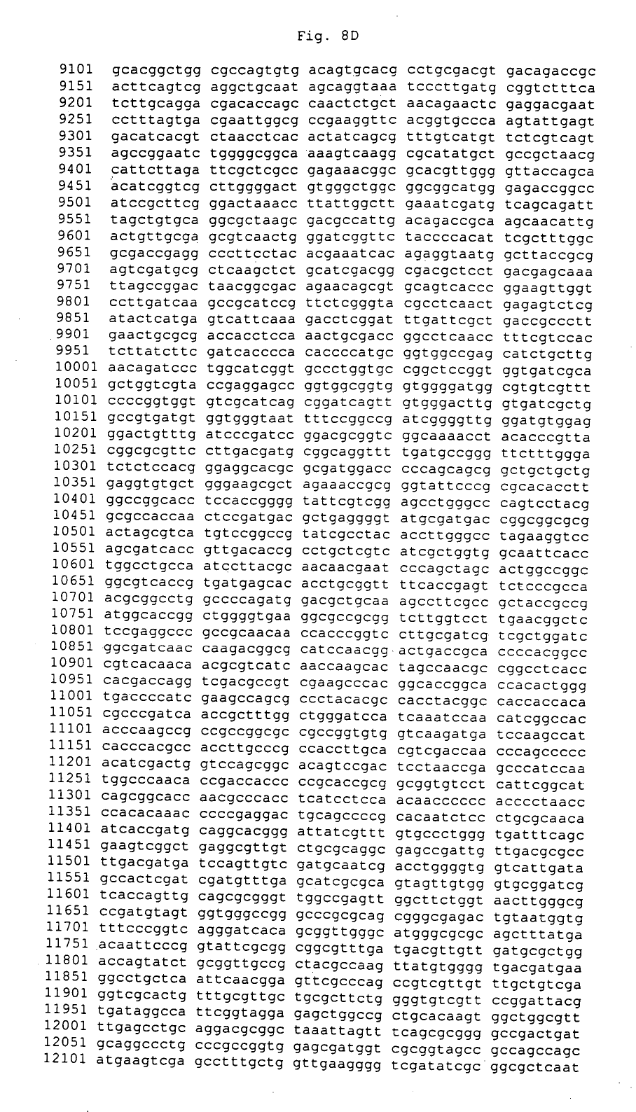

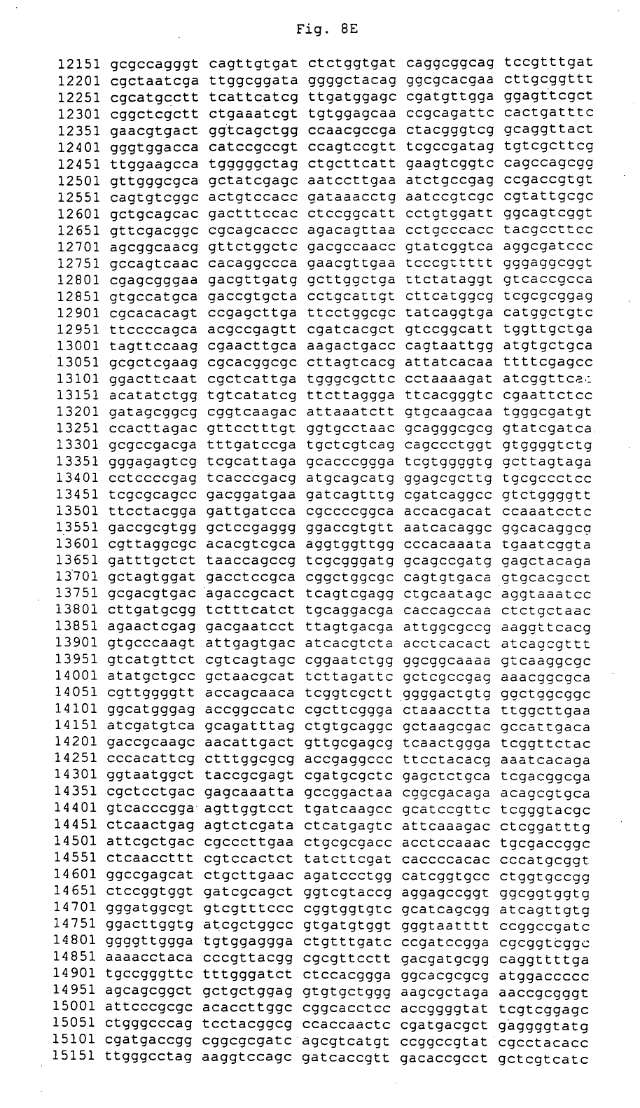

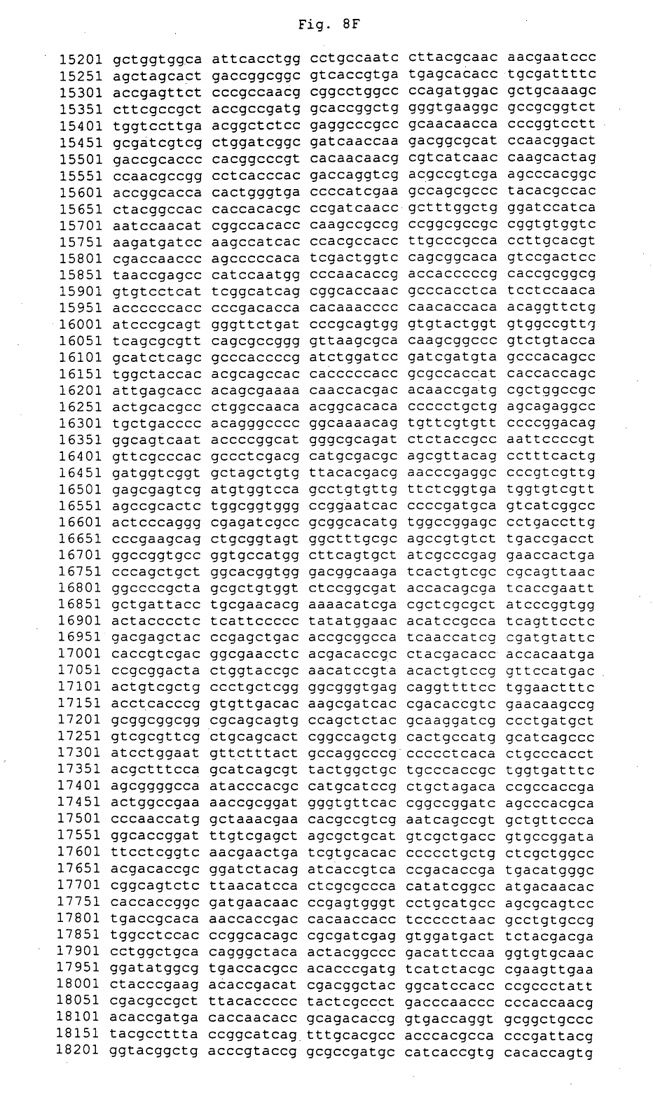

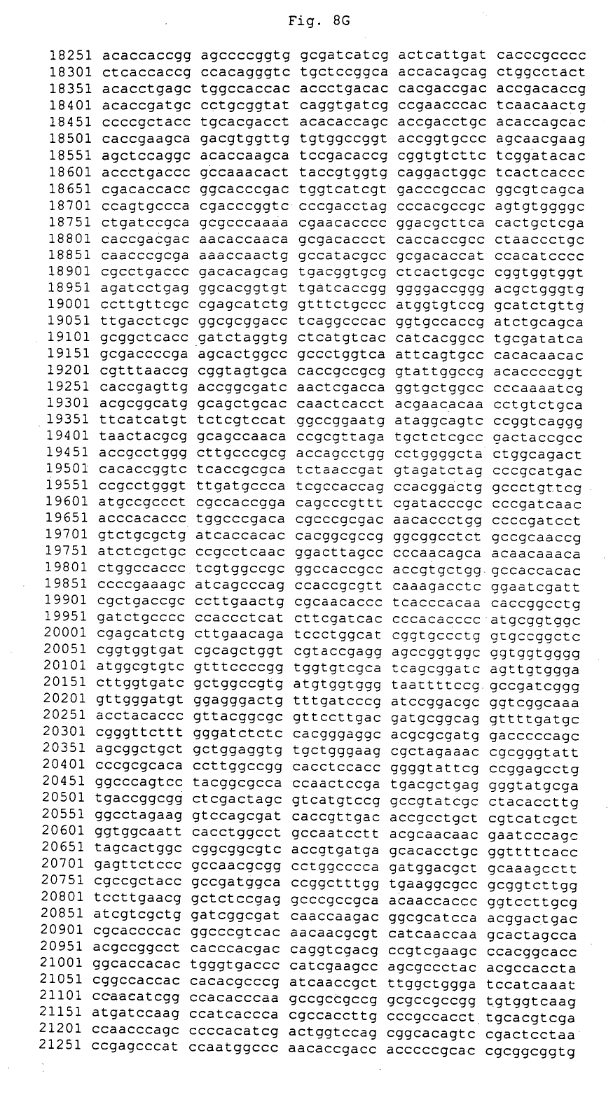

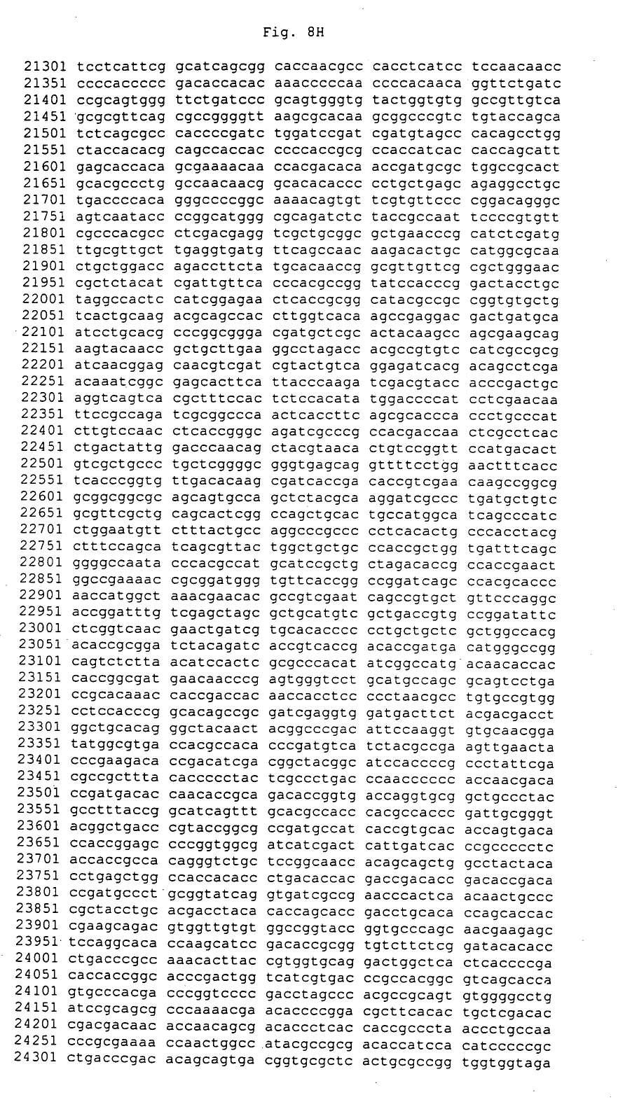

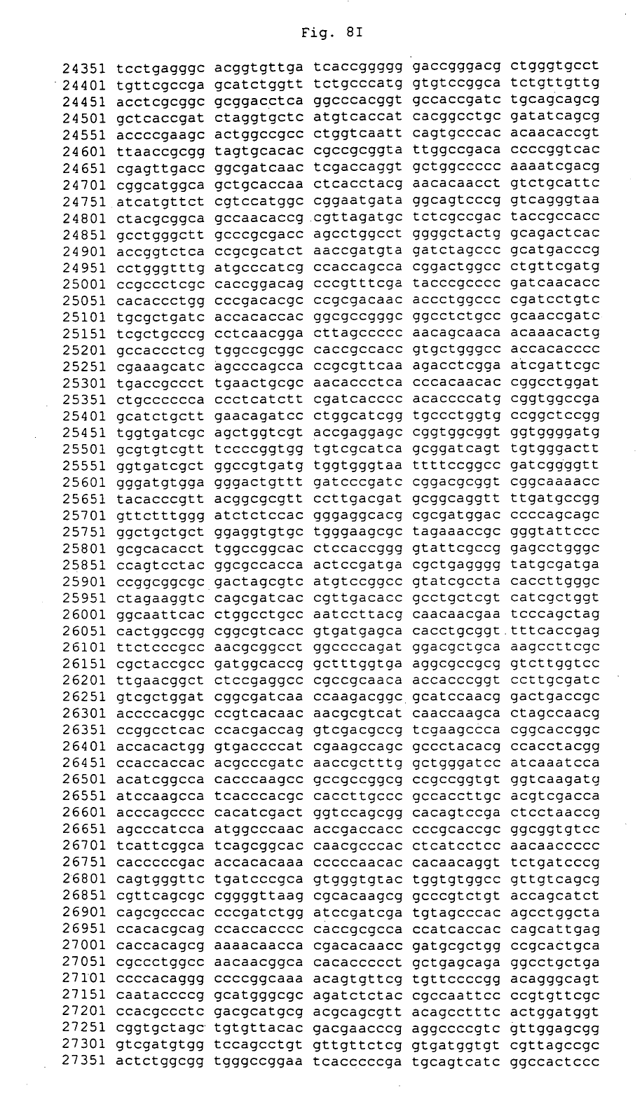

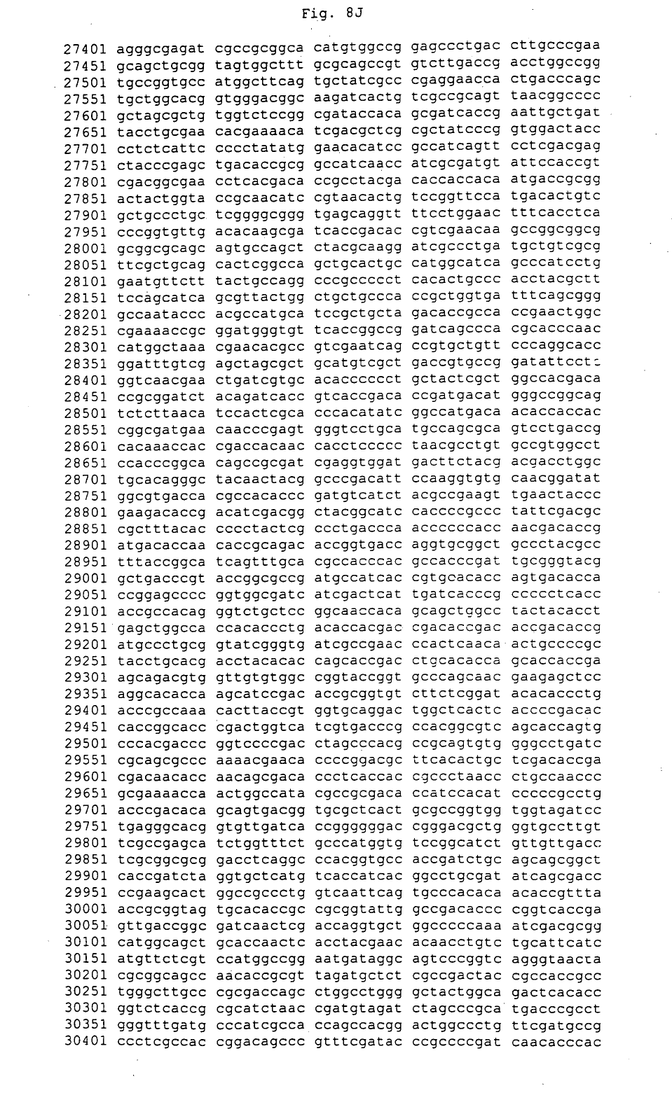

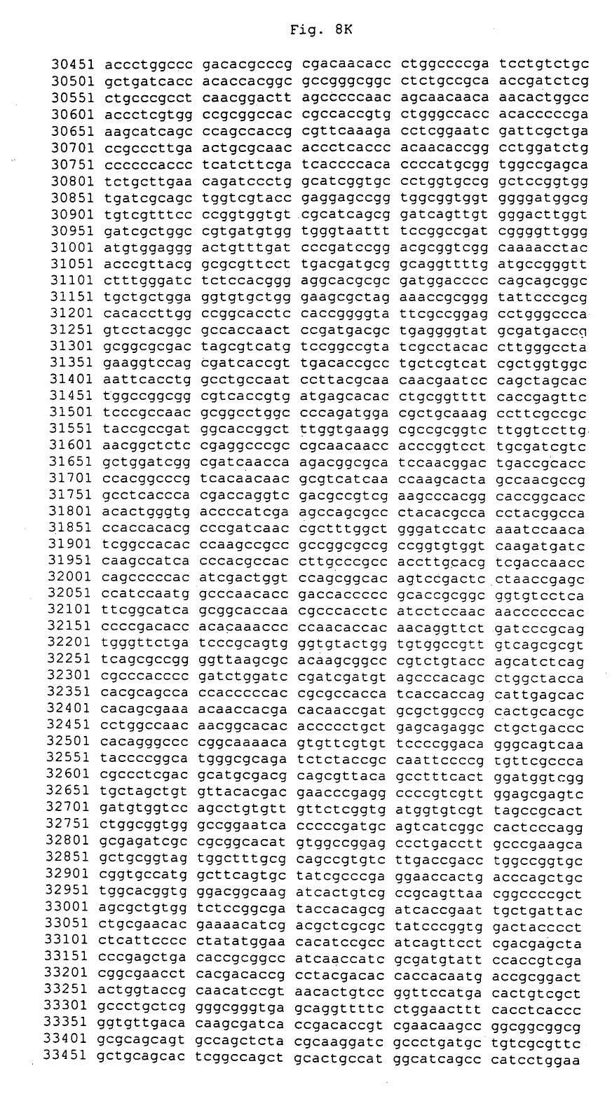

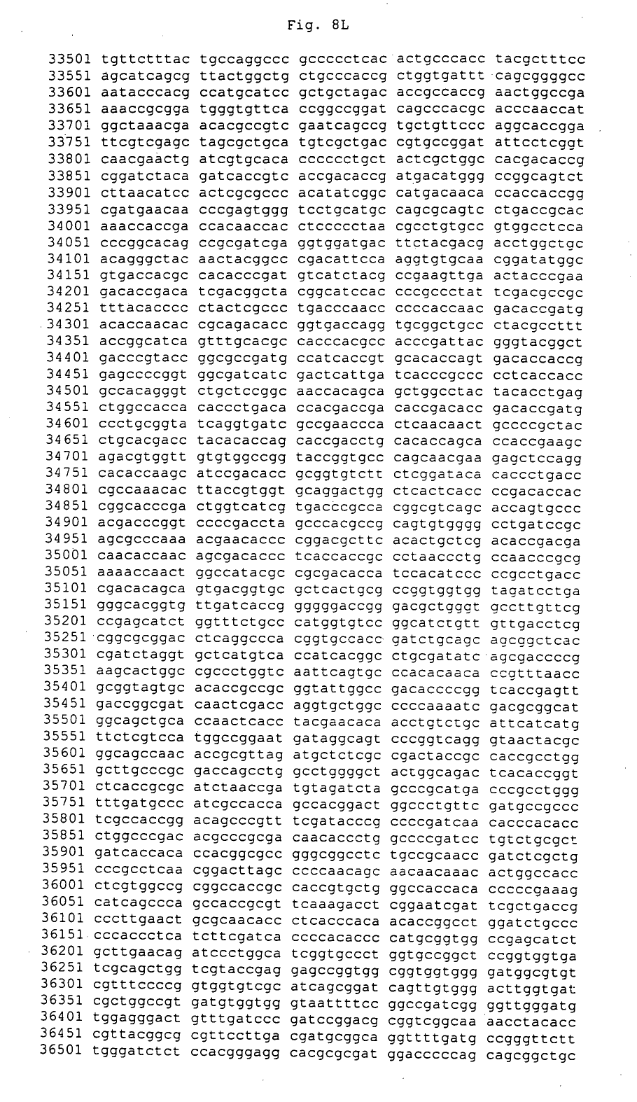

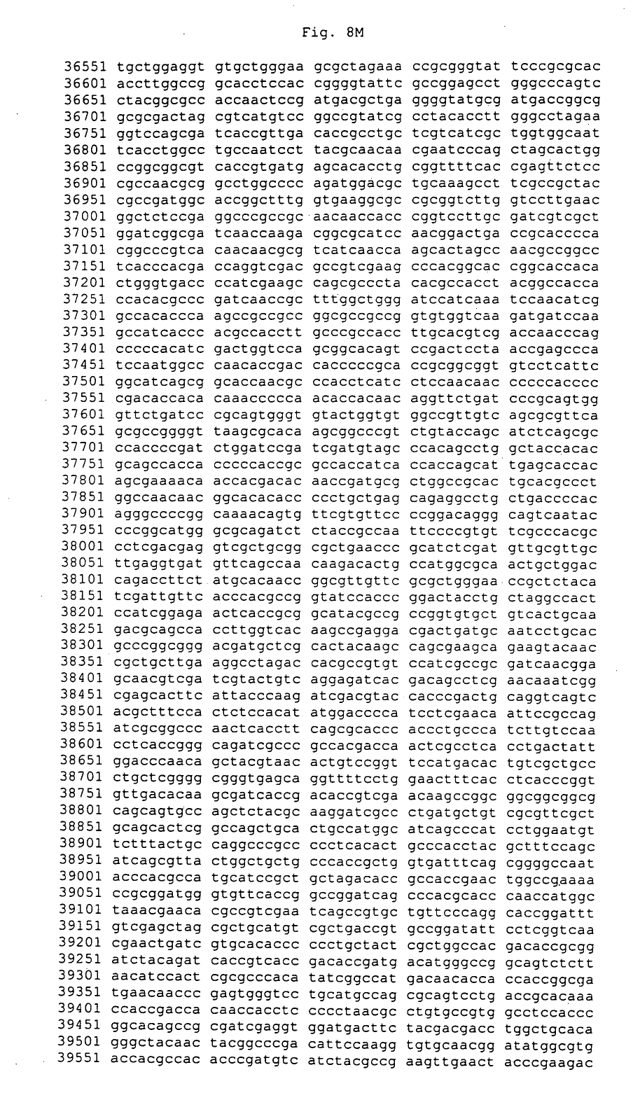

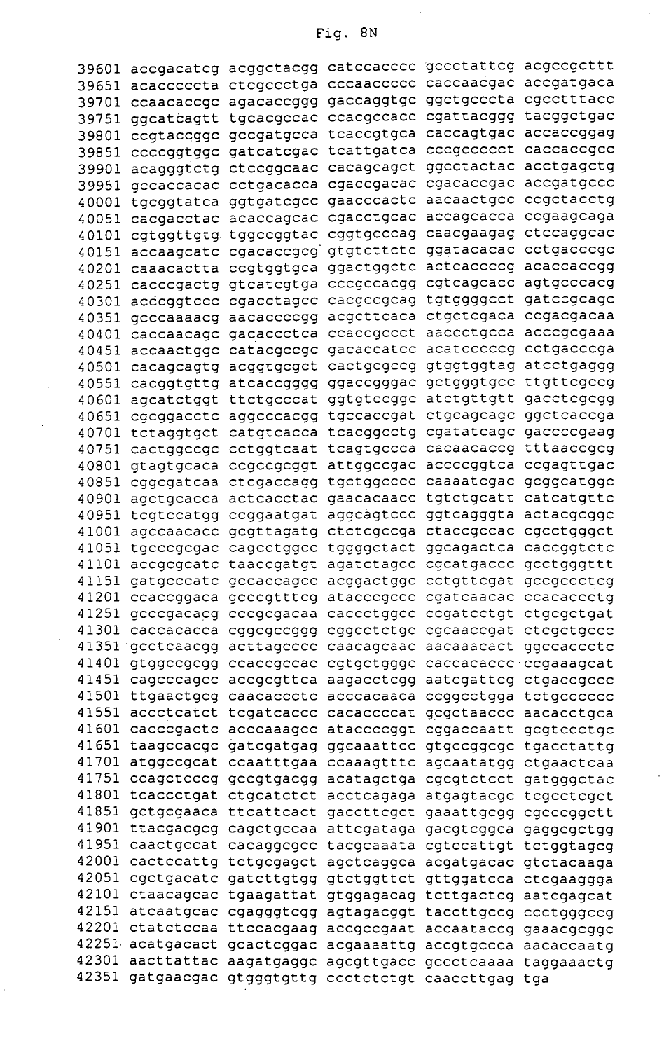

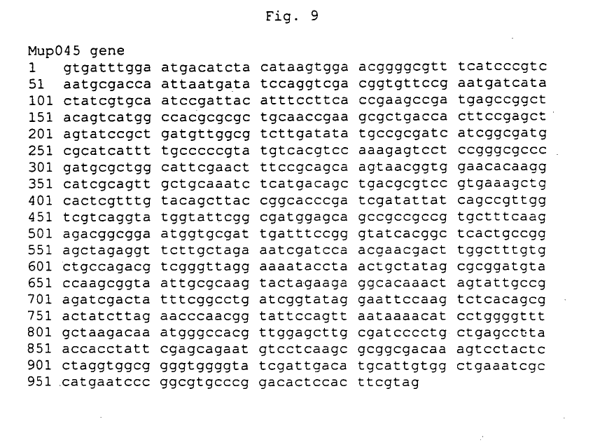

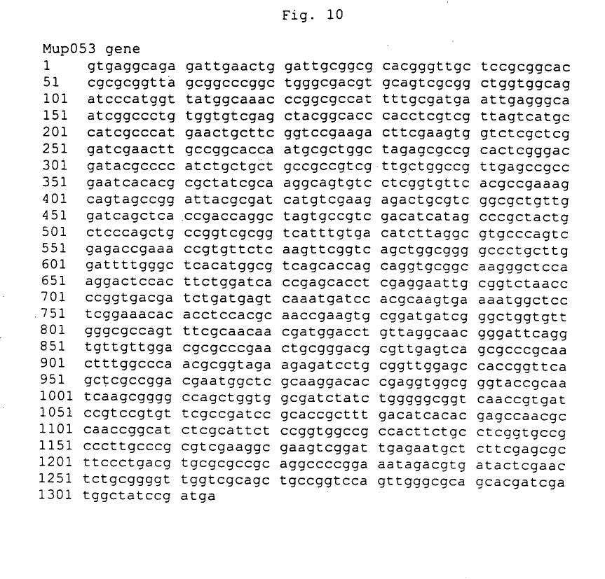

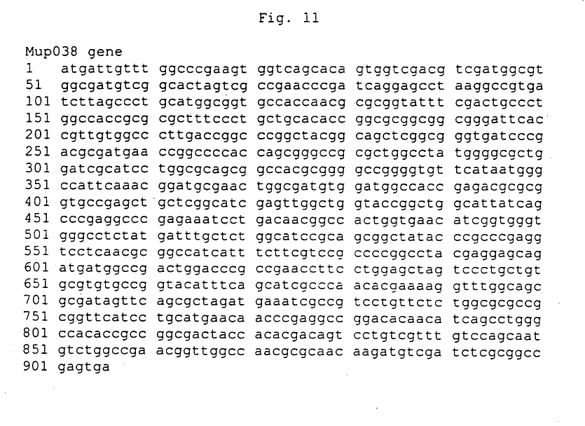

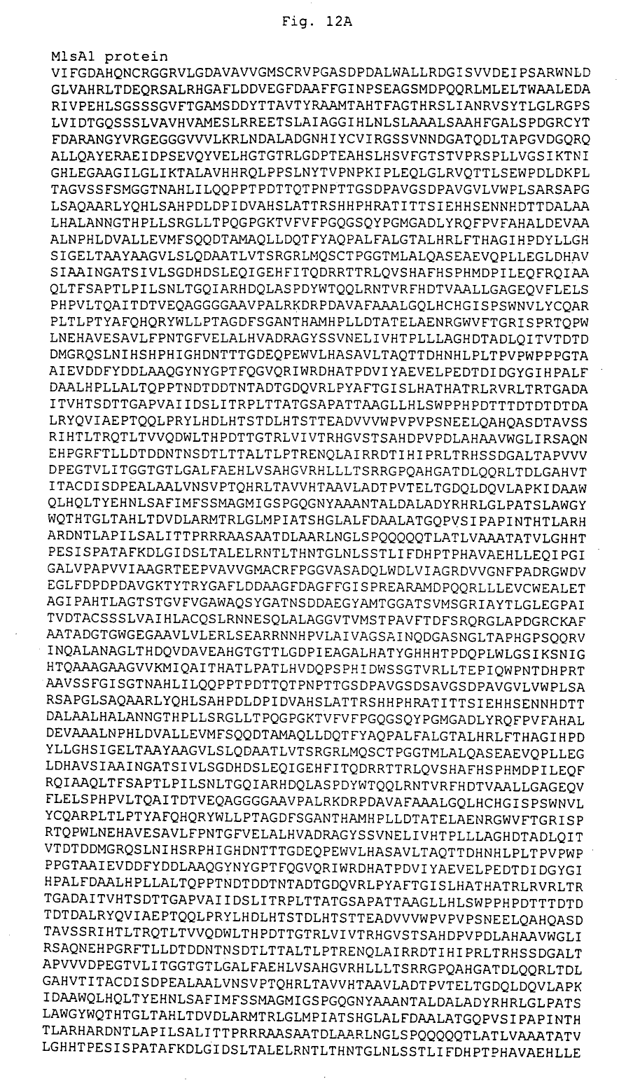

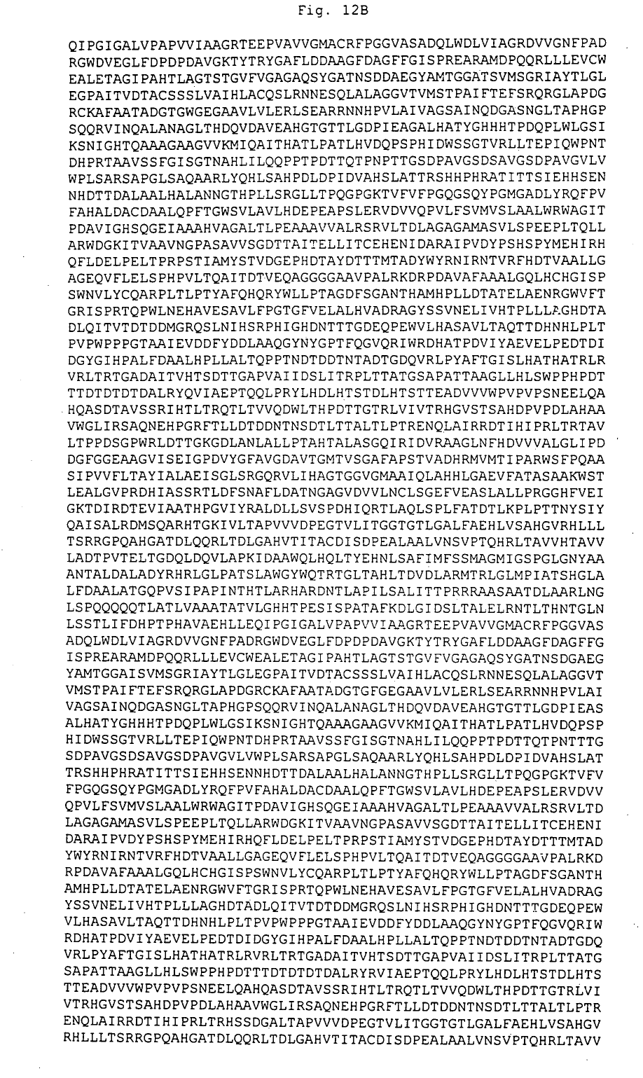

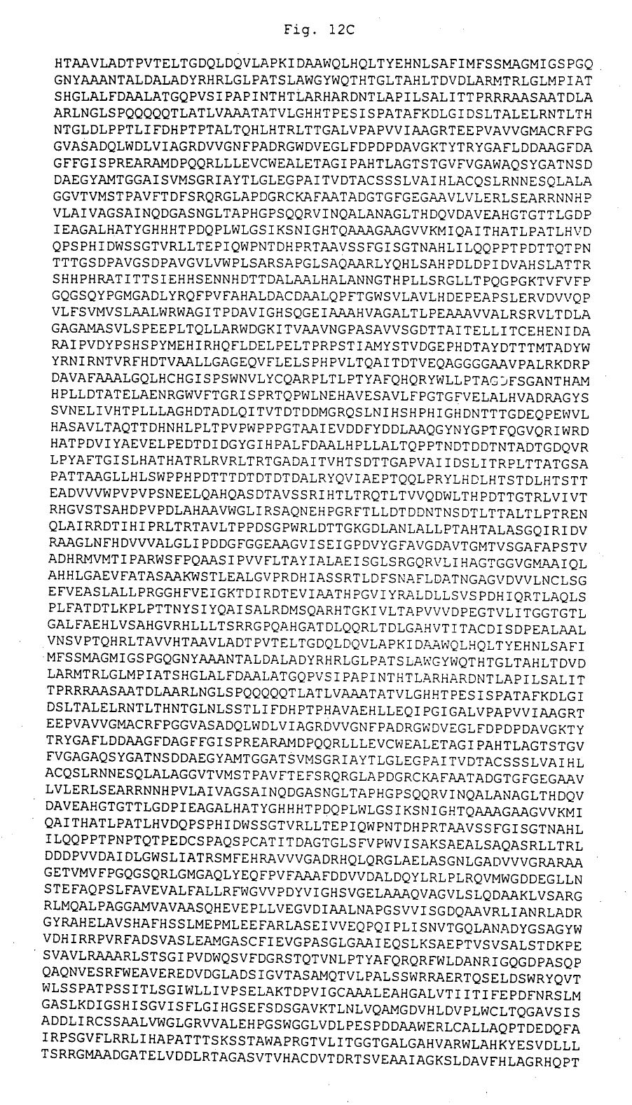

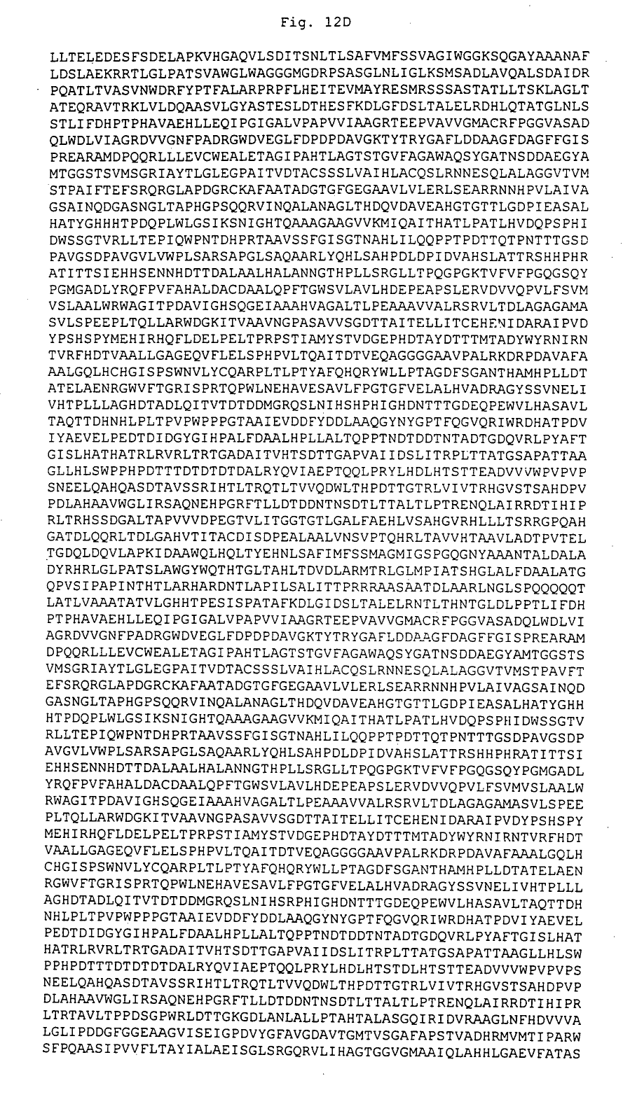

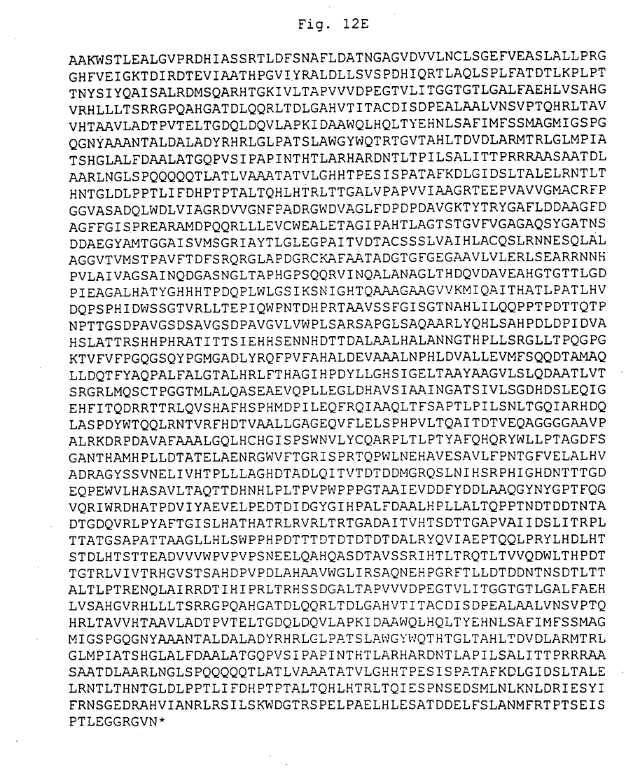

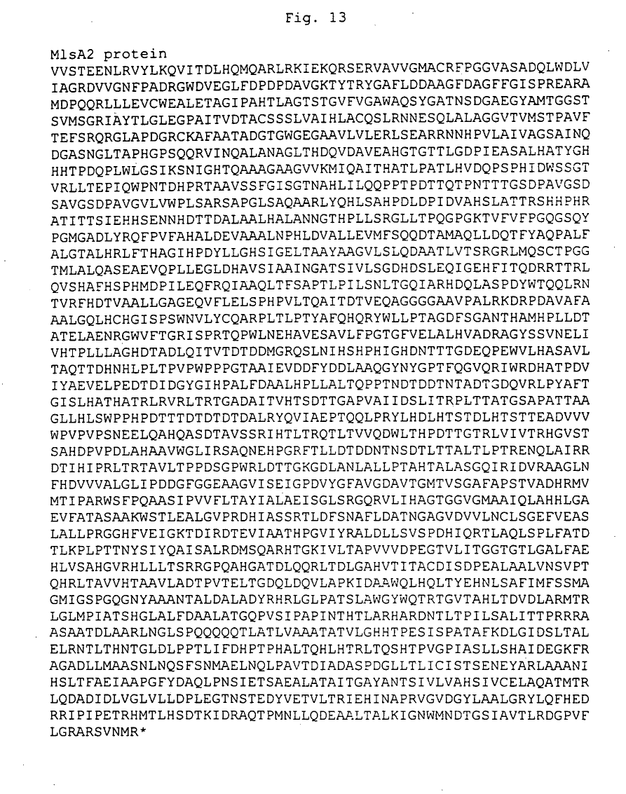

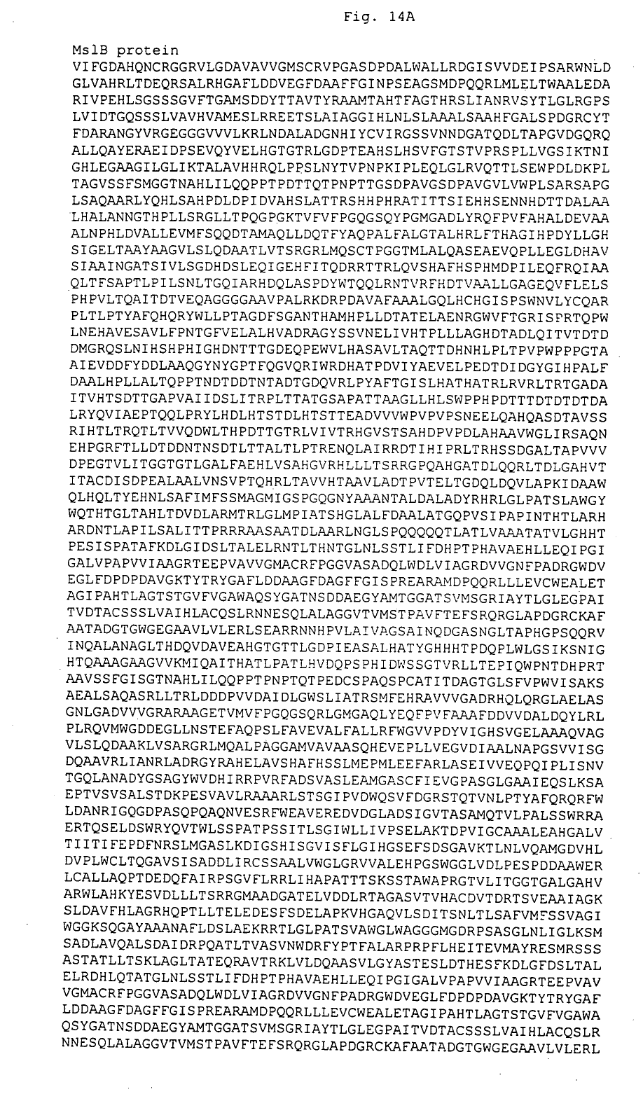

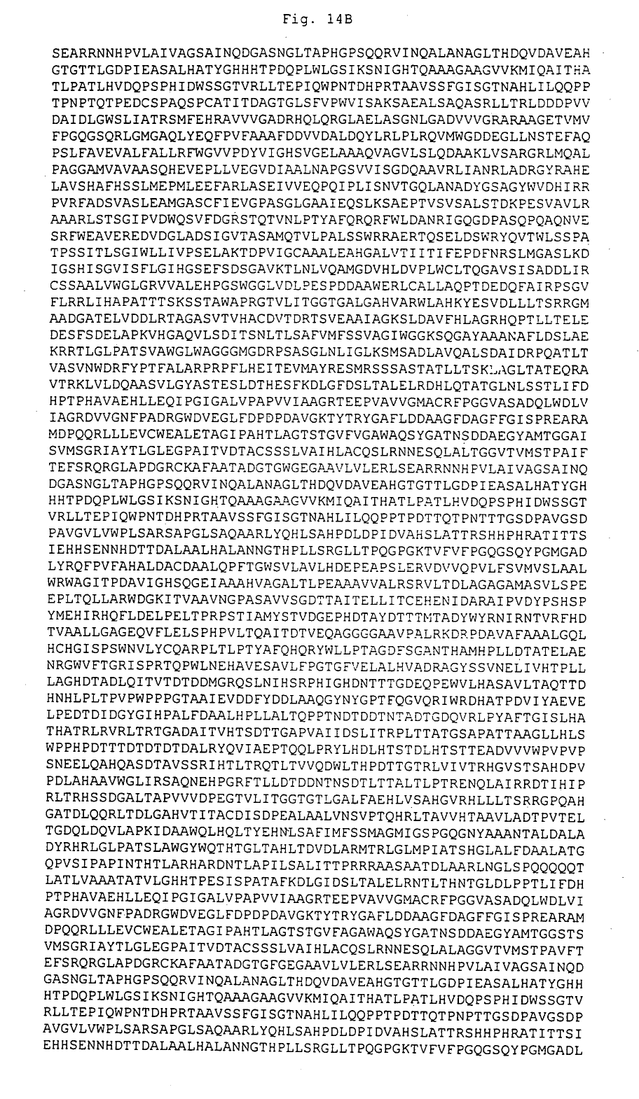

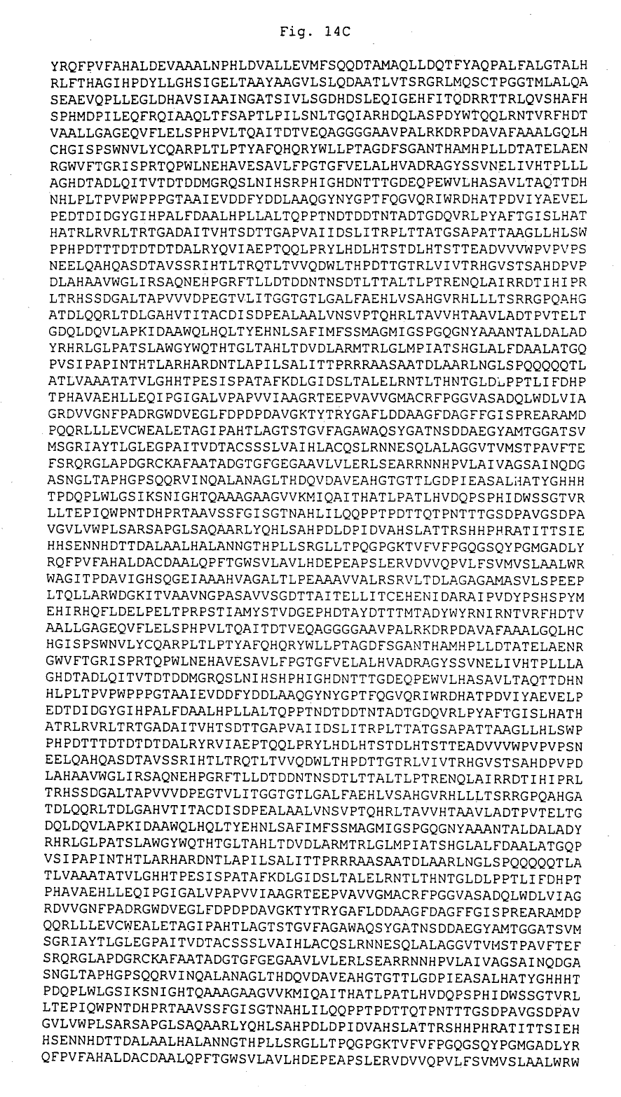

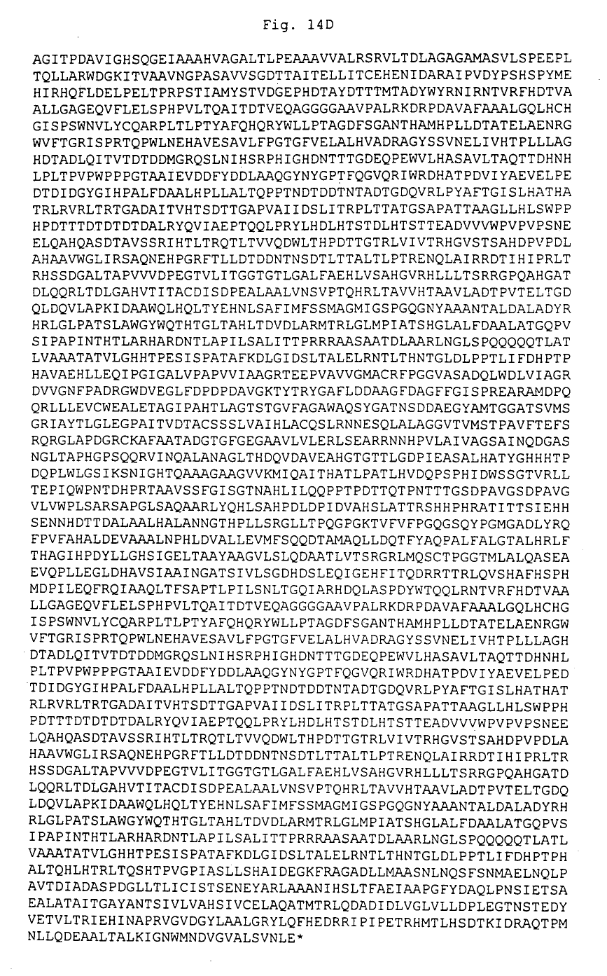

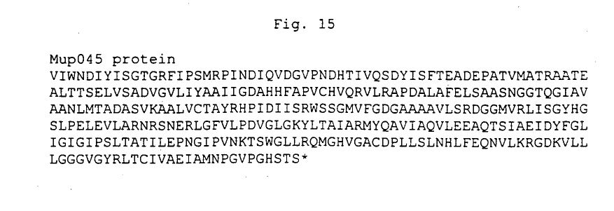

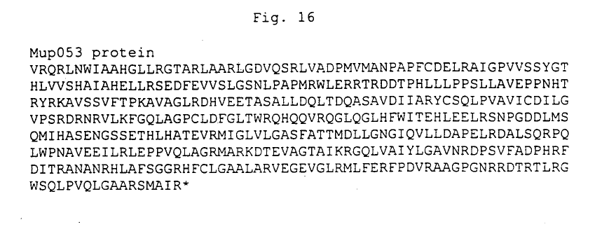

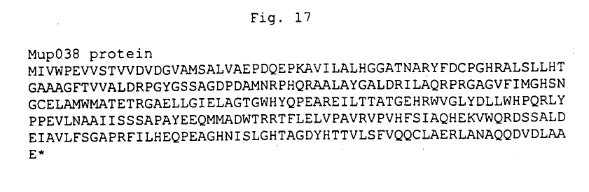

Although the close agreement between the structure-based predictions for the mycolactone genes and the DNA sequence strongly suggested that this was the mycolactone locus, definitive proof was sought by using gene disruption experiments. The genetically tractable MU strain 1615 is highly related to Agy99, and in both strains the mycolactone biosynthesis genes are plasmid-encoded and their available DNA sequences are identical. The plasmid from MU 1615 is 3-4 kb smaller than MU Agy99. This difference has been mapped to the non-PKS region of pMUM001 (FIG. 2), a region rich in insertion sequences. A transposition library of MU1615 was made using a mycobacteriophage carrying a mariner transposon (8) and mycolactone-negative mutants were identified by loss of the yellow colour conferred by the toxin (2). Putative mutants were characterised by DNA sequencing and their inability to produce mycolactone was assessed using cytotoxicity assays and mass spectroscopy of lipid extracts (9) (FIG. 4 and FIG. 5). Nucleotide sequence located the transposon insertion site in MU1615::Tn141, a non-pigmented and non-cytopathic mutant (FIG. 4), to the DH domain of module 7 in mlsA. The side chain produced by MLSB is extremely unstable in the absence of core lactone and its precursor cannot be detected (9). Mass-spectrometry confirmed the absence of both the core lactone as well as intact mycolactone in MU1615::Tn141 (see FIG. 5). Similarly, MU1615::Tn104, was mapped to the KS domain of the loading module in mlsB. Mass spectroscopic analysis confirmed that the insertion was in mlsB as the mutant still produced the core lactone as evidenced by the presence of the lactone core ion at m/z 447, and the absence of the mycolactone ion m/z 765.3 (FIG. 5). Characterization of these mutants proves conclusively that MLSA and MLSB are required to produce mycolactone.

### EXAMPLES 4, 5 and 6

Introduction

No-one skilled in the art would have expected, prior to the present disclosure, mutual sequence similarities/identities as high as the values seen for the mycolactone PKS extension modules (see Example 2 for details). Based on the anticipated need for KSs to select their substrates a minimum of sequence difference was thought to be essential to produce the variation along the polyketide chain which is seen in mycolactone. Secondly, it would have been expected that over time, the DNA for the mycolactone PKS would have accumulated random mutations leading to divergence of sequences between modules; and that variants would have been selected during evolution to optimise protein:protein interactions between individual pairs of KS and ACP domains (and between other domains within different modules), in order to optimise the transfer of the growing polyketide chain between active sites. Finally, such unprecedented very high sequence similarity at the DNA level would have been expected to be incompatible with the continued maintenance of such DNA in the producing organism, in the presence of intracellular mechanisms of recombination which operate in all cells.

The importance of the present disclosure both for the production of novel variants of mycolactone and for combinatorial biosynthesis of polyketides lies in the overturning of all these previous assumptions. It is clear that in this natural example, the KS domains are essentially identical in structure and therefore cannot be responsible for any proof-reading role in rejecting “incorrect” substrates being passed to them from the upstream extension module and will therefore faithfully process them and in turn pass them on. The same is true of the other domains of the mycolactone PKS.

As a result of the recognition of the unprecedented and unexpected properties of the mycolactone PKS it would immediately occur to the person skilled in the art to utilise the PKS genes or portions thereof, to construct genes expressing novel combinatorial arrangements of domains and modules, which in suitable recombinant host strains will produce novel combinatorial libraries of polyketides. Likewise it would immediately occur to the person skilled in the art to utilise the gene products so expressed in purified form to catalyse the production of libraries of polyketides in vitro. The person skilled in the art would instantly appreciate that the high sequence identity/similarity between modules and in particular between all KS, AT and ACP domains, means that in all such combinatorial combinations of mycolactone PKS domains and/or modules there is a very high probablility of compatible protein:protein interactions between any domain and its neighbours, in marked distinction to previously-produced hybrid modular PKSs which have been constructed, whether by module or domain deletion, addition or substitution, or by bringing together different PKS multienzymes, with or without alterations in docking domains (Gokhale R S et al.: Dissecting and exploiting intermodular communication in polyketide synthases. Science 1999, 284:482-485; Tsuji S Y, et al.:Intermodular communication in polyketide syntheses: Comparing the role of protein-protein interactions to those in other multidomain proteins. Biochemistry 2001, 40:2317-2325; Broadhurst R W, Nietlispach D, Wheatcroft M P, Leadlay P F, Weissman K J: The structure of docking domains in modular polyketide synthases. Chem. Biol. 2003, 10:723-731).

Even where previous methods are claimed not to perturb protein:protein interactions, no direct evidence has been produced to substantiate this, and in the closely-related animal fatty acid synthase it has been shown that even point mutations that alter a single amino acid can lead to dissociation of an active homodimeric enzyme into inactive monomers (Rangan V S, Joshi A K, Smith S: Mapping the functional topology of the animal fatty acid synthase by mutant complementation in vitro. Biochemistry 2001, 40:10792-10799).

Further, the essential identity of the KS domains and of the other domains makes it likely that they will faithfully process “unnatural” acyl substrates with which they are presented. Hence the present invention provides multiple hitherto-inaccessible routes to the generation and exploitation of combinatorial modular PKS libraries. Many different embodiments and applications of this invention will occur to the person skilled in the art. In the examples that follow, we set out some examples but we do not wish to be limited by them.

It will be obvious that the mycolactone PKS genes and portions thereof can be utilised in any and all applications where, previously, modular PKS genes have been used to create hybrid genes expressing novel polyketide products, and also including mixed polyketide-peptide products arising from hybrid PKS-NRPS systems, and fatty acids such as polyunsaturated fatty acids (Kaulmann U, Hertweck C: Biosynthesis of polyunsaturated fatty acids by polyketide synthases. Angew. Chem. Int. Ed. 2002 41:1866-1869.). They can be utilised to create designer PKSs capable of synthesising products which are presently obtainable only from non-sustainable natural sources such as marine sponges; or where such supplies are limited. They can be combined with chemical synthesis of polyketides and polyketide libraries, either by providing templates for combinatorial biosynthesis or by utilising as substrates the products of such chemical synthesis. They can be combined either in vivo or in vitro with enzymes carrying out post-PKS modifications to produce libraries of even greater complexity, through the re-targetting of various such modifications (including inter alia hydroxylation/methylation/glycosylation/oxidation/reduction and amination) to these new templates. They can be utilised as components of hybrid PKSs to smooth the transfer of polyketide chains from one natural PKS to the other within the hybrid. They can be utilised in directed evolution experiments to improve the efficiency of the PKS and thus increase the yield of a desired product using a range of established technologies. It will be equally obvious that standard methods can be used to alter the nucleotide sequence of the mycolactone PKS genes so that the degree of sequence identity between modules is reduced, so as to improve the stability of the genes to unwanted homologous recombination; or to optimise codon usage for heterologous expression in host strains such as Escherichia coli, cyanobacteria, pseudomonas, streptomyces, yeast, plant, and other prokaryotic and eukaryotic expression systems; as well as in in vitro expression systems.

Below we set out examples of how such hybrid genes and libraries of hybrid genes are constructed, introduced into suitable host strains and expressed, such that the encoded hybrid PKS proteins produce the polyketide products, which are valuable as potential leads for the development of novel and useful pharmaceuticals.

It will readily occur to the person skilled in the art that there are many other ways available, other than those described in these examples, for the deployment of the mycolactone biosynthetic genes the subject of the present invention for the engineered (combinatorial) biosynthesis of valuable polyketide compounds. For example the genes can be used to create designer PKSs inside suitable host strains which are capable of the production of a desired target molecule, including a molecule not known to be made naturally by a PKS (Ranganathan et al.: Knowledge-based design of bimodular and trimodular polyketide synthases based on domain and module swaps: a route to simple statin analogues. Chem. Biol. (1999) 6:731-741.) This same approach can also be used to access natural polyketides, for example those of marine origin such as the anticancer compound discodermolide, whose availability from natural sources is currently limited and/or whose total chemical synthesis is difficult and costly.

Again, the method for constructing the gene libraries of hybrid PKS genes can be varied. For example, de novo stepwise construction, module by module, of hybrid PKS genes can be carried out, using directional cloning either with two unique restriction enzymes with compatible termini, or using Xba/methylated Xba technology as described in WO 01/79520 and references therein. The resulting hybrid PKS may comprise either wholly or partly of mycolactone PKS modules or domains; may consist of only one or alternatively of two or more proteins among which the requisite extension modules are distributed. The loading module, which may be located on the same polypeptide as the extension modules or which may be located on a separate PKS polypeptide suitable engineered that it docks specifically with the N-terminus of the protein containing the first extension module, may be selected from any one of a large number of loading modules known in the art, including for example the respective loading module of the PKSs for erythromycin, avermectin, rapamycin, rifamycin, soraphen, borrelidin, monensin, epothilone, phospholactomycin and concanamycin, or the loading module may consist of an NRPS module specifying chain initiation by an amino acid as in lankacidin.

The enzyme for polyketide chain release from the hybrid PKS may likewise be present either on the same polypeptide as the last PKS extension module or on a separate polypeptide which is suitably engineered so as to dock specifically onto the PKS at the last extension module. The enzyme for chain release may be selected from any one of a large number of such chain-terminating enzymes known in the art, including thioesterase/cyclases such as those from the erythromycin, pikromycin, tylosin, spiramycin, oleandomycin and soraphen clusters; a diolide thioesterase/cyclase such as that for elaiophylin; a macrotetrolide-forming enzyme such as found in the nonactin PKS; an amide synthetase as found in the rapamycin and rifamycin PKSs; or a hydrolase system as found in the monensin PKS. This list does not exhaust the possibilities. It may also be found advantageous to co-clone the gene for a thioesterase-II enzyme either from the mycolactone biosynthetic gene cluster (ms by Stinear et al) or from any one of a number of PKS gene clusters. Such thioesterases have been shown in vivo to increase the efficiency of PKSs.

Another application would be to use the exploit the substrate tolerance of the MLS KS domains by using the MLS “ACP-KS” region as a mediator to bridge the joins between hybrid PKSs comprised of other natural PKSs. This would overcome existing specificity barriers and increase the yield of a given polyketide product.

It will be obvious to a person skilled in the art and aware of the present invention that the extension modules of the mycolactone PKS derived from all other strains of M. ulcerans, whether pathogenic or not, which contain PKS genes for the synthesis of any mycolactone, will likewise be highly suitable materials for use in the creation of engineered hybrid PKSs and of combinatorial libraries of such hybrid PKSs and for the production of novel mycolactones (and generally of novel and useful polyketides) therefrom. Similarly the other biosynthetic genes of such clusters from other M. ulcerans strains will have equivalent uses and value to those described here, including the cytochrome P450, the thioesterase-II and the FabH-like enzyme.

It will likewise be clear that all methods known in the art for the modification of natural or hybrid PKSs, whether aimed at deletion, addition, or substitution of individual enzyme functions; the alteration of oxidation state within each ketide unit, to produce either ketoacyl or hydroxyacyl functions, carbon-carbon double bonds or fully saturated acyl, or alteration of stereochemistry; the shortening or lengthening of the polyketide chain produced, can be usefully applied to the mycolactone genes.

Likewise, there are many methods known in the art for the targetted substitution of a hydrogen or a methyl or substituted methyl sidechain, derived respectively from the use of malonyl-thioester or methylmalonyl-thioester or substituted methylmalonyl-thioesters as a precursor for extension, by other alkyl or substituted alkyl groups, or by hydrogen. All these can be used to diversify further the combinatorial libraries derived from the use fo the mycolactone PKS genes. For example, the genes for methoxymalonyl-thioester together can be supplied, and an acyltransferase (AT) domain selective for methoxymalonyl thioester can be used to replace one of the existing AT domains in a PKS based on mycolactone PKS-derived units. Again, such chamges can be made not only by domain swapping but by multiple domain swapping, by site-directed mutagenesis to alter selectivity, or by whole module swaps, although in the latter casse there is an increased risk of loss of efficiency in the resulting hybrid PKS.

Likewise, it is clear that the special properties of the mycolactone PKS proteins can be used more generally in the construction of hybrid modular PKSs by substituting with individual mycolactone PKS-derived ACP and KS domains, which are expected to faciltate the crucial intermodular transfer between portions of the hybrid PKS derived from different natural PKSs, the mycolactone domains acting as “superlinkers” and taking advantage of the lack of unfavourable protein:protein contacts between the key ACP and KS domains; and the lack of chemical selectivity of the mycolactone PKS-derived KS domains.

Likewise it is clear that the recombinant cells housing any hybrid PKSs which contain mycolactone PKS-derived domains or modules can be combined with other genes encoding enzymes that are well known in the art to modify the polyketide products of modular PKSs. These include without limitation hydroxylases, methyltransferases, oxidases and glycosyltransferases. The deployment of these additional “post-PKS” genes will potentially allow the further conversion of a single novel polyketide into a combinatorial library of processed molecules, further increasing the diversity and therefore the usefulness of the libraries available as a result of the present invention. Methods are already available for the deployment in recombinant cells of the genes for entire biosynthetic pathways of activated deoxysugars, glycosyltransferases, and other auxiliary enzymes, derived from numerous antibiotic-biosynthesising actinomycetes (see e.g. WO 01/79520).

It is also clear that the mycolactone PKS genes can be expressed at high levels in suitable heterologous cells, and used in the production and purification of their encoded recombinant PKS proteins which can be used in vitro to produce polyketides. This method of production allows more complete control over the substrates presented to the PKS and removes limitations imposed by the cell wall, for example. Until now such in vitro production has not been convincingly demonstrated even from natural PKSs except for simple tri- and tetraketide synthases, and so the present invention makes. If different purified proteins contain one or more PKS extension modules, together with suitable docking domains to impose specificity of module:module interactions, this allows the combinatorial in vitro biosynthesis of libraries of polyketide products, which can be advantageously interfaced with high-throughput screening by chemical or biological means.

### EXAMPLE 4

### Heterologous Expression of the Mycolactone Biosynthetic Genes and Production of Mycolactone in Mycobacterium smegmatis and Mycobacterium marinum

MU is an extremely slow-growing mycobacterium and the production of sufficient quantities of mycolactone to permit detailed studies of the molecule is highly problematic. The M. smegmatis strain Mc²155 is a rapidly-growing and genetically tractable mycobacterium. M. marinum is a strain genetically very closely related to MU but which grows much more quickly and does not produce mycolactone. The method given here describes how to transfer the mycolactone genes from the MU plasmid (pMUM001) either to M. smegmatis MC²155 or to M. marinum (strain M23), and thus permit the convenient production of mycolactone after a fermentation period of only a few days as opposed to several weeks or even months.

Other variations of this example include the heterologous expression of modified mycolactones that exhibit modified in vivo activity with potential or enhanced therapeutic properties.

The method comprises two distinct steps as follows Step 1

Transfer of the genes encoding the enzymes responsable for the synthesis of the mycolactone core structure (mlsA1, mlsA2, mup038) to M. smegmatis and M. marinum.

The bacterial artificial chromosome (BAC) clone Mu0022B04 contains an 80 kbp fragment of pMUM001 that encompasses mlsA1, mlsA2 and mup038, hereinafter called the core fragment. This 80 kbp core fragment is subcloned into a hybrid bacterial artificial chromosome (BAC) vector that has been modified to contain the mycobacterial phage L5 attachment site (attP), the L5 integrase gene, and a gene encoding resistance to the antibiotic apramycin. This hybrid BAC, called pBeL5, therefore functions as a shuttle vector, permitting the cloning of large DNA fragments in E. coli and then facilitating the subsequent stable integration of these fragments into a mycobacterium through the action of the phage integrase. Successful transformant cells are selected for by their conferring of resistance to apramycin on the mycobacterial host cell.

The core fragment is subcloned from Mu0022B04 as an 80 kbp HindIII fragment by:
partial HindIII restriction enzyme digestion of MU0022B04
purification of the resultant 80 kb fragment by pulsed field gel electrophoresis
ligation of this fragment into the unique HindIII site of pBeL5

The resulting clones are then screened by a combination of DNA end-sequencing and of determination of the size of the DNA insert, to confirm that the correct subclone has been obtained. DNA is then prepared from a clone that has been verified as correct and this DNA is used to transform M smegmatis and M. marinum by electroporation following the standard method. Apramycin resistant clones are then subcultured, and at various time points samples are taken, and the acetone-soluble lipids are extracted, and screened by Liquid Chromatography linked to mass spectrometry (LC-MS) for the presence of the mycolactone core molecule. Cultures that test positive for the presence of the mycolactone core are designated M. smegmatis::core and M. marinum::core respectively.

Step 2

Transfer of the genes encoding the enzymes responsable for the synthesis and attachment of the mycolactone side chain structure (mlsB, mup045, mup053) into the strains M. smegmatis::core or M. marinum::core respectively.

The BAC clone Mu0022D03 contains a 110 kb fragment of pMUM001 that encompasses all of mlsB, mup045 and mup053. This clone also contains all the genes required for the autonomous replication of pMUM001. Thus, Mu0022D03, if it is furnished with an appropriate antibiotic resistance gene cassette to permit selection in a mycobacterial background, will represent a shuttle plasmid capable of replicating both in E. coli and in a mycobacterium. A mycobacterium harbouring this plasmid will produce the activated mycolactone side chain as it contains all the genes necessary for side chain synthesis.

To achieve this, Mu0022D03 is subjected to random transposon mutagenesis using the EZ:TN system which randomly inserts a kanamycin resistance cassette into the plasmid. The site of transposon insertion for kanamycin resistant mutants thus obtained is then determined by DNA sequencing. A mutant is selected that contains a transposon insertion in a gene not essential for the biosynthesis of mycolactone. DNA is then prepared from this kanamycin resistant mutant of MU0022D03 and used to transform electrocompetent M. smegmatis::core and M. marinum::core. Transformants found to be resistant to bothapramycin and kanamycin are then screened for the presence of mycolactone and its co-metabolites.

### EXAMPLE 5

### Expression of Mycolactone in Streptomyces coelicolor

The actinomycete filamentous bacteria and in particular the streptomycetes are a natural source of a wide variety of polyketides and have long been used for heterologous expression of polyketide synthase genes. The following method describes the means by which Streptomyces coelicolor can be modified to produce mycolactone. The method is described in three steps.

Step 1

Transfer of the genes encoding the enzymes responsable for the synthesis of the mycolactone core structure (mlsA1, mlsA2, mup038) into S. coelicolor A095.

The core fragment is isolated from the BAC clone Mu0022B04 as a 60 kb PacI fragment. The PacI site is conveniently located immediately upstream of the mlsA1 start codon. This fragment is purified by pulsed field gel electrophoresis and then subcloned into a hybrid BAC vector that has been modified to contain the streptomyces phage phiC31 attP sequence, phage phiC31 integrase gene, and apramycin resistance gene, all derived from the vector pCJR133 (Wilkinson C J et al. Increasing the efficiency of heterologous promoters in actinomycetes J Mol Microbiol Biotechnol. 2002 July; 4 (4):417-26) as a 6 kb apaLI fragment. This hybrid vector is named pTPS001. The PacI core fragment is then cloned into the unique PacI site of pTPS001, which is situated immediately downstream of the streptomyces actI promoter. Clones that are resistant to both chloramphenicol and apramycin are then screened by PCR for the presence of the core fragment in the correct orientation with respect to the actI promoter of pTPS001. DNA is then isolated from a PCR positive clone and used to transform by electroporation the methylation deficient E. coli strain ET12567. Subsequent transformants are then conjugated with S. coelicolor A095 following standard methods. Apramycin resistant exconjugates are then subcultured and tested by PCR and Restriction Enzymes (RE) analysis to ensure the core fragment is present. Positive exconjugates are designated S. coelicolor::core.

Step 2

Modification of the host codon repertoire and addition of the genes encoding the mycolactone modifying enzymes (mup038, mup045, and mup053).

In this step an artificial operon of four genes, under the control of a constitutive streptomyces promoter is constructed using XbaI technology. This system uses the sensitivity of XbaI to overlapping dam methylation to link genes in a single operon as a series of concatenated NdeI/XbaI fragments (see for example. WO 01/79520).

The TTA codon is rare in the streptomyces, the corresponding transfer RNA gene (bidA) is tightly regulated and only expressed during sporulation. The mycolactone genes are relatively rich in TTA codons and so to ensure an adequate supply of the cognate tRNA for efficient translation it is advantageous to modify the host S. coelicolor A095, by the introduction of a plasmid containing the bidA gene under the control of a constitutive promoter. Using the XbaI system outlined above an operon is constructed containing bidA, mup038, mup045, and mup053. This is achieved by PCR amplification and then cloning of these genes into the Streptomyces expression vector pCJW160 (Wilkinson C J et al. Increasing the efficiency of heterologous promoters in actinomycetes J Mol Microbiol Biotechnol. 2002 July; 4 (4):417-26), immediately downstream of the constitutive ermE promoter. This vector contains a thiostrepton resistance cassette. This construct (called pCJW160:poly) is transferred to S. coelicolor::core by conjugation. Apramycin and thiostrepton resistant exconjugates are subcultured and tested by PCR and RE analysis for the presence of the core fragment and pCJW160::poly. Positive cultures are again subcultured and at various time points subsamples are taken, the acetone-soluable lipids are extracted, and then screened by LC-MS for the presence of the mycolactone core molecule. Cultures that test positive for the mycolactone core are designated S. coelicolor::core::poly.

Step 3

Transfer of the genes encoding the enzymes responsable for the synthesis of the mycolactone side chain structure (mlsB) to S. coelicolor::core::poly.

The gene mlsB is isolated as a 45 kb PacI/SspI fragment from the BAC clone Mu0022D03. As for mlsA1, the PacI site is located immediately upstream of the start codon. This 45 kb fragment is purified by PFGE and then subcloned into a hybrid BAC vector that has been modified to contain the streptomyces phage VWB attp sequence, phage VWB integrase, the gene actII-ORF4, the actI promoter region, the streptomyces oriT sequence, a unique SwaI site downstream of the unique PacI site, and the hygromycin resistance gene. This hybrid vector is named pTPS006. The 45 kb PacI/SspI fragment containing mlsB is then cloned into the vector pTPS006, prepared by RE digestion with PacI and SwaI. Clones that are resistant to chloramphenicol and hygromycin are then screened by PCR for the presence of mlsB. DNA is then isolated from a PCR positive clone and used to transform by electroporation the methylation deficient E. coli strain ET12567. Subsequent transformants are then conjugated with S. coelicolor A095::core::poly following standard methods. Apramycin, thiostrepton, hygromycin resistant exconjugates are then subcultured and tested by PCR and RE analysis to ensure that all the mycolactone genes are present. Positive exconjugates are designated S. coelicolor: mls. Positive cultures are again subcultured and at various time points subsamples are taken, the acetone-soluable lipids are extracted, and then screened by LC-MS for the presence of authentic mycolactone.

### EXAMPLE 6

### Construction of a Combinatorial Polyketide Library in E. coli.

The following describes one method of using the mycolactone biosynthetic genes (mls; corresponding proteins denoted as MLS) to construct libraries of modular polyketide synthases, capable of synthesis of novel and therapeutically useful polyketides, by exploiting the high degree of nucleotide sequence similarity between functional domains. The method is described in four steps
1. Modification of E. coli to support the synthesis of polyketides, for which there is ample precedent in the prior art.
2. Construction of novel MLS modules
3. Preparation of an E. coli cosmid expression vector
4. Construction of colinear module combinations, with the number of extension modules present in each hybrid PKS being selected by the packaging requirements of cosmid particles for infection of E. coli.
5. Production of libraries of combinatorial polyketide molecules in E. coli.
   Step 1

Modification of E. coli to Support the Synthesis of Polyketides

The E. coli strain used for expression of the combinatorial libraries is engineered to express a suitable 4′-phosphopantetheinyl transferase (holo-ACP synthase, PPT-ase) which will modify the PKS modules post-translationally. Suitable PPTases are available either from M. ulcerans itself or from the surfactin (srf) gene cluster of Bacillus subtilis. Likewise the E. coli is engineered to contain appropriate pathway genes from Streptomyces spp. co-expressed in order to ensure a supply of both malonyl and methylmalonyl-CoA extender units. This is achieved using previously described methods (see for example Pfeifer, B A, et al.: Biosynthesis of complex polyketides in a metabolically engineered strain of E. coli. Science (2001) 291:1790-1792). Thus, the propionyl-CoA carboxylase (PCC) of Streptomyces coelicolor or of M. ulcerans or of Saccharopolyspora erythraea can be used to increase levels of methylmalonyl-CoA. Other pathway genes are co-expressed, by standard methods, when it is required to ensure the presence in the E. coli cells of alternative precursor molecules, for example phenyl-CoA, cyclohexanecarboxylic acid, CoA ester, or methoxymalonyl-ACP as an extender unit.

Step 2

Construction of Novel MLS Modules.

An analysis of the MLS genes reveals that they contain neither SpeI nor XbaI RE recognition sequences. In addition, the high sequence homology between modules of identical function means that the same pattern of RE digestion is obtained between such modules. These facts are exploited to construct a “universal module” where the AT and the “reductive” domains (KR, DH, ER) can be swapped by a simple ‘cut and paste’ cloning strategy. An example is given in FIG. 1 whereby a module is constructed that contains an AT domain with propionate specificity and a complete reductive loop.

By this same method other universal modules can be constructed by cloning their AT-KR-spanning BamHI-EcoRV fragments into the cloning site of the vector region depicted in FIG. 36. This combination of restriction enzyme sites results in the production of at least 5 different functional modules. The use of other restriction enzymes permits the construction of further modules.

Step 3

Preparation of a modified cosmid E. coli expression vector.

A standard E. coli cosmid vector is modified to include an efficient E. coli promoter, the arabinose-inducible araBAD promoter, immediately upstream of the loading module of the avermectin-producing PKS of Streptomyces avermitilis. The DNA encoding the ave PKS loading domain sequence is engineered to contain a unique 3′ XbaI site and is immediately followed by an offloading module with an integral TE derived from the DEBS PKS of Saccharopolyspora erythraea, preceded by a 5′ SpeI sequence (FIG. 37). SpeI and XbaI have compatible sticky ends. FIG. 37 depicts the Arrangement of modified cosmid vector to support the expression of combinatorial polyketide libraries in E. coli

Step 4

Construction of Co-Linear DNA Molecules Composed of Different Module Combinations

DNA molecules encoding discrete single modules are obtained by digestion with both XbaI and SpeI of the clones prepared in step 2 above. The DNA is pooled and self-ligated in the presence of both XbaI and SpeI, ensuring correct directional cloning of the resultant ligation products. Modules concatemerised in this way are then cloned into the modified cosmid vector, again in the presence of XbaI and SpeI. All resulting ligation products have the constituent PKS modules present in the correct orientation and in multiple combinations and with varying numbers of extension modules. The ligation mixture is packaged using the standard phage lambda packaging methods. Packaging enforces a size selection that results in inserts of approximately 45 kb and therefore generating size-selected library of recombinant E. coli containing mostly 7-9 extension modules.

Step 5

Production of Libraries of Combinatorial Polyketide Molecules in E. coli

Transfection of the E. coli strain of step 1 with phage particles derived from step 4 results in recombinant E. coli clones expressing novel polyketides under suitable conditions of cultivation, as described for example by Pfeifer, B A, et al.: Biosynthesis of complex polyketides in a metabolically engineered strain of E. coli. Science (2001) 291:1790-1792). The polyketide products are analysed by LC-MS or are used for biological screening for target activities.

The presence of a 174 kb plasmid called pMUM001 in Mycobacterium ulcerans (MU) is the first example of a mycobacterial plasmid encoding a virulence determinant. Over half of pMUM001 is devoted to six genes, three of which encode giant polyketide synthases (PKS) that produce mycolactone, an unusual cytotoxic lipid produced by MU. This invention includes an analysis of the remaining 75 non-PKS associated protein-coding sequences (CDS). It was discovered that pMUM001 is a low copy number element with a functional ori that supports replication in Mycobacterium marinum, but not in the fast-growing mycobacteria M. smegmatis and M. fortuitum. Sequence analyses revealed a highly mosaic plasmid gene structure that is reminiscent of other large plasmids. Insertion sequences (IS) and fragments of IS, some previously unreported, are interspersed among functional gene clusters, such as those genes involved in plasmid replication, the synthesis of mycolactone and a potential phosphorelay signal transduction system. Among the IS present on pMUM001 were multiple copies of the high-copy number MU elements, IS2404 and IS2606. No plasmid transfer systems were identified suggesting that trans-acting factors are required for mobilization.

The presence in MU of a 174 kb circular plasmid, named pMUM001 has been discovered. More than half of the plasmid is composed of three highly unusual polyketide synthase genes that are required for the synthesis of mycolactone. There is a precedent for plasmid-borne genes involved in secondary metabolite biosynthesis. The pSLA2-L plasmid from Streptomyces rochei is rich in genes encoding type I and type II PKS clusters, and non-ribosomal peptide sythetases. Mochizuki, S., Hiratsu, K., Suwa, M., Ishii, T., Sugino, F., Yamada, K. & Kinashi, H. (2003). The large linear plasmid pSLA2-L of Streptomyces rochei has an unusually condensed gene organization for secondary metabolism. Mol Microbiol 48, 1501-1510. But the three mycolactone PKS genes (mlsA1, mlsA2 and mlsB) stand out for two reasons. Firstly, they encode some of the largest proteins ever reported (MLSA1: 1.8 MDa, MLSA2: 0.26 MDa and MLSB 1.2 MDa); and secondly there is an extreme level of nucleotide and amino acid sequence conservation (>97% nt identity) among the various functional domains of the 18 modules that comprise the three synthases. This level of sequence conservation is unprecedented and points to the very recent evolution of this locus.

Plasmids have been widely reported among many mycobacterial species. Pashley, C. & Stoker, N. G. (2000). Plasmids in Mycobacteria. In Molecular Genetics of Mycobacteria, pp. 55-67. Edited by G. F. Hatfull & W. R. Jacobs, Jr. Washington D.C.: ASM Press. However, until the discovery of pMUM001, mycobacterial plasmids have never been directly linked to virulence and the absence of plasmids among members of the M. tuberculosis (MTB) complex has led researchers to believe that plasmid-mediated lateral gene transfer is not an important factor for mycobacterial pathogenesis. Very few mycobacterial plasmids have been characterized with complete DNA sequences available for only three mycobacterial episomes: pAL5000 a 4.8 kb circular element from M. fortuitum, Rauzier, J., Moniz-Pereira, J. & Gicquel-Sanzey, B. (1988). Complete nucleotide sequence of pAL5000, a plasmid from Mycobacterium fortuitum. Gene 71, 315-321, pCLP a 23 kb linear element from M celatum, Le Dantec, C., Winter, N., Gicquel, B., Vincent, V. & Picardeau, M. (2001). Genomic sequence and transcriptional analysis of a 23-kilobase mycobacterial linear plasmid: evidence for horizontal transfer and identification of plasmid maintenance systems. J Bacteriol 183, 2157-2164, and pVT2 a 12.9 kb element from M. avium. Kirby, C., Waring, A., Griffin, T. J., Falkinham, J. O., 3rd, Grindley, N. D. & Derbyshire, K. M. (2002). Cryptic plasmids of Mycobacterium avium: Tn552 to the rescue. Mol Microbiol 43, 173-186. There are very few reports of functions being assigned to mycobacterial plasmids although several studies have suggested that genes involved in different forms of hydrocarbon metabolism are plasmid borne. Coleman, N. V. & Spain, J. C. (2003). Distribution of the coenzyme M pathway of epoxide metabolism among ethene- and vinyl chloride-degrading Mycobacterium strains. Appl Environ Microbiol 69, 6041-6046; Guerin, W. F. & Jones, G. E. (1988). Mineralization of phenanthrene by a Mycobacterium sp. Appl Environ Microbiol 54, 937-944; Waterhouse, K. V., Swain, A. & Venables, W. A. (1991). Physical characterisation of plasmids in a morpholine-degrading mycobacterium. FEMS Microbiol Lett 64, 305-309.

There are 81 predicted CDS on pMUM001. The six CDS that are involved with the synthesis of mycolactone have been described. In this invention, the remaining 75 CDS are described with a functional study of the plasmid replication region.

### EXAMPLE 7

### Bacterial Strains and Culture Conditions

The bacterial strains used in this invention were Escherichia coli strains XL2 Blue (Stratagene) and DH10B (Invitrogen), Mycobacterium ulcerans strain Agy99, Mycobacterium smegmatis mc²155, and Mycobacterium fortuitm (NCTC 10394), and Mycobacterium marinum (M strain). E. coli derivatives were cultured on Luria-Bertani agar plates and broth supplemented with antibiotics as required (100 μg ampicillin ml⁻¹ and 50 μg apramycin ml⁻¹). Mycobacteria were cultured in 7H9 broth and 7H10 agar (Becton Dickinson) at 37° C. for M. smegmatis and at 32° C. for M. marinum. For selection of mycobacteria transformed with pMUDNA2.1, apramycin was used at a concentration of 50 μg ml⁻¹.

### EXAMPLE 8

### Nucleic Acid Techniques

General methods for DNA manipulation were as described. Sambrook, J., Fritsch, E. F. & Maniatis, T. (1989). Molecular Cloning. A laboratory Manual.: Cold Spring Harbour Laboratory Press. For Southern hybridization experiments, DNA was extracted from mycobacteria as described. Boddinghaus, B., Rogall, T., Flohr, T., Blocker, H. & Bottger, E. C. (1990). Detection and identification of mycobacteria by amplification of rRNA. J Clin Microbiol 28, 1751-1759. Approximately 1 μg of DNA was digested with SpeI and the resulting fragments were separated by agarose gel electrophoresis. The DNA was then transferred to Hybond N+ membranes by alkaline capillary transfer in the presence of 0.4 M NaOH. A DNA probe based on the repA gene was prepared by PCR-mediated incorporation of Digoxygenin dUTP into the 413 bp repA amplification product. This product was obtained using the primer sequences: RepA-F: 5′-CTACGAGCTGGTCAGCAATG-3′ [SEQ ID NO.:13] (position 665-684) and RepA-R: 5′-ATCGACGCTCGCTACTTCTG-3′ [SEQ ID NO.: 14] (position 1077-1058). Genomic DNA from MUAgy99 was used as template. Southern hybridization conditions were as described previously. Stinear, T., Ross, B. C., Davies, J. K., Marino, L., Robins-Browne, R. M., Oppedisano, F., Sievers, A. & Johnson, P. D. (1999a). Identification and characterization of IS2404 and IS2606: two distinct repeated sequences for detection of Mycobacterium ulcerans by PCR. J Clin Microbiol 37, 1018-1023.

### EXAMPLE 9

### Construction of the Shuttle Plasmid pMUDNA2.1

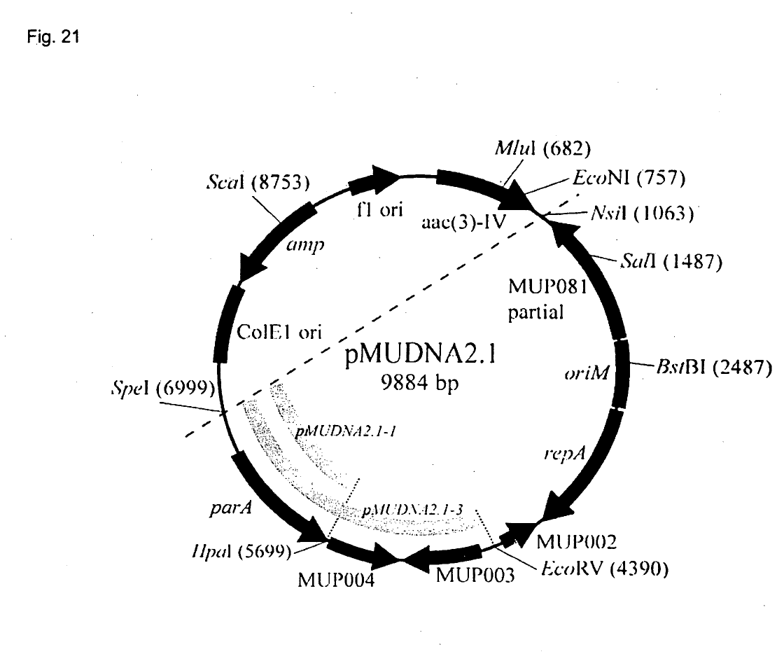

As part of the MU genome sequencing project (http://genopole.pasteur.fr/Mulc/BuruList.html), a whole-genome shotgun clone library of MU strain Agy99 was prepared in E. coli using the vector pcDNA2.1 (Invitrogen). E. coli plasmid DNA was extracted and then subjected to high thru-put automated end-sequencing. Cole, S. T., Brosch, R., Parkhill, J. & other authors (1998). Deciphering the biology of Mycobacterium tuberculosis from the complete genome sequence. Nature 393, 537-544. Sequences were assembled by using Gap4. Bonfield, J. K., Smith, K. F. & Staden, R. (1995). A new DNA sequence assembly program. Nucleic Acids Res 24, 4992-4999, and this resulted in a draft assembly database of 1597 contigs comprising 42,239 sequence reads. Previous genomic subtractive hybridization experiments between MU and M. marinum had identified MU-specific PKS sequences, Jenkin, G. A., Stinear, T. P., Johnson, P. D. & Davies, J. K. (2003). Subtractive hybridization reveals a type I polyketide synthase locus specific to Mycobacterium ulcerans. J Bacteriol 185, 6870-6882, and these sequences were used to screen for the MU PKS (and therefore plasmid-associated) contigs. This led to the identification of several E. coli shotgun clones that contained MU sequences overlapping the predicted origin of replication (or) of pMUM001. Once such clone called mu0260E04 with an insert of 6 kb, was selected for further study. To permit selection in a mycobacterial background, the apramycin resistance gene aac(3)-IV was cloned into muO260E04. Paget, E. & Davies, J. (1996). Apramycin resistance as a selective marker for gene transfer in mycobacteria. J Bacteriol 178, 6357-6360. This was achieved by PCR amplification and modification of the aac(3)-IV cassette using the oligonucleotides ApraF-SpeI (5′ GGACTAGTCCCGGGTTCATGTGCAGCTC 3′) [SEQ ID NO.:15] and ApraR-SpeI (5′ GGACTAGTCCCGGGCATTGAGCGTCAGCAT 3′) [SEQ ID NO.: 16] to incorporate flanking SpeI sites (underlined). The resultant PCR product was digested with SpeI and then cloned into the unique XbaI site of mu0260E04, resulting in the hybrid vector pMUDNA2.1 (refer FIG. 21). The deletion constructs pMUDNA2.1-1 and pMUDNA2.1-3 were prepared by double RE digestion of pMUDNA2.1 with HpaI/SpeI and EcoRV/SpeI, respectively.

Two RE fragments were obtained by each treatment. In each case, the higher molecular weight band was excised from an agarose gel, purified, treated with T4 polymerase and re-ligated. E. coli DH10B was then transformed with each of the ligation products. Transformants were subcultured and plasmid DNA was extracted. Four plasmids from each of the two double-digests were tested by RE digest to confirm the integrity and identity of the resulting deletion constructs.

One of each verified deletion plasmid was then used in mycobacterial transformation experiments. The mycobacteria/E. coli shuttle vector pMV261—which is based on the pAL5000 replicon—was used as a positive control in all transformation experiments. Snapper, S. B., Melton, R. E., Mustafa, S., Kieser, T. & Jacobs, W. R., Jr. (1990). Isolation and characterization of efficient plasmid transformation mutants of Mycobacterium smiegmatis. Mol Microbiol 4, 1911-1919. Conditions for the preparation and electroporation of M. smegmatis were as previously described. Snapper, S. B., Melton, R. E., Mustafa, S., Kieser, T. & Jacobs, W. R., Jr. (1990). Isolation and characterization of efficient plasmid transformation mutants of Mycobacterium smegmatis. Mol Microbiol 4, 1911-1919.

For electroporation of other mycobacteria, cells were harvested at room temperature from late-log phase cultures, washed twice in sterile water, then once in sterile 10% glycerol and finally resuspended in 0.01 volume of 10% glycerol. In all experiments a 200 μl aliquot of freshly-prepared cells was used for each electroporation with a BTX electroporator (Genetronics) at 2.5 kV, 25 μF and 1000 Ω. After pulsing, 1 ml of Middlebrook 7H9 medium was added to the cells and they were incubated overnight at 30° C. with shaking before plating on Middlebrook 7H10 agar containing the appropriate antibiotic. The following quantities of plasmid DNA were used in each transformation in a final volume of 5 μl: pAL5000: 150 ng; pMUDNA2.1: 780 ng; pMUDNA2.1-1: 560 ng; pMUDNA2.1-3: 430 ng. Transformation experiments were conducted in triplicate (i.e. three biological repeats using the same preparation of competent cells). The efficiency of transformation (EOT) was expressed as the average number of transformants±sd per μg of plasmid DNA.

### EXAMPLE 10

### Stability Studies of pMUDNA2.1

A late log-phase culture of M. marinum harbouring pMUDNA2.1, grown in the presence of apramycin was diluted 1:100 into three, 50 ml volumes of fresh media without apramycin and incubation was continued at 32° C. for 12 days. Aliquots of each culture were then removed at successive 3-day time points, appropriate dilutions were made and then plated on solid media with and without apramycin. Colonies were counted after ten days. The total cell number (expressed as colony forming units) and the proportion of the total cell population that had maintained antibiotic resistance at each time point were calculated.

### EXAMPLE 11

### Bioinformatic Analysis

Sequence analysis and annotation of the plasmid was managed using ARTEMIS, release 5 (http://www.sanger.ac.uk/Software). Potential CDS with apppropriate G+C content, correlation scores and codon usage were compared with sequences present in public databases using FASTA, Pearson, W. R. & Lipman, D. J. (1988). Improved tools for biological sequence comparison. Proc Natl Acad Sci USA 85,2444-2448, BLAST Altschul, S. F., Gish, W., Miller, W., Myers, E. W. & Lipman, D. J. (1990). Basic local alignment search tool. J Mol Biol 215, 403-410, and Clustal W., Thompson, J. D., Higgins, D. G. & Gibson, T. J. (1994). CLUSTAL W: improving the sensitivity of progressive multiple sequence alignment through sequence weighting, position-specific gap penalties and weight matrix choice. Nucleic Acid Res 22, 4673-4680. Additional functional insight was gleaned using the Prosite, Hulo, N., Sigrist, C. J., Le Saux, V., Langendijk-Genevaux, P. S., Bordoli, L., Gattiker, A., De Castro, E., Bucher, P. & Bairoch, A. (2004). Recent improvements to the PROSITE database. Nucleic Acids Res 32 Database issue, D134-137, and Pfam, Bateman, A., Birney, E., Cerruti, L. & other authors (2002). The Pfam protein families database. Nucleic Acids Res 30, 276-280, databases, and the TMHMM program, Sonnhammer, E. L., von Heijne, G. & Krogh, A. (1998). A hidden Markov model for predicting transmembrane helices in protein sequences. Proc Int Conf Intell Syst Mol Biol 6, 175-182, was used to predict transmembrane helices. Insertion sequence (IS) family designations were made after reference to the IS database (http://www-is.biotoul.fr/). The sequence of pMUM001 and its annotation have been previously deposited in the EMBL/DDJ/Genebank databases under the accession no: BX649209.

### EXAMPLE 12

### General Features of pMUM001

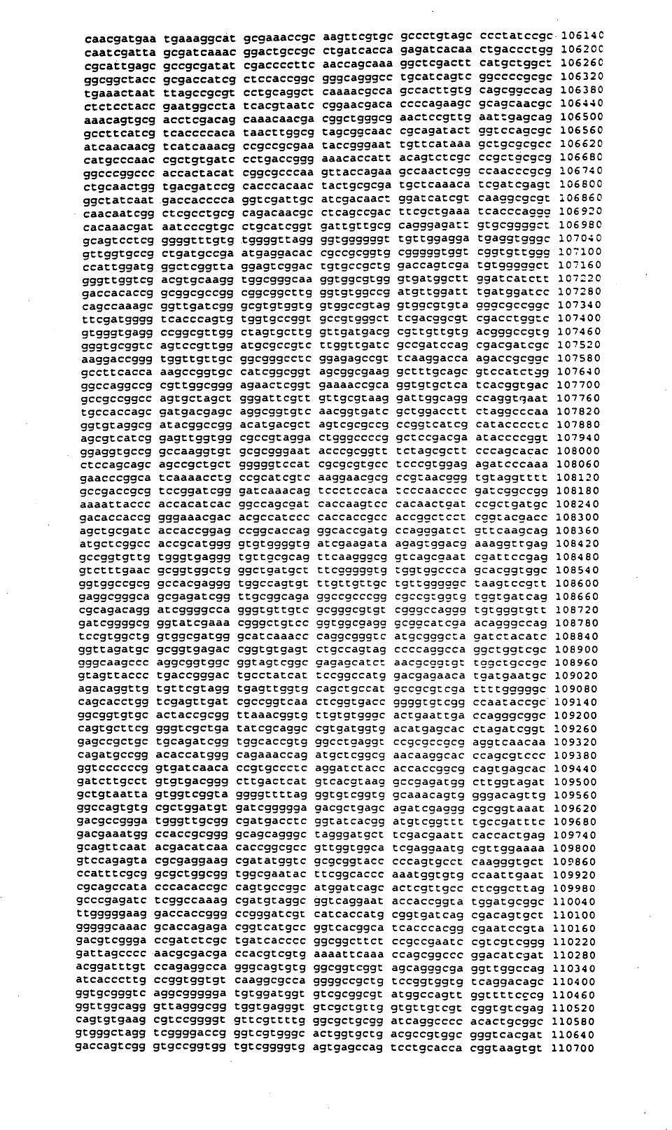

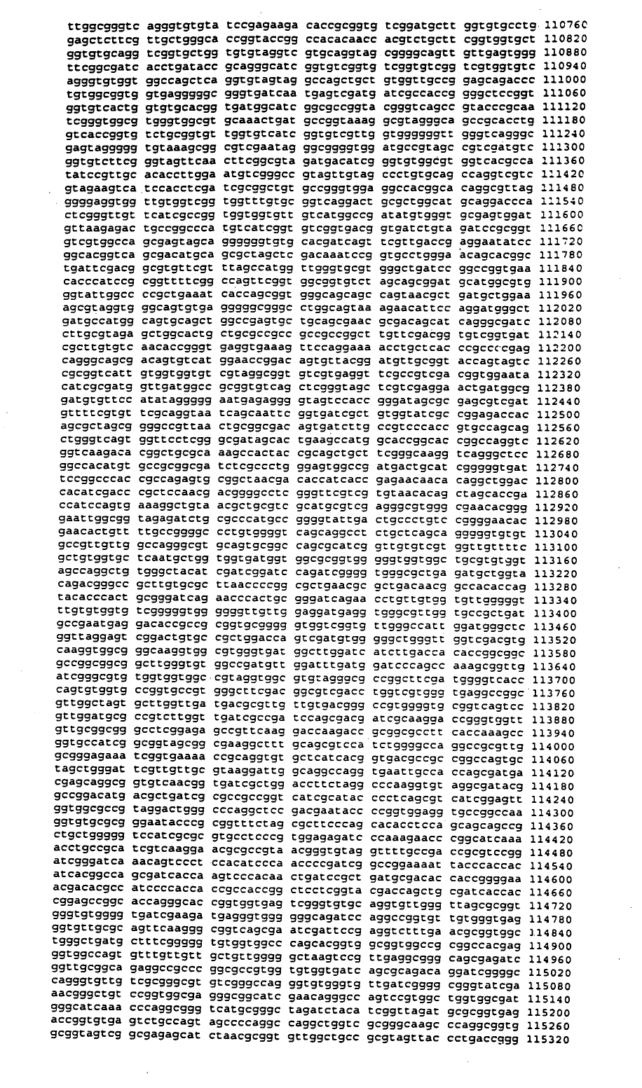

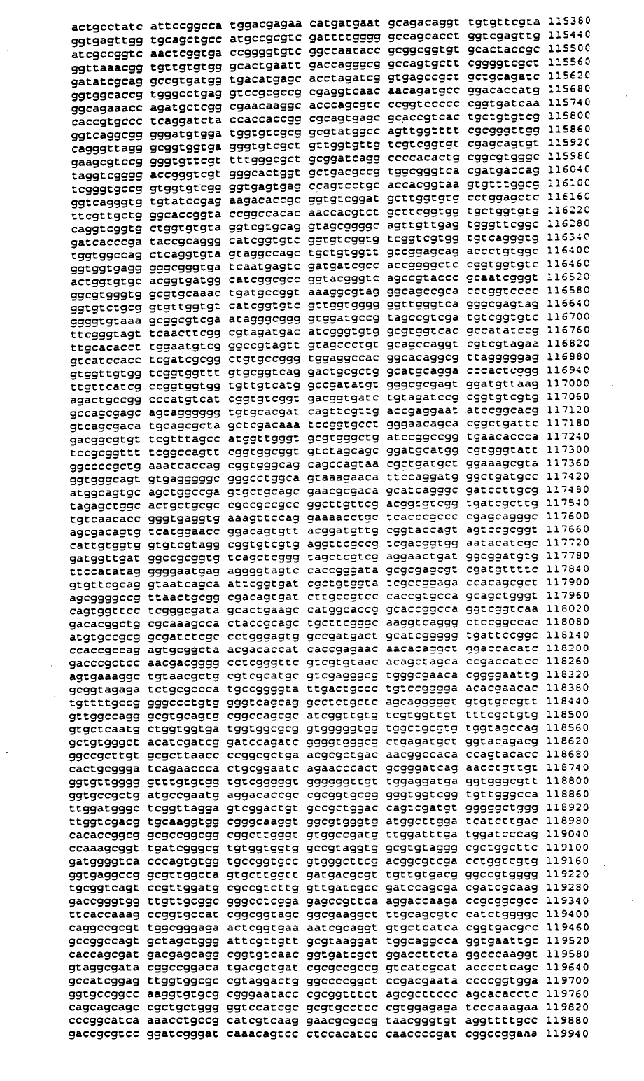

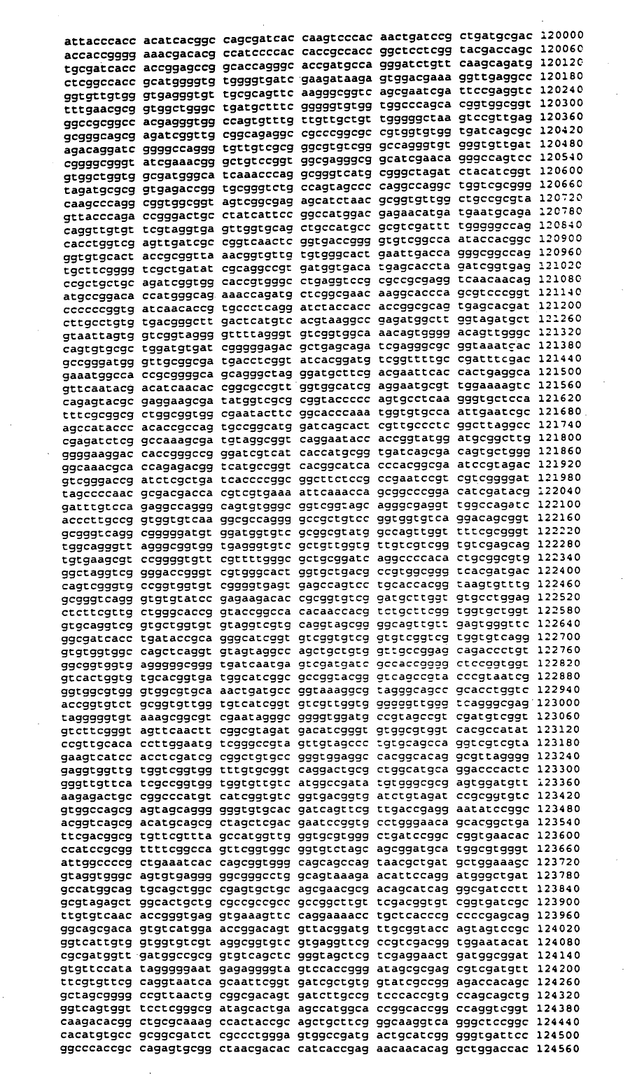

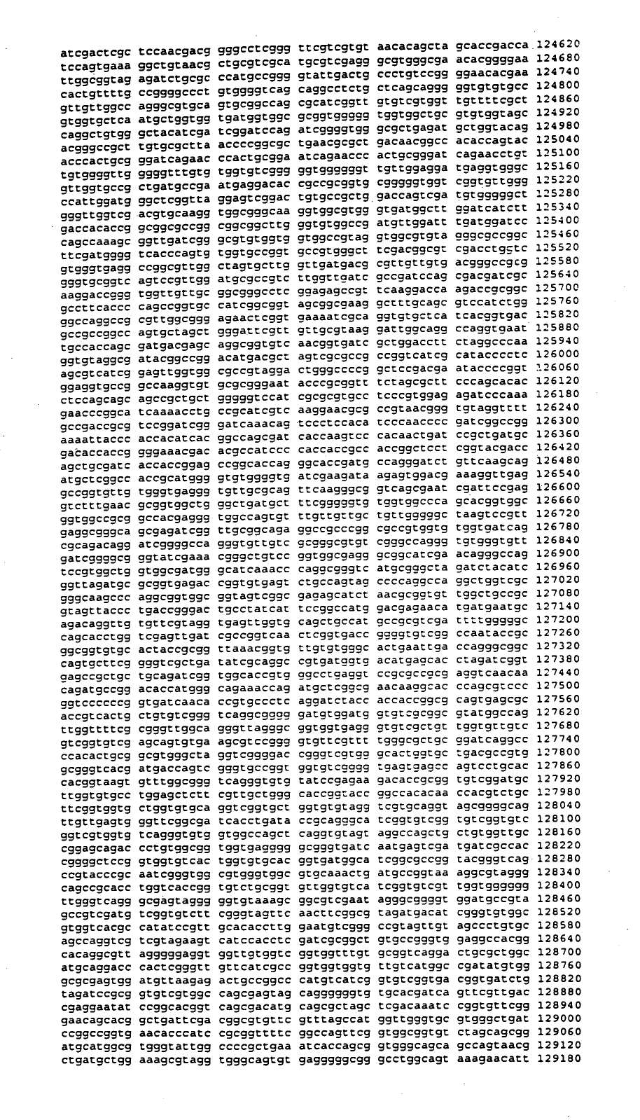

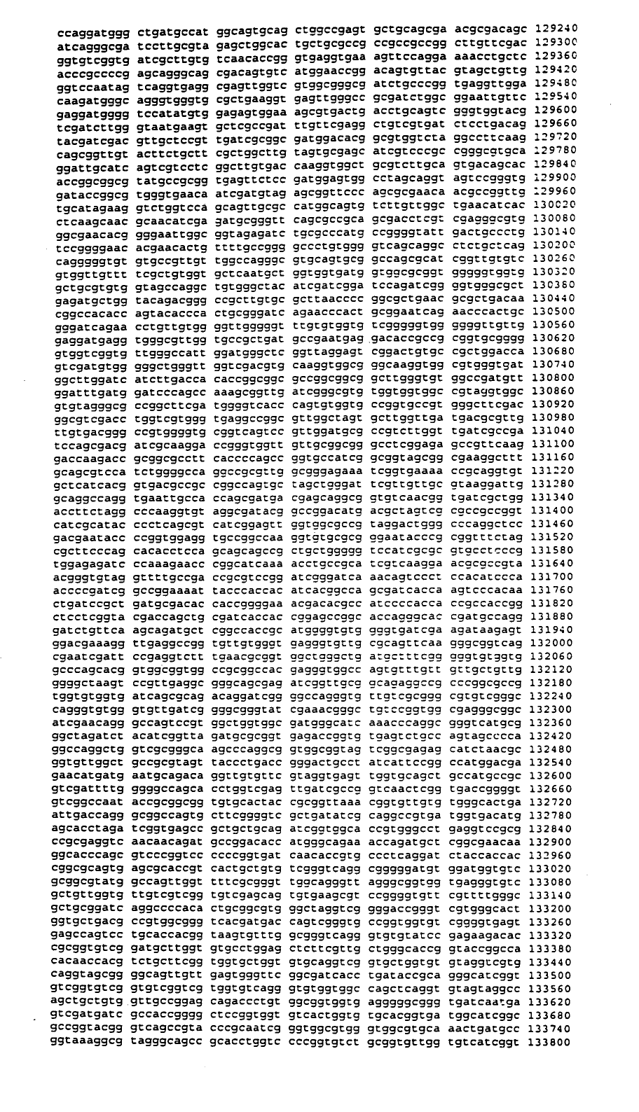

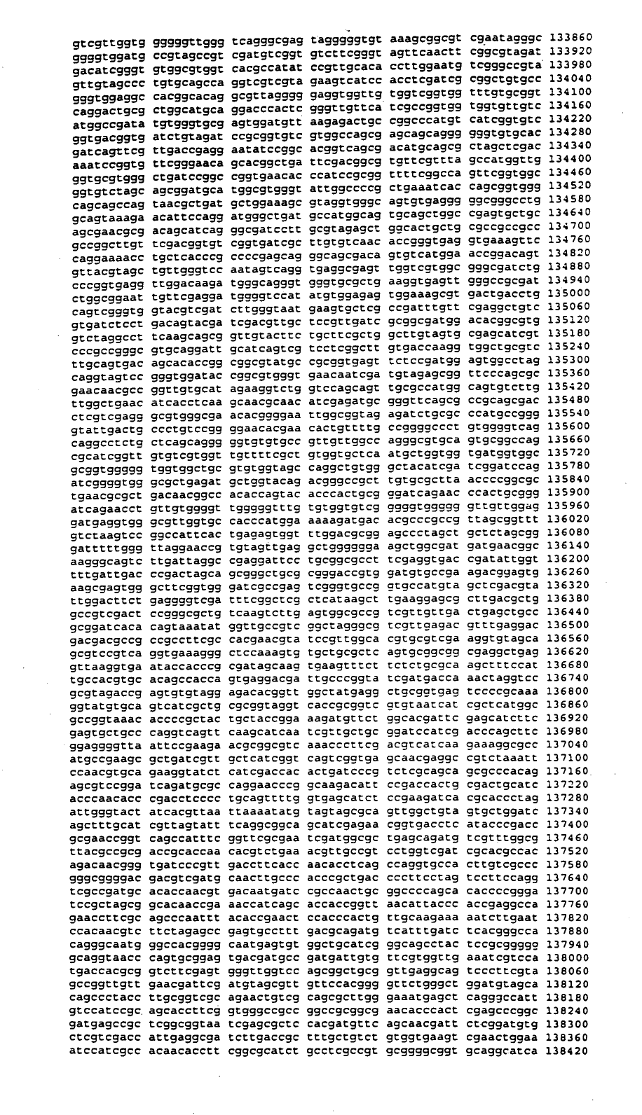

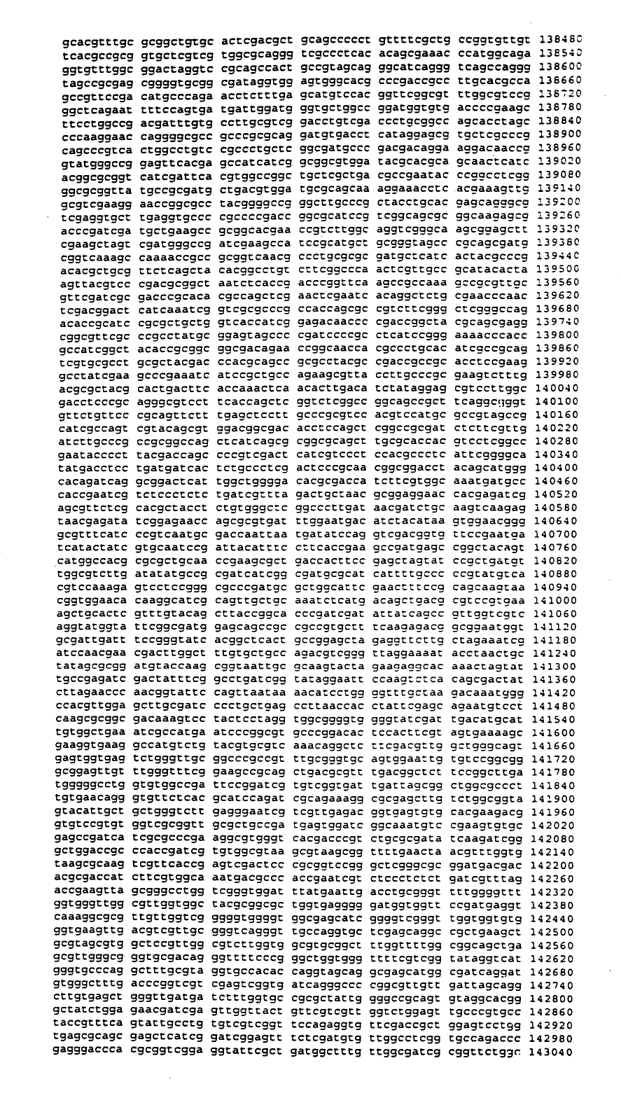

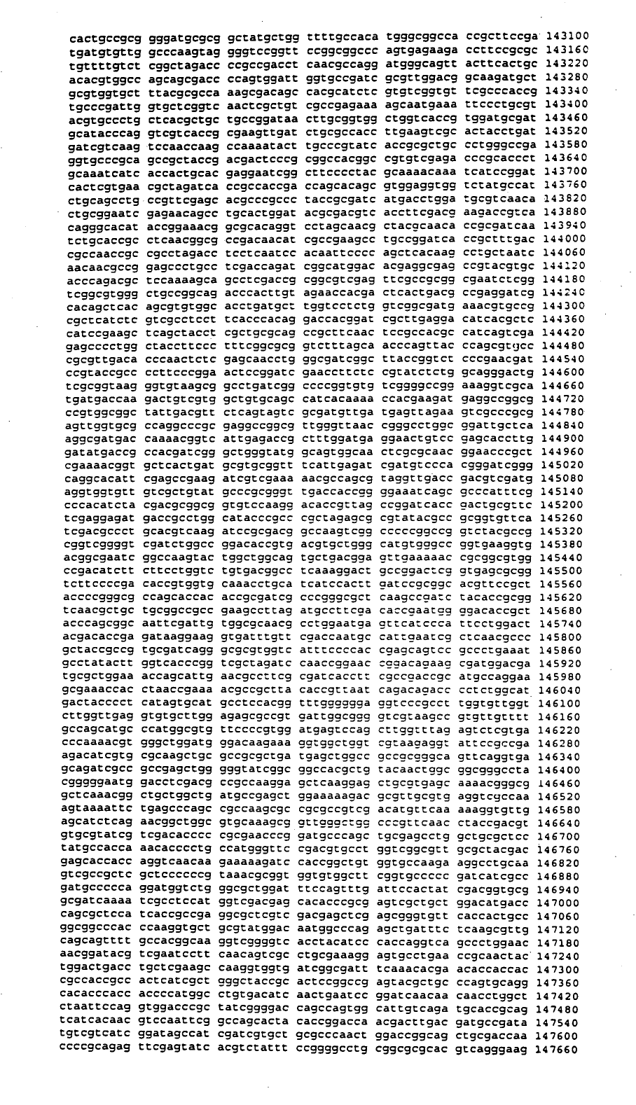

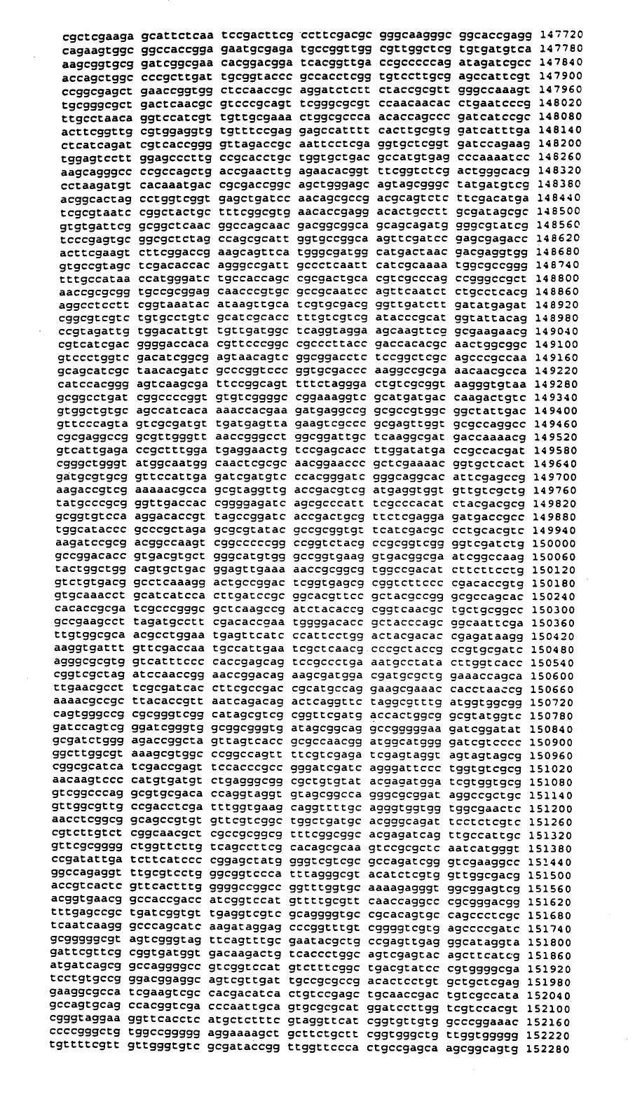

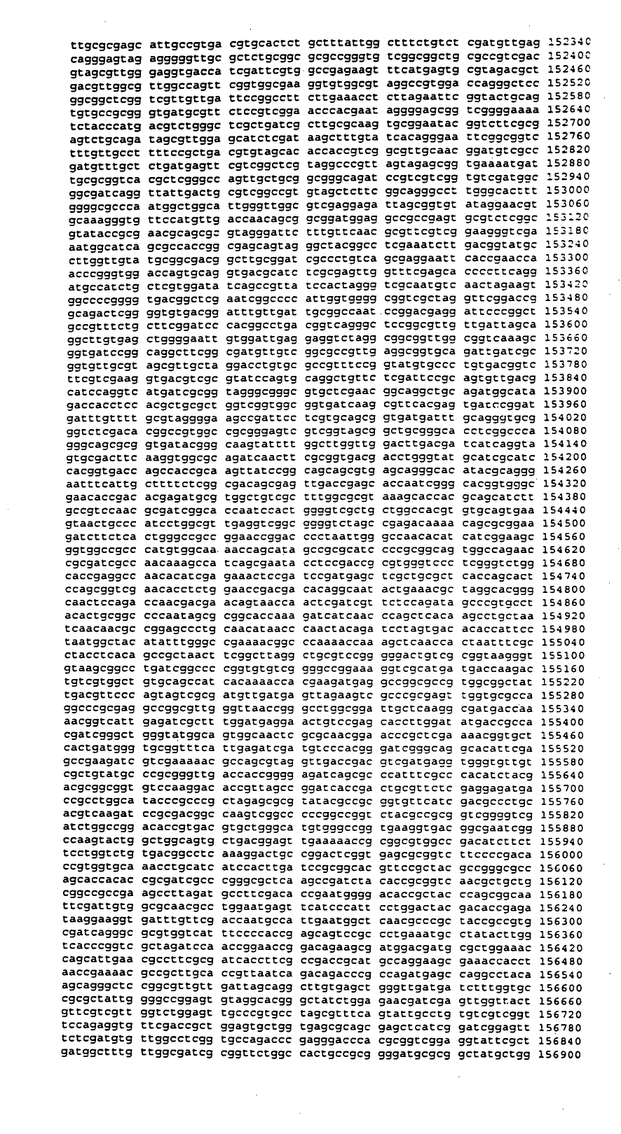

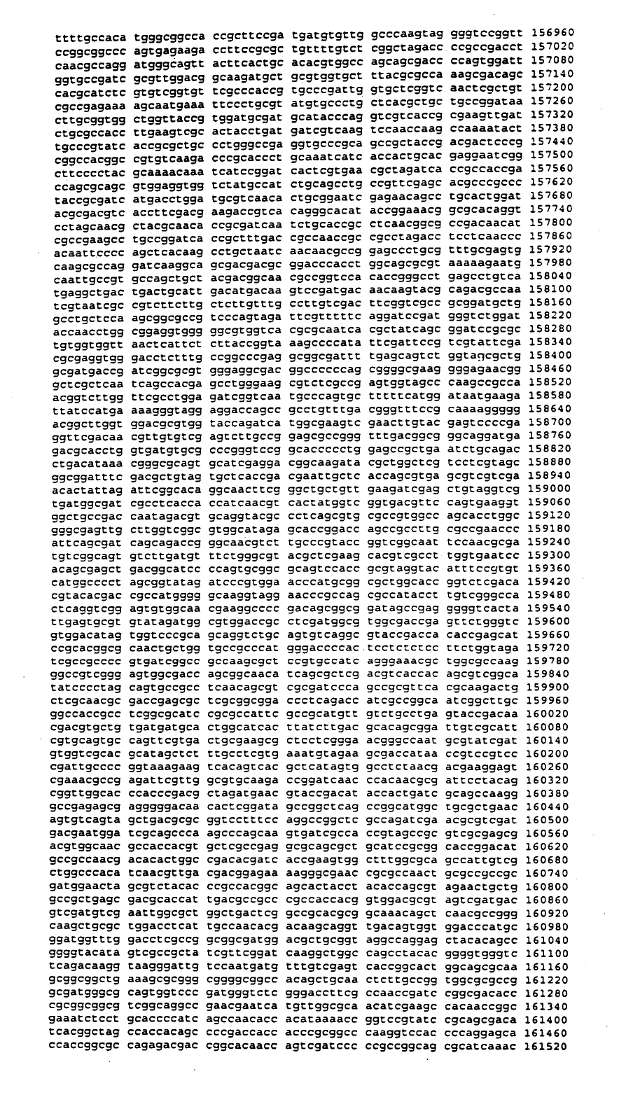

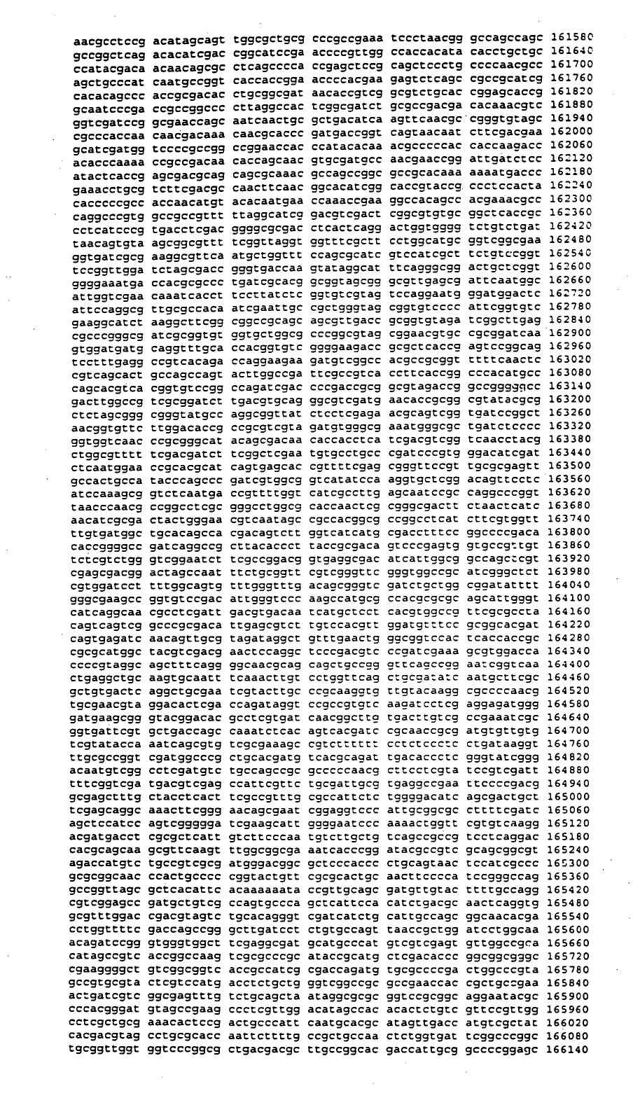

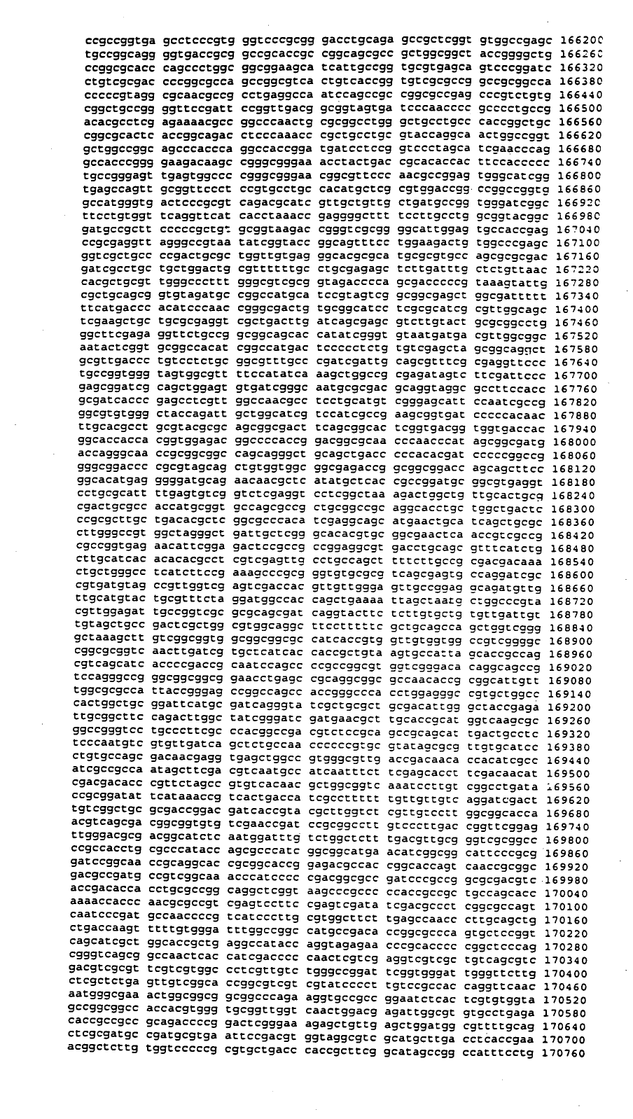

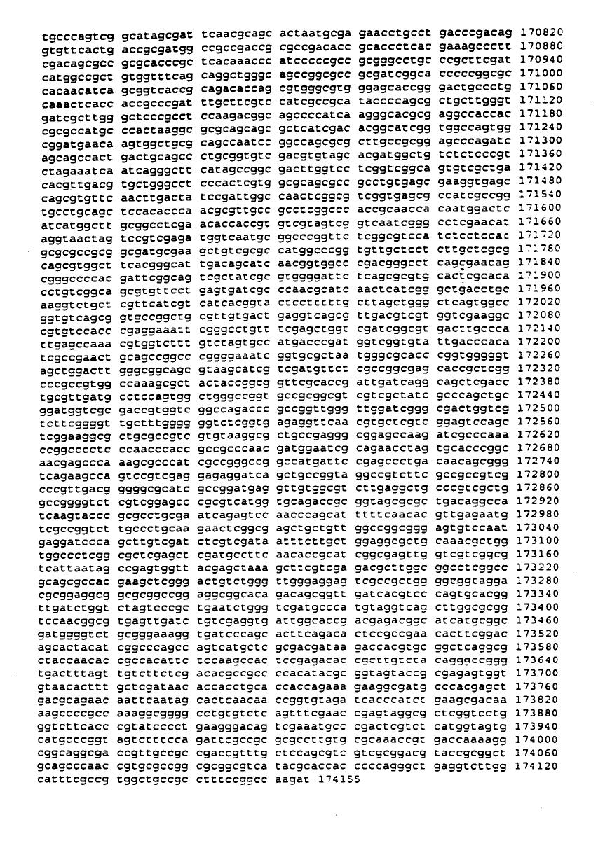

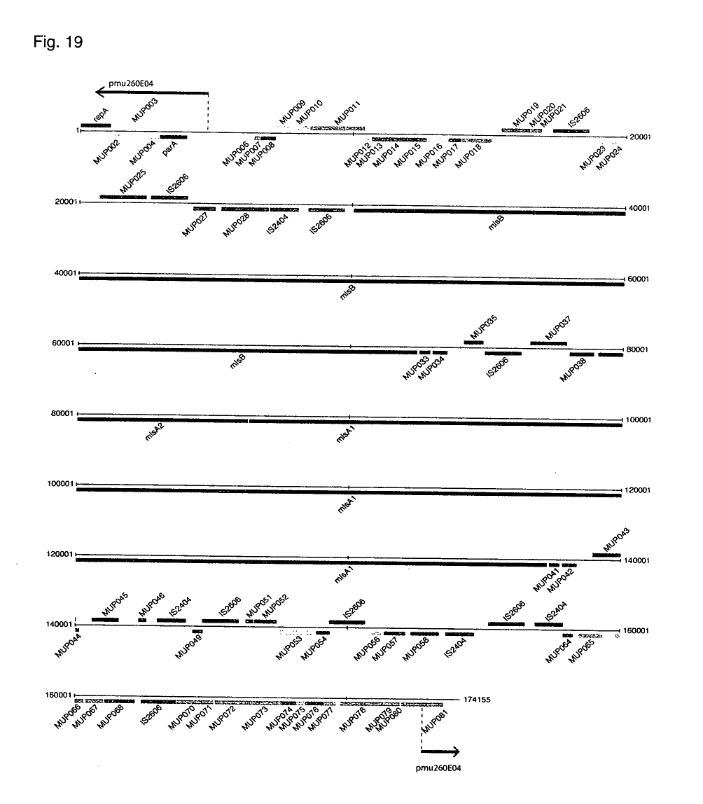

The plasmid pMUM001 is a circular element of 174,155 bp with 81 predicted CDS and a G+C content of 62.7%. The arrangement and key features of these CDS are shown in FIG. 19 and summarised in Table 1.

TABLE 1
Summary of the 81 predicted CDS in pMUM001

| | | | | G + | Predicted | | | % aa ID | |
|---|---|---|---|---|---|---|---|---|---|
| | | | Start | C | protein | | | in | Protein |
| CDS | Coordinates | Sense | codon | (%) | size (aa) | Predicted product | Closest orthologue | overlap | domains/families |
| MUP001 | 1-1107 | + | M | 66.9 | 368 | Replication protein | RepA pJAZ38, | 68 in 366, | |
| repA | | | | | | | RepA pVT2 | 55 in 360 | |
| MUP002 | 1117-1431 | − | M | 62.5 | 104 | Hypothetical protein | | | |
| MUP003 | 1694-2290 | + | V | 62.1 | 198 | Hypothetical protein | | | Contains HTH |
| | | | | | | | | | motif |
| MUP004 | 2310-2924 | − | V | 61.5 | 204 | Hypothetical protein | | | |
| MUP005 | 2921-3901 | − | M | 57.9 | 326 | Chromosome partioning | ParA Arthrobacter | 35 in 308 | ParA Family |
| parA | | | | | | protein | nictovorans | | ATPase |
| MUP006 | 5640-6386 | − | M | 63.7 | 248 | Hypothetical protein | | | |
| MUP007 | 6383-6604 | − | M | 64.9 | 73 | Hypothetical protein | M. tuberculosis Rv0340 | 32 in 43 | |
| MUP008 | 6612-7160 | − | M | 64.1 | 182 | Nucleic acid binding | M. tuberculosis nusA | 42 in 140 | |
| | | | | | | protein | | | |
| MUP009 | 7188-7616 | + | M | 65.5 | 142 | Hypothetical protein | | | |
| MUP010 | 7630-8421 | + | V | 64.0 | 263 | Hypothetical protein | | | |
| MUP011 | 8430-10412 | + | V | 64.5 | 660 | Serine/threonine protein | M. tuberculosi pknJ, | 43 in 523, | Ser/Thr kinase |
| | | | | | | kinase | pknF | 38 in 463 | active site |
| MUP012 | 10429-10692 | − | V | 56.8 | 87 | Hypothetical protein | M. tuberculosis Ag84 | 36 in 49 | |
| MUP013 | 10689-11147 | − | M | 62.3 | 152 | Membrane protein | Membrane protein | 31 in 96 | Membrane |
| | | | | | | | Mycoplasma penetrans | | spanning regions |
| MUP014 | 11149-11922 | − | V | 66.3 | 257 | Integral membrane | | | Membrane |
| | | | | | | protein | | | spanning regions |
| MUP015 | 11916-12692 | − | M | 65.4 | 258 | Membrane protein | | | Probable signal |
| | | | | | | | | | sequence |
| MUP016 | 12689-13480 | − | V | 65.9 | 263 | Hypothetical protein | | | |
| MUP017 | 13477-13929 | − | M | 63.8 | 150 | Conserved membrane | M. tuberculosis Rv3437 | 38 in 112 | C-term |
| | | | | | | protein | | | hydrophobic |
| | | | | | | | | | region |
| MUP018 | 13973-15061 | − | M | 65 | 362 | Fork-head associated | M. tuberculosis Rv3863 | 33 in 372 | FHA domain, |
| | | | | | | protein | | | HTH motif |
| MUP019 | 15406-16440 | + | M | 63.4 | 344 | Conserved membrane | M. tuberculosis pknH, | 29 in 341, | Membrane |
| | | | | | | protein | lppH | 29 in 244 | spanning regions |
| MUP020 | 16430-16612 | + | M | 58.5 | 60 | Hypothetical protein | Rhizobium loti mll8367 | 35 in 40 | |
| MUP021 | 16609-16872 | + | M | 59.5 | 87 | Transcriptional regulator | M. tuberculosis whiB6 | 29 in 85 | whiB tran- |
| | | | | | | | | | scription factor |
| MUP022 | 17287-18621 | + | M | 61.6 | 444 | Transposase | IS2606 | 97 in 376 | |
| MUP023 | 18772-19404 | − | M | 64.5 | 210 | Hypothetical protein | | | |
| MUP024 | 19401-19988 | − | M | 64.6 | 195 | Hypothetical protein | | | |
| MUP025 | 20718-22457 | + | M | 64 | 579 | Transposase | Magnetococcus sp. | 44 in 561 | |
| | | | | | | | transposase | | |
| MUP026 | 22629-23963 | + | M | 61.7 | 444 | Transposase | IS2606 | 98 in 444 | |
| MUP027 | 24162-24980 | − | V | 64.1 | 272 | Transposase | Thermoanaerobacter | 42 in 269 | |
| | | | | | | | tengcongensis | | |
| | | | | | | | transposase | | |
| MUP028 | 25197-26936 | − | M | 63.9 | 579 | Transposase | Magnetococcus sp. | 44 in 561 | |
| | | | | | | | transposase | | |
| MUP029 | 26980-27321 | − | I | 61.7 | 113 | Transposase (fragment) | IS2404 | 94 in 77 | |
| MUP030 | 27322-28026 | − | M | 61 | 234 | Transposase (fragment) | IS2404 | 92 in 312 | |
| MUP031 | 28386-29720 | − | M | 61.8 | 444 | Transposase | IS2606 | 98 in 444 | |
| MUP032 | 30054-72446 | − | V | 62.7 | 14130 | Type I polyketide | | | |
| mlsB | | | | | | synthase | | | |
| MUP033 | 72536-72910 | − | V | 62.7 | 124 | Transposase | S. avermitilis | 54 in 71 | |
| | | | | | | | transposase | | |
| MUP034 | 73008-73547 | − | V | 60.6 | 179 | Transposase | Gordonia westfalica | 68 in 94 | |
| | | | | | | | transposase | | |
| MUP035 | 74138-74851 | + | M | 65.5 | 237 | Transposase | S. avermitilis | 52 in 174 | |
| | | | | | | | transposase | | |
| MUP036 | 74905-76239 | − | M | 61.8 | 444 | Transposase | IS2606 | 98 in 444 | |
| MUP037 | 76556-77911 | + | L | 61.9 | 451 | Transposase (fragment) | Magnetococcus sp. | 44 in 390 | |
| | | | | | | | transposase | | |
| MUP038 | 78019-78924 | − | M | 64 | 301 | Type II thioesterase | S. murayanaensis | 37 in 258 | |
| | | | | | | | LanU-like | | |
| MUP039 | 79080-86312 | − | V | 62 | 2410 | Type I polyketide | | | |
| mlsA2 | | | | | | synthase | | | |
| MUP040 | 86299-137271 | − | V | 63.1 | 16990 | Type I polyketide | | | |
| mlsA1 | | | | | | synthase | | | |
| MUP041 | 137361-137735 | − | V | 62.7 | 124 | Transposase | S. avermitilis | 54 in 71 | |
| | | | | | | | transposase | | |
| MUP042 | 137833-138372 | − | V | 60.6 | 179 | Transposase | Gordonia westfalica | 68 in 94 | integrase core |
| | | | | | | | transposase | | domain |
| MUP043 | 138963-140018 | + | M | 66 | 351 | Transposase | Transposase | 52 in 341 | |
| | | | | | | | S. avermitilis | | |
| MUP044 | 140008-140148 | − | M | 62.4 | 46 | Transposase | IS476 X. campestris | 55 in 34 | |
| MUP045 | 140606-141592 | + | V | 52.8 | 328 | Type III keto-synthase | Keto-synthase | 26 in 312 | |
| | | | | | | | S. griseus | | |
| MUP046 | 142322-142615 | + | V | 65.3 | 97 | Membrane protein | R. meliloti R00794 | 36 in 101 | Membrane |
| | | | | | | | | | spanning domain |
| MUP047 | 143012-143716 | + | M | 61.6 | 234 | Transposase (fragment) | IS2404 | 91 in 234 | |
| MUP048 | 143717-144058 | + | I | 61.4 | 113 | Transposase (fragment) | IS2404 | 94 in 77 | |
| MUP049 | 144304-144693 | − | M | 62.6 | 129 | Transposase | IS1372 S. lividans | 44 in 92 | Contains HTH |
| | | | | | | | | | motif |
| MUP050 | 144660-145994 | + | M | 61.6 | 444 | Transposase | IS2606 | 98 in 444 | |
| MUP051 | 146252-146533 | + | V | 63.8 | 93 | Transposase | Gordonia westfalica | 87 in 93 | Contains HTH |
| | | | | | | | transposase | | motif |
| MUP052 | 146563-147396 | + | M | 61.9 | 277 | Transposase | Gordonia westfalica | 66 in 277 | Integrase core |
| | | | | | | | transposase | | domain |
| MUP053 | 147546-148859 | − | V | 62 | 437 | Cytochrome P450 | M. tuberculosis Rv1880c | 62 in 435 | Cytochrome P450 |
| cyp | | | | | | | | | |
| MUP054 | 148856-149359 | − | V | 60.1 | 167 | Integrase (fragment) | Myxococcus xanthus | 37 in 107 | |
| | | | | | | | Integrase | | |
| MUP055 | 149323-150657 | + | M | 61.8 | 444 | Transposase | IS2606 | 98 in 444 | |
| MUP056 | 150862-151242 | − | V | 63.3 | 126 | Hypothetical protein | | | |
| MUP057 | 151341-152117 | − | V | 62.1 | 258 | Lipoprotein | M. kansasii lipoprotein | 27 in 170 | Signal peptide |
| | | | | | | | MK35 | | lipoprotein |
| | | | | | | | | | attachment |
| MUP058 | 152314-153351 | − | V | 60.1 | 345 | Site-specific recombinase | A. tumefaciens | 26 in 295 | Phage integrase |
| | | | | | | | recombinase | | domain |
| MUP059 | 153595-154641 | − | M | 61.4 | 348 | Transposase | IS2404 | 92 in 312 | |
| MUP060 | 155147-155668 | + | M | 59.8 | 173 | Transposase (fragment) | IS2606 | 84 in 169 | |
| MUP061 | 155574-156482 | + | V | 62.9 | 302 | Transposase (fragment) | IS2606 | 98 in 302 | |
| MUP062 | 156842-157546 | + | M | 61.1 | 234 | Transposase (fragment) | IS2404 | 91 in 234 | |
| MUP063 | 157547-157888 | + | I | 62 | 113 | Transposase (fragment) | IS2404 | 96 in 77 | |
| MUP064 | 157889-158251 | − | V | 60.1 | 120 | Membrane protein | M. tuberculosis Rv3482c | 30 in 86 | Membrane |
| | | | | | | | | | spanning domain |
| MUP065 | 158471-159352 | − | M | 60.7 | 293 | Hypothetical protein | A. tumefaciens | 30 in 170 | DEAD/DEAH |
| | | | | | | | hypothetical | | helicase box |
| MUP066 | 159824-160330 | − | V | 60.4 | 168 | Hypothetical protein | Bacteriophage T7 7,7 | 32 in 106 | |
| | | | | | | | protein | | |
| MUP067 | 160417-161049 | − | M | 65.2 | 210 | Hypothetical protein | S. avermitilis | 34 in 88 | |
| | | | | | | | spermidine synthase | | |
| MUP068 | 161085-162215 | − | V | 64.5 | 376 | Membrane protein | M. tuberculosis Rv1782 | 41 in 341 | Possible signal |
| | | | | | | | | | sequence |
| MUP069 | 162445-163779 | − | M | 61.7 | 444 | Transposase | IS2606 | 98 in 444 | |
| MUP070 | 163727-164824 | − | L | 59.1 | 365 | Hypothetical protein | S. coelicolor SCO6906 | 27 in 282 | Possible |
| | | | | | | | | | pseudogene |
| MUP071 | 164673-165089 | − | M | 58.3 | 138 | Hypothetical protein | S. coelicolor SCO6906 | 29 in 117 | Possible |
| | | | | | | | | | pseudogene |
| MUP072 | 165161-166357 | − | V | 66.5 | 406 | Hypothetical protein | M. tuberculosis Rv3899c | 28 in 415 | |
| MUP073 | 166354-167547 | − | M | 67.2 | 397 | Hypothetical protein | M. leprae ML1556 IF-2 | 27 in 363 | |
| MUP074 | 167568-168152 | − | M | 66.8 | 194 | Membrane protein | M. tuberculosis Rv2473 | 25 in 202 | N-term hydro- |
| | | | | | | | | | phobic region |
| MUP075 | 168149-168487 | − | M | 65.8 | 112 | Hypothetical protein | | | |
| MUP076 | 168487-169158 | − | M | 64.4 | 223 | Membrane protein | | | Membrane |
| | | | | | | | | | spanning domain |
| MUP077 | 169192-169584 | − | V | 61.5 | 130 | Hypothetical protein | M. tuberculosis Rv0030 | 27 in 94 | |
| MUP078 | 169759-171342 | − | V | 67.2 | 527 | Hypothetical protein | M. tuberculosis Rv2083, | 32 in 181, | |
| | | | | | | | Rv0872c, | 25 in 510 | |
| MUP079 | 171361-171660 | − | M | 62.7 | 99 | Hypothetical protein | M. tuberculosis Rv0028 | 32 in 86 | |
| MUP080 | 171667-171939 | − | M | 65.9 | 90 | Hypothetical protein | M. tuberculosis Rv0027 | 27 in 89 | |
| MUP081 | 172002-173546 | − | M | 64.7 | 514 | Hypothetical protein | M. tuberculosis Rv0026 | 32 in 372 | |

Six genes were predicted to be involved in mycolactone biosynthesis and they account for 60% of the total plasmid sequence. These genes have been described elsewhere, but they encode: three type I modular PKS (MUP032, MUP039, MUP040), a type II thioesterase (MUP038), a FabH-like type III ketosynthase (MUP045), and a P450 hydroxylase (MUP053). Stinear, T. P., Mve-Obiang, A., Small, P. L. & other authors (2004). Giant plasmid-encoded polyketide synthases produce the macrolide toxin of Mycobacterium ulcerans. Proc Natl Acad Sci USA 101, 1345-1349.

There were 26 copies of various IS or fragments of IS, including 14 previously unreported elements. The presence of orthologous genes in other bacteria permitted the identification of CDS involved in plasmid functions such as replication, partioning and a potential regulatory cluster that includes, somewhat unusually for a plasmid, a serine-threonine protein kinase (STPK). There were no CDS encoding plasmid transfer functions. Eleven CDS had features suggesting they encode membrane-associated proteins, but other than the STPK, none had identifiable functions. There were 26 CDS encoding hypothetical proteins, 11 of these had no homology with other sequences in the public databases and 15 were classified as conserved hypothetical proteins because they had some homology to hypothetical proteins in MTB (9), M. leprae, Rhizobium loti (1), Agrobacterium tumafaciens (1), bacteriophage T7 (1), S. coelicolor (2) and S. avermitilis (1). The overall structure of pMUM001 is highly mosiac with discrete gene cassettes interspersed with IS. Plasmid copy number was estimated to be 1.9 copies per cell, based on the ratio of the average number of shotgun sequences per 1 kb of pMUM001 relative to the chromosome from the MU genome assembly database (http://genopole.pasteur.fr/Mulc/BuruList.html).

Origin of Replication

The repA gene, encoding the 368 aa RepA is responsible for the initiation of replication and was readily identified by sequence comparisons, sharing 68.3% aa identity in 366 aa with RepA from the M. fortuitum plasmid pJAZ38, Gavigan, J. A., Ainsa, J. A., Perez, E., Otal, I. & Martin, C. (1997). Isolation by genetic labeling of a new mycobacterial plasmid, pJAZ38, from Mycobacterium fortuitum. J Bacteriol 179, 4115-4122, and 55.6% aa identity with RepA from the M avium plasmid pVT2, Kirby, C., Waring, A., Griffin, T. J., Falkinham, J. O., 3rd, Grindley, N. D. & Derbyshire, K. M. (2002). Cryptic plasmids of Mycobacterium avium: Tn552 to the rescue. Mol Microbiol 43, 173-186. There was identity to the predicted RepA proteins from many mycobacterial plasmids with the exception of pAL5000, which appears unrelated. There was also significant identity with the RepA protein from the Rhodococcus plasmid, pSOX. Denis-Larose, C., Bergeron, H., Labbe, D., Greer, C. W., Hawari, J., Grossman, M. J., Sankey, B. M. & Lau, P. C. (1998). Characterization of the basic replicon of Rhodococcus plasmid pSOX and development of a Rhodococcus-Escherichia coli shuttle vector. Appl Environ Microbiol 64, 4363-4367.

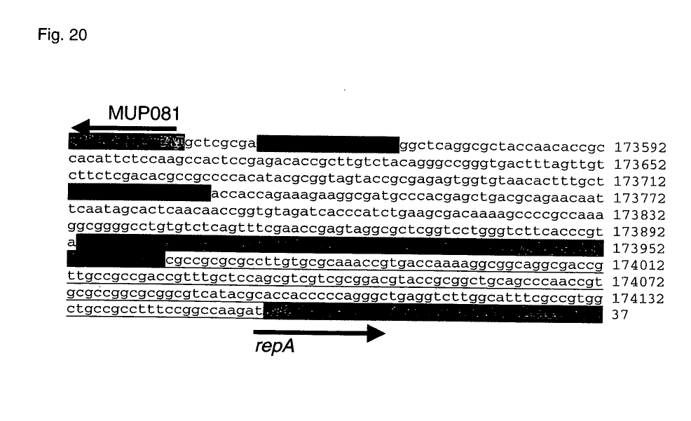

Analysis of the sequence 1-600 bp upstream of repA revealed several features suggestive of an iteron-containing origin of replication. Iterons are direct repeat sequences that bind RepA and exert control over plasmid replication. A single pair of 16 bp iterons were identified in the region 180 bp-550 bp upstream of the repA initiation codon (FIG. 20). The spacing between iterons is usually a multiple of 11, i.e, a distance reflecting the helical periodicity of ds DNA; implying that the binding sites for RepA are on the same face of the DNA. del Solar, G., Giraldo, R., Ruiz-Echevarria, M. J., Espinosa, M. & Diaz-Orejas, R. (1998). Replication and control of circular bacterial plasmids. Microbiol Mol Biol Rev 62, 434-464. The spacing for the iteron identified in pMUM001 is 143 bp, a multiple of 11. Low plasmid copy number is a characteristic of iteron plasmids. It has been proposed that as copy number increases, the RepA molecules bound to the iteron of one origin begin to interact with similar complexes generated on other origins, generating a so-called ‘hand-cuffed’ state that suppresses replication. del Solar, G., Giraldo, R., Ruiz-Echevarria, M. J., Espinosa, M. & Diaz-Orejas, R. (1998). Replication and control of circular bacterial plasmids. Microbiol Mol Biol Rev 62, 434-464. Other features commonly associated with iteron-containing replicons are multiple inverted repeats (IR) of partial-iteron sequences. These are generally situated immediately upstream of the repA start codon in the repA promoter region. del Solar, G., Giraldo, R., Ruiz-Echevarria, M. J., Espinosa, M. & Diaz-Orejas, R. (1998). Replication and control of circular bacterial plasmids. Microbiol Mol Biol Rev 62, 434-464.

In pMUM001 the situation appears somewhat different. A single 12 bp partial IR of the iteron sequence was detected in the region between the iteron. No obvious promoter elements were found in these upstream sequences, however, the region 1-261 bp upstream of the repA ATG shares very high identity with the same region in pJAZ38 (75% nt identity) and a 69 bp sub-section of this region is highly conserved among mycobacterial plasmids (Picardeau et al., 2000), (FIG. 20), suggesting that this region plays an important but as yet unidentified role for plasmid replication.

Several strategies have evolved to ensure maintenance of low-copy-number plasmids within a bacterial population. Killing of plasmid-free segregants by a plasmid-encoded toxin/antitoxin locus is one approach and has been reported for the linear mycobacterial plasmid pCLP, Le Dantec, C., Winter, N., Gicquel, B., Vincent, V. & Picardeau, M. (2001). Genomic sequence and transcriptional analysis of a 23-kilobase mycobacterial linear plasmid: evidence for horizontal transfer and identification of plasmid maintenance systems. J Bacteriol 183, 2157-2164, Another widely employed maintenance system uses active portioning and distribution of plasmid copies to daughter cells. While no candidate ‘killing’ locus was found, approximately 2 kb downstream of repA is parA, a gene encoding a 326 aa putative chromosome portioning protein. Par loci generally comprise two proteins (ParA and ParB) that form a nucleoprotein partition-complex that bind a cis-acting centromere site (ParS). Gerdes, K., Moller-Jensen, J. & Bugge Jensen, R. (2000). Plasmid and chromosome partitioning: surprises from phylogeny. Mol Microbiol 37, 455-466. Par proteins act independently of the replication apparatus and are involved in active segregation of plasmids and chromosomes before cell division. Together with host factors, Par proteins are required to direct and position newly replicated plasmids. ParA contains an ATPase domain and is specifically stimulated by ParB. Par loci share common features among different bacteria but they are quite heterogenous and appear to be acquired to stabilize heterologous replicons. Gerdes, K., Moller-Jensen, J. & Bugge Jensen, R. (2000). Plasmid and chromosome partitioning: surprises from phylogeny. Mol Microbiol 37, 455-466.

The ParA of pMUM001 is most similar to ParA from non-mycobacterial species such as Arthrobacter nicotinovorans (35.1% identity in 308 aa), but it also shares some limited homology with ParA from other mycobacteria, such as ParA from pCLP (48% in 41 aa). The G+C content of parA from pMUM001 is 58%, which is significantly lower than the average for the plasmid (62.7%) or the M. ulcerans chromosome (65.5%), supporting the notion that its origins are not mycobacterial. Par loci are generally arranged as an operon. In pMUM001, a candidate parB (MUP004) was identified immediately downstream of parA. MUP004 encodes a predicted 204 aa protein. BLASTP and PSI-BLAST database searches revealed no similarity to known ParB proteins, or any other proteins. A syntenous Par locus is present in pVT2 from M. avium, with a gene encoding a hypothetical protein immediately downstream of a parA orthologue. Heterogeneity among ParB proteins has been reported. Gerdes, K., Moller-Jensen, J. & Bugge Jensen, R. (2000). Plasmid and chromosome partitioning: surprises from phylogeny. Mol Microbiol 37, 455-466. A candidate ParS sequence was not identified on pMUM001; however three, direct repeats of the 18 bp sequence GGTGCTGCTGGGGCGGTG [SEQ ID NO.:17] were discovered in the non-coding sequence upstream of parA between positions 5314-5410. Iteron-like sequences such as these have been reported in the promoter region for Par operons and can act as binding sites for ParB. Moller-Jensen, J., Jensen, R. B. & Gerdes, K. (2000). Plasmid and chromosome segregation ir prokaryotes. Trends Microbiol 8, 313-320.

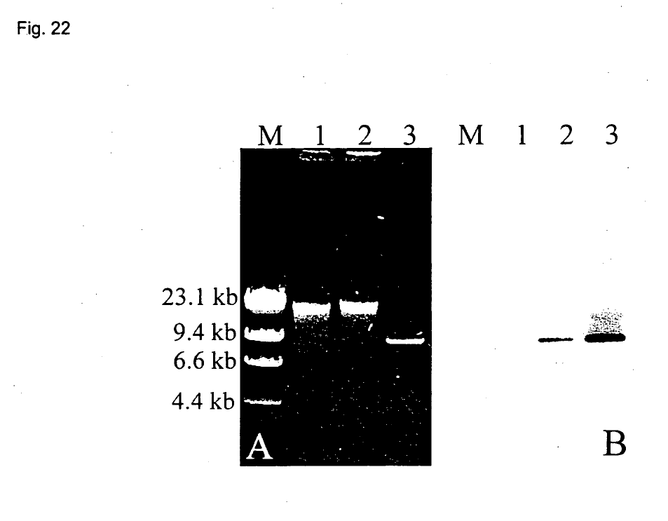

To test the hypothesis that this region contains a functional replication origin, a small-insert (3-6 kb) E. coli shotgun library of pMUM001 was screened and a clone with a 6 kb fragment was selected. This fragment spanned the region from position 172,467 to 4,190 that encompassed the 5′-end of MUP081, and the putative ori, repA and parA genes. The clone, named pmu0260E04, was modified by the insertion of aac(3)-IV, a gene conferring resistance to apramycin and thus permitting selection in a mycobacterial background. Paget, E. & Davies, J. (1996). Apramycin resistance as a selective marker for gene transfer in mycobacteria. J Bacteriol 178, 6357-6360. This construct, named pMUDNA2.1, was used to try and transform M. smegmatis, M. fortuitum, and M. marinum. Transformants were only obtained for M. marinum. The autonomous replication of pMUDNA2.1 in this species was confirmed by repA PCR and Southern hybridization with a repA-derived probe (FIG. 22). The efficiency of transformation (EOT, expressed as the average number of transformants±sd per μg of plasmid DNA from three electroporation experiments) of M. marinum transformed with pMUDNA2.1 was 1.0±0.1×10⁵; equivalent to the EOT obtained using the pAL5000-based shuttle plasmid pMV261 (2.7±0.9×10⁵).

Deletion studies were then conducted to try and define the minimum region of pMUM001 required for replication. Two deletion constructs of pMUDNA2.1 were made. The first construct, (pMUDNA2.1-1) was made by removing the 1300 bp region between the unique SpeI and HpaI sites. This region spans the entire parA gene and 372 bp of upstream sequence (FIG. 21). The second construct (pMUDNA2.1-3) was made by deleting the 2610 bp region between the unique SpeI and EcoRV sites. This 2610 bp segment spanned all of the pMUDNA2.1-1 deletion plus the predicted orfs MUP003 and MUP004. Both of these constructs were capable of transformation of M. marinum with an EOT equal to that of pMUDNA2.1 (data not shown) demonstrating that the 3327 bp of pMUM001 sequence spanning MUP002, repA, oriM and the partial sequence of MUP081 is sufficient to support replication.

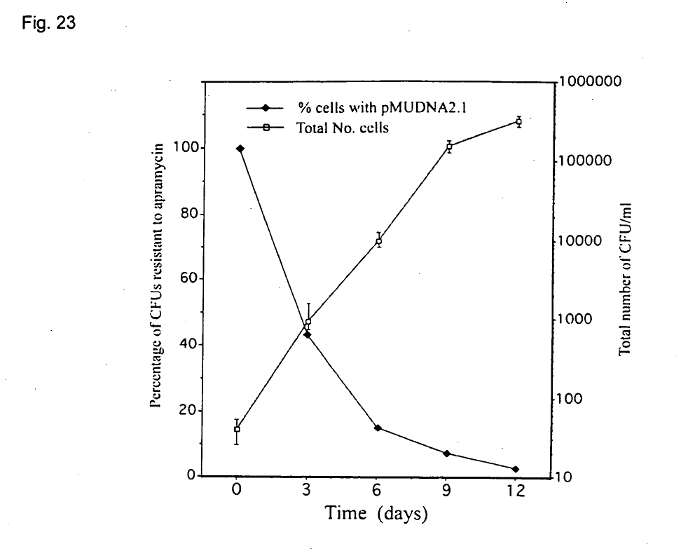

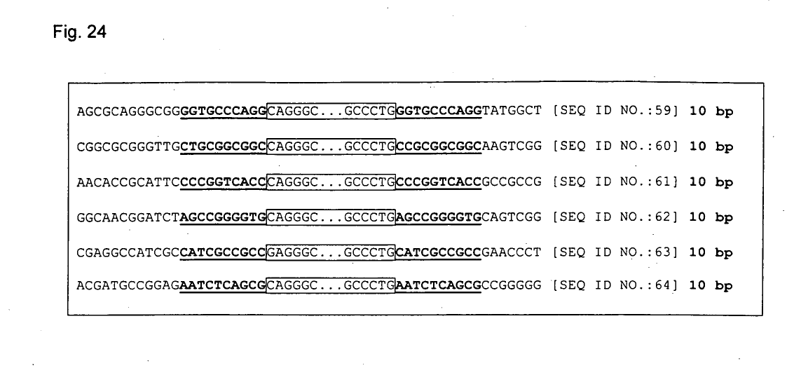

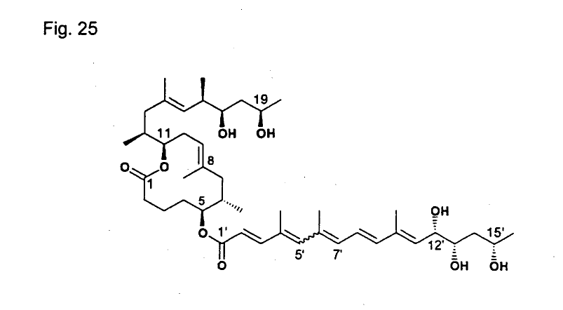

To test the stability of pMUDNA2.1, a late log-phase culture of M. marinum harbouring pMUDNA2.1 grown in the presence of apramycin, was shifted to media without apramycin and then monitored at successive time points by determining plate counts on media with and without the antibiotic. The results of this experiment are summarised in FIG. 23 and show that pMUDNA2.1 was not stably maintained and was rapidly lost from a population of cells in the absence of antibiotic selection. This result suggests that the putative par locus from pMUM001 is either not functional in M. marinum or that additional sequences are required for plasmid maintenance that are outside the 6 kb fragment from pMUM001 used to construct pMUDNA2.1. Once such region may be the 18 bp iteron sequences, proposed above as a candidate parS site. These repeats are 1.4 kb upstream of parA and 1.2 kb outside the region of pMUM001 cloned in pMUDNA2.1.

Regulatory Elements

Between MUP006 and MUP021, in a region without IS disruption, is a curious arrangement of CDS coding for potential regulatory and membrane associated-proteins (FIG. 19). MUP011 is clearly a STPK with a conserved catalytic kinase domain. It is most closely related to PknJ from MTB (43% aa identity in 523 aa).

STPKs are transmembrane signal transduction proteins and in prokaryotes they are known to be involved in the regulation of many cellular processes including virulence, stress responses and cell wall biogenesis. Boitel, B., Ortiz-Lombardia, M., Duran, R., Pompeo, F., Cole, S. T., Cervenansky, C. & Alzari, P. M. (2003). PknB kinase activity is regulated by phosphorylation in two Thr residues and dephosphorylation by PstP, the cognate phospho-Ser/Thr phosphatase, in Mycobacterium tuberculosis. Mol Microbiol 49, 1493-1508. Approximately 3.5 kb downstream of MUP011 is a CDS (MUP018) that may be a phosphorylation substrate for MUP011. MUP018 encodes a hypothetical transmembrane protein that contains an N-terminal fork-head associated (FHA) domain, a C-terminal domain with weak similarity to a 2-keto-3-deoxygluconate permease (an enzyme used by bacterial plant pathogens to transport degraded pectin products into the cell), and between these two regions, a helix-turn-helix motif. FHA domains are phosphopeptide recognition sequences that promote phosphorylation-dependent protein-protein interactions. Durocher, D. & Jackson, S. P. (2002). The FHA domain. FEBS Lett 513, 58-66. The study of FHA-containing proteins in bacteria is a nascent field but a recent report has suggested that the dual FHA domains of an ABC transporter (Rv1747) in MTB represent the cognate partner for the STPK PknF. Moller-Jensen, J., Jensen, R. B. & Gerdes, K. (2000). Plasmid and chromosome segregation in prolcaryotes. Trends Microbiol 8, 313-320. While highly speculative, one possibility is that, given the overall structure of MUP018, it may also be involved in substrate transport into the cell, perhaps of plant degradation products. This is an attractive hypothesis given the recent finding that crude extracts from aquatic plants stimulate the growth of MU. Marsollier, L., Stinear, T., Aubry, J. & other authors (2004). Aquatic plants stimulate the growth of and biofilm formation by Mycobacterium ulcerans in axenic culture and harbor these bacteria in the environment. Appl Environ Microbiol 70, 1097-1103. The final CDS in this cluster is MUP021, an orthologue of the putative transcriptional regulator WhiB6 in MTB. In MTB, immediately upstream of WhiB6 is the divergently transcribed, conserved hypothetical gene, Rv3863. A similar linkage is also seen on pMUM001, as MUP018 is an orthologue of Rv3863. The significance of all these associations remains to be tested but the continuity of this region, free of IS disruption, strengthens the idea that these genes fulfil an important regulatory role. It is also worth noting that, like pMUM001, several mycobacterial phages display a mosaic organization and that one of them Bxzl carries a STPK gene. Pedulla, M. L., Ford, M. E., Houtz, J. M. & other authors (2003). Origins of highly mosaic mycobacteriophage genomes. Cell 113, 171-182. Altered signal transduction pathways may arise from horizontal acquisition of STPK genes by mycobacteria.

Membrane Associated Proteins

Significant amounts of mycolactone can be detected in an MU culture supernatant suggesting that there may be active transport of the molecule out of the bacterial cell. Lipid export in other mycobacteria is known to involve large transmembrane proteins such as the MMPLs. Tekaia, F., Gordon, S. V., Garnier, T., Brosch, R., Barrell, B. G. & Cole, S. T. (1999). Analysis of the proteome of Mycobacterium tuberculosis in silico. Tuber Lung Dis 79, 329-342. In MTB the genes encoding MMPLs are found clustered with genes involved in lipid metabolism, including type I polyketide synthases. Tekaia, F., Gordon, S. V., Garnier, T., Brosch, R., Barrell, B. G. & Cole, S. T. (1999). Analysis of the proteome of Mycobacterium tuberculosis in silico. Tuber Lung Dis 79, 329-342. Analysis of the pMUM001 sequence revealed no mmpL-like genes. Ten hypothetical proteins that may play a role in export were identified as they contained either membrane-spanning domains, signal sequences, lipoprotein attachment sites, or hydrophobic N-terminal sequences (Table 1). However, it is possible that none of these CDS are involved in mycolactone export and that this role is fulfilled by a chromosomally encoded factor or perhaps the molecule (747 Da) is sufficiently small for it to escape by passive diffusion. Whatever their function, the 10 CDS listed in Table 1 may encode surface-exposed antigens and, given the absence of orthologues in available databases, they may be interesting candidates for testing as MU-specific antigens with potential application in serodiagnosis or vaccine development.

Insertion Sequences

Based on the presence of characteristic transposase sequences, 26 copies of various insertion sequences (IS) or IS-like sequences were identified on pMUM001. They are distributed throughout pMUM001 and interspersed among defined functional CDS clusters (e.g. replication, maintenance, toxin production). Twelve IS were copies of the known MU elements, IS2404 and IS2606, Stinear, T., Ross, B. C., Davies, J. K., Marino, L., Robins-Browne, R. M., Oppedisano, F., Sievers, A. & Johnson, P. D. R. (1999b) Identification and characterization of IS2404 and IS2606: Two distinct repeated sequences for detection of Mycobacterium ulcerans by PCR. Journal of Clinical Microbiology 37, 1018-1023, and the remaining 14 were previously unreported (FIG. 19, Table 2).

TABLE 2
Summary of the 26 putative IS elements detected on pMUM001

| IS name or | Copy | T'pse | | High scoring transposase hit |
|---|---|---|---|---|
| MUP CDS No. | No. | length (aa) | IS family | (% aa identity in overlap) |
| IS2404a | 1 | 348 | ISAsI | T'pse (46 in 338) Rhodococcus erythropolis |
| IS2404b¹ | 3 | 348 | ISAsI | |
| IS2606a | 7 | 444 | IS256 | T'pse (67 in 414) Gordonia westfalica |
| IS2606b² | 1 | 173 + 302 | IS256 | |
| 025³, 028³, 037³ | 3 | 579 | IS4 | T'pse (44 in 561) Magnetococcus sp. MC-1 |
| 027 | 1 | 272 | IS110 | T'pse (42 in 269) Thermoanaerobacter |
| | | | | tengcongensis |
| 033, 041 | 2 | 124 | IS6 | T'pse (54 in 71) Streptomyces avermitilis |
| 034, 042 | 2 | 179 | IS3 | T'pse (68 in 94) Gordonia westfalica |
| 035³, 043 | 2 | 351 | IS110 | T'pse (52 in 174) Streptomyces avermitilis |
| 044³ | 1 | 46 | IS3 | IS476 (55 in 34) Xanthamonas campestris |
| 049 | 1 | 129 | IS3 | IS1372 (44 in 92) Streptomyces lividans |
| 051³ | 1 | 93 | IS3 | T'pse (87 in 93) Gordonia westfalica |
| 052 | 1 | 277 | IS3 | T'pse (66 in 277) Gordonia westfalica |

¹contains an internal stop codon
²contains a frame-shift mutation
³truncated

Transposase sequence comparisons revealed related proteins in other actinomycetes and in more distant genera. There were three copies of a putative IS belonging to the IS4 family (MUP025, MUP028, MUP037). However, each copy of this element had been disrupted by insertion of another element. (IS2404 for MUP028 and IS2606 for MUP025 and MUP037) thus precluding delineation of this IS. The sequences bounded by the ends of the loading module domains of mlsA1 and mlsB and extending through to MUP035 and MUP043 represent 8 kb of identical nucleotide sequence (FIG. 19). This region also contains 3 different pairs of putative IS (MUP033 and MUP041, MUP034 and MUP042, MUP035 and MUP043). Since the flanking sequences for these IS are also identical the IS boundaries could not be determined. There is remarkably little distance (90 bp) between the initiation codons of the PKS genes mlsB and mlsA1 and the transposase genes (MUP033 and MUP041) that precede each of them. This raises the possibility that the promoter region for the two PKS genes lies within these IS elements.

MUP051, MUP052 and IS2606 share very high aa identity with transposases found on the 101 kb plasmid pKB1 from the rubber-degrading actinomycete Gordonia westfalica. Broker, D., Arenskotter, M., Legatzki, A., Nies, D. H. & Steinbuchel, A. (2004). Characterization of the 101-kilobase-pair megaplasmid pKB1, isolated from the rubber-degrading bacterium Gordonia westfalica Kb1. J Bacteriol 186, 212-225. The direct significance of this relationship is not known but it does serve to reinforce the idea that there is considerable genetic dynamism between diverse populations of actinomycetes. BLASTN analysis of the 26 IS sequences against the draft MU genome sequence did not reveal any paralogous elements on the MU chromosome with the exception of IS2404 and IS2606. IS2404 and IS2606, have been previously reported as high copy number elements associated with MU. Stinear, T., Ross, B. C., Davies, J. K., Marino, L., Robins-Browne, R. M., Oppedisano, F., Sievers, A. & Johnson, P. D. R. (1999b). Identification and characterization of IS2404 and IS2606: Two distinct repeated sequences for detection of Mycobacterium ulcerans by PCR. Journal of Clinical Microbiology 37, 1018-1023. Four copies of IS2404 were identified on pMUM001. The original description of IS2404 reported an element of 1274 bp, 12 bp inverted repeats, encoding a putative transposase of 348 aa, and producing 6 bp target site duplications. It is now apparent that IS2404 exists in at least two forms, both forms 94 bp longer than previously described. There was one copy of IS2402a, an element of 1368 bp, containing 41 bp perfect inverted repeats (sequence 5′-CAGGGCTCCGGCGTTGTTGATTAGCAGGCTTGTGAGCTGGG-3′) [SEQ D NO.:18] and producing a target site duplication of 10 bp. To verify these features, the draft MU genome sequence was accessed and an analysis was undertaken on a random selection of complete IS2404 sequences and their flanking regions (FIG. 23). This confirmed the extended configuration.

As originally described, IS2404a is predicted to encode a single transposase of 348 aa. There were 3 copies of IS2404b. This form is the same in all respects as IS2404a except that it contains an internal stop codon, resulting in predicted transposase fragments of 234 aa and 113 aa. However there is probably read-through of this stop codon as there are three copies of IS2404b, suggesting that the element may still be capable of tranposition.

Eight copies of the element IS2606 were also identified. It too was found to be larger than the 1406 bp initially reported. Stinear, T., Ross, B. C., Davies, J. K., Marino, L., Robins-Browne, R. M., Oppedisano, F., Sievers, A. & Johnson, P. D. (1999a). Identification and characterization of IS2404 and IS2606: two distinct repeated sequences for detection of Mycobacterium ulcerans by PCR. J Clin Microbiol 37, 1018-1023. It has a size of 1438 bp, with 31 bp imperfect inverted repeats, producing target site duplications of 7 bp and encoding a putative transposase of 444 aa. One copy contained a frame-shift mutation (MUP060 and MUP061) within the transposase region.

In conclusion, mega-plasmids (50-500 kb) are widespread across many bacterial genera and represent a major resource for lateral gene transfer within microbial communities. Genetic mosaicism has emerged as a common structural theme for these elements, Molbak, L., Tett, A., Ussery, D. W., Wall, K., Turner, S., Bailey, M. & Field, D. (2003). The plasmid genome database. Microbiology 149, 3043-3045, and is particularly evident in pMUM001 which is similar in size to certain mycobacteriophages, such as Bxzl, that also display a mosaic arrangement. Pedulla, M. L., Ford, M. E., Houtz, J. M. & other authors (2003). Origins of highly mosaic mycobacteriophage genomes. Cell 113, 171-182. In part, the mosaic arrangement may stem from the large number of IS elements carried by pMUM001. These are present in both direct and inverted orientations, and recombination between these repeats is expected to contribute to variation in both plasmid size and function. An example of this has already been reported, Stinear, T. P., Mve-Obiang, A., Small, P. L. & other authors (2004). Giant plasmid-encoded polyketide synthases produce the macrolide toxin of Mycobacterium ulcerans. Proc Natl Acad Sci USA 101, 1345-1349. In this invention, the Rep locus, required for replication and demonstrated functionality has been identified. The resultant shuttle plasmid, pMUDNA2.1, is useful for genetic analysis of both M. marinum and MU. Furthermore, the replicon of pMUM001 facilitates the production of mycolactone in a heterologous host. Heterologous expression represents an important step forward in the functional analysis of mycolactone biosynthesis and even opens new prophylactic avenues for preventing BU.

The 174 kb virulence plasmid (pMUM001) in Mycobacterium ulcerans (MU) epidemic strain Agy99 harbors three very large and homologous genes that encode giant polyketide synthases (PKS) responsible for the synthesis of the lipid toxin, mycolactone. In another aspect of this invention, deeper investigation of MUAgy99 identified two types of spontaneous deletion variants of pMUM001 within a population of cells that also contained the intact plasmid. These variants arose from recombination between two 8 kb sections of identical plasmid sequence, resulting in the loss of a 65 kb region bearing two of the three mycolactone PKS genes.

Investigation of nine diverse MU strains using PCR and Southern hybridization for eight pMUM001 gene sequences confirmed the presence of pMUM001 like elements (collectively called PMUM) in all MU strains. Physical mapping of these plasmids revealed that, like MUAgy99, three strains had undergone major deletions within their mycolactone PKS loci. On-line LC-MS/MS analysis of lipid extracts confirmed that strains with PKS deletions were unable to produce mycolactone or any related co-metabolites.

Inter-strain comparisons of the plasmid gene sequences showed greater than 98% shared nucleotide identity and the phylogeny inferred from these sequences closely mimicked the phylogeny from a previous multilocus sequence typing study that used chromosomally-encoded loci; a result that is consistent with the hypothesis that MU has diverged from the closely related Mycobacterium marinum by the acquisition of pMUM. This invention shows that pMUM is a defining characteristic of MU, but that in the absence of purifying selection, deletion of plasmid sequences and corresponding loss of mycolactone production readily arise.

More particularly, MU strains from around the world have thus far been shown to produce a very restricted repertoire of mycolactones. A study of 34 MU isolates collected worldwide showed that they all make an identical lactone core with minor variation in the acyl side chain. (Mve-Obiang, A., R. E. Lee, F. Portaels, and P. L. Small. 2003. Heterogeneity of mycolactones produced by clinical isolates of Mycobacterium ulcerans: implications for virulence. Infect Immun 71:774-783.) This variation has been largely attributed to varying degrees of oxidation at C12′ of the side chain (Hong, H., P. J. Gates, J. Staunton, T. Stinear, S. T. Cole, P. F. Leadlay, and J. B. Spencer. 2003. Identification using LC-MSn of co-metabolites in the biosynthesis of the polyketide toxin mycolactone by a clinical isolate of Mycobacterium ulcerans. Chem Commun 21:2822-2823. Mve-Obiang, A., R. E. Lee, F. Portaels, and P. L. Small. 2003. Heterogeneity of mycolactones produced by clinical isolates of Mycobacterium ulcerans: implications for virulence. Infect Immun 71:774-783.) and it has been proposed that this is due to the activity (or lack of activity) of a specific P450 monoxygenase (encoded by the plasmid gene MUP053) (Hong, H., P. J. Gates, J. Staunton, T. Stinear, S. T. Cole, P. F. Leadlay, and J. B. Spencer. 2003. Identification using LC-MSn of co-metabolites in the biosynthesis of the polyketide toxin mycolactone by a clinical isolate of Mycobacterium ulcerans. Chem Commun 21:2822-2823. Stinear, T. P., A. Mve-Obiang, P. L. Small, W. Frigui, M. J. Pryor, R. Brosch, G. A. Jenkin, P. D. Johnson, J. K. Davies, R. E. Lee, S. Adusumilli, T. Garnier, S. F. Haydock, P. F. Leadlay, and S. T. Cole. 2004. Giant plasmid-encoded polyketide synthases produce the macrolide toxin of Mycobacterium ulcerans. Proc Natl Acad Sci USA 101:1345-1349.). This invention involved the use of a large-insert MU DNA clone library to examine the stability of pMUM001. The distribution and structure of this plasmid in other MU strains was they explored using PCR, DNA sequencing, PFGE and Southern hybridization, according to the following Examples.

### EXAMPLE 13

### Bacterial Strains and Culture Conditions

The E. coli strains DH10B (F— mcrA. (mrr-hsdRMS-mcrBC) 80dlacZ.M15.lacX74 deoR recA1 araD139.(ara, leu)7697 galU galK rpsL endA1 nupG), and XL2-Blue (recA1 endA1 gyrA96 thi-1 hsdR17 supE44 relA1 lac [F′ proAB lacI qZ.]) were cultivated in Luria-Bertani broth at 37° C. Mycobacterium marinum (M strain) was cultivated at 32° C. in 7H9 Middlebrook medium (Becton Dickenson) supplemented with OADC (Difco). Ten M. ulcerans clinical isolates were used, identified as follows: Agy99 (origin: Ghana 1999; this strain was used for the MU genome sequencing project); Kob (origin: Ivory Coast 2001); 1615 (origin Malaysia 1963); Chant (origin South East Australia 1993); IP105425 (from the reference collection of the Institut Pasteur and derived from the reference strain ATCC 19428; origin: South East Australia 1948); 01G897 (origin: French Guiana 1991); ITM-5114 (origin: Mexico 1958); ITM-941331 (origin: Papua New Guinea 1994); ITM-98912 (origin: China 1997); ITM-941328 (origin: Malaysia 1994). MU isolates were grown as described for M. marinum. MU isolates prefaced by ITM were kindly provided by Francoise Portaels (Belgian Institute for Tropical Medicine).

### EXAMPLE 14

### LS-MS/MS Analysis of Mycolactones

Lipid fractions from MU were extracted and analysed for mycolactones as previously described (George, K. M., L. P. Barker, D. M. Welty, and P. L. Small. 1998. Partial purification and characterization of biological effects of a lipid toxin produced by Mycobacterium ulcerans. Infection & Immunity 66:587-593. Hong, H., P. J. Gates, J. Staunton, T. Stinear, S. T. Cole, P. F. Leadlay, and J. B. Spencer. 2003. Identification using LC-MSn of co-metabolites in the biosynthesis of the polyketide toxin mycolactone by a clinical isolate of Mycobacterium ulcerans. Chem Commun 21:2822-2823.)

### EXAMPLE 15

### Oligonucleotides and DNA Methods

The oligonucleotides used in this invention are shown in Table 1.

TABLE 1
Oligonucleotides used in this study

| | | [SEQ | | PCR | |
|---|---|---|---|---|---|
| | | ID | Position in | product | Nucleotides |
| Primer | Sequence (5′-3′) | NO.:_] | pMUMOO1 | (bp) | sequenced |
| RepA-F: | CTACGAGCTGGTCAGCAATG | 19 | 665 - 684 | 413 | 762 - 980 |
| RepA-R | ATCGACGCTCGCTACTTCTG | 20 | 1077 - 1058 | | |
| ParA-F | GCAAGCTGGGCAATGTTTAT | 21 | 3840 - 3821 | 501 | 3766 - 3431 |
| ParA-R | GTCCGGTCCUGATAGGTCA | 22 | 3340 - 3359 | | |
| MUPO11-F | ACCACCCAAGAGTGGAACTG | 23 | 9882 - 9901 | 479 | 10008 - 3431 |
| MUPO11-R | TGTCGTGTCGAGGTATGTGG | 24 | 10379 - 10360 | | |
| MLSload-F | GGGCAATCGTCCTCACTG | 25 | 71891 - 71874 | 560 | 71798 - 71409 |
| | | | 136716 - 136699 | | 136623 - 136234 |
| MLSload-R | CAAGGGCAGTCTTGATTAGG | 26 | 71315 - 71334 | | |
| | | | 136665 - 136684 | | |
| MLSAT(II)-F | AACGTTGAATCCCGTTTTTG | 27 | 59656 - 59675 | 504 | 59579 - 59256 |
| | | | 64273 - 64292 | | 64196 - 63873 |
| | | | 105563 - 105582 | | 105486 - 105163 |
| AT(II)-R | GCACCACAAAGGAACGTCTAA | 28 | 59172 - 59192 | | |
| | | | 63789 - 63809 | | |
| | | | 105079 - 105099 | | |
| TEIL-F | ATTCAAACGGATGCGAACTG | 29 | 78553 - 78572 | 500 | 78461 - 78157 |
| TEII-R | ACATTGCTGGACAAACGACA | 30 | 78073 - 78092 | | |
| MUPO45-F | CAGCAAGTAACGGTGGAACA | 31 | 140931 - 140950 | 496 | 141020 - 141340 |
| MUPO45-R | ACGTGGCCCATTTGTCTTAG | 32 | 141407 - 141426 | | |
| P450-F | CCCACCTCGTCGTTAGTCAT | 33 | 148662 - 148681 | 500 | 148592 - 148265 |
| P450-R | GTGCTCGGTGATCCAGAAGT | 34 | 148182 - 148201 | | |

Standard methods were used for subcloning, PCR and automated DNA sequencing. DNA sequences were assembled and annotated using Gap4 and Artemis respectively (Bonfield, J. K., K. F. Smith, and R. Staden. 1995. A new DNA sequence assembly program. Nucleic Acids Res 24:4992-4999. Rutherford, K., J. Parkhill, J. Crook, T. Horsnell, P. Rice, M. A. Rajandream, and B. Barrell. 2000. Artemis: sequence visualization and annotation. Bioinformatics 16:944-945.).

### EXAMPLE 16

### PFGE and Southern Hybridization

Mycobacterial DNA was prepared in agarose plugs as follows: Bacterial cells were grown to midlog phase in 7H9 Middlebrook medium and harvested by centrifugation. The cells were inactivated by the addition of 8001 μl of 70% ethanol for 30 minutes at 22° C. The ethanol was then removed and the cell pellet was washed once in 1% Triton X-100 and resuspended in TE buffer (10 mM Tris, 1 mM EDTA [pH 8.0]), using as a guide 150 μl of TE for every 10 mg cells (wet weight). The cells were mixed with an equal volume of 2% (w/v) low melting temperature agarose (BioRad) at 45° C. and dispensed immediately into plug molds (BioRad).

Up to ten plug slices (4 mm×7 mm) were then incubated for 18 hours at 37° C. in a 30 ml solution containing 0.5M EDTA [pH8.0], 0.5% Sarkosyl, 60 mg deoxycholic acid and 100 mg lysozyme. The plugs were washed once in 1×TE and incubated for a further 48 hours at 50° C. in a 30 ml solution containing 0.5M EDTA [pH8.0], 0.5% Sarkosyl and 30 mg of proteinase K. The plugs were then washed extensively in 1×TE at 4° C. Prior to restriction enzyme (RE) digestion, each plug slice was equilibrated for 30 min at room temperature in 400 μl of the RE buffer. Each plug slice was then incubated for 18 hours at 37° C. in 300 μl of RE buffer with 1% (w/v) BSA and 40 U of XbaI.

PFGE was performed using the BioRad CHEF DRII system (BioRad) with 1.0% agarose in 0.5×TBE at 200V, with 3-15 seconds switch times for 15 hours. DNA was visualized by staining with 0.5 μg/ml ethidium bromide.

Southern hybridization analysis was performed as follows: MU genomic DNA, separated under PFGE as described above, was transferred to Hybond N+ nylon membranes by overnight alkaline transfer in 0.4 M NaOH. Gels were subject to 1200 mjoules UV treatment prior to transfer. DNA was fixed to the nylon membranes by cross-linking (1200 mjoules UV) and then incubated in prehybridization buffer (5×SSC, 0.1% SDS, 1% skim-milk) for at least 2 hours at 68° C.

DNA probes were prepared by random-prime labelling of PCR products using the HighPrime random labelling kit (Stratagene) and incorporation of [.-32P] dCTP. Probes were denatured by heating to 100° C. and were then added to hybridization buffer (5×SSC, 0.1% SDS, 1% skim-milk) to a final concentration of approximately 10 ng/mL. Hybridization proceeded at 68° C. for 18 hours. The hybridization solution was then removed and 3 stringency washes were performed: once for 5 minutes in 2×SSC, 0.1% SDS at room temperature and then twice for 10 minutes in 0.1×SSC, 0.1% SDS at 68° C. The membrane was then washed in 2×SSC and sealed in clear plastic film before detection using a Storm phosphorimager (Molecular Dynamics). Probe stripping was performed by washing the membrane twice for 20 minutes at 68° C. with 0.1% SDS, 0.2M NaOH. The sizes of DNA restriction fragments were estimated with Sigmagel software (Jandel Scientific) using the Lambda low-range DNA size ladder (NEB) to calibrate the gel and blot images.

### EXAMPLE 17

### Bacterial Artificial Chromosome (BAC) Library Construction

A whole-genome MU BAC library was constructed as described previously for Mycobacterium tuberculosis (Brosch, R., S. V. Gordon, A. Billault, T. Garnier, K. Eiglmeier, C. Soravito, B. G. Barrell, and S. Cole. 1998. Use of a Mycobacterium tuberculosis H37Rv bacterial artificial chromosome library for genome mapping, sequencing, and comparative genomics. Infect Immun 66:2221-2229.). Briefly, genomic DNA from MU strain Agy99 was prepared in agarose plugs as described above and subject to partial HindIII digestion. The DNA was separated under PFGE conditions. Partially digested DNA in the size range 40-120 kb was cloned into the unique HindIII site of the vector pBeloBAC11 and then used to transform E. coli DH10B by electroporation. The resulting clones were stored in LB-broth containing 15% glycerol in 96-well format at −80° C.

### EXAMPLE 18

### BAC Plasmid DNA Preparation

BAC DNA for automated sequencing was extracted using the method of Brosch et al (Brosch, R., S. V. Gordon, A. Billault, T. Garnier, K. Eiglmeier, C. Soravito, B. G. Barrell, and S. Cole. 1998. Use of a Mycobacterium tuberculosis H37Rv bacterial artificial chromosome library for genome mapping, sequencing, and comparative genomics. Infect Immun 66:2221-2229.). For subcloning of BACs, DNA was prepared from 40 ml overnight E. coli cultures and the plasmid DNA was extracted as previously described (Brosch, R., S. V. Gordon, A. Billault, T. Garnier, K. Eiglmeier, C. Soravito, B. G. Barrell, and S. Cole. 1998. Use of a Mycobacterium tuberculosis H37Rv bacterial artificial chromosome library for genome mapping, sequencing, and comparative genomics. Infect Immun 66:2221-2229.).

### EXAMPLE 19

### Phylogenetic Analysis

The sequences from the four, plasmid loci (repA, parA, mls, MUP045) that were present in all 10 MU strains were concatenated in-frame to produce a 1266 bp semantide for each strain. These sequences were then aligned with CLUSTALW (Thompson, J. D., D. G. Higgins, and T. J. Gibson. 1994. CLUSTAL W: improving the sensitivity of progressive multiple sequence alignment through sequence weighting, position-specific gap penalties and weight matrix choice. Nucleic Acids Res 22:4673-4680.). In the same way, the plasmid sequences obtained from the seven MU strains that contained the following seven loci were concatenated in frame to produce a 2208 bp semantide composed of repA, parA, MUP011, mls load, mlsAT(II), MUP038 and MUP045.

Phylogenetic analysis was performed with MEGA software version 2.1 (Kumar, S., K. Tamura, I. B. Jakobsen, and M. Nei. 2001. MEGA2: molecular evolutionary genetics analysis software. Bioinformatics 17:1244-1245.). ‘P’ distances were used through out as the overall level of sequence divergence was small. Values for synonymous (dS) and nonsynonymous (dN) mutation frequencies were calculated with Nei and Gojobori's method (Nei, M., and T. Gojobori. 1986. Simple methods for estimating the numbers of synonymous and nonsynonymous nucleotide substitutions. Mol Biol Evol 3:418-426.) and standard errors for the means of these values were estimated by the method of Nei and Jin (Nei, M., and L. Jin. 1989. Variances of the average numbers of nucleotide substitutions within and between populations. Mol Biol Evol 6:290-300.). The calculations of dS and dN were performed using the dSdNqw program (da Silva, J., and A. L. Hughes. 1998. dSdNqw, 1.0 ed. Pennsylvania State University, University Park, Pa.).

The MU Plasmid pMUM001 is Unstable in MU Strain Agy99

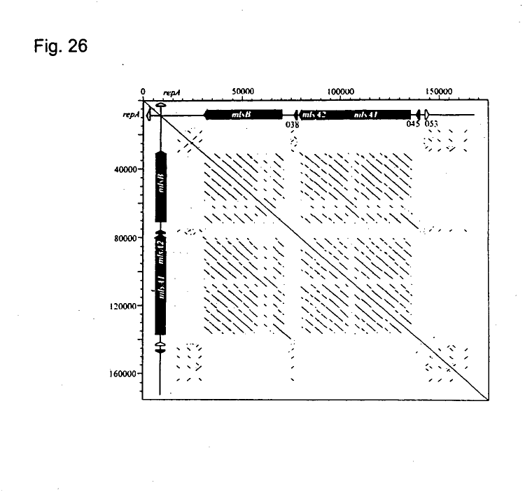

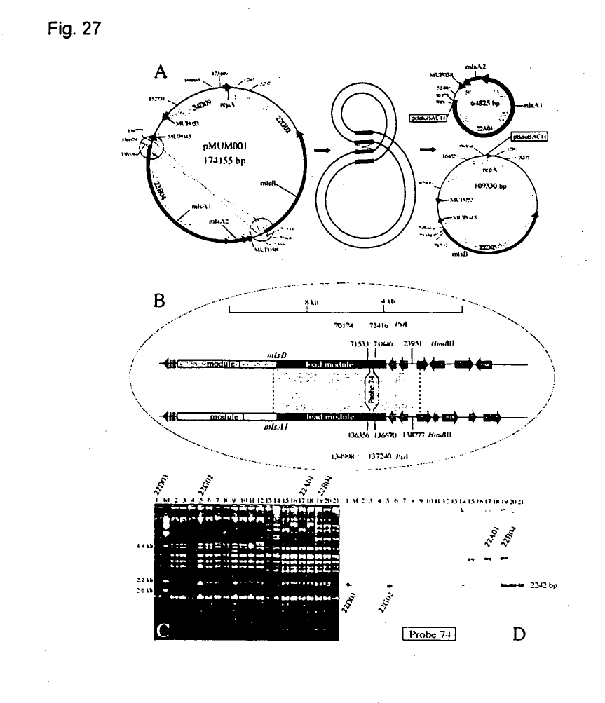

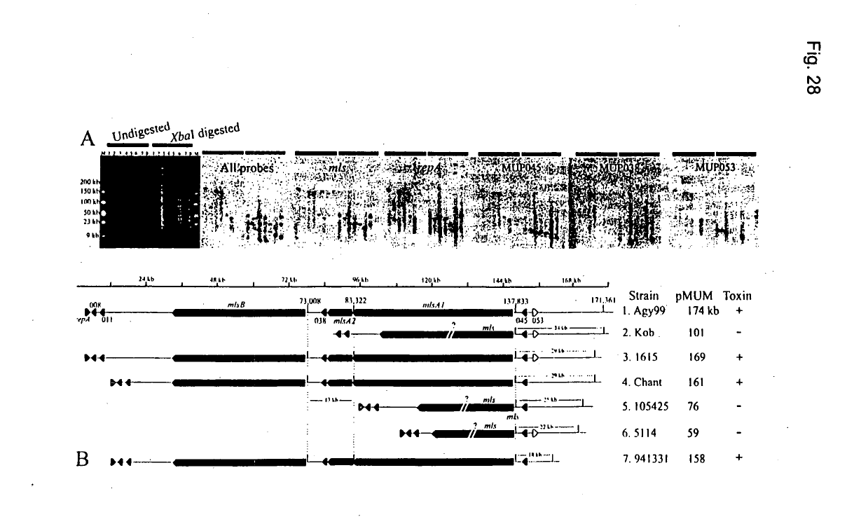

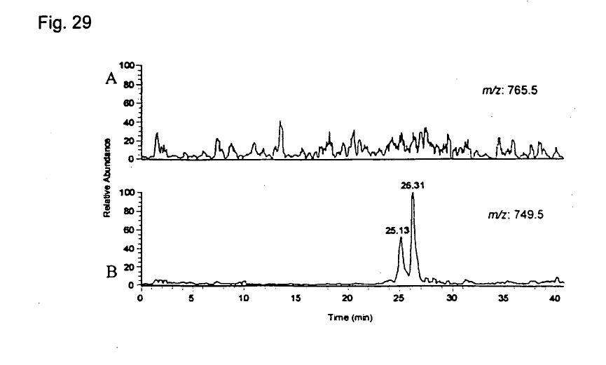

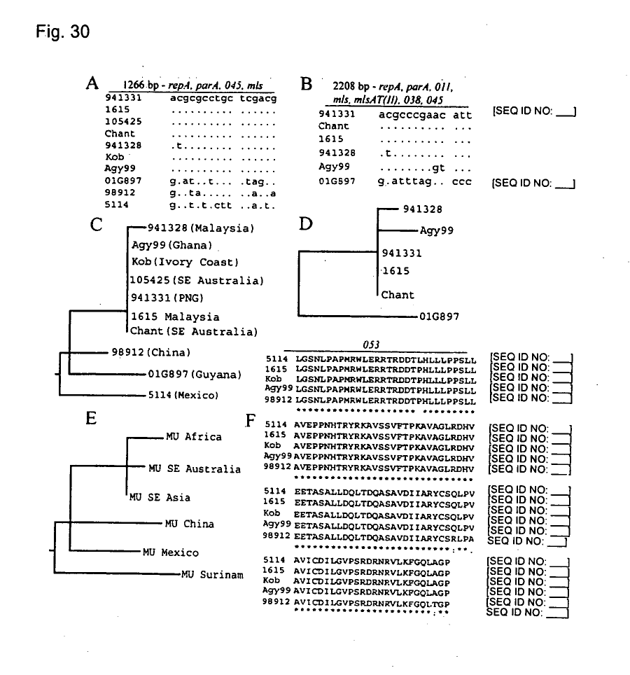

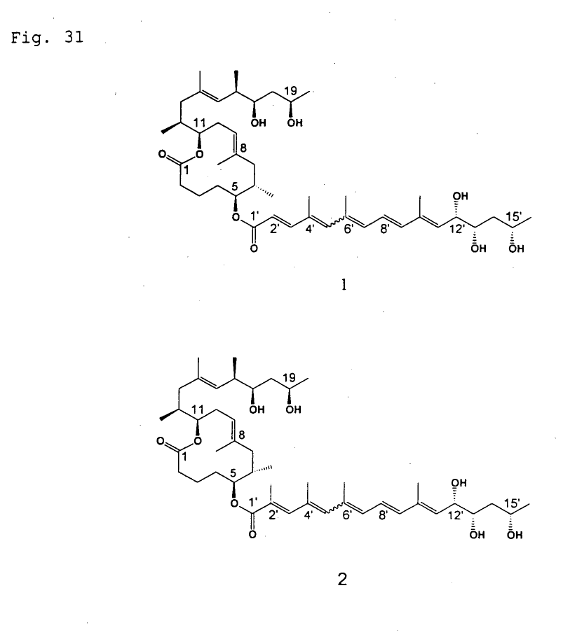

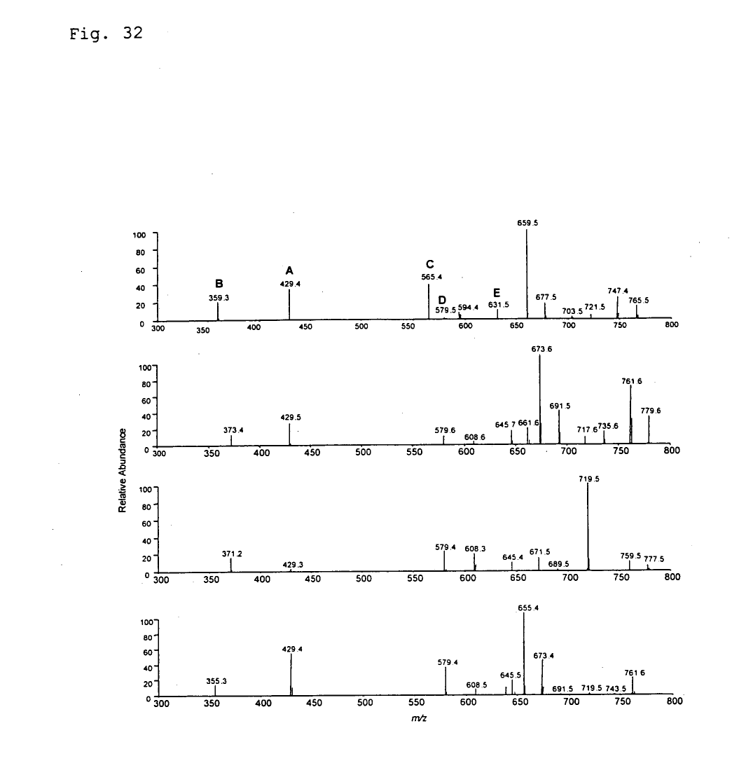

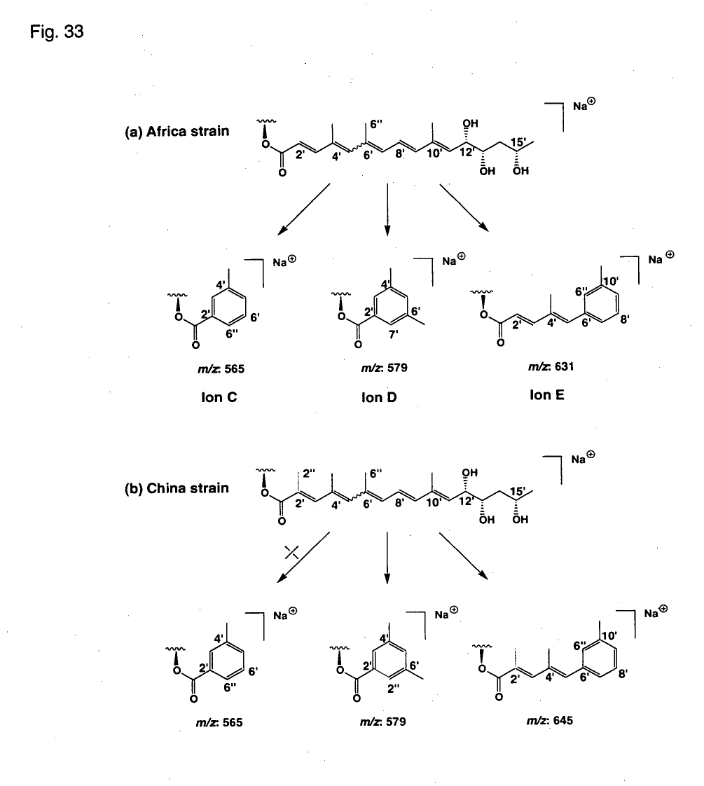

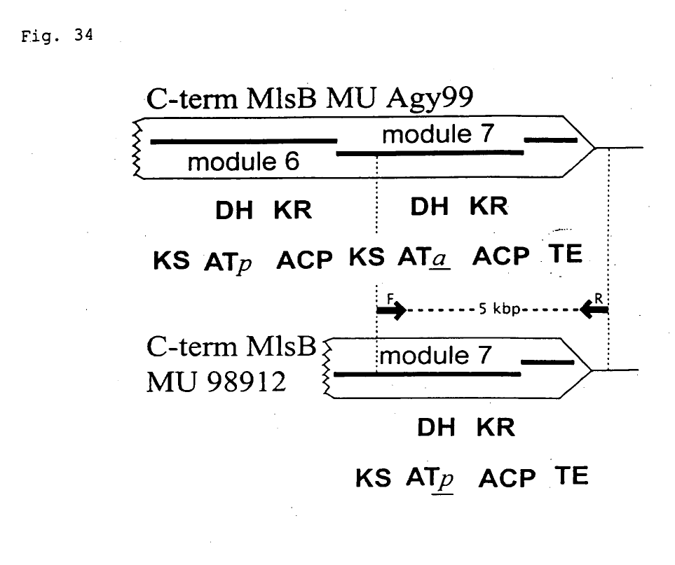

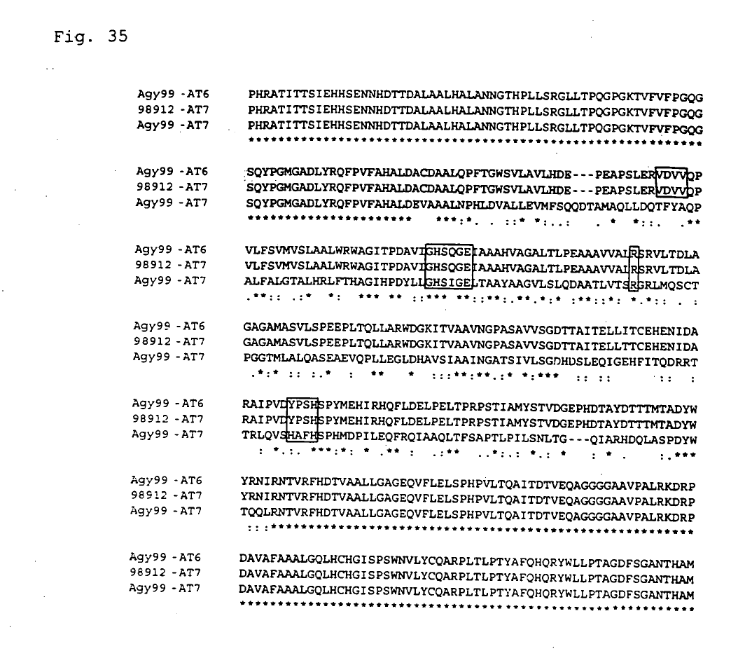

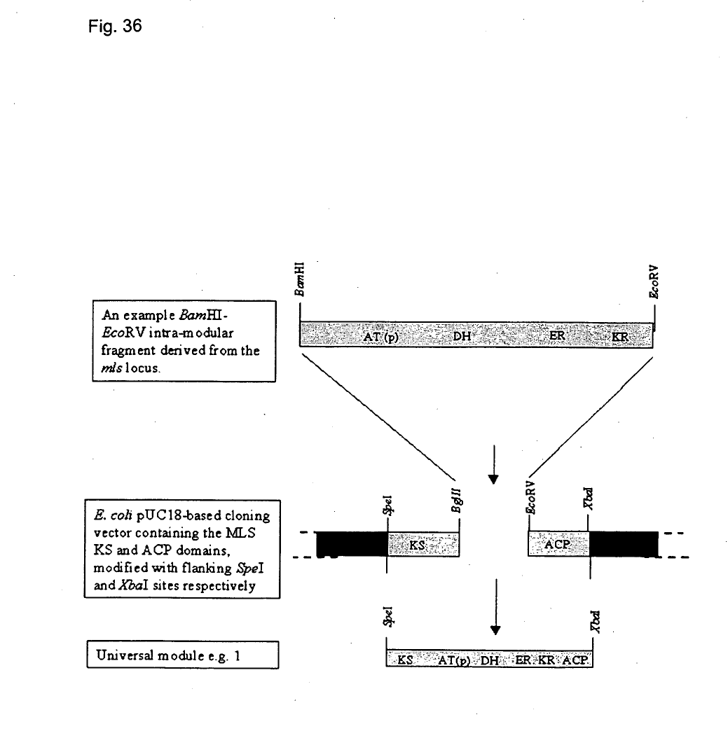

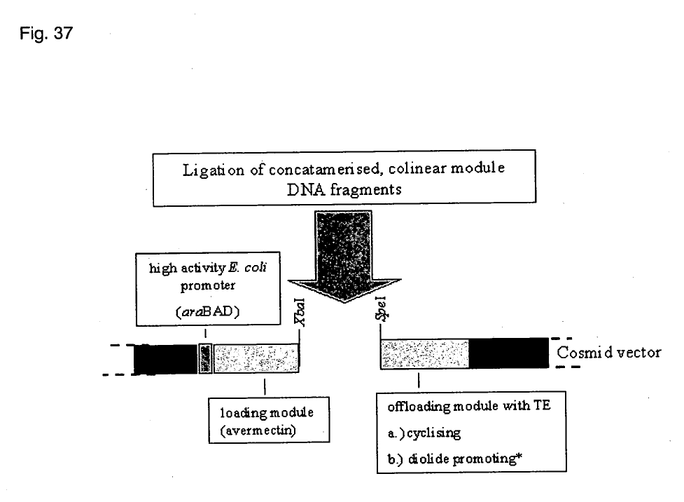

The eleven different functional domains of the mycolactone polyketide synthase genes (mlsA1, mlsA2 and mlsB) contain an unprecedented level of inter-domain nucleotide identity (>97%). The high level of sequence repetition within the locus is displayed in the Dotter plot shown in FIG. 26. It was hypothesized that this DNA homology would act as a substrate for recombination and manifest itself as inherent instability and variability of the mis locus within and between MU strains.

The first evidence that this was indeed the case was obtained in the course of determining the complete sequence of pMUM001 when several MU BAC clones, derived from a single DNA preparation of MU Agy99, were found to represent two different deletion variants of the 174 kb plasmid. These variants are represented by the clones 22A01 and 22D03, and they were discovered by DNA-end sequencing of a MU genomic BAC library of 176 clones. Sequence analysis revealed 22 clones containing pMUM-related sequences. These 22 clones were then further grouped into two sub-families based on two distinct types of PstI RF profile. Some of the clones within each subfamily had end sequences that indicated that they had been cloned into pBeloBAC11 at a single (but varying) MU HindIII site, raising the possibility that the entire MU plasmid had been cloned. However, this hypothesis was discounted as the insert sizes of these clones was either 65 kb or 110 kb, much less than the expected 174 kb. Curiously, the sum of these two BAC clones was 175 kb, leading to the possibility that these clones represented deletion variants of pMUM001.

A representative clone from each family was fully sequenced and annotated. Comparisons of the complete sequence of each clone with the complete sequence of pMUM001 indicated that these were indeed deletion derivatives that had arisen as a result of a recombination event between two identical 8237 bp sequences overlapping the beginning of mlsA1 and mlsB (FIG. 26, FIG. 27A&B). This arrangement was confirmed by PstI RE digestion and Southern hybridization of all BAC clones containing MU plasmid sequences (FIG. 27C&D). These alternate plasmid forms were not detectable by PFGE and Southern hybridization of MU genomic DNA (FIG. 28A) and probably represent sub-populations among the predominant 174 kb plasmid form. It is possible that they may represent deletion variants that arose by recombination in E. coli, but the presence of several examples of the same variations, cloned at different HindIII sites (FIG. 27C) and the existence of similar variants in spontaneous MU mycolactone mutants (FIG. 28) argue against this proposition and support the idea that this is a real phenomenon, reflecting inherent instability of the locus.

All MU strains Contain a Related Plasmid.

To explore inter-strain plasmid variation, a panel of nine MU clinical isolates from geographically diverse origins was screened by PCR for the presence of eight MU plasmid markers. The results of this analysis are summarised in Table 2.

TABLE 2
PCR analysis of 10 different MU strains for the presence
of eight plasmid-asociated genes.

| MU strain | | pMUM001 marker | | | | | | | |
|---|---|---|---|---|---|---|---|---|---|
| (Country of | | | | 011 | mls | | 038 | 045 | 053 |
| origin) | | repA | parA | (STPK) | (load) | mlsAT(II) | (TEII) | (KSIII) | (p450) |
| 1. | Agy99 | + | + | + | + | + | + | + | + |
| | (Ghana) | | | | | | | | |
| 2. | Kob | + | + | + | + | + | − | + | + |
| | (Ivory Coast) | | | | | | | | |
| 3. | 1615 | + | + | + | + | + | + | + | + |
| | (Malaysia) | | | | | | | | |
| 4. | Chant | + | + | + | + | + | + | + | − |
| | (SE Australia) | | | | | | | | |
| 5. | 105425 | + | + | + | + | − | − | + | − |
| | (SE Australia) | | | | | | | | |
| 6. | 5114 | + | + | − | + | − | − | + | + |
| | (Mexico) | | | | | | | | |
| 7. | 941331 | + | + | + | + | + | + | + | − |
| | (PNG) | | | | | | | | |
| 8. | 941328 | + | + | + | + | + | + | + | − |
| | (Malaysia) | | | | | | | | |
| 9. | 98912 | + | + | − | + | + | + | + | + |
| | (China) | | | | | | | | |
| 10. | 01G897 | + | + | + | + | + | + | + | − |
| | (French Guiana) | | | | | | | | |

The presence of key plasmid replication and maintenance genes (repA and parA) and sections of the mycolactone biosynthesis genes (mls loading domain and MUP045) in all isolates indicated that they all contain an element closely related to pMUM001.

Plasmid Variation Between Strains

The absence of several of the other plasmid markers among some of the isolates pointed to plasmid variation. Most notable was the absence among three isolates of key mycolactone accessory genes, such as MUP038 (encoding a type-II thioesterase), and one of the mls acyl-transferase (AT) domains, the absence of the latter sequence indicating that these isolates would be unable to produce mycolactone.

PFGE and Southern hybridization were used to study in more detail the structure of the plasmids among seven of the ten MU strains. MU DNA was separated by PFGE. This DNA was then hybridized with a pool of probes derived from five of the plasmid markers described in Table 2. The results are shown in FIG. 28 and demonstrate that there is considerable difference in plasmid size among isolates, ranging from 59 kb to 174 kb. MU strains harbouring plasmids less than 110 kb would not be expected to produce mycolactone as the Mls biosynthetic cluster is encoded by genes encompassing approximately 110 kb of DNA. Screening of lipid extracts from the seven isolates by LC-MS confirmed this prediction, and that of the PCR analysis, as neither mycolactone nor its co-metabolites were detected in extracts from MU Kob (a recent West African MU isolate with a 101 kb plasmid), MU 5114 (a Mexican MU isolate with a 59 kb plasmid) and MU 105425 (an isolate from the culture collection of the IP, derived from the reference strain ATCC 19428, with a 76 kb plasmid).

Digestion with XbaI and hybridization with the five, pooled, plasmid markers resulted in a profile of two, three or four bands. For each strain, the sum of its XbaI fragments was equal to the size of its linear plasmid form in the absence of XbaI digestion (FIG. 28). This demonstrated that none of the plasmids had new, additional XbaI fragments.

Hybridization experiments with individual probes then permitted linking of plasmid markers to particular XbaI fragments and construction of low-resolution maps (FIG. 28B). The three mycolactone minus strains had large deletions of 75 kb, 98 kb and 115 kb. The hybridization data, showing the absence of MUP038 (encoding the type II thioesterase), together with the PCR data showing an absence of the AT domain of module 5 in mlsA1 and the AT domain of modules 1 and 2 in mlsB, confirming that these deletions had occurred, at least in part, within their respective mls loci.

Only the strains with four XbaI fragments produced mycolactone (MUAgy99, MU1616, MUChant and MU941331), and thus, by definition, they must all contain an intact mls locus. This fact was supported by the presence of conserved 54 kb and 13 kb fragments, corresponding to the locus harbouring the mlsA genes and MUP038. Therefore, the size variations detected amongst these four strains occurred in the regions flanking the mls genes.

Plasmid Variation Correlates with the Presence of Different Mycolactone Co-Metabolites

For the strain MU Chant and MU 941331, some of their plasmid size variation could be attributed to the absence of a region that includes the gene MUP053 (encoding a P450 hydroxylase). The product of MUP053 is predicted to hydroxylate the mycolactone side chain at C12′ to produce mycolactone A/B with a mass of [M+Na]+at m/z 765 (Stinear, T. P., A. Mve-Obiang, P. L. Small, W. Frigui, M. J. Pryor, R. Brosch, G. A. Jenkin, P. D. Johnson, J. K. Davies, R. E. Lee, S. Adusumilli, T. Garnier, S. F. Haydock, P. F. Leadlay, and S. T. Cole. 2004. Giant plasmid-encoded polyketide synthases produce the macrolide toxin of Mycobacterium ulcerans. Proc Natl Acad Sci USA 101:1345-1349.). Strains lacking the hydroxyl group at C12′ have a mass of [M+Na]+at m/z 749. This metabolite has been called mycolactone C (Mve-Obiang, A., R. E. Lee, F. Portaels, and P. L. Small. 2003. Heterogeneity of mycolactones produced by clinical isolates of Mycobacterium ulcerans: implications for virulence. Infect Immun 71:774-783.) and it is a characteristic of Australian strains. The absence of MUP053 in the Australian strain MU Chant correlates well with the presence of mycolactone C and absence of mycolactone A/B (FIG. 29). However, MU941331 also lacks MUP053, yet this strain produces the same mycolactone profile as MUAgy99 (Hong, H., P. J. Gates, J. Staunton, T. Stinear, S. T. Cole, P. F. Leadlay, and J. B. Spencer. 2003. Identification using LC-MSn of co-metabolites in the biosynthesis of the polyketide toxin mycolactone by a clinical isolate of Mycobacterium ulcerans. Chem Commun 21:2822-2823.) (data not shown).

Sequence Analysis Indicates a Common Origin for pMUM

Comparisons of the DNA sequences obtained from the four plasmid markers common among all MU strains revealed shared nucleotide identity scores >98%. For each strain, the four sequences obtained were concatenated in-frame in the order repA, parA, MUP045 and the mls loading domain to produce a 422-codon semantide. The sequences were aligned and a summary of the 16 variable sites detected by this analysis is shown in FIG. 30A. A phylogenetic relationship was then inferred from these sequences and this produced a dendrogram with a topology that closely mimicked the topology produced by the same analysis of seven chromosomally encoded genes in a previous MLST study (FIGS. 30C and 30E and (Stinear, T. P., G. A. Jenkin, P. D. R. Johnson, and J. K. Davies. 2000. Comparative Genetic Analysis of Mycobacterium ulcerans and Mycobacterium marinum Reveals Evidence of Recent Divergence. J. Bacteriol. 182:6322-6330.)). The congruence of these trees strongly suggests that pMUM was acquired as a single event and has co-evolved with its host. Comparisons of the frequencies of synonymous substitution in coding sequences are a measure of the time a given sequence has been extant relative to another (Hughes, A. L., R. Friedman, and M. Murray. 2002. Genomewide pattern of synonymous nucleotide substitution in two complete genomes of Mycobacterium tuberculosis. Emerg Infect Dis 8:1342-1346.). Thus, similar synonymous substitution frequencies for the plasmid-bome gene sequences versus the chromosomally encoded gene sequences would be consisent with the idea that plasmid acquisition coincided with the divergence of MU from a common progenitor.

The calculation of dS (where dS is number of synonymous substitutions per 100 synonymous sites) for both the plasmid and chromosomal sequences was not significantly different (plasmid-bome gene sequences: mean dS=0.59, se=0.24; chromosomal gene sequences: mean dS=0.54, se=0.17). Seven of the ten strains had seven of the eight plasmid markers. Therefore, to try and obtain further discrimination, the sequences from these strains were treated as above. Thus, for a given strain the seven sequences were concatenated in-frame in the order repA, parA, MUP011, mls load, mlsAT(II), MUP038 and MUP045 to produce a 736-codon semantide. These sequences were aligned and shared greater than 99% nucleotide identity (FIG. 30B). Inferred phylogeny was entirely consistent with that produced from the four plasmid markers and MLST (FIG. 30D).

MUP053, encoding a putative P450 monooxygenase with a possible role in modifying mycolactone, displayed an uneven distribution among strains. However, MUP053 is present in strains from Africa, Malaysia, China and Mexico, and these strains span the known genetic diversity of the species. The shared DNA and aa identity for MUP053 between these strains was 98% and 96% respectively; equal to other plasmid sequences (FIG. 30F). This suggests that MUP053 was present in a progenitor MU and has subsequently been lost from some strains as the species has evolved.

MU provides the first direct evidence of the importance, not only of gene loss, but also LGT in the evolution of pathogenesis among the mycobacteria. MU is an example of an emerging mycobacterial pathogen that has evolved by acquiring a plasmid (pMUM) that confers a virulence phenotype and, probably more critically for the organism, a fitness advantage for a particular niche environment. Previous multilocus sequence typing (MLST) studies have shown that at a nucleotide level, MU is highly related to Mycobacterium marinum, the latter species being a natural pathogen of fish and phenotypically quite distinct from MU. However, the two species were shown to share greater than 98% DNA identity across seven non-linked genes and among 40 diverse strains (Stinear, T. P., G. A. Jenkin, P. D. R. Johnson, and J. K. Davies. 2000. Comparative Genetic Analysis of Mycobacterium ulcerans and Mycobacterium marinum Reveals Evidence of Recent Divergence. J. Bacteriol. 182:6322-6330.). Phylogenetic analysis strongly suggested that MU had evolved from a common M. marinum progenitor and from this result it was hypothesised that divergence of MU as a discrete clonal grouping had been assisted by acquisition of foreign DNA. Subsequent work has revealed the presence of the virulence plasmid pMUM in MU, and the present invention shows that pMUM is a key attribute of MU and that it is present in a range of MU strains obtained from around the world. Comparisons of pMUM gene sequences between these strains with chromosomal gene sequences, revealed congruent tree topologies and identical frequencies of synonymous substitution, strongly suggesting that acquisition of pMUM marked the divergence of the species from a single, M. marinum progenitor. Plasmid acquisition has then been followed by other independent genome changes within MU strains from different areas to produce the regiospecific phenotypes and genotypes now seen (Chemlal, K., K. De Ridder, P. A. Fonteyne, W. M. Meyers, J. Swings, and F. Portaels. 2001. The use of IS2404 restriction fragment length polymorphisms suggests the diversity of Mycobacterium ulcerans from different geographical areas. Am J Trop Med Hyg 64:270-273. Stinear, T., J. K. Davies, G. A. Jenkin, F. Portaels, B. C. Ross, F. Oppedisano, M. Purcell, J. A. Hayman, and P. D. R. Johnson. 2000. A simple PCR method for rapid genotype analysis of Mycobacterium ulcerans. J Clin Microbiol 38:1482-1487. Stinear, T. P., G. A. Jenkin, P. D. R. Johnson, and J. K. Davies. 2000. Comparative Genetic Analysis of Mycobacterium ulcerans and Mycobacterium marinum Reveals Evidence of Recent Divergence. J. Bacteriol. 182:6322-6330.).

One of the unusual features of pMUM001 is the unprecedented DNA homology among the functional domains of the mls genes. Whilst the mis genes occupy 105 kb of pMUM001, this region is composed of less than 10 kb of unique sequence (Stinear, T. P., A. Mve-Obiang, P. L. Small, W. Frigui, M. J. Pryor, R. Brosch, G. A. Jenkin, P. D. Johnson, J. K. Davies, R. E. Lee, S. Adusumilli, T. Garnier, S. F. Haydock, P. F. Leadlay, and S. T. Cole. 2004. Giant plasmid-encoded polyketide synthases produce the macrolide toxin of Mycobacterium ulcerans. Proc Natl Acad Sci U S A 101:1345-1349.). This extraordinary economy of sequence is reflected in FIG. 2 and suggests that the mls genes have been created de novo by successive recombination events such as in-frame duplications and deletions from a core set of PKS sequences. The precise origin of such a core gene set remains obscure as DNA database searches have revealed no orthologous genes, but the significant aa identity to PKS sequences from other species of mycobacteria and streptomyces points to a likely origin among the actinomycetes. In addition to suggesting an evolutionary recent origin for mycolactone biosynthesis, the extended DNA sequence homology also implies that such an arrangement would be inherently unstable, acting as a substrate for general recombination. This invention shows that in MUAgy99, pMUM001 is unstable and that recombination between two homologous sequences gave rise to two deletion variants. The larger 109 kb variant, represented by the BAC clone 22D03 contains an intact origin of replication and is thus likely to be maintained within a cell population. Cells harboring the 22D03 variant would be incapable of producing mycolactone, but could theoretically still produce the acyl side chain. However, the smaller 65 kb deletion variant, represented by the BAC clone 22A01, would be lost to the population upon cell division as it is incapable of autonomous replication, despite having the genes required for synthesis of the mycolactone core.

Spontaneous mycolactone-minus and avirulent MU mutants were first reported by George et al. (George, K. M., D. Chatterjee, G. Gunawardana, D. Welty, J. Hayman, R. Lee, and P. L. Small. 1999. Mycolactone: a polyketide toxin from Mycobacterium ulcerans required for virulence. Science 283:854-857.) and were used to demonstrate the key role of mycolactone in virulence. Mycolactone confers a pale yellow color to colonies, and mycolactone-minus mutants are readily observed as white colony variants when grown on Lowenstein-Jensen (LJ) medium. Attempts were made to isolate white colony variants of MUAgy99 to try and identify the 109 kb deleted form of pMUM001. While white colonies were readily detected on LJ media, their growth rate on subculture was highly impaired and it was not possible to generate the biomass required for additional studies, such as PFGE. Nevertheless, investigation of other MU strains revealed deleted forms of pMUM similar to those identified in MUAgy99 (in particular MUKob), and these deleted forms had corresponding toxin-minus phenotypes. Each strain tested had a different plasmid size and the mapping data showed that deletions had occurred to varying extents and in different regions of pMUM. Recombination between homologous sequences is one explanation for this variety, but given the large number of insertion sequences (IS) in pMUM (Stinear, T. P., A. Mve-Obiang, P. L. Small, W. Frigui, M. J. Pryor, R. Brosch, G. A. Jenkin, P. D. Johnson, J. K. Davies, R. E. Lee, S. Adusumilli, T. Garnier, S. F. Haydock, P. F. Leadlay, and S. T. Cole. 2004. Giant plasmid-encoded polyketide synthases produce the macrolide toxin of Mycobacterium ulcerans. Proc Natl Acad Sci USA 101: 1345-1349.), another possibility is that IS are also mediating some of these plasmid rearrangements.

It is probably significant that no pMUM-minus MU strains were found. While such mutants may exist the recent finding that pMUM contains an active partition (par) locus (Stinear et al. submitted), means that spontaneous curing is likely to be an infrequent event. Par loci are cis-acting elements that function to ensure daughter cells faithfully receive a copy of an episome during cell division.

Following the assumption that the clinical isolates used in this invention were originally mycolactone proficient and thus contained intact pMUM, it appears that spontaneous toxin minus mutants, caused by deletion of MU-plasmid DNA, are a common occurrence. The frequency with which deletion mutants arise has not been calculated, but for some strains it appears to be very high. MUAgy99 and MUKob were recent clinical isolates from West Africa with minimal laboratory passaging. The DNA used for the MUAgy99 BAC library was prepared from a liquid culture that was at its fourth passage since primary isolation and MUKob was at its third passage. One outcome of this invention is to highlight the care researchers must take to continually test the plasmid and mycolactone status of the MU strains used in their work.

Plasmid instability contrasts most strikingly with the fact that MU isolates recovered from diverse geographic locations around the world produce a relatively homogeneous range of mycolactones (Mve-Obiang, A., R. E. Lee, F. Portaels, and P. L. Small. 2003. Heterogeneity of mycolactones produced by clinical isolates of Mycobacterium ulcerans: implications for virulence. Infect Immun 71:774-783.). This apparent paradox leads compellingly to the notion that there is strong purifying selection for maintenance of a mycolactone-proficient form of pMUM, presumably because mycolactone is playing a key function for MU in the environment. It is probably unlikely that the cytotoxic properties of mycolactone for human cells are part of a primary survival role for the bacterium. However, one possibility given the highly episodic and geographically compact epidemiology of Buruli ulcer, where waves of MU infection can rapidly appear and then disappear from a given region, is that deleterious recombination and loss of the plasmid function are interrupting the chain of transmission at some point. Perhaps mycolactone is a factor required for colonization or persistence in insect salivary glands (Marsollier, L., R. Robert, J. Aubry, J. P. Saint Andre, H. Kouakou, P. Legras, A. L. Manceau, C. Mahaza, and B. Carbonnelle. 2002. Aquatic Insects as a Vector for Mycobacterium ulcerans. Appl Environ Microbiol 68:4623-4628.) or establishment of a biofilm on plant surfaces (Marsollier, L., T. Stinear, J. Aubry, J. P. Saint Andre, R. Robert, P. Legras, A. L. Manceau, C. Audrain, S. Bourdon, H. Kouakou, and B. Carbonnelle. 2004. Aquatic plants stimulate the growth of and biofilm formation by Mycobacterium ulcerans in axenic culture and harbor these bacteria in the environment. Appl Environ Microbiol 70:1097-1103.). In other clonal bacterial pathogens, such as Yersinia pestis, a modest number of genetic changes have led to a dramatically different route of transmission and mode of pathogenesis compared with their progenitors. Indeed, despite their radically different disease pathologies, there are many parallels between Y. pestis and MU, where in the case of the agent of plague, acquisition of the plasmid encoded genes ymt, and hms have conferred the respective abilities of resistance to digestion in the midgut of fleas and persistence on the surface of spines that line the interior of the proventriculus, thus facilitating an arthropod-linked mode of transmission (Hinnebusch, B. J., A. E. Rudolph, P. Cherepanov, J. E. Dixon, T. G. Schwan, and A. Forsberg. 2002. Role of Yersinia murine toxin in survival of Yersinia pestis in the midgut of the flea vector. Science 296:733-735. Jarrett, C. O., E. Deak, K. E. Isherwood, P. C. Oyston, E. R. Fischer, A. R. Whitney, S. D. Kobayashi, F. R. DeLeo, and B. J. Hinnebusch. 2004. Transmission of Yersinia pestis from an infectious biofilm in the flea vector. J Infect Dis 190:783-792.).

While the repetitive nature of the mls locus has not yet led to heterogeneity among mycolactones, one DNA deletion identified in this invention can be linked with the production of variant toxin. The plasmid gene MUP053 encodes a putative P450 monoxygenase, an enzyme thought to be required for hydroxylation of mycolactone at position C12′ of its fatty-acid side chain to produce mycolactone A/B (m/z 765). As predicted, the Australian strain MU Chant lacks MUP053 and produces a lower mass metabolite at m/z 749 (mycolactone C) that corresponds with the absence of a hydroxyl group. The fact that MU 941331 from PNG also lacks MUP053, but still produces oxidized mycolactones, suggests that in some strains, there may be chromosomal P450 genes encoding hydroxylases active against the molecule.

This invention has shown that there is considerable mutational dynamism in pMUM. It may be that there is constant genetic flux within the Mls genes such that new mycolactones are being continuously created within a given MU population. However, if new metabolites do not confer a fitness advantage, then cells with such changes will not persist.

The genetic basis for mycolactone biosynthesis has recently been revealed, T. Stinear, Mve-Obiang, A., Small, P. L., Frigui, W., Pryor, M. J., Brosch, R., Jenkin, G. A., Johnson, P. D., Davies, J. K., Lee, R. E., Adusumilli, S., Garnier, T., Haydock, S. F., Leadlay, P. F., S. T. Cole, Proc. Natl. Acad. Sci. U.S.A. 2004, 101, 1345-1349: M. ulcerans contains a 174 kb mega-plasmid, which harbours, in addition to a number of auxiliary genes, several very large genes encoding type I modular polyketide synthases closely resembling the actinomycete PKSs that govern the biosynthesis of erythromycin, rapamycin and other macrocyclic polyketides, where each module of fatty acid synthase-related enzyme activities catalyses a specific cycle of polyketide chain extension. L. Katz, S. Donadio, Annu. Rev. Microbiol. 1993 1993, 47, 875-912; J. Staunton, K. J. Weissman, Nat. Prod. Rep. 2001, 18, 380-416. Genes mlsA1 (51 kbp) and mlsA2 (7 kbp) encode the PKS for production of the 12-membered core lactone, while mlsB (42 kbp) encodes the side-chain PKS.

The availability of this sequence led to an investigation of the structural differences between mycolactones A/B, from an African isolate (MUAgy99) and the mycolactones produced by another pathogenic strain of M. ulcerans, to see whether any variant mycolactones in the latter strain might be accounted for by changes within the PKS rather than changes in processing steps. To characterise the mycolactone metabolites, a recently-described method of LC-sequential mass spectrometry (LC-MSⁿ) was used, performed on an ion trap mass spectrometer. H. Hong, P. J. Gates, J. Staunton, T. Stinear, S. T. Cole, P. F. Leadlay, J. B. Spencer, Chem. Commun. 2003, 2822-2823. Ion trap mass spectrometry (using either FTICR or a quadrupole ion trap) allows multi-stage collision fragmentation of target molecules, which yields detailed structural information. It was discovered that mycolactones from a pathogenic strain of M. ulcerans from China (MU98192) all possess an extra methyl group at C2′ compared to mycolactone A (see FIG. 31), as the apparent result of the recruitment of a single catalytic domain of altered specificity in the mycolactone PKS.

For details of the growth of M. ulcerans strains and extraction of metabolites, see Examples 20-21. Preliminary LC-MS analysis of the cell extract showed that normal mycolactones, with characteristic values of m/z 765, 763, 749, and 747, were not produced by the Chinese strain, MU98912. However, at least three new components at m/z 779, 777 and 761, were detected. When on-line LC-MS/MS analyses were performed on these ions, they showed fragmentation patterns surprisingly similar to that of normal mycolactone A/B (see FIG. 32). All the MS/MS spectra of the mycolactones from MU98912 contained fragment ions corresponding to A and B, which are characteristic ions of mycolactone corresponding to the core lactone and to the polyketide side chain, respectively. H. Hong, P. J. Gates, J. Staunton, T. Stinear, S. T. Cole, P. F. Leadlay, J. B. Spencer, Chem. Commun. 2003, 2822-2823. Fragment ion A was conserved in all the spectra, while fragment ion B varied exactly in accordance with the variation in the mass of the precursor ion. It therefore appears that the core lactone is identical in the mycolactones from MUAgy99 and MU98912, and structural variations are restricted to the polyketide side chain.

To obtain further information about such structural variations, off-line accurate-mass analyses and deuterium exchange experiments were performed on these newly-identified mycolactones. The results, when compared to those the classic mycolactones from MUAgy99 (Table 1) clearly showed that mycolactones from MU98912 have the same number of exchangeable protons, but also an extra methylene group, compared to their counterparts from MUAgy99.

TABLE 1
Comparison of molecular formula, and of numbers of exchangeable
protons, of mycolactones from the Africa and the China strain.

| Africa strain* | | | China strain | | | | |
|---|---|---|---|---|---|---|---|
| | | No. of | | | | | No. of |
| | | deuterons | | | | | deuterons |
| Metabolite | | after | Metabolite | | Observed | Error | after |
| [M + Na]⁺ | Formula | exchange | [M + Na]⁺ | Formula | Mass | (ppm) | exchange |
| 765 | C₄₄H₇₀O₉Na | 5 | 779 | C₄₅H₇₂O₉Na | 779.5022 | −6.0 | 5 |
| 763 | C₄₄H₆₈O₉Na | 4 | 777 | C₄₅H₇₀O₉Na | 777.4922 | 1.3 | 4 |
| 747 | C₄₄H₆₈O₈Na | 3 | 761 | C₄₅H₇₀O₈Na | 761.4943 | 3.0 | 3 |

*The data for mycolactones from MUAgy99 are taken from reference [10].

These results might be accounted for if there were an extra C— or O-linked methyl substituent in the side chain of all the mycolactones from the MU98912.

To test this idea, and to locate the exact position of such an extra methyl group within the side chain, detailed comparisons were carried out between the MS/MS spectra of mycolactones from the two strains. In the MS/MS spectra of mycolactones from MUAgy99 (a representative MS/MS spectrum (of m/z 765) is shown in FIG. 32), the fragment ion at m/z 565 is always seen. It has been proposed that this conserved fragment, designated fragment ion C, H. Hong, P. J. Gates, J. Staunton, T. Stinear, S. T. Cole, P. F. Leadlay, J. B. Spencer, Chem. Commun. 2003, 2822-2823, arises as a result of cleavage at the C6′-C7′ bond. In addition to fragment ion C, conserved fragment ions at m/z 579 (ion D) and 631 (ion E) arise from the mycolactones from MUAgy99, and are identified by the deuteriated MS/MS analysis (data not shown) as resulting from cleavage of C7′-C8′, and C10′-C11′, respectively. (See FIG. 33). In comparison, in the MS/MS spectra of mycolactones from MU98912, the deuteriated MS/MS analysis showed the counterpart of ion E (m/z 631) increased by 14 mass units to m/z 645, suggesting that there is an extra methyl, and that it lies within the span C2′ to C10′. However, no fragment 14 mass units higher than fragment ion D (m/z 579) was seen. Instead of both ion C (m/z 565) and ion D (m/z 579), only a fragment ion at m/z 579 (14 mass units higher than fragment C) was seen. This important information provides strong evidence that there is an extra C-linked methyl group, at the C2′ position.

In the light of this specific structural difference between the mycolactones from MUAgy99 and MU98912, respectively, nucleotide sequence analysis of the appropriate part of the mycolactone biosynthetic genes was carried out. Preliminary restriction mapping analysis of the M. ulcerans megaplasmid bearing the mycolactone biosynthetic genes showed (as expected) no evident differences between MUAgy99 and MU98912. The DNA encoding extension module 7 of the PKS MlsB, which governs the insertion of the last polyketide extension unit to provide carbons C1′ and C2′ of the side-chain was amplified by PCR and sequenced. For the bulk of this module, there were no significant amino acid sequence differences between the two strains (overall DNA sequence identity >99.3%). However, the acyltransferase domain AT7 showed highly significant differences, as shown in FIG. 34. The sequence of AT7 from MU98912 is identical to a typical methylmalonyl-CoA specific AT domain from elsewhere in the mycolactone PKS, such as the extension module 6 of MlsB, T. Stinear, Mve-Obiang, A., Small, P. L., Frigui, W., Pryor, M. J., Brosch, R., Jenkin, G. A., Johnson, P. D., Davies, J. K., Lee, R. E., Adusumilli, S., Garnier, T., Haydock, S. F., Leadlay, P. F., S. T. Cole, Proc. Natl. Acad. Sci. U.S.A. 2004, 101, 1345-1349, and differs markedly over much of its length from the sequence of the (malonyl-CoA specific) AT7 of MUAgy99. In particular, the sequence motifs highlighted are all highly diagnostic of differences between substrate specificity for methylmalonyl- or malonyl-CoA, respectively. S. F. Haydock, J. F. Aparicio, I. Molnar, T. Schwecke, L. E. Khaw, A. Konig, A. F. A. Marsden, I. S. Galloway, J. Staunton, P. F. Leadlay, FEBS Lett. 1995, 374, 246-248; Biotica, patent; Kosan, biochemistry; F. Del Vecchio, H. Petkovic, S. G. Kendrew, L. Low, B. Wilkinson, R. Lill, J. Cortes, B. A. Rudd, J. Staunton, P. F. Leadlay, J. Ind. Microbiol. Biotechnol. 2003, 30, 489-494.

It has been recently demonstrated that the substrate specificity of an acyltransferase domain in a modular PKS can be widened, to accommodate both methylmalonyl-CoA and malonyl-CoA, by the specific alteration of a very few key active-site residues. Biotica, patent; Kosan, biochemistry; F. Del Vecchio, H. Petkovic, S. G. Kendrew, L. Low, B. Wilkinson, R. Lill, J. Cortes, B. A. Rudd, J. Staunton, P. F. Leadlay, J. Ind. Microbiol. Biotechnol. 2003, 30, 489-494. FIG. 35 illustrates the fact that AT domains in the mycolactone PKS that are specific for malonyl- and methylmalonyl-CoA, respectively, show much morc deep-seated differences, and are only mutually identical in sequence at their N-termini and (particularly) at their C-termini. There is thus an apparent replacement of a large portion of the side chain PKS module 7 AT domain in one M. ulcerans strain compared to the other. The evolutionary pathway by which these changes occurred remains obscure, but the discovery of this natural difference is prefigured by the strategy of AT “domain swapping” which has been widely used to switch the chemical specificity of modular PKSs. M. Oliynyk, M. J. Brown, J. Cortes, J. Staunton, P. F. Leadlay, Chem. Biol. 1996, 3, 833-939. R. McDaniel, A. Thamchaipenet, C. Gustafsson, H. Fu, M. Betlach, G. Ashley, Proc. Natl. Acad. Sci. U.S.A. 1999, 96, 1846-1851.

### EXAMPLE 20

### Microbiological Methods

The two clinical isolates of M. ulcerans used in this invention, MUAgy99 and MU98912, were obtained from patients in Ghana and China, respectively. W. R. Faber, L. M. Arias-Bouda, J. E. Zeegelaar, A. H. Kolk, P. A. Fonteyne, T. J., P. F., Trans. R. Soc. Trop. Med. Hyg. 2000, 94, 277-279. MU98912 was kindly provided by F. Portaels. The growth of strains and the preparation of cell extracts were performed as previously described. H. Hong, P. J. Gates, J. Staunton, T. Stinear, S. T. Cole, P. F. Leadlay, J. B. Spencer, Chem. Commun. 2003, 2822-2823. For DNA sequence analysis, the DNA encoding module 7 of the PKS MlsB was PCR-amplified from each strain using genomic DNA as template with the forward primer ALLKS-CTERM-F 5′-CCTCATCCTCCAACAACC-3′ [SEQ ID NO.:35](corresponding to the C-terminal end of the KS7 domain of MlsB) and the reverse primer MLSB-intTE-R 5′-GCTCAACCTCGTTTTCCCCATAC-3′ [SEQ ID NO.:36] (corresponding to a position just downstream of the mlsB stop codon as shown in FIG. 34). A 5 kbp product was obtained in both cases and this was fully sequenced on both strands by primer walking. The DNA sequence obtained from MU98912 has been deposited in Genbank under the accession No. AY743331.

### EXAMPLE 21

### LC-MS Analysis

LC-MS and LC-MS/MS analyses were carried out on a Finnigan LCQ instrument, essentially as previously described. H. Hong, P. J. Gates, J. Staunton, T. Stinear, S. T. Cole, P. F. Leadlay, J. B. Spencer, Chem. Commun. 2003, 2822-2823. Accurate mass analyses were performed on an API QSTAR pulsar (Applied Biosystems). Deuterium exchange experiments were carried out as previously described. H. Hong, P. J. Gates, J. Staunton, T. Stinear, S. T. Cole, P. F. Leadlay, J. B. Spencer, Chem. Commun. 2003, 2822-2823.

In summary, this invention also provides new analogues of the toxin mycolactone, identified in a pathogenic Chinese strain of Mycobacterium ulcerans, which possess an extra methyl group at C2′ compared to mycolactone A (see Figure), as a result of the recruitment of a single catalytic domain of altered specificity in the mycolactone PKS, an as shown below.

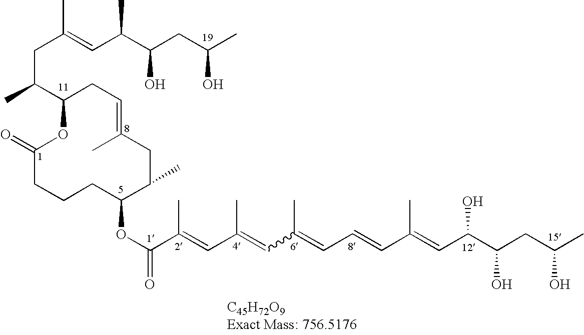

The foregoing references and each of the following references are cited herein. The entire disclosure of each reference is relied upon and incorporated by reference herein.

### REFERENCES

1. Hayman, J. & McQueen, A. (1985) Pathology 17, 594-600.
2. George, K. M., Chatterjee, D., Gunawardana, G., Welty, D., Hayman, J., Lee, R. & Small, P. L. (1999) Science 283, 854-857.
3. Stinear, T. P., Jenkin, G. A., Johnson, P. D. R. & Davies, J. K. (2000) J. Bacteriol 182, 6322-6330.
4. Jenkin, G. A., Stinear, T. P., Johnson, P. D. R. & Davies, J. K. (2003) J. Bacteriol In press.
5. Brosch, R., Gordon, S. V., Billault, A., Garnier, T., Eiglmeier, K., Soravito, C., Barrell, B. G. & Cole, S. (1998) Infect Immun 66, 2221-2229.
6. Cole, S. T., Brosch, R., Parkhill, J., Garnier, T., Churcher, C., Harris, D., Gordon, S. V., Eiglmeier, K., Gas, S., Barry, C. E., 3rd, et al. (1998) Nature 393, 537-44.
7. Bonfield, J. K., Smith, K. F. & Staden, R. (1995) Nucleic Acids Res 24, 4992-4999.
8. Rubin, E. J., Akerley, B. J., Novick, V. N., Lampe, D. J., Husson, R. N. & Mekalanos, J. J. (1999) Proc Natl Acad Sci USA 96, 1645-1650.
9. Mve-Obiang, A., Lee, R. E., Portaels, F. & Small, P. L. (2003) Infect Immun 71, 774-783.
10. Gavigan, J. A., Ainsa, J. A., E., P., Otal, I. & Martin, C. (1997) J Bacteriol 179, 4115-4122.
11. Durocher, D. & Jackson, S. P. (2002) FEBS Lett 513, 58-66.
12. Betts, J. C., Lukey, P. T., Robb, L. C., McAdam, R. A. & Duncan, K. (2002) Mol Microbiol 43, 717-731.
13. Stinear, T., Ross, B. C., Davies, J. K., Marino, L., Robins-Browne, R. M., Oppedisano, F., Sievers, A. & Johnson, P. D. R. (1999) J Clin Microbiol 37, 1018-1023.
14. Kwon, H. J., Smith, W. C., Scharon, A. J., Hwang, S. H., Kurth, M. J. & Shen, B. (2002) Science 297, 1327-1330.
15. Heathcote, M. L., Staunton, J. & Leadlay, P. F. (2001) Chem Biol 8, 207-220.
16. Katz, L. & Donadio, S. (1993) Annu Rev Microbiol 47, 875-912.
17. Staunton, J. & Weissman, K. J. (2001) Nat Prod Rep 18, 380-416.
18. Bisang, C., Long, P. F., Cortes, J., Westcott, J., Crosby, J., Matharu, A. L., Cox, R. J., Simpson, T. J., Staunton, J. & Leadlay, P. F. (1999) Nature 401, 502-505.
19. Aparicio, J. F., Molnar, I., Schwecke, T., Konig, A., Haydock, S. F., Khaw, L. E., Staunton, J. & Leadlay, P. F. (1996) Gene 169, 9-16.
20. Caffrey, P. (2003) Chem Bio Chem 4, 649-662.
21. Broadhurst, R. W., Nietlispach, D., Wheatcroft, M. P., Leadlay, P. F. & Weissman, K. J. (2003) Chem Biol In press.
22. Hong, H., Gates, P., Staunton, J., Stinear, T., Cole, S. T., Leadlay, P. F. & Spencer, J. B. (2003) Chem Comm In press.
23. Marsollier, L., Robert, R., Aubry, J., Saint Andre, J. P., Kouakou, H., Legras, P., Manceau, A. L., Mahaza, C. & Carbonnelle, B. (2002) Appl Environ Microbiol 68, 4623-4628.
24. Finlay, B. B. & Falkow, S. (1997) Microbiol Mol Biol Rev 61, 136-169.
25. McCluskie M. J. et Weeratna R. D. (2001) Current Drug Targets-Infectious Disorders 1, 263-271

1. An isolated or purified polynucleotide comprising the DNA sequence of SEQ ID NO:1-6.

2-55. (canceled)


SEQUENCE LISTING (36 sequences)

SEQ ID NO: 1 - DNA, 50973 bp, Mycobacterium ulcerans
gtgatcttcggagatgctcaccaaaactgcaggggaggtcgggtgttgggtgatgcagtcgcagtggtcggaatgtcttgccgggttcctggcgcatctgatccggacgctctgtgggcgctgctgcgagacgggatcagtgtggtcgatgagataccttctgcacgttggaatttagacggcctcgttgctcaccgactgaccgatgagcaacgatcagcgcttcggcatggcgcctttcttgatgacgtcgaagggtttgacgccgcgttcttcggaattaacccctccgaagctgggtcgatggatccgcagcaacgattgatgcttgaactgacctgggcagcactcgaagatgctcgaatcgtgccagaacatctttccggtagcagtagcggggtgtttaccggcgccatgagcgatgattacacgaccgcggtgacctaccgcgcagcgatgactgcacatacctttgcggggactcaccgcagcctcatagccaaccgtgtctcctacacactcggtctacgcggacctagtttggtcatcgataccgggcaatcgtcctcactggtggctgtgcacgtggcaatggaaagcttgcgcagagaagaaacttcacttgctatcgcgggtggtattcaccttaacctcagcctcgccgccgcactgagcgcagcacactttggagccctttcacctgacggacgctgctacaccttcgacgcacgtgccaacggatacgttcgtggcgaaggcggcggcgtcgtcgtcctcaaacgtctcaacgacgccctagccgacggcaaccatatttactgtgtgatccgcggcagctcagtcaacaacgacggcgccactcaagacttgacagcgcccggagtcgacggccagcgtcaagcgctccttcaagcttatgagcgagccgaaatcgacccctcagaagtccaatacgtcgagctacatggcaccggcacccgactcggcgatcccaccgaagcccactcgcttcactccgtcttcggcacatccacggtcccgcgcagcccgctgctagtcgggtcaatcaaaaccaatatcggtcacctcgaaggcgccgcaggaatcctcggcctaatcaagactgcccttgccgttcatcatcgccagcttccccccagcctcaactacacggttcctaacccaaaaatcccgctagagcagctagggctccgcgtccaaaccactctcagtgaatggccggacttagacaaaccgctaacggcgggcgtgtcatctttttccatgggtggcaccaacgcccacctcatcctccaacaaccccccacccccgacaccacacaaacccccaaccccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgaggtcgctgcggcgctgaacccgcatctcgatgttgcgttgcttgaggtgatgttcagccaacaagacactgccatggcgcaactgctggaccagaccttctatgcacaaccggcgttgttcgcgctgggaaccgctctacatcgattgttcacccacgccggtatccacccggactacctgctaggccactccatcggagaactcaccgcggcatacgccgccggtgtgctgtcactgcaagacgcagccaccttggtcacaagccgaggacgactgatgcaatcctgcacgcccggcgggacgatgctcgcactacaagccagcgaagcagaagtacaaccgctgcttgaaggcctagaccacgccgtgtccatcgccgcgatcaacggagcaacgtcgatcgtactgtcaggagatcacgacagcctcgaacaaatcggcgagcacttcattacccaagatcgacgtaccacccgactgcaggtcagtcacgctttccactctccacatatggaccccatcctcgaacaattccgccagatcgcggcccaactcaccttcagcgcacccaccctgcccatcttgtccaacctcaccgggcagatcgcccgccacgaccaactcgcctcacctgactattggacccaacagctacgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccgaacaccggatttgtcgagctagcgctgcatgtcgctgaccgtgccggatattcctcggtcaacgaactgatcgtgcacacccccctgctgctcgctggccacgacaccgcggatctacagatcaccgtcaccgacaccgatgacatgggccggcagtctcttaacatccactcgcacccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggggaccaggtgcggctgccctacgcctttaccggcatcagtttgcacgccacccacgccacccgattgcgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctcaacctttcgtccactcttatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagcctgggcccagtcctacggcgccaccaactccgatgacgctgaggggtatgcgatgaccggcggcgcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgatttctcccgccaacgcggcctggccccagatggacgctgcaaagccttcgccgctaccgccgatggcaccggctggggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccggcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaaccccacaacaggttctgatcccgcagtgggttctgattccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgaggtcgctgcggcgctgaacccgcatctcgatgttgcgttgcttgaggtgatgttcagccaacaagacactgccatggcgcaactgctggaccagaccttctatgcacaaccggcgttgttcgcgctgggaaccgctctacatcgattgttcacccacgccggtatccacccggactacctgctaggccactccatcggagaactcaccgcggcatacgccgccggtgtgctgtcactgcaagacgcagccaccttggtcacaagccgaggacgactgatgcaatcctgcacgcccggcgggacgatgctcgcactacaagccagcgaagcagaagtacaaccgctgcttgaaggcctagaccacgccgtgtccatcgccgcgatcaacggagcaacgtcgatcgtactgtcaggagatcacgacagcctcgaacaaatcggcgagcacttcattacccaagatcgacgtaccacccgactgcaggtcagtcacgctttccactctccacatatggaccccatcctcgaacaattccgccagatcgcggcccaactcaccttcagcgcacccaccctgcccatcttgtccaacctcaccgggcagatcgcccgccacgaccaactcgcctcacctgactattggacccaacagctacgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccgaacaccggatttgtcgagctagcgctgcatgtcgctgaccgtgccggatattcctcggtcaacgaactgatcgtgcacacccccctgctactcgctggccacgacaccgcggatctacagatcaccgtcaccgacaccgatgacatgggccggcagtctcttaacatccactcgcgcccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggtgaccaggtgcggctgccctacgcctttaccggcatcagtttgcacgccacccacgccacccgattgcgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctcaacctttcgtccactcttatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagccggggcccagtcctacggcgccaccaactccgatgacgctgaggggtatgcgatgaccggcggcgcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcgattttcaccgagttctcccgccaacgcggcctggccccagatggacgctgcaaagccttcgccgctaccgccgatggcaccggctggggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccggcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaaccccacaacaggttctgatcccgcagtgggttctgattccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgcatgcgacgcagcgttacagcctttcactggatggtcggtgctagctgtgttacacgacgaacccgaggccccgtcgttggagcgagtcgatgtggtccagcctgtgttgttctcggtgatggtgtcgttagccgcactctggcggtgggccggaatcacccccgatgcagtcatcggccactcccagggcgagatcgccgcggcacatgtggccggagccctgaccttgcccgaagcagctgcggtagtggctttgcgcagccgtgtcttgaccgacctggccggtgccggtgccatggcttcagtgctatcgcccgaggaaccactgacccagctgctggcacggtgggacggcaagatcactgtcgccgcagttaacggccccgctagcgctgtggtctccggcgataccacagcgatcaccgaattgctgattacctgcgaacacgaaaacatcgacgctcgcgctatcccggtggactacccctctcattccccctatatggaacacatccgccatcagttcctcgacgagctacccgagctgacaccgcggccatcaaccatcgcgatgtattccaccgtcgacggcgaacctcacgacaccgcctacgacaccaccacaatgaccgcggactactggtaccgcaacatccgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggattcgtcgagctagcgctgcatgtcgctgaccgtgccggatattcctcggtcaacgaactgatcgtgcacacccccctgctactcgctggccacgacaccgcggatctacagatcaccgtcaccgacaccgatgacatgggccggcagtctcttaacatccactcgcgcccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggtgaccaggtgcggctgccctacgcctttaccggcatcagtttgcacgccacccacgccacccgattacgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgcaccgctgtcctgacaccaccggacagcggcccctggcgccttgacaccaccggcaagggtgatctggccaacctcgccctgctaccgaccgcccacactgccctggcctctggacaaatccgtatcgatgtccgggccgctggtttgaattttcacgacgtggtcgtcgcgttggggctaatccccgacgacggattcggcggagaagccgccggggtgatcagcgagatcggtcccgacgtctacggattcgccgtgggtgatgccgtgaccggcatgaccgtctctggtgcgtttgcccccagcactgtcgctgatcaccgcatggtgatgacgatcccggcccggtggtccttcccccaagccgcatccataccggtggtattcctgaccgcctacatcgctttggccgagatctcgggcctaagccgagggcaacgagtgctgatccatgccggcactggcggtgtgggtatggctgcgattcaattggcacaccatttgggtgccgaagtattcgccaccgccagcgccgcgaaatggagcacccttgaggcactgggggtaccgcgcgaccatatcgcttcctcgcgtactctggacttttccaacgcattcctcgatgccaccaacggcgccggtgttgatgtcgtattgaactgcctcagtggtgaattcgtcgaagcatccctagccctgctgccccgcggtggccatttcgtcgaaatcggcaaaaccgacatccgtgataccgaggtcatcgccgcaacccatcccggcgtcatttaccgcgccctcgatctgctcagcgtctcccccgatcacatccagcgcacactggcccaactgtccccactgtttgccaccgacaccctaaaacccctaccgaccactaattacagcatctaccaagccatctcggccttacgtgacatgagtcaagcccgtcacacaggcaagatcgtgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgtggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtctgggtaactacgcggcagccaacaccgcgttagatgctctcgccgactaccgccaccgcctgggcttgcccgcgaccagcctggcctggggctactggcagacccgcaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctcaacctttcgtccactcttatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagccggggcccagtcctacggcgccaccaactccgatggcgctgaggggtatgcgatgaccggcggcgcgatcagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcgattttcaccgagttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaacaccacaacaggttctgatcccgcagtgggttctgattccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgcatgcgacgcagcgttacagcctttcactggatggtcggtgctagctgtgttacacgacgaacccgaggccccgtcgttggagcgggtcgatgtggtccagcctgtgttgttctcggtgatggtgtcgttagccgcactctggcggtgggccggaatcacccccgatgcagtcatcggccactcccagggcgagatcgccgcggcacatgtggccggagccctgaccttgcccgaagcagctgcggtagtggctttgcgcagccgtgtcttgaccgacctggccggtgccggtgccatggcttcagtgctatcgcccgaggaaccactgacccagctgctggcacggtgggacggcaagatcactgtcgccgcagttaacggccccgctagcgctgtggtctccggcgataccacagcgatcaccgaattgctgattacctgcgaacacgaaaacatcgacgctcgcgctatcccggtggactacccctctcattccccctatatggaacacatccgccatcagttcctcgacgagctacccgagctgacaccgcggccatcaaccatcgcgatgtattccaccgtcgacggcgaacctcacgacaccgcctacgacaccaccacaatgaccgcggactactggtaccgcaacatccgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggatttgtcgagctagcgctgcatgtcgctgaccgtgccggatattcctcggtcaacgaactgatcgtgcacacccccctgctgctcgctggccacgacaccgcggatctacagatcaccgtcaccgacaccgatgacatgggccggcagtctcttaacatccactcgcgcccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggggaccaggtgcggctgccctacgcctttaccggcatcagtttgcacgccacccacgccacccgattgcgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcgggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacacccaccgcgctaacccaacacctgcacacccgactcaccaccggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagcctgggcccagtcctacggcgccaccaactccgatgacgctgaggggtatgcgatgaccggcggcgcgatcagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgatttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccggcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaacaccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgcatgcgacgcagcgttacagcctttcactggatggtcggtgctagctgtgttacacgacgaacccgaggccccgtcgttggagcgggtcgatgtggtccagcctgtgttgttctcggtgatggtgtcgttagccgcactctggcggtgggccggaatcacccccgatgcagtcatcggccactcccagggcgagatcgccgcggcacatgtggccggagccctgaccttgcccgaagcagctgcggtagtggctttgcgcagccgtgtcttgaccgacctggccggtgccggtgccatggcttcagtgctatcgcccgaggaaccactgacccagctgctggcacggtgggacggcaagatcactgtcgccgcagttaacggccccgctagcgctgtggtctccggcgataccacagcgatcaccgaattgctgattacctgcgaacacgaaaacatcgacgctcgcgctatcccggtggactacccctctcattccccctatatggaacacatccgccatcagttcctcgacgagctacccgagctgacaccgcggccatcaaccatcgcgatgtattccaccgtcgacggcgaacctcacgacaccgcctacgacaccaccacaatgaccgcggactactggtaccgcaacatccgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggatttgtcgagctagcgctgcatgtcgctgaccgtgccggatattcctcggtcaacgaactgatcgtgcacacccccctgctactcgctggccacgacaccgcggatctacagatcaccgtcaccgacaccgatgacatgggccggcagtctcttaacatccactcgcacccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggtgaccaggtgcggctgccctacgcctttaccggcatcagtttgcacgccacccacgccacccgattgcgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgcaccgctgtcctgacaccaccggacagcggcccctggcgccttgacaccaccggcaagggtgatctggccaacctcgccctgctaccgaccgcccacactgccctggcctctggacaaatccgtatcgatgtccgggccgctggtttgaattttcacgacgtggtcgtcgcgttggggctaatccccgacgacggattcggcggagaagccgccggggtgatcagcgagatcggtcccgacgtctacggattcgccgtgggtgatgccgtgaccggcatgaccgtctctggtgcgtttgcccccagcactgtcgctgatcaccgcatggtgatgacgatcccggcccggtggtccttcccccaagccgcatccataccggtggtattcctgaccgcctacatcgctttggccgagatctcgggcctaagccgagggcaacgagtgctgatccatgccggcactggcggtgtgggtatggctgcgattcaattggcacaccatttgggtgccgaagtattcgccaccgccagcgccgcgaaatggagcacccttgaggcactgggggtaccgcgcgaccatatcgcttcctcgcgtactctggacttttccaacgcattcctcgatgccaccaacggcgccggtgttgatgtcgtattgaactgcctcagtggtgaattcgtcgaagcatccctagccctgctgccccgcggtggccatttcgtcgaaatcggcaaaaccgacatccgtgataccgaggtcatcgccgcaacccatcccggcgtcatttaccgcgccctcgatctgctcagcgtctcccccgatcacatccagcgcacactggcccaactgtccccactgtttgccaccgacaccctaaaacccctaccgaccactaattacagcatctaccaagccatctcggccttacgtgacatgagtcaagcccgtcacacaggcaagatcgtgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctcaacctttcgtccactcttatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagccggggcccagtcctacggcgccaccaactccgatgacgctgaggggtatgcgatgaccggcggcgcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgagttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccggcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccctaaccccacacaaacccccgaggactgcagccccgcacaatctccctgcgcaacaatcaccgatgcaggcacgggattatcgtttgtgccctgggtgatttcagcgaagtcggctgaggcgttgtctgcgcaggcgagccgattgttgacgcgccttgacgatgatccagttgtcgatgcaatcgacctggggtggtcattgatagccactcgatcgatgtttgagcatcgcgcagtagttgtgggtgcggatcgtcaccagttgcagcgcgggttggccgagttggcttctggtaacttgggcgccgatgtagtggtgggccgggcccgcgcagcgggcgagactgtaatggtgtttcccggtcagggatcacagcggttgggcatgggcgcgcagctttatgaacaattcccggtattcgcggcggcgtttgatgacgttgttgatgcgctggaccagtatctgcggttgccgctacgccaagttatgtggggtgacgatgaaggcctgctcaattcaacggagttcgcccagccgtcgttgtttgctgtcgaggtcgcactgtttgcgttgctgcgcttctggggtgtcgttccggattacgtgataggccattcggtaggagagctggccgctgcacaagtggctggcgttttgagcctgcaggacgcggctaaattagtttcagcgcggggccgactgatgcaggccctgcccgccggtggagcgatggtcgcggtagccgccagccagcatgaagtcgagcctttgctggttgaaggggtcgatatcgcggcgctcaatgcgccagggtcagttgtgatctctggtgatcaggcggcagtccgtttgatcgctaatcgattggcggataggggctacagggcgcacgaacttgcggtttcgcatgcctttcattcatcgttgatggagccgatgttggaggagttcgctcggctcgcttctgaaatcgttgtggagcaaccgcagattccactgatttcgaacgtgactggtcagctggccaacgccgactacgggtcggcaggttactgggtggaccacatccgccgtccagtccgtttcgccgatagtgtcgcttcgttggaagccatgggggctagctgcttcattgaagtcggtccagccagcgggttgggcgcagctatcgagcaatccttgaaatctgccgagccgaccgtgtcagtgtcggcactgtccaccgataaacctgaatccgtcgccgtattgcgcgctgcagcacgactttccacctccggcattcctgtggattggcagtcggtgttcgacggccgcagcacccagacagttaacctgcccacctacgccttccagcggcaacggttctggctcgacgccaaccgtatcggtcaaggcgatcccgccagtcaaccacaggcccagaacgttgaatcccgtttttgggaggcggtcgagcgggaagacgttgatggcttggctgattctataggtgtcaccgccagtgccatgcagaccgtgctacctgcattgtcttcatggcgtcgcgcggagcgcacacagtccgagcttgattcctggcgctatcaggtgacatggctgtcttccccagcaacgccgagttcgatcacgctgtccggcatttggttgctgatagttccaagcgaacttgcaaagactgacccagtaattggatgtgctgcagcgctcgaagcgcacggcgccttagtcacgattatcacaattttcgagccggacttcaatcgctcattgatgggcgcttccctaaaagatatcggttcacacatatctggtgtcatatcgttcttagggattcacgggtccgaattctccgatagcggcgcggtcaagacattaaatcttgtgcaagcaatgggcgatgtccacttagacgttcctttgtggtgcctaacgcagggcgcggtatcgatcagcgccgacgatttgatccgatgctcgtcagcagccctggtgtggggtctggggagagtcgtcgcattagagcacccgggatcgtggggtggcttagtagacctccccgagtcacccgacgatgcagcatgggagcgcttgtgcgccctcctcgcgcagccgacggatgaagatcagtttgcgatcaggccgtctggggttttcctacggagattgatccacgccccggcaaccacgacatccaaatcctcgaccgcgtgggctccgagggggaccgtgttaatcacaggcggcacaggcgcgttaggcgcacacgtcgcaaggtggttggcccacaaatatgaatcggtagatttgctcttaaccagccgtcgcgggatggcagccgatggagctacagagctagtggatgacctccgcacggctggcgccagtgtgacagtgcacgcctgcgacgtgacagaccgcacttcagtcgaggctgcaatagcaggtaaatcccttgatgcggtctttcatcttgcaggacgacaccagccaactctgctaacagaactcgaggacgaatcctttagtgacgaattggcgccgaaggttcacggtgcccaagtattgagtgacatcacgtctaacctcacactatcagcgtttgtcatgttctcgtcagtagccggaatctggggcggcaaaagtcaaggcgcatatgctgccgctaacgcattcttagattcgctcgccgagaaacggcgcacgttggggttaccagcaacatcggtcgcttggggactgtgggctggcggcggcatgggagaccggccatccgcttcgggactaaaccttattggcttgaaatcgatgtcagcagatttagctgtgcaggcgctaagcgacgccattgacagaccgcaagcaacattgactgttgcgagcgtcaactgggatcggttctaccccacattcgctttggcgcgaccgaggcccttcctacacgaaatcacagaggtaatggcttaccgcgagtcgatgcgctcaagctctgcatcgacggcgacgctcctgacgagcaaattagccggactaacggcgacagaacagcgtgcagtcacccggaagttggtccttgatcaagccgcatccgttctcgggtacgcctcaactgagagtctcgatactcatgagtcattcaaagacctcggatttgattcgctgaccgcccttgaactgcgcgaccacctccaaactgcgaccggcctcaacctttcgtccactcttatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgccggagcctgggcccagtcctacggcgccaccaactccgatgacgctgaggggtatgcgatgaccggcggctcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcgattttcaccgagttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaacaccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgcatgcgacgcagcgttacagcctttcactggatggtcggtgctagctgtgttacacgacgaacccgaggccccgtcgttggagcgggtcgatgtggtccagcctgtgttgttctcggtgatggtgtcgttagccgcactctggcggtgggccggaatcacccccgatgcagtcatcggccactcccagggcgagatcgccgcggcacatgtggccggagccctgaccttgcccgaagcagctgcggtagtggctttgcgcagccgtgtcttgaccgacctggccggtgccggtgccatggcttcagtgctatcgcccgaggaaccactgacccagctgctggcacggtgggacggcaagatcactgtcgccgcagttaacggccccgctagcgctgtggtctccggcgataccacagcgatcaccgaattgctgattacctgcgaacacgaaaacatcgacgctcgcgctatcccggtggactacccctctcattccccctatatggaacacatccgccatcagttcctcgacgagctacccgagctgacaccgcggccatcaaccatcgcgatgtattccaccgtcgacggcgaacctcacgacaccgcctacgacaccaccacaatgaccgcggactactggtaccgcaacatccgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggatttgtcgagctagcgctgcatgtcgctgaccgtgccggatattcctcggtcaacgaactgatcgtgcacacccccctgctactcgctggccacgacaccgcggatctacagatcaccgtcaccgacaccgatgacatgggccggcagtctcttaacatccactcgcacccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggggaccaggtgcggctgccctacgcctttaccggcatcagtttgcacgccacccacgccacccgattacgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgccggagcctgggcccagtcctacggcgccaccaactccgatgacgctgaggggtatgcgatgaccggcggctcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgagttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaacaccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgcatgcgacgcagcgttacagcctttcactggatggtcggtgctagctgtgttacacgacgaacccgaggccccgtcgttggagcgggtcgatgtggtccagcctgtgttgttctcggtgatggtgtcgttagccgcactctggcggtgggccggaatcacccccgatgcagtcatcggccactcccagggcgagatcgccgcggcacatgtggccggagccctgaccttgcccgaagcagctgcggtagtggctttgcgcagccgtgtcttgaccgacctggccggtgccggtgccatggcttcagtgctatcgcccgaggaaccactgacccagctgctggcacggtgggacggcaagatcactgtcgccgcagttaacggccccgctagcgctgtggtctccggcgataccacagcgatcaccgaattgctgattacctgcgaacacgaaaacatcgacgctcgcgctatcccggtggactacccctctcattccccctatatggaacacatccgccatcagttcctcgacgagctacccgagctgacaccgcggccatcaaccatcgcgatgtattccaccgtcgacggcgaacctcacgacaccgcctacgacaccaccacaatgaccgcggactactggtaccgcaacatccgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggatttgtcgagctagcgctgcatgtcgctgaccgtgccggatattcctcggtcaacgaactgatcgtgcacacccccctgctgctcgctggccacgacaccgcggatctacagatcaccgtcaccgacaccgatgacatgggccggcagtctcttaacatccactcgcgcccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggtgaccaggtgcggctgccctacgcctttaccggcatcagtttgcacgccacccacgccacccgattacgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgcaccgctgtcctgacaccaccggacagcggcccctggcgccttgacaccaccggcaagggtgatctggccaacctcgccctgctaccgaccgcccacactgccctggcctctggacaaatccgtatcgatgtccgggccgctggtttgaattttcacgacgtggtcgtcgcgttggggctaatccccgacgacggattcggcggagaagccgccggggtgatcagcgagatcggtcccgacgtctacggattcgccgtgggtgatgccgtgaccggcatgaccgtctctggtgcgtttgcccccagcactgtcgctgatcaccgcatggtgatgacgatcccggcccggtggtccttcccccaagccgcatccataccggtggtattcctgaccgcctacatcgctttggccgagatctcgggcctaagccgagggcaacgagtgctgatccatgccggcactggcggtgtgggtatggctgcgattcaattggcacaccatttgggtgccgaagtattcgccaccgccagcgccgcgaaatggagcacccttgaggcactgggggtaccgcgcgaccatatcgcttcctcgcgtactctggacttttccaacgcattcctcgatgccaccaacggcgccggtgttgatgtcgtattgaactgcctcagtggtgaattcgtcgaagcatccctagccctgctgccccgcggtggccatttcgtcgaaatcggcaaaaccgacatccgtgataccgaggtcatcgccgcaacccatcccggcgtcatttaccgcgccctcgatctgctcagcgtctcccccgatcacatccagcgcacactggcccaactgtccccactgtttgccaccgacaccctaaaacccctaccgaccactaattacagcatctaccaagccatctcggccttacgtgacatgagtcaagcccgtcacacaggcaagatcgtgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagacccgcaccggtgtcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctgaccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacacccaccgcgctaacccaacacctgcacacccgactcaccaccggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggcgggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagccggggcccagtcctacggcgccaccaactccgatgacgctgaggggtatgcgatgaccggcggcgcgatcagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgatttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccggcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaaccccacaacaggttctgatcccgcagtgggttctgattccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgaggtcgctgcggcgctgaacccgcatctcgatgttgcgttgcttgaggtgatgttcagccaacaagacactgccatggcgcaactgctggaccagaccttctatgcacaaccggcgttgttcgcgctgggaaccgctctacatcgattgttcacccacgccggtatccacccggactacctgctaggccactccatcggagaactcaccgcggcatacgccgccggtgtgctgtcactgcaagacgcagccaccttggtcacaagccgaggacgactgatgcaatcctgcacgcccggcgggacgatgctcgcactacaagccagcgaagcagaagtacaaccgctgcttgaaggcctagaccacgccgtgtccatcgccgcgatcaacggagcaacgtcgatcgtactgtcaggagatcacgacagcctcgaacaaatcggcgagcacttcattacccaagatcgacgtaccacccgactgcaggtcagtcacgctttccactctccacatatggaccccatcctcgaacaattccgccagatcgcggcccaactcaccttcagcgcacccaccctgcccatcttgtccaacctcaccgggcagatcgcccgccacgaccaactcgcctcacctgactattggacccaacagctacgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccgaacaccggatttgtcgagctagcgctgcatgtcgctgaccgtgccggatattcctcggtcaacgaactgatcgtgcacacccccctgctgctcgctggccacgacaccgcggatctacagatcaccgtcaccgacaccgatgacatgggccggcagtctcttaacatccactcgcgcccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggggaccaggtgcggctgccctacgcctttaccggcatcagtttgcacgccacccacgccacccgattgcgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacacccaccgcgctaacccaacacctgcacacccgactcacacaaattgagagcccaaattccgaagactcgatgctgaaccttaaaaatttggaccgaattgaatcatatatcttcagaaattcgggagaagatcgagctcacgtaatcgctaatcgtttacggtcaattctctcgaaatgggatggcacccgtagtccagaattacctgcggaactccatcttgaatcggcaacagacgatgagctgttttccctagcaaacatgtttcgcactccaaccagcgaaatttcacctactctagaaggcggccgtggtgtcaactga

SEQ ID NO: 2 - DNA, 7233 bp, Mycobacterium ulcerans
gtggtgtcaactgaagaaaacctacgcgtttacttaaaacaggtcatcacagacctccaccaaatgcaggcacgtctgcggaagatcgaaaagcagagatcagagcgggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagcctgggcccagtcctacggcgccaccaactccgatggcgctgaggggtatgcgatgaccggcggctcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgagttctcccgccaacgcggcctggccccagatggacgctgcaaagccttcgccgctaccgccgatggcaccggctggggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaacaccacaacaggttctgatcccgcagtgggttctgattccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgaggtcgctgcggcgctgaacccgcatctcgatgttgcgttgcttgaggtgatgttcagccaacaagacactgccatggcgcaactgctggaccagaccttctatgcacaaccggcgttgttcgcgctgggaaccgctctacatcgattgttcacccacgccggtatccacccggactacctgctaggccactccatcggagaactcaccgcggcatacgccgccggtgtgctgtcactgcaagacgcagccaccttggtcacaagccgaggacgactgatgcaatcctgcacgcccggcgggacgatgctcgcactacaagccagcgaagcagaagtacaaccgctgcttgaaggcctagaccacgccgtgtccatcgccgcgatcaacggagcaacgtcgatcgtactgtcaggagatcacgacagcctcgaacaaatcggcgagcacttcattacccaagatcgacgtaccacccgactgcaggtcagtcacgctttccactctccacatatggaccccatcctcgaacaattccgccagatcgcggcccaactcaccttcagcgcacccaccctgcccatcttgtccaacctcaccgggcagatcgcccgccacgaccaactcgcctcacctgactattggacccaacagctacgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggatttgtcgagctagcgctgcatgtcgctgaccgtgccggatattcctcggtcaacgaactgatcgtgcacacccccctgctactcgctggccacgacaccgcggatctacagatcaccgtcaccgacaccgatgacatgggccggcagtctcttaacatccactcgcacccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggtgaccaggtgcggctgccctacgcctttaccggcatcagtttgcacgccacccacgccacccgattgcgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgcaccgctgtcctgacaccaccggacagcggcccctggcgccttgacaccaccggcaagggtgatctggccaacctcgccctgctaccgaccgcccacactgccctggcctctggacaaatccgtatcgatgtccgggccgctggtttgaattttcacgacgtggtcgtcgcgttggggctaatccccgacgacggattcggcggagaagccgccggggtgatcagcgagatcggtcccgacgtctacggattcgccgtgggtgatgccgtgaccggcatgaccgtctctggtgcgtttgcccccagcactgtcgctgatcaccgcatggtgatgacgatcccggcccggtggtccttcccccaagccgcatccataccggtggtattcctgaccgcctacatcgctttggccgagatctcgggcctaagccgagggcaacgagtgctgatccatgccggcactggcggtgtgggtatggctgcgattcaattggcacaccatttgggtgccgaagtattcgccaccgccagcgccgcgaaatggagcacccttgaggcactgggggtaccgcgcgaccatatcgcttcctcgcgtactctggacttttccaacgcattcctcgatgccaccaacggcgccggtgttgatgtcgtattgaactgcctcagtggtgaattcgtcgaagcatccctagccctgctgccccgcggtggccatttcgtcgaaatcggcaaaaccgacatccgtgataccgaggtcatcgccgcaacccatcccggcgtcatttaccgcgccctcgatctgctcagcgtctcccccgatcacatccagcgcacactggcccaactgtccccactgtttgccaccgacaccctaaaacccctaccgaccactaattacagcatctaccaagccatctcggccttacgtgacatgagtcaagcccgtcacacaggcaagatcgtgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagacccgcaccggtgtcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctgaccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacaccccatgcgctaacccaacacctgcacacccgactcacccaaagccataccccggtcggaccaattgcgtccctgctaagccacgcgatcgatgagggcaaattccgtgccggcgctgacctattgatggccgcatccaatttgaaccaaagtttcagcaatatggctgaactcaaccagctcccggccgtgacggacatagctgacgcgtctcctgatgggctactcaccctgatctgcatctctacctcagagaatgagtacgctcgcctcgctgctgcgaacattcattcactgaccttcgctgaaattgcggcgcccggcttttacgacgcgcagctgccaaattcgatagagacgtcggcagaggcgctggcaactgccatcacaggcgcctacgcaaatacgtccattgttctggtagcgcactccattgtctgcgagctagctcaggcaacgatgacacgtctacaagacgctgacatcgatcttgtgggtctggttctgttggatccactcgaagggactaacagcactgaagattatgtggagacagtcttgactcgaatcgagcatatcaatgcaccgagggtcggagtagacggttaccttgccgccctgggccgctatctccaattccacgaagaccgccgaataccaataccggaaacgcggcacatgacactgcactcggacacgaaaattgaccgtgcccaaacaccaatgaacttattacaagatgaggcagcgttgaccgccctcaaaataggaaactggatgaacgacacagggagtatcgcagtaacactgagagatggacccgtattcttgggcagggcccgctctgtcaacatgaggtga

SEQ ID NO: 3 - DNA, 42393 bp, Mycobacterium ulcerans
gtgatcttcggagatgctcaccaaaactgcaggggaggtcgggtgttgggtgatgcagtcgcagtggtcggaatgtcttgccgggttcctggcgcatctgatccggacgctctgtgggcgctgctgcgagacgggatcagtgtggtcgatgagataccttctgcacgttggaatttagacggcctcgttgctcaccgactgaccgatgagcaacgatcagcgcttcggcatggcgcctttcttgatgacgtcgaagggtttgacgccgcgttcttcggaattaacccctccgaagctgggtcgatggatccgcagcaacgattgatgcttgaactgacctgggcagcactcgaagatgctcgaatcgtgccagaacatctttccggtagcagtagcggggtgtttaccggcgccatgagcgatgattacacgaccgcggtgacctaccgcgcagcgatgactgcacatacctttgcggggactcaccgcagcctcatagccaaccgtgtctcctacacactcggtctacgcggacctagtttggtcatcgataccgggcaatcgtcctcactggtggctgtgcacgtggcaatggaaagcttgcgcagagaagaaacttcacttgctatcgcgggtggtattcaccttaacctcagcctcgccgccgcactgagcgcagcacactttggagccctttcacctgacggacgctgctacaccttcgacgcacgtgccaacggatacgttcgtggcgaaggcggcggcgtcgtcgtcctcaaacgtctcaacgacgccctagccgacggcaaccatatttactgtgtgatccgcggcagctcagtcaacaacgacggcgccactcaagacttgacagcgcccggagtcgacggccagcgtcaagcgctccttcaagcttatgagcgagccgaaatcgacccctcagaagtccaatacgtcgagctacatggcaccggcacccgactcggcgatcccaccgaagcccactcgcttcactccgtcttcggcacatccacggtcccgcgcagcccgctgctagtcgggtcaatcaaaaccaatatcggtcacctcgaaggcgccgcaggaatcctcggcctaatcaagactgcccttgccgttcatcatcgccagcttccccccagcctcaactacacggttcctaacccaaaaatcccgctagagcagctagggctccgcgtccaaaccactctcagtgaatggccggacttagacaaaccgctaacggcgggcgtgtcatctttttccatgggtggcaccaacgcccacctcatcctccaacaaccccccacccccgacaccacacaaacccccaaccccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgaggtcgctgcggcgctgaacccgcatctcgatgttgcgttgcttgaggtgatgttcagccaacaagacactgccatggcgcaactgctggaccagaccttctatgcacaaccggcgttgttcgcgctgggaaccgctctacatcgattgttcacccacgccggtatccacccggactacctgctaggccactccatcggagaactcaccgcggcatacgccgccggtgtgctgtcactgcaagacgcagccaccttggtcacaagccgaggacgactgatgcaatcctgcacgcccggcgggacgatgctcgcactacaagccagcgaagcagaagtacaaccgctgcttgaaggcctagaccacgccgtgtccatcgccgcgatcaacggagcaacgtcgatcgtactgtcaggagatcacgacagcctcgaacaaatcggcgagcacttcattacccaagatcgacgtaccacccgactgcaggtcagtcacgctttccactctccacatatggaccccatcctcgaacaattccgccagatcgcggcccaactcaccttcagcgcacccaccctgcccatcttgtccaacctcaccgggcagatcgcccgccacgaccaactcgcctcacctgactattggacccaacagctacgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccgaacaccggatttgtcgagctagcgctgcatgtcgctgaccgtgccggatattcctcggtcaacgaactgatcgtgcacacccccctgctgctcgctggccacgacaccgcggatctacagatcaccgtcaccgacaccgatgacatgggccggcagtctcttaacatccactcgcacccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggggaccaggtgcggctgccctacgcctttaccggcatcagtttgcacgccacccacgccacccgattgcgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctcaacctttcgtccactcttatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagcctgggcccagtcctacggcgccaccaactccgatgacgctgaggggtatgcgatgaccggcggcgcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgagttctcccgccaacgcggcctggccccagatggacgctgcaaagccttcgccgctaccgccgatggcaccggctggggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccctaaccccacacaaacccccgaggactgcagccccgcacaatctccctgcgcaacaatcaccgatgcaggcacgggattatcgtttgtgccctgggtgatttcagcgaagtcggctgaggcgttgtctgcgcaggcgagccgattgttgacgcgccttgacgatgatccagttgtcgatgcaatcgacctggggtggtcattgatagccactcgatcgatgtttgagcatcgcgcagtagttgtgggtgcggatcgtcaccagttgcagcgcgggttggccgagttggcttctggtaacttgggcgccgatgtagtggtgggccgggcccgcgcagcgggcgagactgtaatggtgtttcccggtcagggatcacagcggttgggcatgggcgcgcagctttatgaacaattcccggtattcgcggcggcgtttgatgacgttgttgatgcgctggaccagtatctgcggttgccgctacgccaagttatgtggggtgacgatgaaggcctgctcaattcaacggagttcgcccagccgtcgttgtttgctgtcgaggtcgcactgtttgcgttgctgcgcttctggggtgtcgttccggattacgtgataggccattcggtaggagagctggccgctgcacaagtggctggcgttttgagcctgcaggacgcggctaaattagtttcagcgcggggccgactgatgcaggccctgcccgccggtggagcgatggtcgcggtagccgccagccagcatgaagtcgagcctttgctggttgaaggggtcgatatcgcggcgctcaatgcgccagggtcagttgtgatctctggtgatcaggcggcagtccgtttgatcgctaatcgattggcggataggggctacagggcgcacgaacttgcggtttcgcatgcctttcattcatcgttgatggagccgatgttggaggagttcgctcggctcgcttctgaaatcgttgtggagcaaccgcagattccactgatttcgaacgtgactggtcagctggccaacgccgactacgggtcggcaggttactgggtggaccacatccgccgtccagtccgtttcgccgatagtgtcgcttcgttggaagccatgggggctagctgcttcattgaagtcggtccagccagcgggttgggcgcagctatcgagcaatccttgaaatctgccgagccgaccgtgtcagtgtcggcactgtccaccgataaacctgaatccgtcgccgtattgcgcgctgcagcacgactttccacctccggcattcctgtggattggcagtcggtgttcgacggccgcagcacccagacagttaacctgcccacctacgccttccagcggcaacggttctggctcgacgccaaccgtatcggtcaaggcgatcccgccagtcaaccacaggcccagaacgttgaatcccgtttttgggaggcggtcgagcgggaagacgttgatggcttggctgattctataggtgtcaccgccagtgccatgcagaccgtgctacctgcattgtcttcatggcgtcgcgcggagcgcacacagtccgagcttgattcctggcgctatcaggtgacatggctgtcttccccagcaacgccgagttcgatcacgctgtccggcatttggttgctgatagttccaagcgaacttgcaaagactgacccagtaattggatgtgctgcagcgctcgaagcgcacggcgccttagtcacgattatcacaattttcgagccggacttcaatcgctcattgatgggcgcttccctaaaagatatcggttcacacatatctggtgtcatatcgttcttagggattcacgggtccgaattctccgatagcggcgcggtcaagacattaaatcttgtgcaagcaatgggcgatgtccacttagacgttcctttgtggtgcctaacgcagggcgcggtatcgatcagcgccgacgatttgatccgatgctcgtcagcagccctggtgtggggtctggggagagtcgtcgcattagagcacccgggatcgtggggtggcttagtagacctccccgagtcacccgacgatgcagcatgggagcgcttgtgcgccctcctcgcgcagccgacggatgaagatcagtttgcgatcaggccgtctggggttttcctacggagattgatccacgccccggcaaccacgacatccaaatcctcgaccgcgtgggctccgagggggaccgtgttaatcacaggcggcacaggcgcgttaggcgcacacgtcgcaaggtggttggcccacaaatatgaatcggtagatttgctcttaaccagccgtcgcgggatggcagccgatggagctacagagctagtggatgacctccgcacggctggcgccagtgtgacagtgcacgcctgcgacgtgacagaccgcacttcagtcgaggctgcaatagcaggtaaatcccttgatgcggtctttcatcttgcaggacgacaccagccaactctgctaacagaactcgaggacgaatcctttagtgacgaattggcgccgaaggttcacggtgcccaagtattgagtgacatcacgtctaacctcacactatcagcgtttgtcatgttctcgtcagtagccggaatctggggcggcaaaagtcaaggcgcatatgctgccgctaacgcattcttagattcgctcgccgagaaacggcgcacgttggggttaccagcaacatcggtcgcttggggactgtgggctggcggcggcatgggagaccggccatccgcttcgggactaaaccttattggcttgaaatcgatgtcagcagatttagctgtgcaggcgctaagcgacgccattgacagaccgcaagcaacattgactgttgcgagcgtcaactgggatcggttctaccccacattcgctttggcgcgaccgaggcccttcctacacgaaatcacagaggtaatggcttaccgcgagtcgatgcgctcaagctctgcatcgacggcgacgctcctgacgagcaaattagccggactaacggcgacagaacagcgtgcagtcacccggaagttggtccttgatcaagccgcatccgttctcgggtacgcctcaactgagagtctcgatactcatgagtcattcaaagacctcggatttgattcgctgaccgcccttgaactgcgcgaccacctccaaactgcgaccggcctcaacctttcgtccactcttatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagcctgggcccagtcctacggcgccaccaactccgatgacgctgaggggtatgcgatgaccggcggcgcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgagttctcccgccaacgcggcctggccccagatggacgctgcaaagccttcgccgctaccgccgatggcaccggctggggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccctaaccccacacaaacccccgaggactgcagccccgcacaatctccctgcgcaacaatcaccgatgcaggcacgggattatcgtttgtgccctgggtgatttcagcgaagtcggctgaggcgttgtctgcgcaggcgagccgattgttgacgcgccttgacgatgatccagttgtcgatgcaatcgacctggggtggtcattgatagccactcgatcgatgtttgagcatcgcgcagtagttgtgggtgcggatcgtcaccagttgcagcgcgggttggccgagttggcttctggtaacttgggcgccgatgtagtggtgggccgggcccgcgcagcgggcgagactgtaatggtgtttcccggtcagggatcacagcggttgggcatgggcgcgcagctttatgaacaattcccggtattcgcggcggcgtttgatgacgttgttgatgcgctggaccagtatctgcggttgccgctacgccaagttatgtggggtgacgatgaaggcctgctcaattcaacggagttcgcccagccgtcgttgtttgctgtcgaggtcgcactgtttgcgttgctgcgcttctggggtgtcgttccggattacgtgataggccattcggtaggagagctggccgctgcacaagtggctggcgttttgagcctgcaggacgcggctaaattagtttcagcgcggggccgactgatgcaggccctgcccgccggtggagcgatggtcgcggtagccgccagccagcatgaagtcgagcctttgctggttgaaggggtcgatatcgcggcgctcaatgcgccagggtcagttgtgatctctggtgatcaggcggcagtccgtttgatcgctaatcgattggcggataggggctacagggcgcacgaacttgcggtttcgcatgcctttcattcatcgttgatggagccgatgttggaggagttcgctcggctcgcttctgaaatcgttgtggagcaaccgcagattccactgatttcgaacgtgactggtcagctggccaacgccgactacgggtcggcaggttactgggtggaccacatccgccgtccagtccgtttcgccgatagtgtcgcttcgttggaagccatgggggctagctgcttcattgaagtcggtccagccagcgggttgggcgcagctatcgagcaatccttgaaatctgccgagccgaccgtgtcagtgtcggcactgtccaccgataaacctgaatccgtcgccgtattgcgcgctgcagcacgactttccacctccggcattcctgtggattggcagtcggtgttcgacggccgcagcacccagacagttaacctgcccacctacgccttccagcggcaacggttctggctcgacgccaaccgtatcggtcaaggcgatcccgccagtcaaccacaggcccagaacgttgaatcccgtttttgggaggcggtcgagcgggaagacgttgatggcttggctgattctataggtgtcaccgccagtgccatgcagaccgtgctacctgcattgtcttcatggcgtcgcgcggagcgcacacagtccgagcttgattcctggcgctatcaggtgacatggctgtcttccccagcaacgccgagttcgatcacgctgtccggcatttggttgctgatagttccaagcgaacttgcaaagactgacccagtaattggatgtgctgcagcgctcgaagcgcacggcgccttagtcacgattatcacaattttcgagccggacttcaatcgctcattgatgggcgcttccctaaaagatatcggttcacacatatctggtgtcatatcgttcttagggattcacgggtccgaattctccgatagcggcgcggtcaagacattaaatcttgtgcaagcaatgggcgatgtccacttagacgttcctttgtggtgcctaacgcagggcgcggtatcgatcagcgccgacgatttgatccgatgctcgtcagcagccctggtgtggggtctggggagagtcgtcgcattagagcacccgggatcgtggggtggcttagtagacctccccgagtcacccgacgatgcagcatgggagcgcttgtgcgccctcctcgcgcagccgacggatgaagatcagtttgcgatcaggccgtctggggttttcctacggagattgatccacgccccggcaaccacgacatccaaatcctcgaccgcgtgggctccgagggggaccgtgttaatcacaggcggcacaggcgcgttaggcgcacacgtcgcaaggtggttggcccacaaatatgaatcggtagatttgctcttaaccagccgtcgcgggatggcagccgatggagctacagagctagtggatgacctccgcacggctggcgccagtgtgacagtgcacgcctgcgacgtgacagaccgcacttcagtcgaggctgcaatagcaggtaaatcccttgatgcggtctttcatcttgcaggacgacaccagccaactctgctaacagaactcgaggacgaatcctttagtgacgaattggcgccgaaggttcacggtgcccaagtattgagtgacatcacgtctaacctcacactatcagcgtttgtcatgttctcgtcagtagccggaatctggggcggcaaaagtcaaggcgcatatgctgccgctaacgcattcttagattcgctcgccgagaaacggcgcacgttggggttaccagcaacatcggtcgcttggggactgtgggctggcggcggcatgggagaccggccatccgcttcgggactaaaccttattggcttgaaatcgatgtcagcagatttagctgtgcaggcgctaagcgacgccattgacagaccgcaagcaacattgactgttgcgagcgtcaactgggatcggttctaccccacattcgctttggcgcgaccgaggcccttcctacacgaaatcacagaggtaatggcttaccgcgagtcgatgcgctcgagctctgcatcgacggcgacgctcctgacgagcaaattagccggactaacggcgacagaacagcgtgcagtcacccggaagttggtccttgatcaagccgcatccgttctcgggtacgcctcaactgagagtctcgatactcatgagtcattcaaagacctcggatttgattcgctgaccgcccttgaactgcgcgaccacctccaaactgcgaccggcctcaacctttcgtccactcttatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgtcggagcctgggcccagtcctacggcgccaccaactccgatgacgctgaggggtatgcgatgaccggcggcgcgatcagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactgaccggcggcgtcaccgtgatgagcacacctgcgattttcaccgagttctcccgccaacgcggcctggccccagatggacgctgcaaagccttcgccgctaccgccgatggcaccggctggggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaacaccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgcatgcgacgcagcgttacagcctttcactggatggtcggtgctagctgtgttacacgacgaacccgaggccccgtcgttggagcgagtcgatgtggtccagcctgtgttgttctcggtgatggtgtcgttagccgcactctggcggtgggccggaatcacccccgatgcagtcatcggccactcccagggcgagatcgccgcggcacatgtggccggagccctgaccttgcccgaagcagctgcggtagtggctttgcgcagccgtgtcttgaccgacctggccggtgccggtgccatggcttcagtgctatcgcccgaggaaccactgacccagctgctggcacggtgggacggcaagatcactgtcgccgcagttaacggccccgctagcgctgtggtctccggcgataccacagcgatcaccgaattgctgattacctgcgaacacgaaaacatcgacgctcgcgctatcccggtggactacccctctcattccccctatatggaacacatccgccatcagttcctcgacgagctacccgagctgacaccgcggccatcaaccatcgcgatgtattccaccgtcgacggcgaacctcacgacaccgcctacgacaccaccacaatgaccgcggactactggtaccgcaacatccgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggatttgtcgagctagcgctgcatgtcgctgaccgtgccggatattcctcggtcaacgaactgatcgtgcacacccccctgctgctcgctggccacgacaccgcggatctacagatcaccgtcaccgacaccgatgacatgggccggcagtctcttaacatccactcgcgcccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggtgaccaggtgcggctgccctacgcctttaccggcatcagtttgcacgccacccacgccacccgattacgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgccggagcctgggcccagtcctacggcgccaccaactccgatgacgctgaggggtatgcgatgaccggcggctcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgagttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaaccccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgaggtcgctgcggcgctgaacccgcatctcgatgttgcgttgcttgaggtgatgttcagccaacaagacactgccatggcgcaactgctggaccagaccttctatgcacaaccggcgttgttcgcgctgggaaccgctctacatcgattgttcacccacgccggtatccacccggactacctgctaggccactccatcggagaactcaccgcggcatacgccgccggtgtgctgtcactgcaagacgcagccaccttggtcacaagccgaggacgactgatgcaatcctgcacgcccggcgggacgatgctcgcactacaagccagcgaagcagaagtacaaccgctgcttgaaggcctagaccacgccgtgtccatcgccgcgatcaacggagcaacgtcgatcgtactgtcaggagatcacgacagcctcgaacaaatcggcgagcacttcattacccaagatcgacgtaccacccgactgcaggtcagtcacgctttccactctccacatatggaccccatcctcgaacaattccgccagatcgcggcccaactcaccttcagcgcacccaccctgcccatcttgtccaacctcaccgggcagatcgcccgccacgaccaactcgcctcacctgactattggacccaacagctacgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggatttgtcgagctagcgctgcatgtcgctgaccgtgccggatattcctcggtcaacgaactgatcgtgcacacccccctgctgctcgctggccacgacaccgcggatctacagatcaccgtcaccgacaccgatgacatgggccggcagtctcttaacatccactcgcgcccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggtgaccaggtgcggctgccctacgcctttaccggcatcagtttgcacgccacccacgccacccgattgcgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgccggagcctgggcccagtcctacggcgccaccaactccgatgacgctgaggggtatgcgatgaccggcggcgcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgagttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaacaccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgcatgcgacgcagcgttacagcctttcactggatggtcggtgctagctgtgttacacgacgaacccgaggccccgtcgttggagcgggtcgatgtggtccagcctgtgttgttctcggtgatggtgtcgttagccgcactctggcggtgggccggaatcacccccgatgcagtcatcggccactcccagggcgagatcgccgcggcacatgtggccggagccctgaccttgcccgaagcagctgcggtagtggctttgcgcagccgtgtcttgaccgacctggccggtgccggtgccatggcttcagtgctatcgcccgaggaaccactgacccagctgctggcacggtgggacggcaagatcactgtcgccgcagttaacggccccgctagcgctgtggtctccggcgataccacagcgatcaccgaattgctgattacctgcgaacacgaaaacatcgacgctcgcgctatcccggtggactacccctctcattccccctatatggaacacatccgccatcagttcctcgacgagctacccgagctgacaccgcggccatcaaccatcgcgatgtattccaccgtcgacggcgaacctcacgacaccgcctacgacaccaccacaatgaccgcggactactggtaccgcaacatccgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggatttgtcgagctagcgctgcatgtcgctgaccgtgccggatattcctcggtcaacgaactgatcgtgcacacccccctgctactcgctggccacgacaccgcggatctacagatcaccgtcaccgacaccgatgacatgggccggcagtctcttaacatccactcgcacccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggtgaccaggtgcggctgccctacgcctttaccggcatcagtttgcacgccacccacgccacccgattgcgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcgggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgccggagcctgggcccagtcctacggcgccaccaactccgatgacgctgaggggtatgcgatgaccggcggcgcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgagttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaacaccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgcatgcgacgcagcgttacagcctttcactggatggtcggtgctagctgtgttacacgacgaacccgaggccccgtcgttggagcgagtcgatgtggtccagcctgtgttgttctcggtgatggtgtcgttagccgcactctggcggtgggccggaatcacccccgatgcagtcatcggccactcccagggcgagatcgccgcggcacatgtggccggagccctgaccttgcccgaagcagctgcggtagtggctttgcgcagccgtgtcttgaccgacctggccggtgccggtgccatggcttcagtgctatcgcccgaggaaccactgacccagctgctggcacggtgggacggcaagatcactgtcgccgcagttaacggccccgctagcgctgtggtctccggcgataccacagcgatcaccgaattgctgattacctgcgaacacgaaaacatcgacgctcgcgctatcccggtggactacccctctcattccccctatatggaacacatccgccatcagttcctcgacgagctacccgagctgacaccgcggccatcaaccatcgcgatgtattccaccgtcgacggcgaacctcacgacaccgcctacgacaccaccacaatgaccgcggactactggtaccgcaacatccgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggattcgtcgagctagcgctgcatgtcgctgaccgtgccggatattcctcggtcaacgaactgatcgtgcacacccccctgctactcgctggccacgacaccgcggatctacagatcaccgtcaccgacaccgatgacatgggccggcagtctcttaacatccactcgcgcccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggtgaccaggtgcggctgccctacgcctttaccggcatcagtttgcacgccacccacgccacccgattacgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacaccccatgcggtggccgagcatctgcttgaacagatccctggcatcggtgccctggtgccggctccggtggtgatcgcagctggtcgtaccgaggagccggtggcggtggtggggatggcgtgtcgtttccccggtggtgtcgcatcagcggatcagttgtgggacttggtgatcgctggccgtgatgtggtgggtaattttccggccgatcggggttgggatgtggagggactgtttgatcccgatccggacgcggtcggcaaaacctacacccgttacggcgcgttccttgacgatgcggcaggttttgatgccgggttctttgggatctctccacgggaggcacgcgcgatggacccccagcagcggctgctgctggaggtgtgctgggaagcgctagaaaccgcgggtattcccgcgcacaccttggccggcacctccaccggggtattcgccggagcctgggcccagtcctacggcgccaccaactccgatgacgctgaggggtatgcgatgaccggcggcgcgactagcgtcatgtccggccgtatcgcctacaccttgggcctagaaggtccagcgatcaccgttgacaccgcctgctcgtcatcgctggtggcaattcacctggcctgccaatccttacgcaacaacgaatcccagctagcactggccggcggcgtcaccgtgatgagcacacctgcggttttcaccgagttctcccgccaacgcggcctggccccagatggacgctgcaaagccttcgccgctaccgccgatggcaccggctttggtgaaggcgccgcggtcttggtccttgaacggctctccgaggcccgccgcaacaaccacccggtccttgcgatcgtcgctggatcggcgatcaaccaagacggcgcatccaacggactgaccgcaccccacggcccgtcacaacaacgcgtcatcaaccaagcactagccaacgccggcctcacccacgaccaggtcgacgccgtcgaagcccacggcaccggcaccacactgggtgaccccatcgaagccagcgccctacacgccacctacggccaccaccacacgcccgatcaaccgctttggctgggatccatcaaatccaacatcggccacacccaagccgccgccggcgccgccggtgtggtcaagatgatccaagccatcacccacgccaccttgcccgccaccttgcacgtcgaccaacccagcccccacatcgactggtccagcggcacagtccgactcctaaccgagcccatccaatggcccaacaccgaccacccccgcaccgcggcggtgtcctcattcggcatcagcggcaccaacgcccacctcatcctccaacaaccccccacccccgacaccacacaaacccccaacaccacaacaggttctgatcccgcagtgggttctgatcccgcagtgggtgtactggtgtggccgttgtcagcgcgttcagcgccggggttaagcgcacaagcggcccgtctgtaccagcatctcagcgcccaccccgatctggatccgatcgatgtagcccacagcctggctaccacacgcagccaccacccccaccgcgccaccatcaccaccagcattgagcaccacagcgaaaacaaccacgacacaaccgatgcgctggccgcactgcacgccctggccaacaacggcacacaccccctgctgagcagaggcctgctgaccccacagggccccggcaaaacagtgttcgtgttccccggacagggcagtcaataccccggcatgggcgcagatctctaccgccaattccccgtgttcgcccacgccctcgacgaggtcgctgcggcgctgaacccgcatctcgatgttgcgttgcttgaggtgatgttcagccaacaagacactgccatggcgcaactgctggaccagaccttctatgcacaaccggcgttgttcgcgctgggaaccgctctacatcgattgttcacccacgccggtatccacccggactacctgctaggccactccatcggagaactcaccgcggcatacgccgccggtgtgctgtcactgcaagacgcagccaccttggtcacaagccgaggacgactgatgcaatcctgcacgcccggcgggacgatgctcgcactacaagccagcgaagcagaagtacaaccgctgcttgaaggcctagaccacgccgtgtccatcgccgcgatcaacggagcaacgtcgatcgtactgtcaggagatcacgacagcctcgaacaaatcggcgagcacttcattacccaagatcgacgtaccacccgactgcaggtcagtcacgctttccactctccacatatggaccccatcctcgaacaattccgccagatcgcggcccaactcaccttcagcgcacccaccctgcccatcttgtccaacctcaccgggcagatcgcccgccacgaccaactcgcctcacctgactattggacccaacagctacgtaacactgtccggttccatgacactgtcgctgccctgctcggggcgggtgagcaggttttcctggaactttcacctcacccggtgttgacacaagcgatcaccgacaccgtcgaacaagccggcggcggcggcgcagcagtgccagctctacgcaaggatcgccctgatgctgtcgcgttcgctgcagcactcggccagctgcactgccatggcatcagcccatcctggaatgttctttactgccaggcccgccccctcacactgcccacctacgctttccagcatcagcgttactggctgctgcccaccgctggtgatttcagcggggccaatacccacgccatgcatccgctgctagacaccgccaccgaactggccgaaaaccgcggatgggtgttcaccggccggatcagcccacgcacccaaccatggctaaacgaacacgccgtcgaatcagccgtgctgttcccaggcaccggatttgtcgagctagcgctgcatgtcgctgaccgtgccggatattcctcggtcaacgaactgatcgtgcacacccccctgctactcgctggccacgacaccgcggatctacagatcaccgtcaccgacaccgatgacatgggccggcagtctcttaacatccactcgcgcccacatatcggccatgacaacaccaccaccggcgatgaacaacccgagtgggtcctgcatgccagcgcagtcctgaccgcacaaaccaccgaccacaaccacctccccctaacgcctgtgccgtggcctccacccggcacagccgcgatcgaggtggatgacttctacgacgacctggctgcacagggctacaactacggcccgacattccaaggtgtgcaacggatatggcgtgaccacgccacacccgatgtcatctacgccgaagttgaactacccgaagacaccgacatcgacggctacggcatccaccccgccctattcgacgccgctttacaccccctactcgccctgacccaaccccccaccaacgacaccgatgacaccaacaccgcagacaccggggaccaggtgcggctgccctacgcctttaccggcatcagtttgcacgccacccacgccacccgattacgggtacggctgacccgtaccggcgccgatgccatcaccgtgcacaccagtgacaccaccggagccccggtggcgatcatcgactcattgatcacccgccccctcaccaccgccacagggtctgctccggcaaccacagcagctggcctactacacctgagctggccaccacaccctgacaccacgaccgacaccgacaccgacaccgatgccctgcggtatcaggtgatcgccgaacccactcaacaactgccccgctacctgcacgacctacacaccagcaccgacctgcacaccagcaccaccgaagcagacgtggttgtgtggccggtaccggtgcccagcaacgaagagctccaggcacaccaagcatccgacaccgcggtgtcttctcggatacacaccctgacccgccaaacacttaccgtggtgcaggactggctcactcaccccgacaccaccggcacccgactggtcatcgtgacccgccacggcgtcagcaccagtgcccacgacccggtccccgacctagcccacgccgcagtgtggggcctgatccgcagcgcccaaaacgaacaccccggacgcttcacactgctcgacaccgacgacaacaccaacagcgacaccctcaccaccgccctaaccctgccaacccgcgaaaaccaactggccatacgccgcgacaccatccacatcccccgcctgacccgacacagcagtgacggtgcgctcactgcgccggtggtggtagatcctgagggcacggtgttgatcaccggggggaccgggacgctgggtgccttgttcgccgagcatctggtttctgcccatggtgtccggcatctgttgttgacctcgcggcgcggacctcaggcccacggtgccaccgatctgcagcagcggctcaccgatctaggtgctcatgtcaccatcacggcctgcgatatcagcgaccccgaagcactggccgccctggtcaattcagtgcccacacaacaccgtttaaccgcggtagtgcacaccgccgcggtattggccgacaccccggtcaccgagttgaccggcgatcaactcgaccaggtgctggcccccaaaatcgacgcggcatggcagctgcaccaactcacctacgaacacaacctgtctgcattcatcatgttctcgtccatggccggaatgataggcagtcccggtcagggtaactacgcggcagccaacaccgcgttagatgctctcgccgactaccgccaccgcctgggcttgcccgcgaccagcctggcctggggctactggcagactcacaccggtctcaccgcgcatctaaccgatgtagatctagcccgcatgacccgcctgggtttgatgcccatcgccaccagccacggactggccctgttcgatgccgccctcgccaccggacagcccgtttcgatacccgccccgatcaacacccacaccctggcccgacacgcccgcgacaacaccctggccccgatcctgtctgcgctgatcaccacaccacggcgccgggcggcctctgccgcaaccgatctcgctgcccgcctcaacggacttagcccccaacagcaacaacaaacactggccaccctcgtggccgcggccaccgccaccgtgctgggccaccacacccccgaaagcatcagcccagccaccgcgttcaaagacctcggaatcgattcgctgaccgcccttgaactgcgcaacaccctcacccacaacaccggcctggatctgccccccaccctcatcttcgatcaccccacaccccatgcgctaacccaacacctgcacacccgactcacccaaagccataccccggtcggaccaattgcgtccctgctaagccacgcgatcgatgagggcaaattccgtgccggcgctgacctattgatggccgcatccaatttgaaccaaagtttcagcaatatggctgaactcaaccagctcccggccgtgacggacatagctgacgcgtctcctgatgggctactcaccctgatctgcatctctacctcagagaatgagtacgctcgcctcgctgctgcgaacattcattcactgaccttcgctgaaattgcggcgcccggcttttacgacgcgcagctgccaaattcgatagagacgtcggcagaggcgctggcaactgccatcacaggcgcctacgcaaatacgtccattgttctggtagcgcactccattgtctgcgagctagctcaggcaacgatgacacgtctacaagacgctgacatcgatcttgtgggtctggttctgttggatccactcgaagggactaacagcactgaagattatgtggagacagtcttgactcgaatcgagcatatcaatgcaccgagggtcggagtagacggttaccttgccgccctgggccgctatctccaattccacgaagaccgccgaataccaataccggaaacgcggcacatgacactgcactcggacacgaaaattgaccgtgcccaaacaccaatgaacttattacaagatgaggcagcgttgaccgccctcaaaataggaaactggatgaacgacgtgggtgttgccctctctgtcaaccttgagtga

SEQ ID NO: 4 - DNA, 987 bp, Mycobacterium ulcerans
gtgatttggaatgacatctacataagtggaacggggcgtttcatcccgtcaatgcgaccaattaatgatatccaggtcgacggtgttccgaatgatcatactatcgtgcaatccgattacatttccttcaccgaagccgatgagccggctacagtcatggccacgcgcgctgcaaccgaagcgctgaccacttccgagctagtatccgctgatgttggcgtcttgatatatgccgcgatcatcggcgatgcgcatcattttgcccccgtatgtcacgtccaaagagtcctccgggcgcccgatgcgctggcattcgaactttccgcagcaagtaacggtggaacacaaggcatcgcagttgctgcaaatctcatgacagctgacgcgtccgtgaaagctgcactcgtttgtacagcttaccggcacccgatcgatattatcagccgttggtcgtcaggtatggtattcggcgatggagcagccgccgccgtgctttcaagagacggcggaatggtgcgattgatttccgggtatcacggctcactgccggagctagaggttcttgctagaaatcgatccaacgaacgacttggctttgtgctgccagacgtcgggttaggaaaatacctaactgctatagcgcggatgtaccaagcggtaattgcgcaagtactagaagaggcacaaactagtattgccgagatcgactatttcggcctgatcggtataggaattccaagtctcacagcgactatcttagaacccaacggtattccagttaataaaacatcctggggtttgctaagacaaatgggccacgttggagcttgcgatcccctgctgagccttaaccacctattcgagcagaatgtcctcaagcgcggcgacaaagtcctactcctaggtggcggggtggggtatcgattgacatgcattgtggctgaaatcgccatgaatcccggcgtgcccggacactccacttcgtag

SEQ ID NO: 5 - DNA, 1314 bp, Mycobacterium ulcerans
gtgaggcagagattgaactggattgcggcgcacgggttgctccgcggcaccgcgcggttagcggcccggctgggcgacgtgcagtcgcggctggtggcagatcccatggttatggcaaacccggcgccattttgcgatgaattgagggcaatcggccctgtggtgtcgagctacggcacccacctcgtcgttagtcatgccatcgcccatgaactgcttcggtccgaagacttcgaagtggtctcgctcggatcgaacttgccggcaccaatgcgctggctagagcgccgcactcgggacgatacgccccatctgctgctgccgccgtcgttgctggccgttgagccgccgaatcacacgcgctatcgcaaggcagtgtcctcggtgttcacgccgaaagcagtagccggattacgcgatcatgtcgaagagactgcgtcggcgctgttggatcagctcaccgaccaggctagtgccgtcgacatcatagcccgctactgctcccagctgccggtcgcggtcatttgtgacatcttaggcgtgcccagtcgagaccgaaaccgtgttctcaagttcggtcagctggcggggccctgcttggattttgggctcacatggcgtcagcaccagcaggtgcggcaagggctccaaggactccacttctggatcaccgagcacctcgaggaattgcggtctaaccccggtgacgatctgatgagtcaaatgatccacgcaagtgaaaatggctcctcggaaacacacctccacgcaaccgaagtgcggatgatcgggctggtgttgggcgccagtttcgcaacaacgatggacctgttaggcaacgggattcaggtgttgttggacgcgcccgaactgcgggacgcgttgagtcagcgcccgcaactttggcccaacgcggtagaagagatcctgcggttggagccaccggttcagctcgccggacgaatggctcgcaaggacaccgaggtggcgggtaccgcaatcaagcggggccagctggtggcgatctatctgggggcggtcaaccgtgatccgtccgtgttcgccgatccgcaccgctttgacatcacacgagccaacgccaaccggcatctcgcattctccggtggccgccacttctgcctcggtgccgcccttgcccgcgtcgaaggcgaagtcggattgagaatgctcttcgagcgcttccctgacgtgcgcgccgcaggccccggaaatagacgtgatactcgaactctgcggggttggtcgcagctgccggtccagttgggcgcagcacgatcgatggctatccgatga

SEQ ID NO: 6 - DNA, 906 bp, Mycobacterium ulcerans
atgattgtttggcccgaagtggtcagcacagtggtcgacgtcgatggcgtggcgatgtcggcactagtcgccgaacccgatcaggagcctaaggccgtgatcttagccctgcatggcggtgccaccaacgcgcggtatttcgactgccctggccaccgcgcgctttccctgctgcacaccggcgcggcggcgggattcaccgttgtggcccttgaccggcccggctacggcagctcggcgggtgatcccgacgcgatgaaccggccccaccagcgggccgcgctggcctatggggcgctggatcgcatcctggcgcagcggccacgcggggccggggtgttcataatgggccattcaaacggatgcgaactggcgatgtggatggccaccgagacgcgcggtgccgagctgctcggcatcgagttggctggtaccggctggcattatcagcccgaggcccgagaaatcctgacaacggccactggtgaacatcggtgggtgggcctctatgatttgctctggcatccgcagcggctatacccgcccgaggtcctcaacgcggccatcatttcttcgtccgccccggcctacgaggagcagatgatggccgactggacccgccgaaccttcctggagctagtccctgctgtgcgtgtgccggtacatttcagcatcgcccaacacgaaaaggtttggcagcgcgatagttcagcgctagatgaaatcgccgtcctgttctctggcgcgccgcggttcatcctgcatgaacaacccgaggccggacacaacatcagcctgggccacaccgccggcgactaccacacgacagtcctgtcgtttgtccagcaatgtctggccgaacggttggccaacgcgcaacaagatgtcgatctcgcggccgagtga

SEQ ID NO: 7 - protein, 16990 aa, Mycobacterium ulcerans
VIFGDAHQNCRGGRVLGDAVAVVGMSCRVPGASDPDALWALLRDGISVVDEIPSARWNLDGLVAHRLTDEQRSALRHGAFLDDVEGFDAAFFGINPSEAGSMDPQQRLMLELTWAALEDARIVPEHLSGSSSGVFTGAMSDDYTTAVTYRAAMTAHTFAGTHRSLIANRVSYTLGLRGPSLVIDTGQSSSLVAVHVAMESLRREETSLAIAGGIHLNLSLAAALSAAHFGALSPDGRCYTFDARANGYVRGEGGGVVVLKRLNDALADGNHIYCVIRGSSVNNDGATQDLTAPGVDGQRQALLQAYERAEIDPSEVQYVELHGTGTRLGDPTEAHSLHSVFGTSTVPRSPLLVGSIKTNIGHLEGAAGILGLIKTALAVHHRQLPPSLNYTVPNPKIPLEQLGLRVQTTLSEWPDLDKPLTAGVSSFSMGGTNAHLILQQPPTPDTTQTPNPTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDEVAAALNPHLDVALLEVMFSQQDTAMAQLLDQTFYAQPALFALGTALHRLFTHAGIHPDYLLGHSIGELTAAYAAGVLSLQDAATLVTSRGRLMQSCTPGGTMLALQASEAEVQPLLEGLDHAVSIAAINGATSIVLSGDHDSLEQIGEHFITQDRRTTRLQVSHAFHSPHMDPILEQFRQIAAQLTFSAPTLPILSNLTGQIARHDQLASPDYWTQQLRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPNTGFVELALHVADRAGYSSVNELIVHTPLLLAGHDTADLQITVTDTDDMGRQSLNIHSHPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLNLSSTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFVGAWAQSYGATNSDDAEGYAMTGGATSVMSGRIAYTLGLEGPAITVDTACSSSLVAIHLACQSLRNNESQLALAGGVTVMSTPAVFTDFSRQRGLAPDGRCKAFAATADGTGWGEGAAVLVLERLSEARRNNHPVLAIVAGSAINQDGASNGLTAPHGPSQQRVINQALANAGLTHDQVDAVEAHGTGTTLGDPIEAGALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNPTTGSDPAVGSDSAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDEVAAALNPHLDVALLEVMFSQQDTAMAQLLDQTFYAQPALFALGTALHRLFTHAGIHPDYLLGHSIGELTAAYAAGVLSLQDAATLVTSRGRLMQSCTPGGTMLALQASEAEVQPLLEGLDHAVSIAAINGATSIVLSGDHDSLEQIGEHFITQDRRTTRLQVSHAFHSPHMDPILEQFRQIAAQLTFSAPTLPILSNLTGQIARHDQLASPDYWTQQLRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPNTGFVELALHVADRAGYSSVNELIVHTPLLLAGHDTADLQITVTDTDDMGRQSLNIHSRPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLNLSSTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFVGAGAQSYGATNSDDAEGYAMTGGATSVMSGRIAYTLGLEGPAITVDTACSSSLVAIHLACQSLRNNESQLALAGGVTVMSTPAIFTEFSRQRGLAPDGRCKAFAATADGTGWGEGAAVLVLERLSEARRNNHPVLAIVAGSAINQDGASNGLTAPHGPSQQRVINQALANAGLTHDQVDAVEAHGTGTTLGDPIEAGALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNPTTGSDPAVGSDSAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDACDAALQPFTGWSVLAVLHDEPEAPSLERVDVVQPVLFSVMVSLAALWRWAGITPDAVIGHSQGEIAAAHVAGALTLPEAAAVVALRSRVLTDLAGAGAMASVLSPEEPLTQLLARWDGKITVAAVNGPASAVVSGDTTAITELLITCEHENIDARAIPVDYPSHSPYMEHIRHQFLDELPELTPRPSTIAMYSTVDGEPHDTAYDTTTMTADYWYRNIRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSRPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRTAVLTPPDSGPWRLDTTGKGDLANLALLPTAHTALASGQIRIDVRAAGLNFHDVVVALGLIPDDGFGGEAAGVISEIGPDVYGFAVGDAVTGMTVSGAFAPSTVADHRMVMTIPARWSFPQAASIPVVFLTAYIALAEISGLSRGQRVLIHAGTGGVGMAAIQLAHHLGAEVFATASAAKWSTLEALGVPRDHIASSRTLDFSNAFLDATNGAGVDVVLNCLSGEFVEASLALLPRGGHFVEIGKTDIRDTEVIAATHPGVIYRALDLLSVSPDHIQRTLAQLSPLFATDTLKPLPTTNYSIYQAISALRDMSQARHTGKIVLTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAVVLADTPVTELTGDQLDQVLAPKIDAAWQLHQLTYEHNLSAFIMFSSMAGMIGSPGLGNYAAANTALDALADYRHRLGLPATSLAWGYWQTRTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLNLSSTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFVGAGAQSYGATNSDGAEGYAMTGGAISVMSGRIAYTLGLEGPAITVDTACSSSLVAIHLACQSLRNNESQLALAGGVTVMSTPAIFTEFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNTTTGSDPAVGSDSAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDACDAALQPFTGWSVLAVLHDEPEAPSLERVDVVQPVLFSVMVSLAALWRWAGITPDAVIGHSQGEIAAAHVAGALTLPEAAAVVALRSRVLTDLAGAGAMASVLSPEEPLTQLLARWDGKITVAAVNGPASAVVSGDTTAITELLITCEHENIDARAIPVDYPSHSPYMEHIRHQFLDELPELTPRPSTIAMYSTVDGEPHDTAYDTTTMTADYWYRNIRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSRPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYRVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLDLPPTLIFDHPTPTALTQHLHTRLTTGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFVGAWAQSYGATNSDDAEGYAMTGGAISVMSGRIAYTLGLEGPAITVDTACSSSLVAIHLACQSLRNNESQLALAGGVTVMSTPAVFTDFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEAGALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNTTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDACDAALQPFTGWSVLAVLHDEPEAPSLERVDVVQPVLFSVMVSLAALWRWAGITPDAVIGHSQGEIAAAHVAGALTLPEAAAVVALRSRVLTDLAGAGAMASVLSPEEPLTQLLARWDGKITVAAVNGPASAVVSGDTTAITELLITCEHENIDARAIPVDYPSHSPYMEHIRHQFLDELPELTPRPSTIAMYSTVDGEPHDTAYDTTTMTADYWYRNIRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSHPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRTAVLTPPDSGPWRLDTTGKGDLANLALLPTAHTALASGQIRIDVRAAGLNFHDVVVALGLIPDDGFGGEAAGVISEIGPDVYGFAVGDAVTGMTVSGAFAPSTVADHRMVMTIPARWSFPQAASIPVVFLTAYIALAEISGLSRGQRVLIHAGTGGVGMAAIQLAHHLGAEVFATASAAKWSTLEALGVPRDHIASSRTLDFSNAFLDATNGAGVDVVLNCLSGEFVEASLALLPRGGHFVEIGKTDIRDTEVIAATHPGVIYRALDLLSVSPDHIQRTLAQLSPLFATDTLKPLPTTNYSIYQAISALRDMSQARHTGKIVLTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLNLSSTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFVGAGAQSYGATNSDDAEGYAMTGGATSVMSGRIAYTLGLEGPAITVDTACSSSLVAIHLACQSLRNNESQLALAGGVTVMSTPAVFTEFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEAGALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPNPTQTPEDCSPAQSPCATITDAGTGLSFVPWVISAKSAEALSAQASRLLTRLDDDPVVDAIDLGWSLIATRSMFEHRAVVVGADRHQLQRGLAELASGNLGADVVVGRARAAGETVMVFPGQGSQRLGMGAQLYEQFPVFAAAFDDVVDALDQYLRLPLRQVMWGDDEGLLNSTEFAQPSLFAVEVALFALLRFWGVVPDYVIGHSVGELAAAQVAGVLSLQDAAKLVSARGRLMQALPAGGAMVAVAASQHEVEPLLVEGVDIAALNAPGSVVISGDQAAVRLIANRLADRGYRAHELAVSHAFHSSLMEPMLEEFARLASEIVVEQPQIPLISNVTGQLANADYGSAGYWVDHIRRPVRFADSVASLEAMGASCFIEVGPASGLGAAIEQSLKSAEPTVSVSALSTDKPESVAVLRAAARLSTSGIPVDWQSVFDGRSTQTVNLPTYAFQRQRFWLDANRIGQGDPASQPQAQNVESRFWEAVEREDVDGLADSIGVTASAMQTVLPALSSWRRAERTQSELDSWRYQVTWLSSPATPSSITLSGIWLLIVPSELAKTDPVIGCAAALEAHGALVTIITIFEPDFNRSLMGASLKDIGSHISGVISFLGIHGSEFSDSGAVKTLNLVQAMGDVHLDVPLWCLTQGAVSISADDLIRCSSAALVWGLGRVVALEHPGSWGGLVDLPESPDDAAWERLCALLAQPTDEDQFAIRPSGVFLRRLIHAPATTTSKSSTAWAPRGTVLITGGTGALGAHVARWLAHKYESVDLLLTSRRGMAADGATELVDDLRTAGASVTVHACDVTDRTSVEAAIAGKSLDAVFHLAGRHQPTLLTELEDESFSDELAPKVHGAQVLSDITSNLTLSAFVMFSSVAGIWGGKSQGAYAAANAFLDSLAEKRRTLGLPATSVAWGLWAGGGMGDRPSASGLNLIGLKSMSADLAVQALSDAIDRPQATLTVASVNWDRFYPTFALARPRPFLHEITEVMAYRESMRSSSASTATLLTSKLAGLTATEQRAVTRKLVLDQAASVLGYASTESLDTHESFKDLGFDSLTALELRDHLQTATGLNLSSTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFAGAWAQSYGATNSDDAEGYAMTGGSTSVMSGRIAYTLGLEGPAITVDTACSSSLVAIHLACQSLRNNESQLALAGGVTVMSTPAIFTEFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNTTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDACDAALQPFTGWSVLAVLHDEPEAPSLERVDVVQPVLFSVMVSLAALWRWAGITPDAVIGHSQGEIAAAHVAGALTLPEAAAVVALRSRVLTDLAGAGAMASVLSPEEPLTQLLARWDGKITVAAVNGPASAVVSGDTTAITELLITCEHENIDARAIPVDYPSHSPYMEHIRHQFLDELPELTPRPSTIAMYSTVDGEPHDTAYDTTTMTADYWYRNIRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSHPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLDLPPTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFAGAWAQSYGATNSDDAEGYAMTGGSTSVMSGRIAYTLGLEGPAITVDTACSSSLVAIHLACQSLRNNESQLALAGGVTVMSTPAVFTEFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNTTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDACDAALQPFTGWSVLAVLHDEPEAPSLERVDVVQPVLFSVMVSLAALWRWAGITPDAVIGHSQGEIAAAHVAGALTLPEAAAVVALRSRVLTDLAGAGAMASVLSPEEPLTQLLARWDGKITVAAVNGPASAVVSGDTTAITELLITCEHENIDARAIPVDYPSHSPYMEHIRHQFLDELPELTPRPSTIAMYSTVDGEPHDTAYDTTTMTADYWYRNIRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSRPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRTAVLTPPDSGPWRLDTTGKGDLANLALLPTAHTALASGQIRIDVRAAGLNFHDVVVALGLIPDDGFGGEAAGVISEIGPDVYGFAVGDAVTGMTVSGAFAPSTVADHRMVMTIPARWSFPQAASIPVVFLTAYIALAEISGLSRGQRVLIHAGTGGVGMAAIQLAHHLGAEVFATASAAKWSTLEALGVPRDHIASSRTLDFSNAFLDATNGAGVDVVLNCLSGEFVEASLALLPRGGHFVEIGKTDIRDTEVIAATHPGVIYRALDLLSVSPDHIQRTLAQLSPLFATDTLKPLPTTNYSIYQAISALRDMSQARHTGKIVLTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTRTGVTAHLTDVDLARMTRLGLMPIATSHGLALFDAALATGQPVSIPAPINTHTLARHARDNTLTPILSALITTPRRRAASAATDLAARLNGLSPQQQQQTLATLVAAATATVLGHHTPESISPATAFKDLGIDSLTALELRNTLTHNTGLDLPPTLIFDHPTPTALTQHLHTRLTTGALVPAPVVIAAGRTEEPVAVVGMACRFPGGVASADQLWDLVIAGRDVVGNFPADRGWDVAGLFDPDPDAVGKTYTRYGAFLDDAAGFDAGFFGISPREARAMDPQQRLLLEVCWEALETAGIPAHTLAGTSTGVFVGAGAQSYGATNSDDAEGYAMTGGAISVMSGRIAYTLGLEGPAITVDTACSSSLVAIHLACQSLRNNESQLALAGGVTVMSTPAVFTDFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEAGALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNPTTGSDPAVGSDSAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDEVAAALNPHLDVALLEVMFSQQDTAMAQLLDQTFYAQPALFALGTALHRLFTHAGIHPDYLLGHSIGELTAAYAAGVLSLQDAATLVTSRGRLMQSCTPGGTMLALQASEAEVQPLLEGLDHAVSIAAINGATSIVLSGDHDSLEQIGEHFITQDRRTTRLQVSHAFHSPHMDPILEQFRQIAAQLTFSAPTLPILSNLTGQIARHDQLASPDYWTQQLRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPNTGFVELALHVADRAGYSSVNELIVHTPLLLAGHDTADLQITVTDTDDMGRQSLNIHSRPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLDLPPTLIFDHPTPTALTQHLHTRLTQIESPNSEDSMLNLKNLDRIESYIFRNSGEDRAHVIANRLRSILSKWDGTRSPELPAELHLESATDDELFSLANMFRTPTSEISPTLEGGRGVN

SEQ ID NO: 8 - protein, 2410 aa, Mycobacterium ulcerans
VVSTEENLRVYLKQVITDLHQMQARLRKIEKQRSERVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFVGAWAQSYGATNSDGAEGYAMTGGSTSVMSGRIAYTLGLEGPAITVDTACSSSLVAIHLACQSLRNNESQLALAGGVTVMSTPAVFTEFSRQRGLAPDGRCKAFAATADGTGWGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNTTTGSDPAVGSDSAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDEVAAALNPHLDVALLEVMFSQQDTAMAQLLDQTFYAQPALFALGTALHRLFTHAGIHPDYLLGHSIGELTAAYAAGVLSLQDAATLVTSRGRLMQSCTPGGTMLALQASEAEVQPLLEGLDHAVSIAAINGATSIVLSGDHDSLEQIGEHFITQDRRTTRLQVSHAFHSPHMDPILEQFRQIAAQLTFSAPTLPILSNLTGQIARHDQLASPDYWTQQLRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSHPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRTAVLTPPDSGPWRLDTTGKGDLANLALLPTAHTALASGQIRIDVRAAGLNFHDVVVALGLIPDDGFGGEAAGVISEIGPDVYGFAVGDAVTGMTVSGAFAPSTVADHRMVMTIPARWSFPQAASIPVVFLTAYIALAEISGLSRGQRVLIHAGTGGVGMAAIQLAHHLGAEVFATASAAKWSTLEALGVPRDHIASSRTLDFSNAFLDATNGAGVDVVLNCLSGEFVEASLALLPRGGHFVEIGKTDIRDTEVIAATHPGVIYRALDLLSVSPDHIQRTLAQLSPLFATDTLKPLPTTNYSIYQAISALRDMSQARHTGKIVLTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTRTGVTAHLTDVDLARMTRLGLMPIATSHGLALFDAALATGQPVSIPAPINTHTLARHARDNTLTPILSALITTPRRRAASAATDLAARLNGLSPQQQQQTLATLVAAATATVLGHHTPESISPATAFKDLGIDSLTALELRNTLTHNTGLDLPPTLIFDHPTPHALTQHLHTRLTQSHTPVGPIASLLSHAIDEGKFRAGADLLMAASNLNQSFSNMAELNQLPAVTDIADASPDGLLTLICISTSENEYARLAAANIHSLTFAEIAAPGFYDAQLPNSIETSAEALATAITGAYANTSIVLVAHSIVCELAQATMTRLQDADIDLVGLVLLDPLEGTNSTEDYVETVLTRIEHINAPRVGVDGYLAALGRYLQFHEDRRIPIPETRHMTLHSDTKIDRAQTPMNLLQDEAALTALKIGNWMNDTGSIAVTLRDGPVFLGRARSVNMR

SEQ ID NO: 9 - protein, 14130 aa, Mycobacterium ulcerans
VIFGDAHQNCRGGRVLGDAVAVVGMSCRVPGASDPDALWALLRDGISVVDEIPSARWNLDGLVAHRLTDEQRSALRHGAFLDDVEGFDAAFFGINPSEAGSMDPQQRLMLELTWAALEDARIVPEHLSGSSSGVFTGAMSDDYTTAVTYRAAMTAHTFAGTHRSLIANRVSYTLGLRGPSLVIDTGQSSSLVAVHVAMESLRREETSLAIAGGIHLNLSLAAALSAAHFGALSPDGRCYTFDARANGYVRGEGGGVVVLKRLNDALADGNHIYCVIRGSSVNNDGATQDLTAPGVDGQRQALLQAYERAEIDPSEVQYVELHGTGTRLGDPTEAHSLHSVFGTSTVPRSPLLVGSIKTNIGHLEGAAGILGLIKTALAVHHRQLPPSLNYTVPNPKIPLEQLGLRVQTTLSEWPDLDKPLTAGVSSFSMGGTNAHLILQQPPTPDTTQTPNPTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDEVAAALNPHLDVALLEVMFSQQDTAMAQLLDQTFYAQPALFALGTALHRLFTHAGIHPDYLLGHSIGELTAAYAAGVLSLQDAATLVTSRGRLMQSCTPGGTMLALQASEAEVQPLLEGLDHAVSIAAINGATSIVLSGDHDSLEQIGEHFITQDRRTTRLQVSHAFHSPHMDPILEQFRQIAAQLTFSAPTLPILSNLTGQIARHDQLASPDYWTQQLRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPNTGFVELALHVADRAGYSSVNELIVHTPLLLAGHDTADLQITVTDTDDMGRQSLNIHSHPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLNLSSTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFVGAWAQSYGATNSDDAEGYAMTGGATSVMSGRIAYTLGLEGPAITVDTACSSSLVAIHLACQSLRNNESQLALAGGVTVMSTPAVFTEFSRQRGLAPDGRCKAFAATADGTGWGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPNPTQTPEDCSPAQSPCATITDAGTGLSFVPWVISAKSAEALSAQASRLLTRLDDDPVVDAIDLGWSLIATRSMFEHRAVVVGADRHQLQRGLAELASGNLGADVVVGRARAAGETVMVFPGQGSQRLGMGAQLYEQFPVFAAAFDDVVDALDQYLRLPLRQVMWGDDEGLLNSTEFAQPSLFAVEVALFALLRFWGVVPDYVIGHSVGELAAAQVAGVLSLQDAAKLVSARGRLMQALPAGGAMVAVAASQHEVEPLLVEGVDIAALNAPGSVVISGDQAAVRLIANRLADRGYRAHELAVSHAFHSSLMEPMLEEFARLASEIVVEQPQIPLISNVTGQLANADYGSAGYWVDHIRRPVRFADSVASLEAMGASCFIEVGPASGLGAAIEQSLKSAEPTVSVSALSTDKPESVAVLRAAARLSTSGIPVDWQSVFDGRSTQTVNLPTYAFQRQRFWLDANRIGQGDPASQPQAQNVESRFWEAVEREDVDGLADSIGVTASAMQTVLPALSSWRRAERTQSELDSWRYQVTWLSSPATPSSITLSGIWLLIVPSELAKTDPVIGCAAALEAHGALVTIITIFEPDFNRSLMGASLKDIGSHISGVISFLGIHGSEFSDSGAVKTLNLVQAMGDVHLDVPLWCLTQGAVSISADDLIRCSSAALVWGLGRVVALEHPGSWGGLVDLPESPDDAAWERLCALLAQPTDEDQFAIRPSGVFLRRLIHAPATTTSKSSTAWAPRGTVLITGGTGALGAHVARWLAHKYESVDLLLTSRRGMAADGATELVDDLRTAGASVTVHACDVTDRTSVEAAIAGKSLDAVFHLAGRHQPTLLTELEDESFSDELAPKVHGAQVLSDITSNLTLSAFVMFSSVAGIWGGKSQGAYAAANAFLDSLAEKRRTLGLPATSVAWGLWAGGGMGDRPSASGLNLIGLKSMSADLAVQALSDAIDRPQATLTVASVNWDRFYPTFALARPRPFLHEITEVMAYRESMRSSSASTATLLTSKLAGLTATEQRAVTRKLVLDQAASVLGYASTESLDTHESFKDLGFDSLTALELRDHLQTATGLNLSSTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFVGAWAQSYGATNSDDAEGYAMTGGATSVMSGRIAYTLGLEGPAITVDTACSSSLVAIHLACQSLRNNESQLALAGGVTVMSTPAVFTEFSRQRGLAPDGRCKAFAATADGTGWGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPNPTQTPEDCSPAQSPCATITDAGTGLSFVPWVISAKSAEALSAQASRLLTRLDDDPVVDAIDLGWSLIATRSMFEHRAVVVGADRHQLQRGLAELASGNLGADVVVGRARAAGETVMVFPGQGSQRLGMGAQLYEQFPVFAAAFDDVVDALDQYLRLPLRQVMWGDDEGLLNSTEFAQPSLFAVEVALFALLRFWGVVPDYVIGHSVGELAAAQVAGVLSLQDAAKLVSARGRLMQALPAGGAMVAVAASQHEVEPLLVEGVDIAALNAPGSVVISGDQAAVRLIANRLADRGYRAHELAVSHAFHSSLMEPMLEEFARLASEIVVEQPQIPLISNVTGQLANADYGSAGYWVDHIRRPVRFADSVASLEAMGASCFIEVGPASGLGAAIEQSLKSAEPTVSVSALSTDKPESVAVLRAAARLSTSGIPVDWQSVFDGRSTQTVNLPTYAFQRQRFWLDANRIGQGDPASQPQAQNVESRFWEAVEREDVDGLADSIGVTASAMQTVLPALSSWRRAERTQSELDSWRYQVTWLSSPATPSSITLSGIWLLIVPSELAKTDPVIGCAAALEAHGALVTIITIFEPDFNRSLMGASLKDIGSHISGVISFLGIHGSEFSDSGAVKTLNLVQAMGDVHLDVPLWCLTQGAVSISADDLIRCSSAALVWGLGRVVALEHPGSWGGLVDLPESPDDAAWERLCALLAQPTDEDQFAIRPSGVFLRRLIHAPATTTSKSSTAWAPRGTVLITGGTGALGAHVARWLAHKYESVDLLLTSRRGMAADGATELVDDLRTAGASVTVHACDVTDRTSVEAAIAGKSLDAVFHLAGRHQPTLLTELEDESFSDELAPKVHGAQVLSDITSNLTLSAFVMFSSVAGIWGGKSQGAYAAANAFLDSLAEKRRTLGLPATSVAWGLWAGGGMGDRPSASGLNLIGLKSMSADLAVQALSDAIDRPQATLTVASVNWDRFYPTFALARPRPFLHEITEVMAYRESMRSSSASTATLLTSKLAGLTATEQRAVTRKLVLDQAASVLGYASTESLDTHESFKDLGFDSLTALELRDHLQTATGLNLSSTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFVGAWAQSYGATNSDDAEGYAMTGGAISVMSGRIAYTLGLEGPAITVDTACSSSLVAIHLACQSLRNNESQLALTGGVTVMSTPAIFTEFSRQRGLAPDGRCKAFAATADGTGWGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNTTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDACDAALQPFTGWSVLAVLHDEPEAPSLERVDVVQPVLFSVMVSLAALWRWAGITPDAVIGHSQGEIAAAHVAGALTLPEAAAVVALRSRVLTDLAGAGAMASVLSPEEPLTQLLARWDGKITVAAVNGPASAVVSGDTTAITELLITCEHENIDARAIPVDYPSHSPYMEHIRHQFLDELPELTPRPSTIAMYSTVDGEPHDTAYDTTTMTADYWYRNIRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSRPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLDLPPTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFAGAWAQSYGATNSDDAEGYAMTGGSTSVMSGRIAYTLGLEGPAITVDTACSSSLVAIHLACQSLRNNESQLALAGGVTVMSTPAVFTEFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNPTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDEVAAALNPHLDVALLEVMFSQQDTAMAQLLDQTFYAQPALFALGTALHRLFTHAGIHPDYLLGHSIGELTAAYAAGVLSLQDAATLVTSRGRLMQSCTPGGTMLALQASEAEVQPLLEGLDHAVSIAAINGATSIVLSGDHDSLEQIGEHFITQDRRTTRLQVSHAFHSPHMDPILEQFRQIAAQLTFSAPTLPILSNLTGQIARHDQLASPDYWTQQLRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSRPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLDLPPTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFAGAWAQSYGATNSDDAEGYAMTGGATSVMSGRIAYTLGLEGPAITVDTACSSSLVAIHLACQSLRNNESQLALAGGVTVMSTPAVFTEFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNTTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDACDAALQPFTGWSVLAVLHDEPEAPSLERVDVVQPVLFSVMVSLAALWRWAGITPDAVIGHSQGEIAAAHVAGALTLPEAAAVVALRSRVLTDLAGAGAMASVLSPEEPLTQLLARWDGKITVAAVNGPASAVVSGDTTAITELLITCEHENIDARAIPVDYPSHSPYMEHIRHQFLDELPELTPRPSTIAMYSTVDGEPHDTAYDTTTMTADYWYRNIRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSHPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYRVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLDLPPTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFAGAWAQSYGATNSDDAEGYAMTGGATSVMSGRIAYTLGLEGPAITVDTACSSSLVAIHLACQSLRNNESQLALAGGVTVMSTPAVFTEFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNTTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDACDAALQPFTGWSVLAVLHDEPEAPSLERVDVVQPVLFSVMVSLAALWRWAGITPDAVIGHSQGEIAAAHVAGALTLPEAAAVVALRSRVLTDLAGAGAMASVLSPEEPLTQLLARWDGKITVAAVNGPASAVVSGDTTAITELLITCEHENIDARAIPVDYPSHSPYMEHIRHQFLDELPELTPRPSTIAMYSTVDGEPHDTAYDTTTMTADYWYRNIRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSRPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLDLPPTLIFDHPTPHAVAEHLLEQIPGIGALVPAPVVIAAGRTEEPVAVVGMACRFPGGVASADQLWDLVIAGRDVVGNFPADRGWDVEGLFDPDPDAVGKTYTRYGAFLDDAAGFDAGFFGISPREARAMDPQQRLLLEVCWEALETAGIPAHTLAGTSTGVFAGAWAQSYGATNSDDAEGYAMTGGATSVMSGRIAYTLGLEGPAITVDTACSSSLVAIHLACQSLRNNESQLALAGGVTVMSTPAVFTEFSRQRGLAPDGRCKAFAATADGTGFGEGAAVLVLERLSEARRNNHPVLAIVAGSAINQDGASNGLTAPHGPSQQRVINQALANAGLTHDQVDAVEAHGTGTTLGDPIEASALHATYGHHHTPDQPLWLGSIKSNIGHTQAAAGAAGVVKMIQAITHATLPATLHVDQPSPHIDWSSGTVRLLTEPIQWPNTDHPRTAAVSSFGISGTNAHLILQQPPTPDTTQTPNTTTGSDPAVGSDPAVGVLVWPLSARSAPGLSAQAARLYQHLSAHPDLDPIDVAHSLATTRSHHPHRATITTSIEHHSENNHDTTDALAALHALANNGTHPLLSRGLLTPQGPGKTVFVFPGQGSQYPGMGADLYRQFPVFAHALDEVAAALNPHLDVALLEVMFSQQDTAMAQLLDQTFYAQPALFALGTALHRLFTHAGIHPDYLLGHSIGELTAAYAAGVLSLQDAATLVTSRGRLMQSCTPGGTMLALQASEAEVQPLLEGLDHAVSIAAINGATSIVLSGDHDSLEQIGEHFITQDRRTTRLQVSHAFHSPHMDPILEQFRQIAAQLTFSAPTLPILSNLTGQIARHDQLASPDYWTQQLRNTVRFHDTVAALLGAGEQVFLELSPHPVLTQAITDTVEQAGGGGAAVPALRKDRPDAVAFAAALGQLHCHGISPSWNVLYCQARPLTLPTYAFQHQRYWLLPTAGDFSGANTHAMHPLLDTATELAENRGWVFTGRISPRTQPWLNEHAVESAVLFPGTGFVELALHVADRAGYSSVNELIVHTPLLLAGHDTADLQITVTDTDDMGRQSLNIHSRPHIGHDNTTTGDEQPEWVLHASAVLTAQTTDHNHLPLTPVPWPPPGTAAIEVDDFYDDLAAQGYNYGPTFQGVQRIWRDHATPDVIYAEVELPEDTDIDGYGIHPALFDAALHPLLALTQPPTNDTDDTNTADTGDQVRLPYAFTGISLHATHATRLRVRLTRTGADAITVHTSDTTGAPVAIIDSLITRPLTTATGSAPATTAAGLLHLSWPPHPDTTTDTDTDTDALRYQVIAEPTQQLPRYLHDLHTSTDLHTSTTEADVVVWPVPVPSNEELQAHQASDTAVSSRIHTLTRQTLTVVQDWLTHPDTTGTRLVIVTRHGVSTSAHDPVPDLAHAAVWGLIRSAQNEHPGRFTLLDTDDNTNSDTLTTALTLPTRENQLAIRRDTIHIPRLTRHSSDGALTAPVVVDPEGTVLITGGTGTLGALFAEHLVSAHGVRHLLLTSRRGPQAHGATDLQQRLTDLGAHVTITACDISDPEALAALVNSVPTQHRLTAVVHTAAVLADTPVTELTGDQLDQVLAPKIDAAWQLHQLTYEHNLSAFIMFSSMAGMIGSPGQGNYAAANTALDALADYRHRLGLPATSLAWGYWQTHTGLTAHLTDVDLARMTRLGLMPIATSHGLALFDAALATGQPVSIPAPINTHTLARHARDNTLAPILSALITTPRRRAASAATDLAARLNGLSPQQQQQTLATLVAAATATVLGHHTPESISPATAFKDLGIDSLTALELRNTLTHNTGLDLPPTLIFDHPTPHALTQHLHTRLTQSHTPVGPIASLLSHAIDEGKFRAGADLLMAASNLNQSFSNMAELNQLPAVTDIADASPDGLLTLICISTSENEYARLAAANIHSLTFAEIAAPGFYDAQLPNSIETSAEALATAITGAYANTSIVLVAHSIVCELAQATMTRLQDADIDLVGLVLLDPLEGTNSTEDYVETVLTRIEHINAPRVGVDGYLAALGRYLQFHEDRRIPIPETRHMTLHSDTKIDRAQTPMNLLQDEAALTALKIGNWMNDVGVALSVNLE

SEQ ID NO: 10 - protein, 328 aa, Mycobacterium ulcerans
VIWNDIYISGTGRFIPSMRPINDIQVDGVPNDHTIVQSDYISFTEADEPATVMATRAATEALTTSELVSADVGVLIYAAIIGDAHHFAPVCHVQRVLRAPDALAFELSAASNGGTQGIAVAANLMTADASVKAALVCTAYRHPIDIISRWSSGMVFGDGAAAAVLSRDGGMVRLISGYHGSLPELEVLARNRSNERLGFVLPDVGLGKYLTAIARMYQAVIAQVLEEAQTSIAEIDYFGLIGIGIPSLTATILEPNGIPVNKTSWGLLRQMGHVGACDPLLSLNHLFEQNVLKRGDKVLLLGGGVGYRLTCIVAEIAMNPGVPGHSTS

SEQ ID NO: 11 - protein, 437 aa, Mycobacterium ulcerans
VRQRLNWIAAHGLLRGTARLAARLGDVQSRLVADPMVMANPAPFCDELRAIGPVVSSYGTHLVVSHAIAHELLRSEDFEVVSLGSNLPAPMRWLERRTRDDTPHLLLPPSLLAVEPPNHTRYRKAVSSVFTPKAVAGLRDHVEETASALLDQLTDQASAVDIIARYCSQLPVAVICDILGVPSRDRNRVLKFGQLAGPCLDFGLTWRQHQQVRQGLQGLHFWITEHLEELRSNPGDDLMSQMIHASENGSSETHLHATEVRMIGLVLGASFATTMDLLGNGIQVLLDAPELRDALSQRPQLWPNAVEEILRLEPPVQLAGRMARKDTEVAGTAIKRGQLVAIYLGAVNRDPSVFADPHRFDITRANANRHLAFSGGRHFCLGAALARVEGEVGLRMLFERFPDVRAAGPGNRRDTRTLRGWSQLPVQLGAARSMAIR

SEQ ID NO: 12 - protein, 301 aa, Mycobacterium ulcerans
MIVWPEVVSTVVDVDGVAMSALVAEPDQEPKAVILALHGGATNARYFDCPGHRALSLLHTGAAAGFTVVALDRPGYGSSAGDPDAMNRPHQRAALAYGALDRILAQRPRGAGVFIMGHSNGCELAMWMATETRGAELLGIELAGTGWHYQPEAREILTTATGEHRWVGLYDLLWHPQRLYPPEVLNAAIISSSAPAYEEQMMADWTRRTFLELVPAVRVPVHFSIAQHEKVWQRDSSALDEIAVLFSGAPRFILHEQPEAGHNISLGHTAGDYHTTVLSFVQQCLAERLANAQQDVDLAAE

SEQ ID NO: 13 - DNA, 20 bp, Artificial Sequence
  Description of Artificial Sequence Synthetic polynucleotide sequence
ctacgagctggtcagcaatg

SEQ ID NO: 14 - DNA, 20 bp, Artificial Sequence
  Description of Artificial Sequence Synthetic polynucleotide sequence
atcgacgctcgctacttctg

SEQ ID NO: 15 - DNA, 28 bp, Artificial Sequence
  Description of Artificial Sequence Synthetic polynucleotide sequence
ggactagtcccgggttcatgtgcagctc

SEQ ID NO: 16 - DNA, 30 bp, Artificial Sequence
  Description of Artificial Sequence Synthetic polynucleotide sequence
ggactagtcccgggcattgagcgtcagcat

SEQ ID NO: 17 - DNA, 18 bp, Artificial Sequence
  Description of Artificial Sequence Synthetic polynucleotide sequence
ggtgctgctggggcggtg

SEQ ID NO: 18 - DNA, 41 bp, Artificial Sequence
  Description of Artificial Sequence Synthetic polynucleotide sequence
cagggctccggcgttgttgattagcaggcttgtgagctggg

SEQ ID NO: 19 - DNA, 20 bp, Artificial Sequence
  Description of Artificial Sequence Synthetic polynucleotide sequence
ctacgagctggtcagcaatg

SEQ ID NO: 20 - DNA, 20 bp, Artificial Sequence
  Description of Artificial Sequence Synthetic polynucleotide sequence
atcgacgctcgctacttctg

SEQ ID NO: 21 - DNA, 20 bp, Artificial Sequence
  Description of Artificial Sequence Synthetic polynucleotide sequence
gcaagctgggcaatgtttat

SEQ ID NO: 22 - DNA, 20 bp, Artificial Sequence
  Description of Artificial Sequence Synthetic polynucleotide sequence
gtccggtccttgataggtca

SEQ ID NO: 23 - DNA, 20 bp, Artificial Sequence
  Description of Artificial Sequence Synthetic polynucleotide sequence
accacccaagagtggaactg

SEQ ID NO: 24 - DNA, 20 bp, Artificial Sequence
  Description of Artificial Sequence Synthetic polynucleotide sequence
tgtcgtgtcgaggtatgtgg

SEQ ID NO: 25 - DNA, 18 bp, Artificial Sequence
  Description of Artificial Sequence Synthetic polynucleotide sequence
gggcaatcgtcctcactg

SEQ ID NO: 26 - DNA, 20 bp, Artificial Sequence
  Description of Artificial Sequence Synthetic polynucleotide sequence
caagggcagtcttgattagg

SEQ ID NO: 27 - DNA, 20 bp, Artificial Sequence
  Description of Artificial Sequence Synthetic polynucleotide sequence
aacgttgaatcccgtttttg

SEQ ID NO: 28 - DNA, 21 bp, Artificial Sequence
  Description of Artificial Sequence Synthetic polynucleotide sequence
gcaccacaaaggaacgtctaa

SEQ ID NO: 29 - DNA, 20 bp, Artificial Sequence
  Description of Artificial Sequence Synthetic polynucleotide sequence
attcaaacggatgcgaactg

SEQ ID NO: 30 - DNA, 20 bp, Artificial Sequence
  Description of Artificial Sequence Synthetic polynucleotide sequence
acattgctggacaaacgaca

SEQ ID NO: 31 - DNA, 20 bp, Artificial Sequence
  Description of Artificial Sequence Synthetic polynucleotide sequence
cagcaagtaacggtggaaca

SEQ ID NO: 32 - DNA, 20 bp, Artificial Sequence
  Description of Artificial Sequence Synthetic polynucleotide sequence
acgtggcccatttgtcttag

SEQ ID NO: 33 - DNA, 20 bp, Artificial Sequence
  Description of Artificial Sequence Synthetic polynucleotide sequence
cccacctcgtcgttagtcat

SEQ ID NO: 34 - DNA, 20 bp, Artificial Sequence
  Description of Artificial Sequence Synthetic polynucleotide sequence
gtgctcggtgatccagaagt

SEQ ID NO: 35 - DNA, 18 bp, Artificial Sequence
  Description of Artificial Sequence Synthetic polynucleotide sequence
cctcatcctccaacaacc

SEQ ID NO: 36 - DNA, 23 bp, Artificial Sequence
  Description of Artificial Sequence Synthetic polynucleotide sequence
gctcaacctcgttttccccatac